SEQUENCE LISTING (113 sequences)

SEQ ID NO: 1 - protein, 53 aa, NEISSERIA GONORRHOEAE
MSFHPETAYNGGGETEPYGPSPEEIKYRQSPETAETRRMTEKQAEGHIKSIIR

SEQ ID NO: 2 - protein, 102 aa, NEISSERIA GONORRHOEAE
MNKNIAAALAGALSLSLAAGAVAAHKPASNATGVQKSAQGSCGASKSAEGSCGAAASKAGEGKCGEGKCGATVKKAHKHTKASKAKAKSAEGKCGEGKCGSK

SEQ ID NO: 3 - protein, 109 aa, NEISSERIA GONORRHOEAE
MPRQSCHSSPFPRKRESGTQTRQESIGKNNPTAVIPAQAGIQTHNVKAVYQKKPKPNALDFRLRGNDEELGSDERREQPRKKPPTPSDGIGNKNRTAETARERIAGRAR

SEQ ID NO: 4 - protein, 114 aa, NEISSERIA GONORRHOEAE
MKKLLIAAMMAAALAACSQEAKQEVKEAAQAVESDVKDTAASAAESAASAVEEAKGQVKDAAADAKASAEEAVTEAKDAAAETKEAVSEAAKDTLNKAADAAQEAADKMKDAAK

SEQ ID NO: 5 - protein, 216 aa, NEISSERIA GONORRHOEAE
MPFEPPSDGIARHPKSTIKMAKKPNKPFRLTPKLLIRAVLLICITAIGALAVGIVSTFNPNGDKTLQTEPQHTDSPRETEFWLPNGAVGQDAAQPEHHHAASSEPAQPDGTEESGSGLPPPAAPKKNRVKPRPSDAARAADSLTGTGTQAENTLKETPVLPTNAPHPEPRKETPEKQAQPKETPKEKETPKENHTKPDTPKNTPAKPHKEILDNLF

SEQ ID NO: 6 - protein, 228 aa, NEISSERIA GONORRHOEAE
MFDFGLGELIFVGIIALIVLGPERLPEAARTAGRLIGRLQRFVGSVKQELDTQIELEELRKVKQAFEAAAAQVRDSLKETDTDMQNSLHDISDGLKPWEKLPEQRTPADFGVDENGNPLPDTANTVSDGISDVMPSERSDTSAETLGDDRQTGSTAEPAETDKDRAWREYLTASAAAPVVQAVEVSYIDTAVETPVPHTTSLRKQAINRKRDFRPKHRAKPKLRVRKS

SEQ ID NO: 7 - protein, 283 aa, NEISSERIA GONORRHOEAE
MDKERILTPAVVFSVALLHLAIVALLWQAHKLPVIESGNVIEFVDLGDFGGGGGAPEGAGAPAAPEPQPAPDPPKPVEPPKPVLKPAVTKKADADIQQPKEKPKPEEKPKPEPEPEAKPAPKPAEKPAEKPSEKPAEHSGNASAKAGSEQGNGEGKGTGTKGDGTGRGEGSGKGSGGAKGEHGEGAGGSGGGTGVGSSKGNPLRANGSIPRPAYPALSMENDEQGMVVLSVLVSPGGHVESVKVVKSSGFSRLDNAARKAAQNGHFQANAWTEFKVPVKFELN

SEQ ID NO: 8 - protein, 289 aa, NEISSERIA GONORRHOEAE
MFMNKFSQSGKGLSGFFFGLILATVIIAGILLYLNQGGQNAFKIPAPSKQPAETEILKLKNQPKEDIQPEPADQNALSEPDVAKEAEQSDAEKAADKQPVADKADEVEEKAGEPEREEPDGQAVRKKALTEEREQTVREKAQKKDAETVKKQAVKPSKETEKKASKEEKKAAKEKVAPKPTPEQILNSGSIEKARSAAAKEVQKMKTFGKAEATHYLQMGAYADRRSAEGQRAKLAILGISSEVVGYQAGHKTLYRVQSGNMSADAVKKMQDELKKHGVASLIRAIEGK

SEQ ID NO: 9 - protein, 337 aa, NEISSERIA GONORRHOEAE
MKKNLPALALASMLILSGCDRLGIGNPFSGKEISCGSEETKEILVKLVRDNVEGETVKTFDDDAFKDQAFADIGISHIRRMVERLGITVDEVRTTEKTDTSSKLKCEAALKLDVPDDVVDYAVAANQSIGNSHKKTPDFFEPYYRKEGAYYVKTISYSVQPTDDKSKIFAELSQAHDIIHPLSELVSMALIKEPLDKAKQRNEKLEAAEATAQEAREAEEAAAQEALGREQEAARVSEWEERYKLSRSEFEQFWKGLPQTVQNKLQASQKTWKSGMDKICANNAKAEGETPNGIKVSELACKTAETEARLEELHNRKKALIDEMVREEDKKELPKRL

SEQ ID NO: 10 - protein, 346 aa, NEISSERIA GONORRHOEAE
MSENKQNEVLTGYEQLKRRNRRRLVTASSLVAASCILLAAALSSDPADSNPAPQAGETGATESQTANTAQTPALKSAAENGETAADKPQDLAGEDKPSAADSEISEPENVGAPLVLINDRLEDSNIKGLEESEKLQQAETAKTEPKQAKQRAAEKVSATADSTDTVAVEKPKRTAEPKPQKAERTAEAKPKAKETKTAEKVADKPKTAAEKTKPDTAKSDSAVKEAKKADKAEGKKTAEKDRSDGKKHETAQKTDKADKTKTAEKEKSGKAGKKAAIQAGYAEKERALSLQRKMKAAGIDSTITEIMTDNGKVYRVKSSNYKNARDAERDLNKLRVHGIAGQVTNE

SEQ ID NO: 11 - protein, 377 aa, NEISSERIA GONORRHOEAE
MAGLSNRQRRTKLSQWYNQCQTSQHLHNLRRTAKPTNHPSSSQSSPSESNSSRRQNYPYCRRCGEQSNINITGSGVSGRAGTGLIADKQIHLQSAEQSNTERSQNKSAGWNAGAAVSFGQGGWSLGVAAGGNVGKGYGNGDSVTHRHSHIGDKGSQTLIQSGGDTIIKGAQVRGKGVQVNAKNLSIQSVQDRETYQSKQQNAGAQVTVGYGFSASGDYSQSKIRADHASVTEQSGIYAGEDSYQIKVGNHTGLKGGIITSSQSAKDKGKNRFSTGTLAGSDIQNYSQYEGKSFGLGASVAVSGKTLGQGAKNKPQDKHLTSIADKNGASSSVGYGSDSDSQSSITKSGINTQKHSNHRRSRTNQADRQNSGTNQSRY

SEQ ID NO: 12 - protein, 398 aa, NEISSERIA GONORRHOEAE
MGLFEPSAGDFWEMKEKEKKEKARKGAEERERAAAQAHRADAVRRTVANYEAGPARYRNVMDLSRNNIEDGARRLRRAGAFERGADAGLGFSGGDKALSPDARAGADFARRDTRPTDAGGRTPPPLGFDGNVYRDGKPVRDFDAQRPLVSAGPDALSPEERELYRRATTPYAGALNGQLTAAQLNAARGIVAEHNKNAAVRELGRERLAAAAAENAANREAVLQKGRFDAAVKANEGALNREMAQRNADRAFDVQQAELGMKRQGFEMKREADALELEDRKRIADLTRAYGFAKSDGQRGEIARQIDALNGKFERQGEKGFDPNVFKTISYEVADPDTGLTAKREGIVDLRTGKPLDVEFAGEREKRYAALGFKPNGQKTAGGKIIYENEKGEKRVEQ

SEQ ID NO: 13 - protein, 422 aa, NEISSERIA GONORRHOEAE
MKQKKTVQCILLGFAAASMHAQGAAAANSGTIEKTDKYTLVLAKQGQENNYTLNGGTEVKPLNSLIIAANGGTNNITIKGKLADGPADAPPTIDNNSIERNINKNGYTYAWQNWSGAVMLVDQSYEGENKVTFENVTIAAHNAPAGILSDDRHKSSSLAPAMLAFKGRNTINMDADSNANSSNEGILLLNNGEKMGEYRLVSEEGSTLNINIKSGKDKGQGITANHYGNSDINFNKASPNITTMEFKGDVNIKIDRNGQEEAESNGFGFYSSRKLGNKKQIPEGSKMEAIFRGNVDIVATPVYDEQGRPKSIGSAFAIDGKYSKVEVVGGEGKVVKIKGDIFAYNGGSVSVNLANKDSYFEGEAHIGKRSFAKGKDMFALTVDADGYELTPDTKSIEKKKKELNVRGCTRLSLNSTPIPQDF

SEQ ID NO: 14 - protein, 426 aa, NEISSERIA GONORRHOEAE
MFKRSVIAMACIFPLSACGGGGGGSPDVKSADTPSKPAAPVVAENAGEGVLPKEKKDEEAAGGAPQADTQDATAGEGSQDMAAVSAENTGNGGAATTDNPKNEDAGAQNDMPQNAAESANQTGNNQPAGSSDSAPASNPAPANGGSDFGRTNVGNSVVIDGPSQNITLTHCKGDPCNGDNLLDEEAPPKSEFESLSDEEKIKKYKKDGEKFTGLVAIKVENNGLNKYTIIYQAQPTRSARSRRSLPAEIPLIPVNQADTLIVDGEAVSLTGHSGNIFAPEGNYRYLTYGAEKLPGGSYALRVQGEPAKGEMLVGTAVYNGEVLHFHMENGRPYPSGGRFAAKVDFGSKSVDGIIDSGDDLHMGTQKFKAAIDGNGFKGTWTENGGGDVSGRFYGPAGEEVAGKYSYRPTDAEKGGFGVFAGKKDRD

SEQ ID NO: 15 - protein, 439 aa, NEISSERIA GONORRHOEAE
MKPLRRLTNLLAACAVAAAALIQPALAADLAQDPFITDNTQRQHYEPGGKYHLFGDPRGSVSDRTGKINVIQDYTHQMGNLLIQQAAIQGNLGYTVRFSGHGHEEHAPFDNHAADSASEEKGNVDDGFTVYRLNWEGHEHHPADAYDGPKGGNYPKPTGARDEYTYHVNGTARSIKLNPTDTRSIRQRISDNYNNLGSNFSDRADEANRKMFEHNAKLDRRGNSMEFVNGVAAGALNPFISAGEALGIGDILYGTGYAIDKAAMRNIAPLPAEGKFAVIGGLGSAAGFEKNTREAVDRWIQENPNAAETVEAVFNVAAAAKVAKLAKAAKPGKAAVSGDFSKSYTCSFHGSTLVRTADGYKAIAHIQAGDRVLSKDEASGETGYKPVTARYGNPYQETVYIKVSDGIGNSQTLISNRIHPFYSDGKWIKAEDLKAGSRL

SEQ ID NO: 16 - protein, 468 aa, NEISSERIA GONORRHOEAE
MNLPIQKFMMLFAAAISLLQIPISHANGLDARLRDDMQAKHYEPGGKYHLFGNGRGSVKNRVCAVQTFDATAVGPILPITHERTGFEGIIGYETHFSGHGHEVHSPFDNHDSKSTSDFSGGVDGGFTVYQLHRTGSEIHPEDGYDGPQGSDYPPPGGARDIYSYYVKGTSTKTKINTVPQAPFSDRWLKENAGAASGFLSRADEAGKLIWENDPDKNWRANRMDDIRGIVQGAVNPFLTGFQGLGVGAITDSAVNPVTDTAAQQTLQGINDLGNLSPEAQLAAASLLQDSAFAVKDGINSARQWADAHPNITATAQTALAVAEAAGTVWRGKKVELNPAKWDWVKNTGYKKPAARHMQTVDGEMAGGNRPLESKNTVTTNNFFENTGYTEKVLRQASNGDYHGFPQSVDAFSENGTVIQIVGGDNIVRHKLYIPGSYKGKDGNFEYIREADGKINHRLFVPNQQLPEK

SEQ ID NO: 17 - protein, 498 aa, NEISSERIA GONORRHOEAE
MNLPIQKFMMLFAAAISLLQIPISHANGLDARLRDDMQAKHYEPGGKYHLFGNGRGSVKNRVCAVQTFDATAVGPILPITHERTGFEGIIGYETHFSGHGHEVHSPFDNHDSKSTSDFSGSVDGGFTVYQLHRTGSEIHPADGYDGPQGGGYPEPQGARDIYSYHIKGTSTKTKINTVPQAPFSDRWLKENAGAASGFLSRADEAGKLIWENDPDKNWRANRMDDIRGIVQGAVNPFLTGFQGLGVGAITDSAVNPVTYAAARKTLQGIHNLGNLSPEAQLAAASLLQDSAFAVKDGINSARQWADAHPNITATAQTALAVAEAAGTVWGGKKVELNPAKWDWVKNTGYKKPAARHMQTVDGEMAGGNRPPKSITSEGKANAATYPKLVNQLNEQNLNNIAAQDPRLSLAIHEGKKNFPIGTATYEEADRLGKIWVGEGARQTSGGGWLSRDGTRQYRPPTEKKSQFATTGIQANFETYTIDSNEKRNKIKNGHLNIR

SEQ ID NO: 18 - protein, 522 aa, NEISSERIA GONORRHOEAE
MKHRTFFSLCAKFGCLLALGACSPKIVDAGTATVPHTLSTLKTADNRPASVYLKKDKPTLIKFWASWCPLCLSELGQAEKWAQDAKFSSANLITVASPGFLHEKKDGEFQKWYAGLNYPKLPVVTDNGGTIAQNLNISVYPSWALIGKDGDVQRIVKGSINEAQALALIRNPNADLGSLKHSFYKPDTQKKDSAIMNTRTIYLAGGCFWGLEAYFQRIDGVVDAVSGYANGNTENPSYEDVSYRHTGHAETVKVTYDADKLSLDDILQYYFRVVDPTSLNKQGNDTGTQYRSGVYYTDPAEKAVIAAALKREQQKYQLPLVVENEPLKNFYDAEEYHQDYLIKNPNGYCHIDIRKADEPLPGKTKAAPQGKGFDAATYKKPSDAELKRTLTEEQYQVTQNSATEYAFSHEYDHLFKPGIYVDVVSGEPLFSSADKYDSGCGWPSFTRPIDAKSVTEHDDFSFNMRRTEVRSRAADSHLGHVFPDGPRDKGRLRYCINGASLKFIPLEQMDAAGYGALKGKVK

SEQ ID NO: 19 - protein, 527 aa, NEISSERIA GONORRHOEAE
MRAITSLLVMICHFMLITSASAAALRESAACTRTSSVCVDGPSTKNINGVDVTKDCWEYKEEYQCLEKDSADYCAPLKDPSAKCEVQGQTCLEQSNEGECLRYTHKYSCDVDLRTLHQGRLPTKVEEMEHTHLISSQWDESSCQVQGKKCKAVATECLEPGSTKTINGVPVTRDCWKERRTVQCTDGSDSETCSAYTSSDQCRLIGDKCTHQLPDGTCQAREKQFECTEKGETTKEVSGCQDRDFAKTMTTMEFARETQRFYDPEKQRFFNGEAGQCSIKLDGALDSVFGGDCCRTKADPGKFVDFAVQTGTTMATTYFMASVASHYTFTTMFVSSAAQAMGTTLSAAGGITGTSQIGALGFSAAGQQGMGVIVGFNPAVFAAAIAVIAIQQWLKCPQSEILVAMKRKADLCHYVGSYCGSKILGACVTMIESQCCFISKLAKIVNVGGKEQLKRGWGTPENPKCEGFTAQELEQLDFSKLDLSGFYEEIYANMDNVAKQGQKVSQKIREASVNGKNLEVKNYYEYQ

SEQ ID NO: 20 - protein, 576 aa, NEISSERIA GONORRHOEAE
MKPLRRLIKLLAACAVAAAALIQPALAADLAQDPFITDNTQRQHYEPGGKYHLFGDPRGSVSDRTGKINVIQDYTHQMGNLLIQQANINGTIGYHTRFSGHGHEEHAPFDNHAADSASEEKGNVDDGFTVYRLNWEGHEHHPADAYDGPKGGNYPKPTGARDEYTYHVNGTARSIKLNPTDTRSIRQRISDNYNNLGSNFSDRADEANRKMFEHNAKLDRRGNSMEFVNGVAAGALNPFISAGEALGIGDILYGTRYAIDKAAMRNIAPLPAEGKFAAIGGLGSVAGFEKNTREAVDRWIQENPNAAETVEAVFNVAAAAKVAKLAKAAKPGKAAVSGDFSKSYTCSFHGSTLVRTADGYKAIAHIQAGDRVLSKDEASGETGYKPVTARYGNPYQETVYIKVSDGIGNSQTLISNRIHPFYSDGKWIKAEDLKAGSRLFAENGAEQTVQSVTVKPEPLKAYNLTVADWHTYFVKGSQAETEGVWVHNDCPPKPKPTNHAQQRKEEAKNDSHRSVGDSNRVVREGKQYLDSDTGNHVYVKGDKVVILTPDGRQVTQFKNSKANTSKRVKNGKWTPK

SEQ ID NO: 21 - protein, 598 aa, NEISSERIA GONORRHOEAE
MQYKPLLLALMLVFSAPAVAAHDAAHNRSAEVKKQAKNKKEQPEAAEGKKEKGKNAAVKDKKTGGKEAAKEFKKTAKNRKEAEKEATSRQSARKGREGDKESKAEHKKAHGKPVSGSKEKNAKTQPENKQGKKGAKGQGNPRKGGKAEKDTVSANKKARSDKNGKAVKQDKKYREEKNAKTDSDELKAAVAAATNDVENKKALLKQSEGMLLHVSNSLKQLQEERIRQERIRQERIRQARGNLASVNRKQREAWDKFQKLNTELNRLKTEIAATKAQISRFVSGNYKNSQPNAVALFLKNAEPGQKNRFLRYTRYVNASNREVVKDLEKQQKALAVQEQKINNELARLKKIQANVQSLLKKQGVTDAAEQTESRRQNAKISKDARKLLEQKGNEQQLNKLLSNLEKKKAEHRIQDAEAKRKLAEARLAAAEKARKEAAQQKAEARRAEMSNLTAEDRNIQAPSVMGIGSADGFSRMQGRLKKPVDGVPTGLFGQNRSGGDVWKGVFYSTAPATVESIAPGTVSYADELDGYGKVVVIDHGENYISIYAGLSEISAGKGYTVAAGSKIGTSGSLPDGEEGLYLQIRYQGQVLNPSGWIR

SEQ ID NO: 22 - protein, 628 aa, NEISSERIA GONORRHOEAE
MGISRKISLILSILAVCLPMHAHASDLANDPFIRQVLDRQHFEPDGKYHLFGSRGELAERSGHIGLGNIQSHQLGNLMIQQAAIKGNIGYIVRFSDHGHEVHSPFDNHASHSDSDEAGSPVDGFSLYRIHWDGYEHHPADGYDGPQGGGYPAPKGARDIYSYDIKGVAQNIRLNLTDNRSTGQRLADRFHNAGAMLTQGVGDGFKRATRYSPELDRSGNAAEAFNGTADIVKNIIGAAGEIVGAGDAVQGISEGSNIAVMHGLGLLSTENKMARINDLADMAQLKDYAAAAIRDWAVQNPNAAQGIEAVSNIFMAAIPIKGIGAVRGKYGLGGITAHPVKRSQMGAIALPKGKSAVSDNFADAAYAKYPSPYHSRNIRSNLEQRYGKENITSSTVPPSNGKNVKLADQRHPKTGVPFDGKGFPNFEKHVKYDTKLDIQELSGGGIPKAKPVFDAKPRWEVDRKLNKLTTREQVEKNVQETRRRSQSSQFKAHAQREWENKTGLDFNHFIGGDINKKGTVTGGHSLTRGDVRVIQQTSAPDKHGVYQATVEIKKPDGSWEVKTKKGGKVMTKHTMFPKDWDEARIRAEVTSAWESRIMLKDNKWQGTSKSGIKIEGFTEPNRTAYPIYE

SEQ ID NO: 23 - protein, 693 aa, NEISSERIA GONORRHOEAE
MNNPLVNQAAMVLPVFLLSACLGGGGSFDLDSVDTEAPRPAPKYKDVPSKKPEARKDQGGYGFAMRFKRRNWYPPSNPKENEIRLSEGNWEQTDDGEIKTPSKQKNIINALSGNEGVSLQDSSQQGEGISKVTDHHDFKYVWSGFFYKRIGITTKKDDLSNKIIEARNGPDGYIFYKGTDPSRKLPVSGSVEYKGTWDFLTDVKANQKFTGLGNTSTKSGDRYSAFSGELDYIVKKESDKKDGHVGLGLTTEITVDFGKKTLSGKLIKNNMVINNGDEPTTQYYSLEAQVTGNRFNGKAIATDKPKVNETKEHPFVSDSSSLSGGFFGPQGEELGFRFLSHDNKVAVVGSAKTKDKNANGNTAAAGTAGAAGMSSEDTKLTTVLDAVELTPDGKKVKNLDNFSDATQLVVDGIMIPLLPTESGNGQADKGENGKTAFIYETTYTPESDKKDTQTGMATNGVQTVSNTAGGTSGKTKTHYKVQACCSNLNYLKYGLLTRENSNSVMQTVRNSSQAAARTEQGAQSMFLQGERTDEKEIPKEQKVVYLGTWYGHIAANGTSWTGKASDQQSGNRAKFDVNFKDKKITGTLTAANRQAETFTISGMIDGNGFEGTAKTGNGGFALDANNTAATHKAHIAEAKVRGGFYGPNAEELGGWFAYPGNGQAKNAQASSGNENSAGSATVVFGAKRQQLVQ

SEQ ID NO: 24 - protein, 720 aa, NEISSERIA GONORRHOEAE
MNAPFFRLSLLSLTLAAGFAHAAENNANVALDTVTVKGDRQGSKIRTNIVTLQQKDESTATDMRELLKEEPSIDFGGGNGTSQFLTLRGMGQNSVDIKVDNAYSDSQILYHQGRFIVDPALVKVVSVQKGAGSASAGIGATNGAIIAKTVDAQDLLKGLDKNWGVRLNSGFAGNNGVSYGASVFGKEGNFDGLFSYNRNDEKDYEAGKGFRNDNGGKTVPYSALDKRSYLAKIGTTFGDGDHRIVLSHMKDQHRGIRTVREEFAVSEKNSRITIKRQAPSYRETTQSNTNLAYTGKDLGFVEKLDANAYVLEKKRYSADDKDNGYAGNVKGPNHTRIATRGMNFNFDSRLAEQTLLKYGINYRHQEIKPQAFLNSEFSIPIKEKKNGQEVDKPMEQQKKDRADEAIVRSYRLTNPTKTDTGAYIEAIHEIDGFTLTGGLRYDRFKVKTHDGKTVSSSSLNPSFGVIWQPREHWSFSASHNYAGRSPRLYDALQTHGKRGIISIADGTKAERARNTEIGFNYNDGTFAANGSYFRQTIKDALANPQNRHDSVAVREAVNAGYIKNHGYELGASYRTGGLTAKVGVSRSKPRFYDTHPKKLLSANPEFGAQTGRTWTASLAYRFKNPNLEIGWRGRYVQKATGSILAAGQKDRDGKLENVVRQGFGVNDVFANWKPLGKDTLNVNLSVNNVFDKFYYPHSQRWTNTLPGVGRDVRLGVNYKF

SEQ ID NO: 25 - protein, 792 aa, NEISSERIA GONORRHOEAE
MKLKQIASALMMLGISPLAFADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSDRFDRQKFAQDMEKVTDFYQNNGYFDFRILDTDIQTNEDKTRQTIKITVHEGGRFRWGKVSIEGDTNEVPKAELEKLLTMKPGKWYERQQMTAVLGEIQNRMGSAGYAYSEISVQPLPNAGTKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDIYGKAFDPRKASTSVKQYKTTTAGGGVRMGIPVTEYDRVNFGLAAEHLTVNTYNKAPKRYADFIKQYGKTDGADGSFKGLLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGRTYTAAENGNNKSVYSENAHKSTFTNELRYSAGGAVTWLSPLGPMKFSYAYPLKKKPEDEIQRFQFQLGTTF

SEQ ID NO: 26 - protein, 801 aa, NEISSERIA GONORRHOEAE
MARLFSLKPLVLALGFCFGTHCAADTVAAEEADGRVAEGGAQGASESAQASDLTLGSTCLFCSNESGSPERTEAAVQGSGEASVPEDYTRIVADRMEGQSQVKVRAEGSVIIERDGAVLNTDWADYDQSGDTVTVGDRFALQQDGTLIRGETLTYNLDQQTGEAHNVRMETEQGGRRLQSVSRTAEMLGEGRYKLTETQFNTCSAGDAGWYVKAASVEADRGKGIGVAKHAAFVFGGVPLFYTPWADFPLDGNRKSGLLVPSVSAGSDGVSLSVPYYFNLAPNFDATFAPGIIGERGATFDGQIRYLRPDYSGQTDLTWLPHDKKSGRNNRYQAKWQHRHDISDTLQAGVDFNQVSDSGYYRDFYGGEEIAGNVNLNRRVWLDYGGRAAGGSLNAGLSVQKYQTLANQSGYKDEPYAIMPRLSADWHKNAGRAQIGVSAQFTRFSHDGRQDGSRLVVYPGIKWDFSNSWGYVRPKLGLHATYYSLDSFGGKASRSVGRVLPVVNIDGGTTFERNTRLFGGGVVQTIEPRLFYNYIPAKSQNDLPNFDSSESSFGYGQLFRENLYYGNDRINAANSLSTAVQSRILDGATGEERFRAGIGQKFYFKDDAVMLDGSVGKNPRSRSDWVAFASGGIGGRFTLDSSIHYNQNDKRAEHYAVGAGYRPAPGKVLNARYKYGRNEKIYLQADGSYFYDKLSQLDLSAQWPLTRNLSAVVRYNYGFEAKKPIEMLAGAEYKSSCGCWGAGVYAQRYVTGENTYKNAVFFSLQLKDLSSVGRNPAGRMDVAVPGYIPAHSLSAGRNKRP

SEQ ID NO: 27 - protein, 809 aa, NEISSERIA GONORRHOEAE
MPIPFKPVLAAAAIAQAFPAFAADPAPQSAQTLNEITVTGTHKTQKLGEEKIRRKTLDKLLTNDEHDLVRYDPGISVVEGGRAGSNGFTIRGVDKDRVAINVDGLAQAESRSSEAFQELFGAYGNFNANRNTSEPENFSEVTITKGADSLKSGSGALGGAVNYQTKSASDYVSEDKPYHLGIKGGSVGKNSQKFSSITAAGRLFGLDALLVYTRRFGKETKNRSTEGDVEIKNDGYVFDPANPSPSRYLTYKATGVARSQPDPQEWVNKSTLFKLGYNFNDRNRIGWIFEDSRTDRFTNELSNLWTGTTTSAATGDYRHRQDVSYRRRTGVEYKNELEHGPWDSLKLRYDKQRIDMNTWTWDIPKNYDTNGINGEVYHSFRHIRQNTAQWTADFEKQLDFSKAVWAAQYGLGGGRGDNANSDYSYFAKLYDPKILASNQAKITMLIENRSKYKFAYWNNVFHLGGNDRFRLNAGIRYDKNSSSAKDDPKYTTAIRGQIPHLGSERAHAGFSYGTGFDWRFTKHLHLLAKYSTGFRAPTSDETWLLFPHPDFYLKTNPELKAEKAKNWELGLAGSGKAGSFKLSGFKTKYRDFIELTYMGVSSDDKNNPRYAPLSDGTALVSSPVWQNQNRTAAWVEGIEFNGTWNLDSIGLPKGLHTGLNVSYIKGKATQNNGKETPINALSPWTAVYSLGYDAPSKRWGVNAYAARTAAKKPSDTVHSNDDLNKPWPYAKHSKAYTLFDLSAYLNIGKQVTLRAAAYNITNKQYYTWESLRSVREFGTVNRVNNKTHAGIQRFTSPGRSYNFTIEAKF

SEQ ID NO: 28 - protein, 912 aa, NEISSERIA GONORRHOEAE
MQQQHLFRFNILCLSLMTALPAYAENVQAGQAQEKQLDTIQVKAKKQKTRRDNEVTGLGKLVKTADTLSKEQVLDIRDLTRYDPGIAVVEQGRGASSGYSIRGMDKNRVALTVDGLAQIQSYTAQAALGGTRTAGSSGAINEIEYENVKAVEISKGSNSVEQGSGALAGSVAFQTKTADDVIGEGRQWGIQSKTAYSGKNRGLTQSIALAGRIGGAEALLIRTGRHAGEIRAHEAAGRGVQSFNRLVPVDDASTYAYFIVEEECKNGGYEKCKAKKDVDGKDERQTVSTRDYTGPNRFLADPLSYESRSWLFRPGFRFENKRHYIGGILERTQQTFDTRDMTVPAFLTKAVFDENKKYGSIRGYGKYAGDHRYGGLITNSENGAQVGAEYGTGVFYDETHTKSRYGLEYVYTNADKDTWADYARLSYDRQGIGLDNHFQQTHCSADGSDKYCRPSADKPSSYYKSDRVIYGESHRLLQAAFKKSFDTAKIRHNLSVNLGYDRFGSDLRHQDYYYQHANRAYSSKTPPQNNGKKTSPNGSNTSPYWVSIGGGNVVTGQICLFGNNTYTDCTPRSINGKSYYAAVRDNVRLGRWADVGAGLRYDYRSTHSDDGSVSTGTHRTLSWNAGIVLKPADWLDLTYRTSTGFRLPSFAEMYGWRSGDKIKAVKIDPEKSFNKEAGIVFKGDFGNLEASWFDNAYRDLIVRGYEVQIKDGKEQVKGDPAYLNAQSARITGINILGKIDWNGVWDKLPEGWYSTFAYNRVRVRDIKKRADRTDIQSHLFDAIQPSRYVVGSGYDQPEGKWGVNGMLTYSKAKEITELLGSRALLNGNSRNTKATARRTRPWYIVDVSGYYTVKKHFTLRAGVYNLLNHRYVTWENVRQTAAGAVNQHKNVGVYNRYAAPGRNYTFSLEMKF

SEQ ID NO: 29 - protein, 919 aa, NEISSERIA GONORRHOEAE
MKRMLFNATQAEELRVAIVDGQNLLDLDIETLGKEQRKGNIYKGIITRIEPSLEACFVDYGTDRHGFLPFKEVSRSYFLGYEGGRARIQDVLKEGMEVIVQVEKDERGNKGAALTTFISLAGRYLVLMPNNPRGGGVSRRIEGEERQELKAAMAQLDIPNGMSIIARTAGIGRSAEELEWDLNYLKQLWQAIEEAGKAHHDPYLLFMESSLLIRAIRDYFRPDIGEILVDNQEVYDQVAEFMSYVMPGNAGRLKLYEDHTPLFSRFQIEHQIESAFSRSVSLPSGGAIVIDHTEALVSIDVNSARATRGADIEDTAFKTNMEAAEEVARQMRLRDLGGLVVIDFIDMENPKHQRDVENVLRDALKKDRARVQMGKLSRFGLLELSRQRLKPALGESSHAACPRCAGTGVIRGIESTALHVLRMVQEEAMKDNTGEVRAQVPVDVATFLLNEKRAELFAMEERLDVNVVLIPNIHLENPHYEINRIRTDDVEEDGEPSYKRVAEPEEDESAKPFGGEKAKAARPEPAVKGVRHTSPAPTAAPEKKTSWWDSFKAWLKRIFGGSETQAVPAAETSEKRSTANRSGSRANNRRQNPRRSKREGSKIEVREAAGKTAGQKARADKAETRNNGNRRRNERGDRATERANEAEIQSRNVQPAAPVADAAPPETEGQTGKRRRNGSRNERGQTAPETAAVAETAVQTAENTPPEPYTAEDKGSKPKSERNRRERDSRDAKERRERNNQRDRRQNGKKRNIPSAAKIEQYLNIHDTADKVRSAAAHVFGETDANAPITVSIADPLIATPVQTASSAVSNGDALIYDAAEKIRRAAADILPEGAAPKAAAQEMPSETATFTAAAEQARETAQTGGLVLIETDPAALKAWAAQPEVQAGRGLRRSEQPKPSEAATVPAEEMIQVETRQG

SEQ ID NO: 30 - protein, 922 aa, NEISSERIA GONORRHOEAE
MRSSFRLKPICFYLMGVMLYHHSYAEDAGRAGSEAQIQVLEDVHVKAKRVPKDKKVFTDARAVSTRQDIFKSGENLDNIVRSIPGAFTQQDKSSGIVSLNIRGDSGFGRVNTMVDGITQTFYSTSTDAGRAGGSSQFGASVDSNFIAGLDVVKGSFSGSAGINSLAGSANLRTLGVDDVVQGNNTYGLLLKGLTGTNSTKGNAMAAIGARKWLESGASVGVLYGHSRRGVAQNYRVGGGGQHIGNFGAEYLERRKQRYFVQEGGLKFNSNSGKWERDFQRPYWKTKWYQKYNDPQELQKYIEGHDKSWRENLAPQYDITPIDPSGLKQQSAGNLFKLEYDGVFNKYTAQFRDLNTKIGSRKIINRNYQFNYGLSLNPYTNLNLTAAYNSGRQKYPKGSKFTGWGLLKDFETYNNAKILDLNNTATFRLPRETELQTTLGFNYFHNEYGKNRFPEELGLFFDGPDQDNGLYSYLGRFKGDKGLLPQKSTIVQPAGSQYFNTFYFDAALKKDIYRLNYSTNAINYRFGGEYTGYYGSENEFKRAFGENSPAYKEHCDPSCGLYEPVLKKYGKKRANNHSVSISADFGDYFMPFAGYSRTHRMPNIQEMYFSQIGDSGVHTALKPERANTWQFGFNTYKKGLLKQDDILGLKLVGYRSRIDNYIHNVYGKWWDLNGDIPSWVGSTGLAYTIRHRNFKDKVHKHGFELELNYDYGRFFTNLSYAYQKSTQPTNFSDASESPNNASKEDQLKQGYGLSRVSALPRDYGRLEVGTRWLGNKLTLGGAMRYFGKSIRATAEERYIDGTNGGNTSNVRQLGKRSIKQTETLARQPLIFDFYAAYEPKKNLIFRAEVKNLFDRRYIDPLDAGNDAATQRYYSSFDPKDKDEDVTCNADKTLCNGKYGGTSKSVLTNFARGRTFLMTMSYKF

SEQ ID NO: 31 - protein, 943 aa, NEISSERIA GONORRHOEAE
MSNTTVEQFAAELKRPVEDLLKQLKEAGVSKNSGSDSLTLDDKQLLNAYLTKKNGSNGGTISIRRTKTEVSTVDGVKVETRKRGRTVNIPSAEELAAQVKAAQTQAAPVQPEQTAEDAVKARAEAAARAEARAKAEAEAAKLKAAKAGNKAKPAAQKPTEAKAETAPVAAETKPAEPKEKAVKPKHERNGKGKDAKKPAKPAAPAVPQPVVSAEEQAQRDEEARRAAALRAHQEALLKEKQERQARREAMKQQAEQQAKAAQEAKTGRQRPAKPAEKPQAAAPAVENKPVNPAKAKKEDRRNRDDEGQGRNAKGKGAKGGRDRNNARNGGDERVRGGKKGKKLKLEPNQHAFQAPTEPVVHEVLVPETITVADLAHKMAVKGVEVVKALMKMGMMVTINQSIDQDTALIVVEELGHIGKPAAADDPEAFLGEGAEAVEAEALPRPPVVTVMGHVDHGKTSLLDYIRRAKVVQGEAGGITQHIGAYHVKTPRGVITFLDTPGHEAFTAMRARGAKATDIVILVVAADDGVMPQTIEAIAHAKAAGVPIVVAVNKIDKDTANPERIRQELTQHEVIPDDWGGTVQFIDVSAKKGTNIDALLEAVLLEAEVLELTAPVDAPAKGIIVEARLDKGRGAVATLLVQNGTLKKGDMLLAGTAFGKIRAMVDENGKSITEAGPSIPVEILGLSDVPNAGEDAMVLADEKKAREIALFRQGKYRDVRLAKQQAAKLENMFNNMGETQAQSLSVIIKADVQGSYEALAGSLKKLSADEVKVNVLHSGVGGITESDVNLAIASGAFIIGFNVRADASSRKLAENENVEIRYYNIIYDAIDDVKAAMSGMLSPEKKEQVTGTVEIRQVISVSKVGNIAGCMVTDGVVKRDSHIRLIRNNVVIHTGELASLKRYKDDVKEVRMGFECGLMLKGYNEIMEGDQLECFDIVEVARTL

SEQ ID NO: 32 - protein, 1014 aa, NEISSERIA GONORRHOEAE
MFWIVLIVILLLALAGLFFVRAQSEREWMREVSAWQEKKGEKQAELPEIKDGMPDFPEFSLMLFHAVKTAVYWLFVGVVRFCRNYLAHESEPDRPVPPASANRADVPTASDGYSDSGNGTEEAETEAAEAAEEEAADTEDIATAVIDNRRIPFDRSIAEGLMQSESKTSPVRPVFKEITLEEATRALSSAALRETKKRYIDAFEKNGTAVPKVRVSDTPMEGLQIIGLDDPVLQRTYSRMFDADKEAFSESADYGFEPYFEKQHPSAFSAVKAENARNAPFRRHAGQEKGQAEAKSPDVSQGQSVSDGTAVRDARRRVSVNLKEPNKATVSAEARISRLIPESRTVVGKRDVEMPSETENVFTETVSSVGYGGPVYDEAADIHIEEPAAPDAWVVEPPEVPEVAVPEIDILPPPPVSEIYNRTYEPPAGFEQAQRSRIAETDHLAADVLNGGWQEETAAIADDGSEGAAERSSGQYLSETEAFGHDSQAVCPFEDVPSERPSCRVSDTEADEGAFQSEETGAVSEHLPTTDLLLPPLFNPEATQTEEELLENSITIEEKLAEFKVKVKVVDSYSGPVITRYEIEPDVGVRGNSVLNLEKDLARSLGVASIRVVETIPGKTCMGLELPNPKRQMIRLSEIFNSPEFAESKSKLTLALGQDITGQPVVTDLGKAPHLLVAGTTGSGKSVGVNAMILSMLFKAAPEDVRMIMIDPKMLELSIYEGITHLLAPVVTDMKLAANALNWCVNEMEKRYRLMSFMGVRNLAGFNQKIAEAAARGEKIGNPFSLTPDDPEPLEKLPFIVVVVDEFADLMMTAGKKIEELIARLAQKARAAGIHLILATQRPSVDVITGLIKANIPTRIAFQVSSKIDSRTILDQMGAENLLGQGDMLFLPPGTAYPQRVHGAFASDEEVHRVVEYLKQFGEPDYVDDILSGGGSEELPGIGRSGDGETDPMYDEAVSVVLKTRKASISGVQRALRIGYNRAARLIDQMEAEGIVSAPEHNGNRTILVPLDNA

SEQ ID NO: 33 - protein, 1075 aa, NEISSERIA GONORRHOEAE
MKAKRFKINAISLSIFLAYALTPYSEAALVRDDVDYQIFRDFAENKGKFFVGATDLSVKNKRGQNIGNALSNVPMIDFSVADVNKRIATVVDPQYAVSVKHAKAEVHTFYYGQYNGHNDVADKENEYRVVEQNNYEPHKAWGASNLGRLEDYNMARFNKFVTEVAPIAPTDAGGGLDTYKDKNRFSSFVRIGAGRQLVYEKGVYHQEGNEKGYDLRDLSQAYRYAIAGTPYKDINIDQTMNTEGLIGFGNHNKQYSAEELKQALSQDALTNYGVLGDSGSPLFAFDKQKNQWVFLGTYDYWAGYGKKSWQEWNIYKKEFADKIKQHDNAGTVKGNGEHHWKTTGTNSHIGSTAVRLANNEGDANNGQNVTFEDNGTLVLDQNINQGAGGLFFKGDYTVKGANNDITWLGAGIDVADGKKVVWQVKNPNGDRLAKIGKGTLEINGTGVNQGQLKVGDGTVILNQKADADKKVQAFSQVGIVSGRGTLVLNSSNQINPDNLYFGFRGGRLDANGNDLTFEHIRNVDEGARIVNHNTDHASTITLTGKSLITNPNSLSVHSIQNDYDEDDYSYYYRPRRPIPQGKDLYYKNYRYYALKSGGNVNAPMPENGVAENNDWVFMGYTQEEAKKNAMNHKNNQRISGFSGFFGEENEKGHNGALNLNFNGKSAQNRFLLTGGANLNGKISVTQGNVLLSGRPTPHARDFVNKSSARKDAHFSKNNEVVFEDDWINRTFKAAEIAVNQSASFSSGRNVSDITANITATDNAKVNLGYKNGDEVCVRSDYTGYVTCNTGNLSDKALNSFDATRINGNVNLSQNAALVLGKAALWGQIQGQGNSRVSLNQHSKWHLTGDSQVHNLSLADSHIHLNNASDAQSANKYHTIKINHLSGNGHFHYLTDLAKNLGDKVLVKESASGHYQLHVQNKTGEPNQEGLDLFDASSVQDRSRLFVSLANHYVDLGALRYTIKTENGITRLYNPYAGNRRPVKPAPSPAANTASQAQKATQTDGAQIAKPQDIVVAPPSPQANQAEEAKRQQAKAEQVKRQQAEAGRKSAELSAKQRAGEEERRQTAQSQPQRRK

SEQ ID NO: 34 - protein, 1468 aa, NEISSERIA GONORRHOEAE
MKTTDKRTTETHRKAPKTGRIRFSPAYLAICLSFGILPQARAGHTYFGINYQYYRDFAENKGKFAVGAKDIEVYNKKGELVGKSMTKAPMIDFSVVSRNGVAALAGDQYIVSVAHNGGYNNVDFGAEGSNPDQHRFSYQIVKRNNYKAGTNGHPYGGDYHMPRLHKFVTDAEPVEMTSYMDGWKYADLNKYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPHQNGKYFFNDNNNGAGKIDAKHKHYSLPYRLKTRTVQLFNVSLSETAREPVYHAAGGVNSYRPRLNNGENISFIDKGKGELILTSNINQGAGGLYFEGNFTVSPKNNETWQGAGVHISDGSTVTWKVNGVANDRLSKIGKGTLLVQAKGENQGSVSVGDGKVILDQQADDQGKKQAFSEIGLVSGRGTVQLNADNQFNPDKLYFGFRGGRLDLNGHSLSFHRIQNTDEGAMIVNHNQDKESTVTITGNKDITTTGNNNNLDSKKEIAYNGWFGEKDATKTNGRLNLNYQPEEADRTLLLSGGTNLNGNITQTNGKLFFSGRPTPHAYNHLGSGWSKMEGIPQGEIVWDNDWIDRTFKAENFHIQGGQAVVSRNVAKVEGDWHLSNHAQAVFGVAPHQSHTICTRSDWTGLTSCTEKTITDDKVIASLSKTDIRGNVSLADHAHLNLTGLATLNGNLSAGGDTHYTVTRNATQNGNLSLVGNAQATFNQATLNGNTSASDNASFNLSNNAVQNGSLTLSDNAKANVSHSALNGNVSLADKAVFHFENSRFTGKISGGKDTALHLKDSEWTLPSGTELGNLNLDNATITLNSAYRHDAAGAQTGSAADAPRRRSRRSLLSVTPPTSAESRFNTLTVNGKLNGQGTFRFMSELFGYRSGKLKLAESSEGTYTLAVNNTGNEPVSLEQLTVVEGKDNTPLSENLNFTLQNEHVDAGAWRYQLIRKDGEFRLHNPVKEQELSDKLGKAGETEAALTAKQAQLAAKQQAEKDNAQSLDALIAAGRNATEKAESVAEPARQAGGENAGIMQAEEEKKRVQADKDTALAKQREAETRPATTAFPRARRARRDLPQPQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTGNTFDDGIGNSARLAHGAVFGQYGIGRFDIGISAGAGFSSGSLSDGIRGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 35 - protein, 1977 aa, NEISSERIA GONORRHOEAE
MPDGLARQYADMAAKYRAKPSEAVGIDPDDGAVSAAAALAADSGAAVPSAVSDDMEARSVADDAPSGRSADADRGGVPSAYGNVRPGGAPRGAASVAPGGSAAAASGGIARVAPLPAGQYFDGLDTRGRKALAKEAGLDIKGVADFGQIAAPVRRKIEQAYHARIEADYQAASEAKQGYLPPPVRMADAVPVPKKGFSVPADALDKESRRRFDALPEWVRRHAQTVADYTADGIMRREAGMADMRGHYPEGLAESAGAVRAYRAQHPESADVLDRLNRAVYGYRRNNGWSVPLLSREGERLQGVRTALPDDGASEAVVGGGRGLTRALPTEDKGLAQDVRQDVRQGLTQGGRGLTPDAGADANAAALQGLPGSAVASGNAPARRQNLQVRARAEGAAPGLSASENLAGTDGGKRAPVAGKRPDTVLPVLNPQVAESAGRVSPKKRMADAAADFTRRLAADRRRPEKAGVPLGGGEYRFEHTDRRHIDALAGVPGRPGKGGMPEEFADMAGPSNSDGLVSDGRRYLKGREAETLRAGGLSEAVPSEPGRDYRPTQEARAPAKVMARPRDAAADGKPAGRAQPARAKDTPVAGKAAAAKNAATEKPSSDKVRNIEAGKSRFDGGKGKSAAAQGAATEKPSEKTGKAKPETFAKTASDNPEEARRKARVLQGGPVYTVKERQAPQGFKALREHAESIKKRLAESIGGLAERVDVAAVSETAPDKAQMLLSQRVEGWFDGRTGKITLVAENLTPERAVWAAWHELGHRGFAADGFAKYREELERADGNGLIRRIADAVQEGREGTGDAAASVRPAAVEEAVAELYAAQRTGGWAGIENRYGVKVGNGLKRGIAGVLARIGALLRRVLQRLAGKAGGAMSDADVFAMLADLHGNVEGARDAPWGGNHRAVMFARAEDGAAERSKSESLEKLRRAETIRISGREVPEGGNLREYKRNALEYGKSLRGPYVNKDTGREISLGRSGITEILRHDYKDAEHLQSIAAIPQIIENAVYIDTLPNEDLAKNGDIQGYEYYVSGLNVGGADYTVRAAVAVSRNGNRYYDHKLTKIEKGNLLSLLDRVSTTGASESKSPLSGIDDKRLLQILQDKDAGKGGIADFDTEAVRFSRAANIEAAIGRITGKKSDLRNALKDRWDASKGIQLQFLGRRQIEDIYGGVLDGLKEYGRLSELFGADANKAVTEADKVVREWGRLKEEDAKALADLMHDATLAKVDADPLMRKDAQKRLDGIRTALDIADGKIEKAEAAVASAGARIARADAAYNKAQRAADKAAYALEKAQEKHGREILADEADMRLRRLFYADSEAKRALRRAGADVAAESRAKTDAVRMLEQARADVKRLEKDEVGAQKALEGLALLNRRFAGLPDAAQRVYRKARDDYRAHFGQVRDALAERLARAGQDAETVRRLKERFDNELGGVYFPLARFGDYLVVVKDADGNSANVSRAETLSEAEKLRDALKADFGAGFKVSPVMKSRDYIRSRDAVGSGFMRELGEAVGMLDLDPAQRARLNDTLTQLYLNSLPDTSWAKHGIHRKGVPGFSDDARRAYAQNMGSGANYLAKLRYADRMAEQLDVMQDFVDGRKYEEGFDQRQLQRVADEMRKRHEAVMNPNPSKLAQALTGFGFLWMMGMSPASAVVNLSQTAMVAYPVMAAKWGYAGAARELLRASKQIGLRFGEKFNTIEDSLNGDEKAAFRKAADYGVIDLSQAHDLAGVANGDPGLAGSAWQKVMDKAAWLFHHAEKFNRQVTFVAAYRLAKRAGADSEAAFEQAKKATYDGHFDYAAQNRPRFMMGNAAKVVFLFKQYSQNILYALGRNAYLAFKGDKEARKTLAGLLVSHAMASGILGLPFVSTLLAVASMLGSDDDDPWDAEAALRNMLADAFGDKAGEVLAKGFSRLTPLDVSGRLGLNQLVFPDIQDGLEGKKWAESLVVGSTGAVVGAGIGAADGVRTRSSVPRTANTLSLMKSW

SEQ ID NO: 36 - DNA, 210 bp, Streptococcus pneumoniae
atgaaaatcaaagggcaaactagaaaactagccgcaggttgctcaaaacactgttttgaggttgtggatagaactgacgaagtcagctcaaaatactgttttgaggttgtagatagaactgacgaagtcagctcaaaacactgttttgaggttgtagatagaactgacgaagtcagtaaccatacctacggcaaggcgacgttgacgtga

SEQ ID NO: 37 - DNA, 651 bp, Streptococcus pneumoniae
atggaagcaattatcgagaaaatcaaagagtataaaatcatcgtcatctgtactggtctgggcttgcttgtaggaggatttttcctgctaaaaccagctccacaaacacctgtcaaagagacgaatttgcaggctgaagtcgcagctgtttccaaggatttggtatccgaaaaggaagtgaacaaggaagaaaaggaagaaccccttgaacaagatctaatcacagtagatgtcaaaggtgctgtcaaatcgccagggatttatgacttgcctgtaggtagtcgagtcaatgatgctgttcagaaggctggtggcttgacagagcaagcagacagcaagtcgctcaatctagctcagaaagttagtgatgaggctctggtttacgttcctactaagggagaagaagcagttagccaacagactggtttggggacagcttcttcaataagcaaggaaaagaaggtcaatctcaacaaggccagtctggaagaactcaagcaggtcaagggactgggaggaaaacgagctcaggacattatcgaccatcgtgaggcaaatggcaagttcaagtcagtagacgagctcaagaaggtctctggcattggtggcaaaacaatagaaaagcttaaagactatgttacagtggattaa

SEQ ID NO: 38 - DNA, 666 bp, Streptococcus pneumoniae
atgacgattctaggaaaagatacagttcaacaatctgcgaaaggtgaatctgtaactcaagaagctacaccagagtataagctagaaactacaccaggtggagataagggaggcaatactggaagctcagatgctaatgcgaatgaaggcggtggtagccaggcgggtggatcagctcacacaggttcacaaaactcagctcaatcacaagcttctaagcaattagctactgaaaaagaatcagctaaaaatgccattgaaaaagcagccaagaacaagcaggatgaaatcaaaggcgcaccgctttctgataaagaaaaagcagaacttttagcaagagtggaagcagaaaaacaagcagctctcaaagagattgaaaatgcgaaaactatggaagatgtgaaggaagcagaaacgattggagtgcaagccattgccatggttacagttcctaagagaccagtggctcctaatgctgctcctaagacaacaagtgcaccgcaagcaactgcaggaacaatgcaagatgttacctaccagtcacctgctggcaaacaattacctaacacaggttcagcatcaagtgcagcacttgctagtcttggtctagtggtggcaacaagtggttttgctttgctaggaagaaagactagacgtagaaaatag

SEQ ID NO: 39 - DNA, 942 bp, Streptococcus pneumoniae
atgaagaaaaaattattggcaggtgccatcacactattatcagtagcaactttagcagcttgttcgaaagggtcagaaggtgcagaccttatcagcatgaaaggggatgtcattacagaacatcaattttatgagcaagtgaaaagcaacccttcagcccaacaagtcttgttaaatatgaccatccaaaaagtttttgaaaaacaatatggctcagagcttgatgataaagaggttgatgatactattgccgaagaaaaaaaacaatatggcgaaaactaccaacgtgtcttgtcacaagcaggtatgactcttgaaacacgtaaagctcaaattcgtacaagtaaattagttgagttggcagttaagaaggtagcagaagctgaattgacagatgaagcctataagaaagcctttgatgagtacactccagatgtaacggctcaaatcatccgtcttaataatgaagataaggccaaagaagttctcgaaaaagccaaggcagaaggtgctgattttgctcaattagccaaagataattcaactgatgaaaaaacaaaagaaaatggtggagaaattacctttgattctgcttcaacagaagtacctgagcaagtcaaaaaagccgctttcgctttagatgtggatggtgtttctgatgtgattacagcaactggcacacaagcctacagtagccaatattacattgtaaaactcactaagaaaacagaaaaatcatctaatattgatgactacaaagaaaaattaaaaactgttatcttgactcaaaaacaaaatgattcaacatttgttcaaagcattatcggaaaagaattgcaagcagccaatatcaaggttaaggaccaagccttccaaaatatctttacccaatatatcggtggtggagattcaagctcaagcagtagtacatcaaacgaatag

SEQ ID NO: 40 - DNA, 942 bp, Streptococcus pneumoniae
atggagaacaaatttgtagaatttatcgaaaacaataaaaaagtaatcatttcatcagttgcagttggtgttgtattggtattagggtttggatggtattcatataaccaacaacaagcagaacaacaagcaaaaattgtacaattagaaaaagatagcaaatcagacaaagaacaagttgataaactatttgaatcatttgatgcatcttcagatgaatctatttctaaattaaaagaactatctgaaacttcacttaaaaccgatgcaggtaaagactatcttaataacaaagtcaaagaatcatctaaagcaattgtagattttcatttgcaaaaaggtttggcttatgatgttaaagattcagatgacaaatttaaagataaagcaactcttgaaacaaatgtaaaagaaattacaaaacaaattgattttatcaaaaaagttgatgaaacttttaaacaagagaatttggaagaaactcttaaatctctaaatgatcttgttgataaatatcaaaaacaaatcgaacttttgaagaaagaagaagaaaaagctgctgaaaaagctgctgaaaaagcaaaggaatcttctagtcaaagtaattcttctggtagtgcttctaatgagtcttataatggatcttccaattcaaatgtagattatagttcatctgaacaaactaatggatattcaaataattatggcggtcaagattattctggttcaggagatagttcaacaaatggtggatcatcagaacaatattcatctagcaattcaaacagcggagcaaataatgtctacagatataaaggcactggtgctgacggctatcaaagatactactacaaagatcataataatggagatgtgtatgatgacgatggaaattaccttgggaactttggtggcggcattgcagaacctagtcaacgctaa

SEQ ID NO: 41 - DNA, 1113 bp, Streptococcus pneumoniae
atgaagaaaagaatgttattagcgtcaacagtagccttgtcatttgccccagtattggcaactcaagcagaagaagttctttggactgcacgtagtgttgagcaaatccaaaacgatttgactaaaacggacaacaaaacaagttataccgtacagtatggtgatactttgagcaccattgcagaagccttgggtgtagatgtcacagtgcttgcgaatctgaacaaaatcactaatatggacttgattttcccagaaactgttttgacaacgactgtcaatgaagcagaagaagtaacagaagttgaaatccaaacacctcaagcagactctagtgaagaagtgacaactgcgacagcagatttgaccactaatcaagtgaccgttgatgatcaaactgttcaggttgcagacctttctcaaccaattgcagaagttacaaagacagtgattgcttctgaagaagtggcaccatctacgggcacttctgtcccagaggagcaaacgaccgaaacaactcgcccagttgaagaagcaactcctcaggaaacgactccagctgagaagcaggaaacacaagcaagccctcaagctgcatcagcagtggaagtaactacaacaagttcagaagcaaaagaagtagcatcatcaaatggagctacagcagcagtttctacttatcaaccagaagagacgaaaataatttcaacaacttacgaggctccagctgcgcccgattatgctggacttgcagtagcaaaatctgaaaatgcaggtcttcaaccacaaacagctgcctttaaagaagaaattgctaacttgtttggcattacatcctttagtggttatcgtccaggagacagtggagatcacggaaaaggtttggctatcgactttatggtaccagaacgttcagaattaggggataagattgcggaatatgctattcaaaatatggccagccgtggcattagttacatcatctggaaacaacgtttctatgctccattcgatagcaaatatgggccagctaacacttggaacccaatgccagaccgtggtagtgtgacagaaaatcactatgatcacgttcacgtttcaatgaatggataa

SEQ ID NO: 42 - DNA, 1113 bp, Streptococcus pneumoniae
atgaatgataagttaaaaatcttcttgttgctaggagtattttttctagccataaccggtttctatgttctattgatacgaaatgcagggcagacagatgcctcgcaaattgaaaaggcggcagttagccaaggaggaaaagcagtgaaaaaaacagaaattagtaaagacgcagacttgcacgaaatttatctagctggaggttgtttctggggagtggaggaatatttctcacgtgttcccggggtgacggatgccgtttcaggctatgcaaatggtagaggagaaacaaccaagtacgaattgattaaccaaacaggtcatgcagaaaccgtccatgtcacctatgatgccaagcaaatttctctcaaggaaatcctgcttcactatttccgcattatcaatccaaccagcaaaaataaacaaggaaatgatgtggggacccagtaccgtactggtgtttattacacagatgacaaggatttggaagtgattaaccaagtctttgatgaggtggctaagaaatacgatcaacctctagcagttgaaaaggaaaacttgaagaattttgtggtggctgaggattaccatcaagactatctcaagaaaaatccaaatggctactgccatatcaatgttaatcaggcggcctatcctgtcattgatgccagcaaatatccaaaaccaagtgatgaggaattgaaaaagaccctgtcacctgaggagtatgcagttacccaggaaaatcaaacagaacgagctttctcaaaccgttactgggataaatttgaatccggtatctatgtggatatagcaactggggaacctctcttttcatcaaaagacaaatttgagtctggttgtggctggcctagttttacccaacccatcagtccagatgttgtcacctacaaggaagataagtcctacaatatgacgcgtatggaagtgcggagccgagtaggagattctcaccttgggcatgtctttacggatggtccacaggacaagggcggcttacgttactgtatcaatagcctctctatccgctttattcccaaagaccaaatggaagaaaaaggctacgcttatttactagattatgttgattaa

SEQ ID NO: 43 - DNA, 1179 bp, Streptococcus pneumoniae
atgaagaaaaaaatcttagcgtcacttttattaagtacagtaatggtttctcaagtagctgttttaacaactgcgcatgcagaaacgactgatgacaaaattgctgctcaagataataaaattagtaacttaacagcacaacaacaagaagcccaaaaacaagttgaccaaattcaggagcaagtatcagctattcaagctgagcagtctaacttgcaagctgaaaatgatagattacaagcagaatctaagaaactcgagggtgagattacagaactttctaaaaacattgtttctcgtaaccaatcgttggaaaaacaagctcgtagtgctcaaacaaatggagccgtaactagctatatcaataccattgtaaactcaaaatcaattacagaagctatttcacgtgttgctgcaatgagtgaaatcgtatctgcaaacaacaaaatgttagaacaacaaaaggcagataaaaaagctatttctgaaaaacaagtagcaaataatgatgctatcaatactgtaattgctaatcaacaaaaattggctgatgatgctcaagcattgactacgaaacaggcagaactaaaagctgctgaattaagtcttgctgctgagaaagcgacagctgaaggggaaaaagcaagtctattagagcaaaaagcagcagctgaggcagaggctcgtgcagctgcggtagcagaagcagcttataaagaaaaacgagctagccaacaacaatcagtacttgcttcagcaaacactaacttaacagctcaagtgcaagcagtatctgaatctgcagcagcacctgtccgtgcaaaagttcgtccaacatacagtacaaacgcttcaagttatccaattggagaatgtacatggggagtaaaaacattggcaccttgggctggagactactggggtaatggagcacagtgggctacaagtgcagcagcagcaggtttccgtacaggttcaacacctcaagttggagcaattgcatgttggaatgatggtggatatggtcacgtagcggttgttacagctgttgaatcaacaacacgtatccaagtatcagaatcaaattatgcaggtaatcgtacaattggaaatcaccgtggatggttcaatccaacaacaacttctgaaggttttgttacatatatttatgcagattaa

SEQ ID NO: 44 - DNA, 1200 bp, Streptococcus pneumoniae
atgaagaaaaagaatggtaaagctaaaaagtggcaactgtatgcagcaatcggtgctgcgagtgtagttgtattgggtgctggggggattttactctttagacaaccttctcagactgctctaaaagatgagcctactcatcttgttgttgccaaggaaggaagcgtggcctcctctgttttattgtcagggacagtaacagcaaaaaatgaacaatatgtttattttgatgctagtaagggtgatttagatgaaatccttgtttctgtgggcgataaggtcagcgaagggcaggctttagtcaagtacagtagttcagaagcgcaggcggcctatgattcagctagtcgagcagtagctagggcagatcgtcatatcaatgaactcaatcaagcacgaaatgaagccgcttcagctccggctccacagttaccagcgccagtaggaggagaagatgcaacggtgcaaagcccaactccagtggctggaaattctgttgcttctattgacgctcaattgggtgatgcccgtgatgcgcgtgcagatgctgcggcgcaattaagcaaggctcaaagtcaattggatgcaacaactgttctcagtaccctagagggaactgtggtcgaagtcaatagcaatgtttctaaatctccaacaggggcgagtcaagttatggttcatattgtcagcaatgaaaatttacaagtcaagggagaattgtctgagtacaatctagccaacctttctgtaggtcaagaagtaagctttacttctaaagtgtatcctgataaaaaatggactgggaaattaagctatatttctgactatcctaaaaacaatggtgaagcagctagtccagcagccgggaataatacaggttctaaatacccttatactattgatgtgacaggcgaggttggtgatttgaaacaaggtttttctgtcaacattgaggttaaaagcaaaactaaggctattcttgttcctgttagcagtctagtaatggatgatagtaaaaattatgtctggattgtggatgaacaacaaaaggctaaaaaagttgaggtttcattgggaaatgctgacgcagaaaatcaagaaatcacttctggtttaacgaacggtgctaaggtcatcagtaatccaacatcttccttggaagaaggaaaagaggtgaaggctgatgaagcaactaattag

SEQ ID NO: 45 - DNA, 1272 bp, Streptococcus pneumoniae
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaataa

SEQ ID NO: 46 - DNA, 1329 bp, Streptococcus pneumoniae
atgaaatttagaaaattagcttgtacagtacttgcgggtgctgcggttcttggtcttgctgcttgtggcaattctggcggaagtaaagatgctgccaaatcaggtggtgacggtgccaaaacagaaatcacttggtgggcattcccagtatttacccaagaaaaaactggtgacggtgttggaacttatgaaaaatcaatcatcgaagcgtttgaaaaagcaaacccagatataaaagtgaaattggaaaccatcgacttcaagtcaggtcctgaaaaaatcacaacagccatcgaagcaggaacagctccagacgtactctttgatgcaccaggacgtatcatccaatacggtaaaaacggtaaattggctgagttgaatgacctcttcacagatgaatttgttaaagatgtcaacaatgaaaacatcgtacaagcaagtaaagctggagacaaggcttatatgtatccgattagttctgccccattctacatggcaatgaacaagaaaatgttagaagatgctggagtagcaaaccttgtaaaagaaggttggacaactgatgattttgaaaaagtattgaaagcacttaaagacaagggttacacaccaggttcattgttcagttctggtcaagggggagaccaaggaacacgtgcctttatctctaacctttatagcggttctgtaacagatgaaaaagttagcaaatatacaactgatgatcctaaattcgtcaaaggtcttgaaaaagcaactagctggattaaagacaatttgatcaataatggttcacaatttgacggtggggcagatatccaaaactttgccaacggtcaaacatcttacacaatcctttgggcaccagctcaaaatggtatccaagctaaacttttagaagcaagtaaggtagaagtggtagaagtaccattcccatcagacgaaggtaagccagctcttgagtaccttgtaaacgggtttgcagtattcaacaataaagacgacaagaaagtcgctgcatctaagaaattcatccagtttatcgcagatgacaaggagtggggacctaaagacgtagttcgtacaggtgctttcccagtccgtacttcatttggaaaactttatgaagacaaacgcatggaaacaatcagcggctggactcaatactactcaccatactacaacactattgatggatttgctgaaatgagaacactttggttcccaatgttgcaatctgtatcaaatggtgacgaaaaaccagcagatgctttgaaagccttcactgaaaaagcgaacgaaacaatcaaaaaagctatgaaacaatag

SEQ ID NO: 47 - DNA, 1395 bp, Streptococcus pneumoniae
atgagtaaaaaaagacgaaatcgtcataaaaaagaaggtcaagaaccgcaatttgattttgatgaagcaaaagagctaacagttggtcaagctattcgtaaaaatgaagaagtggaagcaggagtcttgcctgaggattccattttggacaagtatgttaagcaacacagagatgaaattgaggcggataagtttgcgactcgtcaatacaaaaaagaggagttcgttgaaactcagagtctggatgatttaattcaagagatgcgtgaggctgtagagaagtcagaagcttcttcggaggaagttccatcttctgaagacatcttactacccttgcctctggacgatgaggagcaaggcttggatcctctattgctagatgatgaaaatccaacagaaatgactgaagaagtggaagaggagcaaaacctttctcgtctggatcaagaggactcagaaaagaaaagtaaaaaaggctttattttgaccgttttggcgcttgtatcagtaattatttgtgtcagtgcttattatgtctaccgtcaagtggctcgttcgaccaaggaaattgaaacttctcaatcaactacagccaatcaatcagatgtggatgattttaatacactttatgacgccttttacacagatagcaataaaacggctttgaaaaatagccagtttgataaactgagtcaactcaaaaccttgcttgataaactggaaggtagtcgtgaacatacgcttgccaaatctaaatatgatagtctagcaacgcaaatcaaggctattcaagatgtcaatgcacaatttgaaaaaccagctattgtggatggtgtgttggataccaatgccaaagccaaatcggatgctaaatttacggatattaaaactggaaatacggagcttgataaagtgctagataaggctatcagtcttggtaagagccagcaaacaagtacttctagctcaagttcaagtcaaactagcagctcaagctctagtcaagcaagttcaaatacgactagtgagccaaaaccaagtagttcaaatgagactagaagtagtcgcagtgaagtcaatatgggtctctcgagtgcaggggttgctgttcaaagaagtgccagtcgtgttgcctataatcagtctgctattgatgatagtaataactctgcctgggattttgcggatggtgtcttggaacaaattctagcgacttcacgttcacgtggctatatcactggagaccaatatatccttgaacgtgtcaatatcgttaacggcaatggttattacaacctctataagccagatggaacctatctctttacccttaactgtaagacaggctactttgtcggaaatggcgctggtcatgcggatgacttggactactaa

SEQ ID NO: 48 - DNA, 1614 bp, Streptococcus pneumoniae
atggaaatcatgtcgcttgcgattgctgtttttgccgtcatcattggtttagtcattggatatgtcagcatctcagctaagatgaaatcatctcaggaagctgcagagttgatgcttttaaatgctgaacaagaagcaactaatttacgtggacaagctgagcgtgaagcggatttacttgttaatgaagccaaacgtgaaagcaagtctcttaaaaaagaagcactattggaggccaaagaagaagccagaaaataccgtgaagaagtggacgctgaattcaaatcagaacgtcaagaactcaaacaaatcgaaagtcgtttgacagagagagctactagccttgaccgtaaggacgacaatttgacgagtaaagaacaaacacttgaacaaaaagaacaaagtatttctgatagagcgaaaaaccttgatgcgcgtgaagagcaattagaggaagtcgaaagacaaaaagaagcagaactagagcgtattggtgcgctgtctcaggcagaagcacgagatattatcttggctcagacagaggaaaacttgaccagggagattgccagtcgcattcgcgaagctgagcaagaggtcaaggaacgttctgacaaaatggccaaggacatcttggttcaagctatgcaacgtatcgctggtgaatatgtagcggagtcaacaaactcaacagttcatctgccagacgatactatgaagggacgcattattggtcgtgaaggtcgtaacattcgtacctttgaaagtttgacaggggtcgatgtgattatcgacgatacaccagaagtggtgaccttgtcaggatttgatccgattcgtcgtgagattgcccgtatgactatggaaatgttgctcaaagatggtcgtatacatccagctcgtatcgaagagttggttgagaaaaaccgtcaagagattgacaataagattcgtgaatacggtgaggctgctgcctatgaaattggtgcgccaaaccttcatccagacttgatgaagattatgggacgtttgcagttccgtacttcatatggacaaaatgttttgcgccattcgattgaggttgctaagttggctggtatcatggcgagcgaacttggtgaaaatgcggctcttgcccgtcgtgctggattccttcacgatatcgggaaagccattgaccatgaggttgaaggtagccacgttgaaatcggtatggaattggcccgtaagtacaaggaacccccagttgtggtgaatacgattgctagtcaccacggagatgttgaagctgagagcgtgatagcagttatcgtcgctgcagcagatgccttgagcgcagcccgtccaggtgctcgtagtgagtctcttgaaagctacatcaagcgtctccatgatttggaagaaattgctaacggctttgaaggagtgcaaactagctttgcccttcaagcaggacgtgaaattcgtatcatggtcaatccaggaaaaatcaaggacgacaaagtcacaatcttggctcacaaagttcgtaagaaaattgaaaacaatctcgattatccaggaaatatcaaggtaaccgtgattcgcgagcttcgtgcagtagattatgctaaataa

SEQ ID NO: 49 - DNA, 1884 bp, Streptococcus pneumoniae
atgaaaaagaaattaactagtttagcacttgtaggcgcttttttaggtttgtcatggtatgggaatgttcaggctcaagaaagttcaggaaataaaatccactttatcaatgttcaagaaggtggcagtgatgcgattattcttgaaagcaatggacattttgccatggtggatacaggagaagattatgatttcccagatggaagtgattctcgctatccatggagagaaggaattgaaacgtcttataagcatgttctaacagaccgtgtctttcgtcgtttgaaggaattgggtgtccaaaaacttgattttattttggtgacccatacccacagtgatcatattggaaatgttgatgaattactgtctacctatccagttgaccgagtctatcttaagaaatatagtgatagtcgtattactaattctgaacgtctatgggataatctgtatggctatgataaggttttacagactgctgcagaaaaaggtgtttcagttattcaaaatatcacacaaggggatgctcattttcagtttggggacatggatattcagctctataattatgaaaatgaaactgattcatcgggtgaattaaagaaaatttgggatgacaattccaattccttgattagcgtggtgaaagtcaatggcaagaaaatttaccttgggggcgatttagataatgttcatggagcagaagacaagtatggtcctctcattggaaaagttgatttgatgaagtttaatcatcaccatgataccaacaaatcaaataccaaggatttcattaaaaatttgagtccgagtttgattgttcaaacttcggatagtctaccttggaaaaatggtgttgatagtgagtatgttaattggctcaaagaacgaggaattgagagaatcaacgcagccagcaaagactatgatgcaacagtttttgatattcgaaaagacggttttgtcaatatttcaacatcctacaagccgattccaagttttcaagctggttggcataagagtgcatatgggaactggtggtatcaagcgcctgattctacaggagagtatgctgtcggttggaatgaaatcgaaggtgaatggtattactttaaccaaacgggtatcttgttacagaatcaatggaaaaaatggaacaatcattggttctatttgacagactctggtgcttctgctaaaaattggaagaaaatcgctggaatctggtattattttaacaaagaaaaccagatggaaattggttggattcaagataaagagcagtggtattatttggatgttgatggttctatgaagacaggatggcttcaatatatggggcaatggtattactttgctccatcaggggaaatgaaaatgggctgggtaaaagataaagaaacctggtactatatggattctactggtgtcatgaagacaggtgagatagaagttgctggtcaacattattatctggaagattcaggagctatgaagcaaggctggcataaaaaggcaaatgattggtatttctacaagacagacggttcacgagctgtgggttggatcaaggacaaggataaatggtacttcttgaaagaaaatggtcaattacttgtgaacggtaagacaccagaaggttatactgtggattcaagtggtgcctggttagtggatgtttcgatcgagaaatctgctacaattaaaactacaagtcattcagaaataaaagaatccaaagaagtagtgaaaaaggatcttgaaaataaagaaacgagtcaacatgaaagtgttacaaatttttcaactagtcaagatttgacatcctcaacttcacaaagctctgaaacgagtgtaaacaaatcggaatcagaacagtag

SEQ ID NO: 50 - DNA, 1980 bp, Streptococcus pneumoniae
atgaaaaaaaatcgtgtatttgctacagcaggtcttgttttattagcagcaggtgtacttgcagcatgcagttcttcaaaatcatctgattcatcagcccctaaagcttatggctatgtttatacagcagacccagaaaccttggactacctgatttcaagtaaaaatagtacaacagtagtgacttcaaatgggattgatggtttattcactaacgataattacggtaatcttgctcctgcagttgcagaggattgggaagtctctaaggatggtttgacctacacttataagattcgtaaaggggttaaatggtttacctctgatggagaagaatatgcagaggtgacggctaaagatttcgtgaacggtttaaaacacgcagcagataaaaaatcagaagctatgtatttagctgaaaattcggttaaaggcttggcagattatctatcaggaacttcaacagatttttcaacagttggtgtcaaggcggttgatgattatacgttacaatacactttgaaccagcctgaaccgttctggaactctaagttgacctattctattttctggcctctgaatgaagaattcgaaacatcaaaaggaagcgattttgctaaaccaacagatccgacatccttgctttataatggtccattcttgttgaaagggttgactgcaaaatcttctgtagagtttgtaaaaaatgagcaatattgggataaagaaaatgtccacctagatactatcaatctagcttactatgatggatcagatcaggagtcgctagagcgtaacttcactagtggagcttatagttatgcccgtctttaccctaccagctccaactattctaaggttgcagaagaatacaaggacaatatctattacacacaatcaggctctgggattgctggtctgggtgtgaatattgatcgccaaagttacaactatacttctaaaactacagattcagagaaagtagctactaagaaggcattgcttaacaaagatttccgtcaagccttgaattttgctcttgatcgctcagcttactcagctcaaatcaatggtaaagatggagcagctttagcagttcgtaatttatttgtaaaaccagactttgtttcagctggtgagaagacctttggtgatttagtcgctgctcaacttcctgcttatggtgatgagtggaaaggtgtgaatttagctgatgggcaggatggtttattcaatgctgacaaggccaaggcagagtttgcgaaagctaagaaagctttagaagcagacggcgttcagtttcctattcatctggacgttccagtagaccaagcatcaaaaaactacatatctcgtattcagtcctttaaacaatctgtagaaacagttcttggtgttgaaaatgtcgttgttgatattcaacaaatgacaagtgatgaattccttaatattacttactatgctgccaatgcttcatctgaggattgggatgtatcaggaggagtttcatgggggccagactatcaagacccatctacttacctggatattttaaaaacaactagcagtgaaactacaaaaacatatttaggatttgataatccaaatagcccttcagtagttcaagttggtttgaaagaatacgataaattagttgatgaagctgccagagagacaagcgacttgaatgtccgttatgaaaaatatgcagcggctcaagcatggttgacagatagttcactctttattcctgctatggcttcttctggtgcagcaccagtgctttcacgaattgttccatttactggagcttctgcgcaaacaggctctaaggggtcagatgtttacttcaaatatttgaaatcacaagataaagtggtgactaaggaagagtatgaaaaagctcgtgaaaaatggttgaaagaaaaagctgaatcaaatgagaaagctcaaaaagaattggcaagtcatgtgaagtaa

SEQ ID NO: 51 - DNA, 1983 bp, Streptococcus pneumoniae
atgaaaagttcaaaactatttgcccttgcgggcgtgacattattggcggcgactactttagctgcatgctctggatcaggttcaagcactaaaggtgagaagacattctcatacatttatgagacagaccctgataacctcaactatttgacaactgctaaggctgcgacagcaaatattaccagtaacgtggttgatggtttgctagaaaatgatcgctacgggaactttgtgccgtctatggctgaggattggtctgtatccaaggatggattgacttacacttatactatccgtaaggatgcaaaatggtatacttctgaaggtgaagaatacgcggcagtcaaagctcaagactttgtaacaggattaaaatatgctgctgataaaaaatcagatgctctttaccttgttcaagaatcaatcaaagggttggatgcctatgtaaaaggggaaatcaaagatttctcacaagtaggaattaaggctctggatgaacagacagttcagtacactttgaacaaaccagaaagcttctggaattctaagacaaccatgggtgtgcttgcgccagttaatgaagagtttttgaattcaaaaggagatgattttgccaaagctacggatccaagtagtctcttgtataacggtccttatttgttgaaatccattgtgaccaaatcctctgttgaatttgcgaaaaatccgaactactgggataaggacaatgtgcatgttgacaaagttaaattgtcattctgggatggtcaagataccagcaaacctgcagaaaactttaaagatggtagccttacagcagctcgtctctatccaacaagtgcaagtttcgcagaacttgagaagagtatgaaggacaatattgtctatactcaacaagactctattacgtatctagttggtacaaatattgaccgtcagtcctataaatacacatctaagaccagcgacgaacaaaaggcatcgactaaaaaggctctcttaaacaaggatttccgtcaggctattgcctttggatttgaccgtacagcctatgcctctcagttgaatggacaaactggagcaagtaaaatcttgcgtaatctctttgtgccaccaacatttgttcaagcagatggtaaaaactttggcgatatggtcaaagagaaattggtcacttatggggatgaatggaaggatgttaatcttgcagattctcaggatggtctttacaatccagaaaaagccaaggctgaatttgctaaagctaaatcagccttacaagcagaaggagtccaattcccaattcatttggatatgccagttgaccaaacagcaactacaaaagttcagcgcgtccaatctatgaaacaatccttggaagcaactttaggagctgataatgtcattattgatattcaacaactacaaaaagacgaagtaaacaatattacatattttgctgaaaatgctgctggcgaagactgggatttatcagataatgtcggttggggtccagactttgccgatccatcaacctaccttgatattatcaaaccttctgtaggagaaagtactaaaacatatttagggtttgactcaggggaagataatgtagctgctaaaaaagtaggtctatatgactacgaaaaattggttactgaggctggtgatgagactacagatgttgctaaacgctatgataaatacgctgcagcccaagcttggttgacagatagtgctttgattattccaactacatctcgtacagggcgtccaatcttgtctaagatggtaccatttacaataccatttgcattgtcaggaaataaaggtacaagtgaaccagtcttgtataaatacttggaacttcaagacaaggcagtcactgtagatgaataccaaaaagctcaggaaaaatggatgaaagaaaaagaagagtctaataaaaaggctcaagaagatctcgcaaaacatgtgaaataa

SEQ ID NO: 52 - DNA, 1998 bp, Streptococcus pneumoniae
atgaaatcaatcaacaaatttttaacaatgcttgctgccttattactgacagcgagtagcctgttttcagctgcaacagtttttgcggctgggacgacaacaacatctgttaccgttcataaactattggcaacagatggggatatggataaaattgcaaatgagttagaaacaggtaactatgctggtaataaagtgggtgttctacctgcaaatgcaaaagaaattgccggtgttatgttcgtttggacaaatactaataatgaaattattgatgaaaatggccaaactctaggagtgaatattgatccacaaacatttaaactctcaggggcaatgccggcaactgcaatgaaaaaattaacagaagctgaaggagctaaatttaacacggcaaatttaccagctgctaagtataaaatttatgaaattcacagtttatcaacttatgtcggtgaagatggagcaaccttaacaggttctaaagcagttccaattgaaattgaattaccattgaacgatgttgtggatgcgcatgtgtatccaaaaaatacagaagcaaagccaaaaattgataaagatttcaaaggtaaagcaaatccagatacaccacgtgtagataaagatacacctgtgaaccaccaagttggagatgttgtagagtacgaaattgttacaaaaattccagcacttgctaattatgcaacagcaaactggagcgatagaatgactgaaggtttggcattcaacaaaggtacagtgaaagtaactgttgatgatgttgcacttgaagcaggtgattatgctctaacagaagtagcaactggttttgatttgaaattaacagatgctggtttagctaaagtgaatgaccaaaacgctgaaaaaactgtgaaaatcacttattcggcaacattgaatgacaaagcaattgtagaagtaccagaatctaatgatgtaacatttaactatggtaataatccagatcacgggaatactccaaagccgaataagccaaatgaaaacggcgatttgacattgaccaagacatgggttgatgctacaggtgcaccaattccggctggagctgaagcaacgttcgatttggttaatgctcagactggtaaagttgtacaaactgtaactttgacaacagacaaaaatacagttactgttaacggattggataaaaatacagaatataaattcgttgaacgtagtataaaagggtattcagcagattatcaagaaatcactacagctggagaaattgctgtcaagaactggaaagacgaaaatccaaaaccacttgatccaacagagccaaaagttgttacatatggtaaaaagtttgtcaaagttaatgataaagataatcgtttagctggggcagaatttgtaattgcaaatgctgataatgctggtcaatatttagcacgtaaagcagataaagtgagtcaagaagagaagcagttggttgttacaacaaaggatgctttagatagagcagttgctgcttataacgctcttactgcacaacaacaaactcagcaagaaaaagagaaagttgacaaagctcaagctgcttataatgctgctgtgattgctgccaacaatgcatttgaatgggtggcagataaggacaatgaaaatgttgtgaaattagtttctgatgcacaaggtcgctttgaaattacaggccttcttgcaggtacatattacttagaagaaacaaaacagcctgctggttatgcattactaactagccgtcagaaatttgaagtcactgcaacttcttattcagcgactggacaaggcattgagtatactgctggttcaggtaaagatgacgctacaaaagtagtcaacaaaaaaatcactatcccacaaacgggtggtattggtacaattatctttgctgtagcgggggctgcgattatgggtattgcagtgtacgcatatgttaaaaacaacaaagatgaggatcaacttgcttaa

SEQ ID NO: 53 - DNA, 2223 bp, Streptococcus pneumoniae
atgaaaaaaaatattaaacaatatgtaaccttaggtactgtagtggtattatcagcatttgttgctaactcagttgcagctcaggagactgaaacttctgaagtatcaacaccaaagttggtgcaacctgttgcaccaacgactccgatttcggaagtacaacctacatcggataactcttcggaagttactgtacaacctcgaacagttgaaactactgttaaggatccatcttctacagcggaagaaactcctgtcttagaaaaaaataatgttactttaacagggggcggagaaaatgttactaaagagttaaaggataaatttactagcggtgactttactgtagtgattaagtacaatcagtcaagtgagaaaggcttacaagctctgtttggaatatctaattccaaacccggtcaacaaaatagttatgtagatgtgttccttagagacaatggtgagttggggatggaagcgcgtgatacttcttccaataaaaataacctagtatccagacctgcttcagtttggggtaagtacaaacaagaggctgtgactaacactgttgcagtagtagcagattcagtcaaaaaaacatattctttatacgcaaatggtacaaaagtagtagaaaagaaagtggataatttcctaaacatcaaggatattaaaggtattgattactatatgcttgggggagtgaaacgtgcaggaaaaacggcgtttggttttaacggaacactagaaaatatcaaattctttaatagtgcattggatgaagaaactgttaaaaagatgacaacaaacgctgttactggacatttaatttatacggctaatgatacaacaggttctaactatttccgtattccagttctgtatacttttagcaatggtcgggtattttcaagcattgacgctcgttacggtggaactcatgatttcttgaataaaattaatattgctacaagttatagtgatgataatggtaagacatggactaaaccaaaattaacattggcattcgatgattttgcgccagtaccattagaatggcctcgtgaagttggtggacgtgacttacaaatcagcggtggtgcaacctatattgactctgttattgttgaaaaaaagaacaaacaagtactcatgtttgctgatgtgatgcctgctggagtaagttttagagaagcaactagaaaagattcaggttataaacaaattgatggtaattattaccttaaattaaggaaacaaggtgatactgattacaattatactattcgtgagaatggtactgtatacgacgatcgtaccaacagaccaactgaattttcagtagataaaaatttcggtattaaacaaaatggtaattatttgacggtagagcaatattcggtttcatttgaaaataataaaaagactgaatatcgtaatgggactaaagtccatatgaatatcttctacaaagatgcattgtttaaagtagtgccaacgaactatattgcatatatttctagcaatgatcatggcgaatcttggtctgcaccaactttattacctcctataatgggacttaatcggaatgcgccatatttaggtcctggacgtggaatcattgaaagctcaactggacgtattcttattccgtcttacactggtaaagagtctgcgttcatttatagtgacgataatggagcatcttggaaagttaaagtagtgccacttccttctagttggtcagcagaagcacaatttgtagaattgagtccaggagtaattcaagcatatatgcgtacaaataatggtaaaattgcatatttaacaagtaaagacgcaggtactacttggagtgcaccggaatatttgaaatttgtttcaaatccaagttatggaacacaattatcaatcatcaattatagccaattgattgatggtaaaaaggctgtcattttaagtactccaaactccacaaatggtcgtaaacacggacaaatttggattggtctaattaatgatgataatacaattgattggcgttatcatcacgacgttgattatagtaactatggatactcatattcaacattgacagagttaccaaatcatgaaattggattgatgtttgaaaaatttgattcatggtctcgtaatgaacttcatatgaaaaatgttgtaccatatataacatttaagattgaagatctgaaaaagaattaa

SEQ ID NO: 54 - DNA, 2235 bp, Streptococcus pneumoniae
atgaataagaaaaaaatgattttaacaagtctagccagcgtcgctatcttaggggctggttttgttacgtctcagcctacttttgtaagagcagaagaatctccacaagttgtcgaaaaatcttcattagagaagaaatatgaggaagcaaaagcaaaagctgatactgccaagaaagattacgaaacggctaaaaagaaagcagaagacgctcagaaaaagtatgaagatgatcagaagagaactgaggagaaagctcgaaaagaagcagaagcatctcaaaaattgaatgatgtggcgcttgttgttcaaaatgcatataaagagtaccgagaagttcaaaatcaacgtagtaaatataaatctgacgctgaatatcagaaaaaattaacagaggtcgactctaaaatagagaaggctaggaaagagcaacaggacttgcaaaataaatttaatgaagtaagagcagttgtagttcctgaaccaaatgcgttggctgagactaagaaaaaagcagaagaagctaaagcagaagaaaaagtagctaagagaaaatatgattatgcaactctaaaggtagcactagcgaagaaagaagtagaggctaaggaacttgaaattgaaaaacttcaatatgaaatttctactttggaacaagaagttgctactgctcaacatcaagtagataatttgaaaaaacttcttgctggtgcggatcctgatgatggcacagaagttatagaagctaaattaaaaaaaggagaagctgagctaaacgctaaacaagctgagttagcaaaaaaacaaacagaacttgaaaaacttcttgacagccttgatcctgaaggtaagactcaggatgaattagataaagaagcagaagaagctgagttggataaaaaagctgatgaacttcaaaataaagttgctgatttagaaaaagaaattagtaaccttgaaatattacttggaggggctgatcctgaagatgatactgctgctcttcaaaataaattagctgctaaaaaagctgagttagcaaaaaaacaaacagaacttgaaaaacttcttgacagccttgatcctgaaggtaagactcaggatgaattagataaagaagcagaagaagctgagttggataaaaaagctgatgaacttcaaaataaagttgctgatttagaaaaagaaattagtaaccttgaaatattacttggaggggctgattctgaagatgatactgctgctcttcaaaataaattagctactaaaaaagctgaattggaaaaaactcaaaaagaattagatgcagctcttaatgagttaggccctgatggagatgaagaagaaactccagcgccggctcctcaaccagagcaaccagctcctgcaccaaaaccagagcaaccagctccagctccaaaaccagagcaaccagctcctgcaccaaaaccagagcaaccagctccagctccaaaaccagagcaaccagctccagctccaaaaccagagcaaccagctaagccggagaaaccagctgaagagcctactcaaccagaaaaaccagccactccaaaaacaggctggaaacaagaaaacggtatgtggtatttctacaatactgatggttcaatggcaataggttggctccaaaacaacggttcatggtactacctaaacgctaacggcgctatggcaacaggttgggtgaaagatggagatacctggtactatcttgaagcatcaggtgctatgaaagcaagccaatggttcaaagtatcagataaatggtactatgtcaacagcaatggcgctatggcgacaggctggctccaatacaatggctcatggtactacctcaacgctaatggtgatatggcgacaggatggctccaatacaacggttcatggtattacctcaacgctaatggtgatatggcgacaggatgggctaaagtcaacggttcatggtactacctaaacgctaacggtgctatggctacaggttgggctaaagtcaacggttcatggtactacctaaacgctaacggttcaatggcaacaggttgggtgaaagatggagatacctggtactatcttgaagcatcaggtgctatgaaagcaagccaatggttcaaagtatcagataaatggtactatgtcaatggcttaggtgcccttgcagtcaacacaactgtagatggctataaagtcaatgccaatggtgaatgggtttaa

SEQ ID NO: 55 - DNA, 2460 bp, Streptococcus pneumoniae
atgaaaattaataaaaaatatctagcaggttcagtggcagtccttgccctaagtgtttgttcctatgagcttggacgttaccaagctggtcaggataagaaagagtctaatcgagttgcttatatagatggtgatcaggctggtcaaaaggcagaaaacttgacaccagatgaagtcagtaagagggaggggatcaacgccgaacaaattgttatcaagattacggatcaaggttatgtgacctctcatggagaccattatcattactataatggcaaggttccttatgatgccatcatcagtgaagagctcctcatgaaagatccgaattatcagttgaaggattcagacattgtcaatgaaatcaagggtggttatgtcattaaggtaaacggtaaatactatgtttaccttaaggatgcagctcatgcggataatattcggacaaaagaagagattaaacgtcagaagcaggaacgcagtcataatcataactcaagagcagataatgctgttgctgcagccagagcccaaggacgttatacaacggatgatgggtatatcttcaatgcatctgatatcattgaggacacgggtgatgcttatatcgttcctcacggcgaccattaccattacattcctaagaatgagttatcagctagcgagttagctgctgcagaagcctattggaatgggaagcagggatctcgtccttcttcaagttctagttataatgcaaatccagctcaaccaagattgtcagagaaccacaatctgactgtcactccaacttatcatcaaaatcaaggggaaaacatttcaagccttttacgtgaattgtatgctaaacccttatcagaacgccatgtggaatctgatggccttattttcgacccagcgcaaatcacaagtcgaaccgccagaggtgtagctgtccctcatggtaaccattaccactttatcccttatgaacaaatgtctgaattggaaaaacgaattgctcgtattattccccttcgttatcgttcaaaccattgggtaccagattcaagaccagaagaaccaagtccacaaccgactccagaacctagtccaagtccgcaaccagctccaagcaatccaattgatgagaaattggtcaaagaagctgttcgaaaagtaggcgatggttatgtctttgaggagaatggagtttctcgttatatcccagccaaggatctttcagcagaaacagcagcaggcattgatagcaaactggccaagcaggaaagtttatctcataagctaggaactaagaaaactgacctcccatctagtgatcgagaattttacaataaggcttatgacttactagcaagaattcaccaagatttacttgataataaaggtcgacaagttgattttgaggctttggataacctgttggaacgactcaaggatgtctcaagtgataaagtcaagttagtggaagatattcttgccttcttagctccgattcgtcatccagaacgtttaggaaaaccaaatgcgcaaattacctacactgatgatgagattcaagtagccaagttggcaggcaagtacacaacagaagacggttatatctttgatcctcgtgatataaccagtgatgagggggatgcctatgtaactccacatatgacccatagccactggattaaaaaagatagtttgtctgaagctgagagagcggcagcccaggcttatgctaaagagaaaggtttgacccctccttcgacagaccatcaggattcaggaaatactgaggcaaaaggagcagaagctatctacaaccgcgtgaaagcagctaagaaggtgccacttgatcgtatgccttacaatcttcaatatactgtagaagtcaaaaacggtagtttaatcatacctcattatgaccattaccataacatcaaatttgagtggtttgacgaaggcctttatgaggcacctaaggggtatactcttgaggatcttttggcgactgtcaagtactatgtcgaacatccaaacgaacgtccgcattcagataatggttttggtaacgctagcgaccatgttcaaagaaacaaaaatggtcaagctgataccaatcaaacggaaaaaccaagcgaggagaaacctcagacagaaaaacctgaggaagaaacccctcgagaagagaaaccgcaaagcgagaaaccagagtctccaaaaccaacagaggaaccagaagaatcaccagaggaatcagaagaacctcaggtcgagactgaaaaggttgaagaaaaactgagagaggctgaagatttacttggaaaaatccaggatccaattatcaagtccaatgccaaagagactctcacaggattaaaaaataatttactatttggcacccaggacaacaatactattatggcagaagctgaaaaactattggctttattaaaggagagtaagtaa

SEQ ID NO: 56 - DNA, 2520 bp, Streptococcus pneumoniae
atgaaaattaataaaaaatatctagcaggttcagtggcagtccttgccctaagtgtttgttcctatgaacttggtcgtcaccaagctggtcaggttaagaaagagtctaatcgagtttcttatatagatggtgatcaggctggtcaaaaggcagaaaacttgacaccagatgaagtcagtaagagggaggggatcaacgccgaacaaatcgtcatcaagattacggatcaaggttatgtgacctctcatggagaccattatcattactataatggcaaggtcccttatgatgccatcatcagtgaagagctcctcatgaaagatccgaattatcagttgaaggattcagacattgtcaatgaaatcaagggtggttatgttatcaaggtagatggaaaatactatgtttaccttaaggatgcagctcatgcggataatattcggacaaaagaagagattaaacgtcagaagcaggaacacagtcataatcacgggggtggttctaacgatcaagcagtagttgcagccagagcccaaggacgctatacaacggatgatggttatatcttcaatgcatctgatatcattgaggacacgggtgatgcttatatcgttcctcacggcgaccattaccattacattcctaagaatgagttatcagctagcgagttagctgctgcagaagcctattggaatgggaagcagggatctcgtccttcttcaagttctagttataatgcaaatccagctcaaccaagattgtcagagaaccacaatctgactgtcactccaacttatcatcaaaatcaaggggaaaacatttcaagccttttacgtgaattgtatgctaaacccttatcagaacgccatgtggaatctgatggccttattttcgacccagcgcaaatcacaagtcgaaccgccagaggtgtagctgtccctcatggtaaccattaccactttatcccttatgaacaaatgtctgaattggaaaaacgaattgctcgtattattccccttcgttatcgttcaaaccattgggtaccagattcaagaccagaacaaccaagtccacaatcgactccggaacctagtccaagtccgcaacctgcaccaaatcctcaaccagctccaagcaatccaattgatgagaaattggtcaaagaagctgttcgaaaagtaggcgatggttatgtctttgaggagaatggagtttctcgttatatcccagccaaggatctttcagcagaaacagcagcaggcattgatagcaaactggccaagcaggaaagtttatctcataagctaggagctaagaaaactgacctcccatctagtgatcgagaattttacaataaggcttatgacttactagcaagaattcaccaagatttacttgataataaaggtcgacaagttgattttgaggctttggataacctgttggaacgactcaaggatgtcccaagtgataaagtcaagttagtggatgatattcttgccttcttagctccgattcgtcatccagaacgtttaggaaaaccaaatgcgcaaattacctacactgatgatgagattcaagtagccaagttggcaggcaagtacacaacagaagacggttatatctttgatcctcgtgatataaccagtgatgagggggatgcctatgtaactccacatatgacccatagccactggattaaaaaagatagtttgtctgaagctgagagagcggcagcccaggcttatgctaaagagaaaggtttgacccctccttcgacagaccatcaggattcaggaaatactgaggcaaaaggagcagaagctatctacaaccgcgtgaaagcagctaagaaggtgccacttgatcgtatgccttacaatcttcaatatactgtagaagtcaaaaacggtagtttaatcatacctcattatgaccattaccataacatcaaatttgagtggtttgacgaaggcctttatgaggcacctaaggggtatactcttgaggatcttttggcgactgtcaagtactatgtcgaacatccaaacgaacgtccgcattcagataatggttttggtaacgctagcgaccatgttcgtaaaaataaggtagaccaagacagtaaacctgatgaagataaggaacatgatgaagtaagtgagccaactcaccctgaatctgatgaaaaagagaatcacgctggtttaaatccttcagcagataatctttataaaccaagcactgatacggaagagacagaggaagaagctgaagataccacagatgaggctgaaattcctcaagtagagaattctgttattaacgctaagatagcagatgcggaggccttgctagaaaaagtaacagatcctagtattagacaaaatgctatggagacattgactggtctaaaaagtagtcttcttctcggaacgaaagataataacactatttcagcagaagtagatagtctcttggctttgttaaaagaaagtcaaccggctcctatacagtag

SEQ ID NO: 57 - DNA, 2574 bp, Streptococcus pneumoniae
atgaaattcaatccaaatcaaagatatactcgttggtctattcgccgtctcagtgtcggtgttgcctcagttgttgtggctagtggcttctttgtcctagttggtcagccaagttctgtacgtgccgatgggctcaatccaaccccaggtcaagtcttacctgaagagacatcgggaacgaaagagggtgacttatcagaaaaaccaggagacaccgttctcactcaagcgaaacctgagggcgttactggaaatacgaattcacttccgacacctacagaaagaactgaagtgagcgaggaaacaagcccttctagtctggatacactttttgaaaaagatgaagaagctcaaaaaaatccagagctaacagatgtcttaaaagaaactgtagatacagctgatgtggatgggacacaagcaagtccagcagaaactactcctgaacaagtaaaaggtggagtgaaagaaaatacaaaagacagcatcgatgttcctgctgcttatcttgaaaaagctgaagggaaaggtcctttcactgccggtgtaaaccaagtaattccttatgaactattcgctggtgatggtatgttaactcgtctattactaaaagcttcggataatgctccttggtctgacaatggtactgctaaaaatcctgctttacctcctcttgaaggattaacaaaagggaaatacttctatgaagtagacttaaatggcaatactgttggtaaacaaggtcaagctttaattgatcaacttcgcgctaatggtactcaaacttataaagctactgttaaagtttacggaaataaagacggtaaagctgacttgactaatctagttgctactaaaaatgtagacatcaacatcaatggattagttgctaaagaaacagttcaaaaagccgttgcagacaacgttaaagacagtatcgatgttccagcagcctacctagaaaaagccaagggtgaaggtccattcacagcaggtgtcaaccatgtgattccatacgaactcttcgcaggtgatggcatgttgactcgtctcttgctcaaggcatctgacaaggcaccatggtcagataacggcgacgctaaaaacccagccctatctccactaggcgaaaacgtgaagaccaaaggtcaatacttctatcaagtagccttggacggaaatgtagctggcaaagaaaaacaagcgctcattgaccagttccgagcaaatggtactcaaacttacagcgctacagtcaatgtctatggtaacaaagacggtaaaccagacttggacaacatcgtagcaactaaaaaagtcactattaacataaacggtttaatttctaaagaaacagttcaaaaagccgttgcagacaacgttaaagacagtatcgatgttccagcagcctacctagaaaaagccaagggtgaaggtccattcacagcaggtgtcaaccatgtgattccatacgaactcttcgcaggtgatggtatgttgactcgtctcttgctcaaggcatctgacaaggcaccatggtcagataacggtgacgctaaaaacccagccctatctccactaggtgaaaacgtgaagaccaaaggtcaatacttctatcaattagccttggacggaaatgtagctggcaaagaaaaacaagcgctcattgaccagttccgagcaaacggtactcaaacttacagcgctacagtcaatgtctatggtaacaaagacggtaaaccagacttggacaacatcgtagcaactaaaaaagtcactattaacataaacggtttaatttctaaagaaacagttcaaaaagccgttgcagacaacgttaaggacagtatcgatgttccagcagcctacctagaaaaggccaagggtgaaggtccattcacagcaggtgtcaaccatgtgattccatacgaactcttcgcaggtgatggcatgttgactcgtctcttgctcaaggcatctgacaaggcaccatggtcagataacggcgacgctaaaaacccagctctatctccactaggtgaaaacgtgaagaccaaaggtcaatacttctatcaagtagccttggacggaaatgtagctggcaaagaaaaacaagcgctcattgaccagttccgagcaaacggtactcaaacttacagcgctacagtcaatgtctatggtaacaaagacggtaaaccagacttggacaacatcgtagcaactaaaaaagtcactattaagataaatgttaaagaaacatcagacacagcaaatggttcattatcaccttctaactctggttctggcgtgactccgatgaatcacaatcatgctacaggtactacagatagcatgcctgctgacaccatgacaagttctaccaacacgatggcaggtgaaaacatggctgcttctgctaacaagatgtctgatacgatgatgtcagaggataaagctatgctaccaaatactggtgagactcaaacatcaatggcaagtattggtttccttgggcttgcgcttgcaggtttactcggtggtctaggtttgaaaaacaaaaaagaagaaaactaa

SEQ ID NO: 58 - DNA, 2661 bp, Streptococcus pneumoniae
atgaaaaaagtaagaaagatatttcagaaggcagttgcaggactgtgctgtatatctcagttgacagctttttcttcgatagttgctttagcagaaacgcctgaaaccagtccagcgataggaaaagtagtgattaaggagacaggcgaaggaggagcgcttctaggagatgccgtctttgagttgaaaaacaatacggatggcacaactgtttcgcaaaggacagaggcgcaaacaggagaagcgatattttcaaacataaaacctgggacatacaccttgacagaagcccaacctccagttggttataaaccctctactaaacaatggactgttgaagttgagaagaatggtcggacgactgtccaaggtgaacaggtagaaaatcgagaagaggctctatctgaccagtatccacaaacagggacttatccagatgttcaaacaccttatcagattattaaggtagatggttcggaaaaaaacggacagcacaaggcgttgaatccgaatccatatgaacgtgtgattccagaaggtacactttcaaagagaatttatcaagtgaataatttggatgataaccaatatggaatcgaattgacggttagtgggaaaacagtgtatgaacaaaaagataagtctgtgccgctggatgtcgttatcttgctcgataactcaaatagtatgagtaacattcgaaacaagaatgctcgacgtgcggaaagagctggtgaggcgacacgttctcttattgataaaattacatctgattcagaaaatagggtagcgcttgtgacttatgcttccactatctttgatgggaccgagtttacagtagaaaaaggggtagcagataaaaacggaaagcgattgaatgattctcttttttggaattatgatcagacgagttttacaaccaataccaaagattatagttatttaaagctgactaatgataagaatgacattgtagaattaaaaaataaggtacctaccgaggcagaagaccatgatggaaatagattgatgtaccaattcggtgccacttttactcagaaagctttgatgaaggcagatgagattttgacacaacaagcgagacaaaatagtcaaaaagtcattttccatattacggatggtgtcccaactatgtcgtatccgattaattttaatcatgctacgtttgctccatcatatcaaaatcaactaaatgcattttttagtaaatctcctaataaagatggaatactattaagtgattttattacgcaagcaactagtggagaacatacaattgtacgcggagatgggcaaagttaccagatgtttacagataagacagtttatgaaaaaggtgctcctgcagctttcccagttaaacctgaaaaatattctgaaatgaaggcggctggttatgcagttataggcgatccaattaatggtggatatatttggcttaattggagagagagtattctggcttatccgtttaattctaatactgctaaaattaccaatcatggtgaccctacaagatggtactataacgggaatattgctcctgatgggtatgatgtctttacggtaggtattggtattaacggagatcctggtacggatgaagcaacggctactagttttatgcaaagtatttctagtaaacctgaaaactataccaatgttactgacacgacaaaaatattggaacagttgaatcgttatttccacaccatcgtaactgaaaagaaatcaattgagaatggtacgattacagatccgatgggtgagttaattgatttgcaattgggcacagatggaagatttgatccagcagattacactttaactgcaaacgatggtagtcgcttggagaatggacaagctgtaggtggtccacaaaatgatggtggtttgttaaaaaatgcaaaagtgctctatgatacgactgagaaaaggattcgtgtaacaggtctgtaccttggaacggatgaaaaagttacgttgacctacaatgttcgtttgaatgatgagtttgtaagcaataaattttatgataccaatggtcgaacaaccttacatcctaaggaagtagaacagaacacagtgcgcgacttcccgattcctaagattcgtgatgtgcggaagtatccagaaatcacaatttcaaaagagaaaaaacttggtgacattgagtttattaaggtcaataaaaatgataaaaaaccactgagaggtgcggtctttagtcttcaaaaacaacatccggattatccagatatttatggagctattgatcaaaatggcacttatcaaaatgtgagaacaggtgaagatggtaagttgacctttaaaaatctgtcagatgggaaatatcgattatttgaaaattctgaaccagctggttataaacccgttcaaaataagcctatcgttgccttccaaatagtaaatggagaagtcagagatgtgacttcaatcgttccacaagatataccagcgggttacgagtttacgaatgataagcactatattaccaatgaacctattcctccaaagagagaatatcctcgaactggtggtatcggaatgttgccattctatctgataggttgcatgatgatgggaggagttctattatacacacggaaacatccgtaa

SEQ ID NO: 59 - DNA, 2877 bp, Streptococcus pneumoniae
atgtctaagaaaagattgtacgaaatcgcaaaagaacttggaaaagaaagtaaagaagttgtagcgcgtgcaaaagagttgggcttggatgtgaaaagccactcatcaagtgtggaagaagctgtcgctgcaaaaattgctgccagctttaagcctgcagctgctccgaaagtagaagcaaaacctgcagccccaaaagtaagtgcagaaaagaaagccgaaaaatctgagccagctaaaccagctgtagctaaggaagaggcaaaacctgcagccccaaaagcaagtgcagaaaagaaagccgaaaagtctgaaccagtaaaaccagctgtagccaaggaagaggcaaaaccagctgagccagtcactccgaaaacagaaaaagtagcggctaaaccgcaaagtcgtaatttcaaggctgagcgtgaagcacgtgctaaagagcaggcagagcgacgcaagcaaaataagggcaataaccgtgaccaacaacaaaacggaaaccgtcagaaaaacgacggccgtaatggtggaaaacaaggtcaaagcaaccgcgacaatcgtcgctttaatgaccaagctaagaagcagcaaggtcagcaaaaacgtagaaatgagcgccgtcagcaagaggataaacgttcaaatcaagcggctccacgtattgactttaaagcccgtgcagcagccctaaaagcagagcaaaatgcagagtacgctcgttcaagtgaggaacgcttcaagcagtatcaggctgctaaagaagccttggctcaagctaacaaacgcaaggaaccagaggaaatctttgaagaagcggctaagttagctgaacaagcacagcaagttcaagcagtggttgaagtcgtccctgagaaaaaagaacctgcagtggatacacgtcgtaaaaaacaagctcgaccagacaaaaatcgtgacgattatgatcatgaagaagatggtcctagaaaacaacaaaagaatcgaagtagtcaaaatcaagtgagaaatcaaaagaatagtaactggaataacaacaaaaagaacaaaaaaggcaataacaagaacaaccgtaatcagactccaaaacctgttacggagcgtaaattccatgaattgccaacagaatttgaatatacagatggtatgaccgttgcggaaatcgcaaaacgtatcaaacgtgaaccagctgaaattgttaagaaacttttcatgatgggtgtcatggccacacaaaaccaatccttggatggggaaacaattgaactcctcatggtggattacggtatcgaagccaaacaaaaggttgaagtggataatgctgacatcgaacgtttctttgtcgaagatggttatctcaatgaagatgaattggttgagcgtccaccagttgttactatcatgggacacgttgaccacggtaaaacaacccttttggatactcttcgtaactcacgtgttgcgacaggtgaagcaggtggtattactcagcatatcggtgcctaccaaatcgtggaaaatggtaagaagattaccttccttgatacaccaggacacgcggcctttacatcaatgcgtgcgcgtggtgcttctgttaccgatattacgatcttggtcgtagcggcagatgacggggttatgcctcagactattgaagccatcaaccactcaaaagcagctaacgttccaatcatcgtagctattaacaagattgataaaccaggtgctaacccagaacgcgttatcggtgaattggcagagcatggtgtgatgtcaactgcttggggtggagattctgaatttgttgaaatttcggctaaattcaaccaaaatatcgaagaattgttggaaacagtccttcttgtggctgaaatccaagaactcaaagcagacccaacagttcgtgcgatcggtacggttatcgaagcgcgcttggataaaggaaaaggtgcggtcgcaacccttcttgtacaacaaggtaccttgaatgttcaagacccaatcgttgtcggaaataccttcggtcgtgtccgtgctatgaccaacgaccttggtcgtcgtgttaaagttgctggaccatcaacaccagtctctatcacaggtttgaacgaagcaccgatggcgggtgaccactttgccgtttacgaggatgaaaaatctgcgcgtgcagcaggtgaagagcgtgccaaacgtgccctcatgaaacaacgtcaagctacccaacgtgttagccttgaaaacctctttgatacccttaaagctggggaactcaaatctgttaatgttatcatcaaggctgatgtacaaggttctgttgaagccctttctgcctcacttcaaaagattgacgtggaaggtgtcaaagtgactatcgtccactcagcggtcggtgctatcaacgaatcagacgtgacccttgccgaagcttcaaatgcctttatcgttggtttcaacgtacgccctacaccacaagctcgtcaacaagcagaagctgacgatgtggaaatccgtcttcacagcattatctacaaggttatcgaagagatggaagaagctatgaaagggatgcttgatccagaatttgaagaaaaagttattggtgaagcggttatccgtgaaaccttcaaggtgtctaaagtgggaactatcggtggatttatggttatcaacggtaaggttgcccgtgactctaaagtccgtgttatccgtgatggtgtcgttatctatgatggtgaactcgcaagcttgaaacactataaagacgacgtgaaagaagtgacaaacggtcgtgaaggtggattgatgatcgacggctacaatgatattaagatggatgatgtgattgaggcgtatgtcatggaagaaatcaagagataa

SEQ ID NO: 60 - DNA, 3117 bp, Streptococcus pneumoniae
atgaataaagaaaaaataaaaagaaaattaatcacaatactgtttgtatgtattgggatgttatgttttggattgttagcaggagttaaggctgataatcgtgttcaaatgagaacgacgattaataatgaatcgccattgttgctttctccgttgtatggcaatgataatggtaacggattatggtgggggaacacattgaagggagcatgggaagctattcctgaagatgtaaaaccatatgcagcgattgaacttcatcctgcaaaagtctgtaaaccaacaagttgtattccacgagatacgaaagaattgagagaatggtatgtcaagatgttggaggaagctcaaagtctaaacattccagttttcttggttattatgtcggctggagagcgtaatacagttcctccagagtggttagatgaacaattccaaaagtatagtgtgttaaaaggtgttttaaatattgagaattattggatttacaataaccagttagctccgcatagtgctaaatatttggaagtttgtgccaaatatggagcgcattttatctggcatgatcatgaaaaatggttctgggaaactattatgaatgatccgacattctttgaagcgagtcaaaaatatcataaaaatttggtgttggcaactaaaaatacgccaataagagatgatgcgggtacagattctatcgttagtggattttggttgagtggcttatgtgataactggggctcatcaacagatacatggaaatggtgggaaaaacattatacaaacacatttgaaactggaagagctagggatatgagatcctatgcatcggaaccagaatcaatgattgctatggaaatgatgaatgtatatactgggggaggcacagtttataatttcgaatgtgccgcgtatacatttatgacaaatgatgtaccaactccagcatttactaaaggtattattcctttctttagacatgctatacaaaatccagctccaagtaaggaagaagttgtaaatagaacaaaagctgtattttggaatggagaaggtaggattagttcattaaacggattttatcaaggactttattcgaatgatgaaacaatgcctttatataataatgggagatatcatattcttcctgtaatacatgagaaaattgataaggaaaagatttcatctatattccctaatgcaaaaattttgactaaaaatagtgaggaattgtctagtaaagtcaactatttaaactcgctttatccaaaactttatgaaggagatgggtatgctcagcgtgtaggtaattcctggtatatttataatagtaatgctaatatcaataaaaatcagcaagtaatgttgcctatgtatactaataatacaaagtcgttatcgttagatttgacgccacatacttacgctgttgttaaagaaaatccaaataatttacatattttattgaataattacaggacagataagacagctatgtgggcattatcaggaaattttgatgcatcaaaaagttggaagaaagaagaattagagttagcgaactggataagcaaaaattattccatcaatcctgtagataatgactttaggacaacaacacttacattaaaagggcatactggtcataaacctcagataaatataagtggcgataaaaatcattatacttatacagaaaattgggatgagaatacccatgtttataccattacggttaatcataatggaatggtagagatgtctataaatactgaggggacaggtccagtctctttcccaacaccagataaatttaatgatggtaatttgaatatagcatatgcaaaaccaacaacacaaagttctgtagattacaatggagaccctaatagagctgtggatggtaacagaaatggtaattttaactctggttcggtaacacacactagggcagataatccctcttggtgggaagtcgatttgaaaaaaatggataaagttgggcttgttaaaatttataatcgcacagatgctgagactcaacgtctatctaattttgatgtgattctatatgacaataatagaaacgaagttgctaagaaacatgttaataatttgtcgggtgaatctgttagtctagatttcaaagaaaaaggagcaaggtatattaaagttaaattactaacgagtggagtgcctttgagtttagcagaagtagaggtttttagagaatcagatggtaagcaatctgaagaggatatagataaaataacagaagataaagtagtctctacaaataaggtagctactcaaagttcaaccaattatgagggtgtagctgctttagcagttgatggtaataaagatggagattacggacatcattcggtgactcatactaaggcagattctaacgcttggtggcaggtcgatctgggagaagagtttacggtttctaaagttgatatttataatagaacagatgccgaacctcagcgtttatctaattttgatgttatttttctatcttcatcaggagaagaagtttttagaagacattttgataaagtagttgatggtttgttatctttaaaagtaccttctgtaggggctaagctagtcaaaatagaattaaaatcagcagctattccgttaagtttagcggaagttgaagtctatggttcaaagagaactccgaagaaactttctaatattgcattaaccaaagaaactcgacagagttcaacggattacaatggtttttctcgtctagcagttgatggaaataaaaacggagattatggtcatcattcagtgactcataccaaaggagattctccttcatggtgggagatagatttagcacaaaccgaagaattagaaaagttaattatttataatagaacagatgctgaaattcagagattatcaaattttgatattattatatatgattcaaataatcatgaagtttttaaacaacatattgacagtttagaaagcaataatctatccatagacttaaaagggctgaagggaaaaaaggttagaatttctttgagaaacgcaggaattcctttaagtttggcagaggtagaggtttatacttataagtaa

SEQ ID NO: 61 - DNA, 3120 bp, Streptococcus pneumoniae
atgaaatttagtaaaaaatatatagcagctggatcagctgttatcgtatccttgagtctatgtgcctatgcactaaaccagcatcgttcgcaggaaaataaggacaataatcgtgtctcttatgtggatggcagccagtcaagtcagaaaagtgaaaacttgacaccagaccaggttagccagaaagaaggaattcaggctgagcaaattgtaatcaaaattacagatcagggctatgtaacgtcacacggtgaccactatcattactataatgggaaagttccttatgatgccctctttagtgaagaactcttgatgaaggatccaaactatcaacttaaagacgctgatattgtcaatgaagtcaagggtggttatatcatcaaggtcgatggaaaatattatgtctacctgaaagatgcagctcatgctgataatgttcgaactaaagatgaaatcaatcgtcaaaaacaagaacatgtcaaagataatgagaaggttaactctaatgttgctgtagcaaggtctcagggacgatatacgacaaatgatggttatgtctttaatccagctgatattatcgaagatacgggtaatgcttatatcgttcctcatggaggtcactatcactacattcccaaaagcgatttatctgctagtgaattagcagcagctaaagcacatctggctggaaaaaatatgcaaccgagtcagttaagctattcttcaacagctagtgacaataacacgcaatctgtagcaaaaggatcaactagcaagccagcaaataaatctgaaaatctccagagtcttttgaaggaactctatgattcacctagcgcccaacgttacagtgaatcagatggcctggtctttgaccctgctaagattatcagtcgtacaccaaatggagttgcgattccgcatggcgaccattaccactttattccttacagcaagctttctgccttagaagaaaagattgccagaatggtgcctatcagtggaactggttctacagtttctacaaatgcaaaacctaatgaagtagtgtctagtctaggcagtctttcaagcaatccttcttctttaacgacaagtaaggagctctcttcagcatctgatggttatatttttaatccaaaagatatcgttgaagaaacggctacagcttatattgtaagacatggtgatcatttccattacattccaaaatcaaatcaaattgggcaaccgactcttccaaacaatagtctagcaacaccttctccatctcttccaatcaatccaggaacttcacatgagaaacatgaagaagatggatacggatttgatgctaatcgtattatcgctgaagatgaatcaggttttgtcatgagtcacggagaccacaatcattatttcttcaagaaggacttgacagaagagcaaattaaggctgcgcaaaaacatttagaggaagttaaaactagtcataatggattagattctttgtcatctcatgaacaggattatccaagtaatgccaaagaaatgaaagatttagataaaaaaatcgaagaaaaaattgctggcattatgaaacaatatggtgtcaaacgtgaaagtattgtcgtgaataaagaaaaaaatgcgattatttatccgcatggagatcaccatcatgcagatccgattgatgaacataaaccggttggaattggtcattctcacagtaactatgaactgtttaaacccgaagaaggagttgctaaaaaagaagggaataaagtttatactggagaagaattaacgaatgttgttaatttgttaaaaaatagtacgtttaataatcaaaactttactctagccaatggtcaaaaacgcgtttcttttagttttccgcctgaattggagaaaaaattaggtatcaatatgctagtaaaattaataacaccagatggaaaagtattggagaaagtatctggtaaagtatttggagaaggagtagggaatattgcaaactttgaattagatcaaccttatttaccaggacaaacatttaagtatactatcgcttcaaaagattatccagaagtaagttatgatggtacatttacagttccaacctctttagcttacaaaatggccagtcaaacgattttctatcctttccatgcaggggatacttatttaagagtgaaccctcaatttgcagtgcctaaaggaactgatgctttagtcagagtgtttgatgaatttcatggaaatgcttatttagaaaataactataaagttggtgaaatcaaattaccgattccgaaattaaaccaaggaacaaccagaacggccggaaataaaattcctgtaaccttcatggcaaatgcttatttggacaatcaatcgacttatattgtggaagtacctatcttggaaaaagaaaatcaaactgataaaccaagtattctaccacaatttaaaaggaataaagcacaagaaaacttaaaacttgatgaaaaggtagaagaaccaaagactagtgagaaggtagaaaaagaaaaactttctgaaactgggaatagtactagtaattcaacgttagaagaagttcctacagtggatcctgtacaagaaaaagtagcaaaatttgctgaaagttatgggatgaagctagaaaatgtcttgtttaatatggacggaacaattgaattatatttaccatcgggagaagtcattaaaaagaatatggcagattttacaggagaagcacctcaaggaaatggtgaaaataaaccatctgaaaatggaaaagtatctactggaacagttgagaaccaaccaacagaaaataaaccagcagattctttaccagaggcaccaaacgaaaaacctgtaaaaccagaaaactcaacggataatggaatgttgaatccagaagggaatgtggggagtgaccctatgttagatccagcattagaggaagctccagcagtagatcctgtacaagaaaaattagaaaaatttacagctagttacggattaggcttagatagtgttatattcaatatggatggaacgattgaattaagattgccaagtggagaagtgataaaaaagaatttatctgatctcatagcgtaa

SEQ ID NO: 62 - DNA, 3234 bp, Streptococcus pneumoniae
atgattttacaatatgtttattggagtgtatacatgcaaacaaaaacaaagaagctcattgtgagtttgtcttcacttgttttatcaggatttttattaaaccattatatgacaattggagcggaagaaacgactacgaataccattcagcaaagccagaaggaagttcagtatcagcaaagggatacaaaaaatttagttgaaaatggtgattttggtcagacggaggacggaagcagtccgtggacaggaagcaaagctcaggggtggtcagcttgggtagaccagaagaatagtgcagatgcctcaactcgagtcattgaggctaaggatggggctatcactatctcaagccatgagaaattaagggcagcgcttcaccgtatggttcctattgaagctaagaaaaagtataaactgcgtttcaagattaaaacagataataaaatcgggattgccaaagttcgtatcattgaggaaagtggtaaggacaagcgattgtggaattctgcaacgacgtcaggaacaaaggactggcagaccattgaagcagactatagcccgactttagatgttgataaaatcaagctggagttattctatgaaacaggaactgggactgtttcctttaaggatattgagctggtagaggtagcagaccagctttctgaggattctcaaacagataaacagcttgaggaaaagattgatttaccaattggaaaaaaacatgttttttctcttgcggactatacttataaggtagaaaatcctgacgttgcttcagtcaaaaatggaattttagaacctcttaaggaagggacaaccaatgtcattgtcagtaaagatggcaaggaagtgaaaaagattcctttgaagattctggcctctgttaaggatgcatacacagaccgtttggatgactggaatggcatcatcgctgggaatcaatactatgattctaaaaatgaacagatggccaaattaaaccaggaattggaaggaaaggtagctgatagcctatccagtatttcaagtcaggcggaccgcacctatttgtgggaaaaattttcaaattataaaacgtctgcaaatctgactgccacttatcggaaattggaggagatggccaagcaagtgaccaatccttcttctcgttattatcaagatgaaactgtcgttcgaacagtcagggattccatggaatggatgcataaacatgtctacaatagtgaaaagagcattgttgggaactggtgggattatgaaatcggtacacctcgtgccatcaacaataccttgtctctgatgaaagaatacttctctgatgaggaaattaaaaaatatacagatgtgattgaaaaatttgtaccagatcccgaacatttccgaaagacgactgataacccattcaaggctctaggtggaaacttagttgatatgggaagggtaaaagtaatagctggtttactgcgtaaggatgatcaagaaatttcttctaccattcgctcgattgagcaagtgttcaagttggtagaccaaggtgaaggtttttatcaagatggatcctatatcgaccacaccaatgttgcctatacgggtgcttatgggaatgttttgattgatggcctgtctcaactgttgccagtcattcaaaagaccaagaatccaatcgataaagataaaatgcaaaccatgtaccactggattgataaatcgtttgctcctttgctggtgaatggagagttgatggatatgagtcgtggacgctcgatcagtcgtgcaaatagcgaggggcacgtggccgcagtagaagtactaagagggattcaccgaatagcggatatgtctgaaggagaaaccaaacaatgtttgcagagtcttgtgaagaccattgttcaatcggatagttattatgatgtctttaagaatttgaagacttataaggatatcagtttgatgcaatccttgttaagtgatgcaggagtcgcaagtgttccaagaccaagttacctatctgcctttaacaagatggataaaacagccatgtacaatgcagagaaagggtttggatttggcttgtcactcttttccagtcgtaccttgaattacgaacacatgaacaaggaaaataaacgtggttggtatacgagtgatgggatgttctatctttacaatggcgatttgagtcactatagcgatggctactggccaacagttaatccatataagatgcctggtacaacagagacggatgctaagagagcggatagcgatacaggtaaagttttaccgtctgctttcgttggaacgagcaaactagatgatgccaatgcgacagcaaccatggatttcaccaactggaatcaaacattgactgctcataagagctggtttatgctaaaggataagatcgcctttttaggaagcaatatccaaaacacttcaacagatactgctgcaactacaattgaccagagaaaactggaatcaggtaatccatataaagtctatgtcaatgataaagaagcctcccttacagaacaagaaaaggattatcctgaaacccaaagtgtctttttagaatcgttcgattcgaaaaagaatattggttactttttctttaagaagagttcaatcagtatgagtaaggctttgcaaaagggagcctggaaggatatcaatgaaggacagtcagacaaggaagttgaaaatgaatttcttacgattagtcaggctcataagcaaaatagagattcttatggctatatgctcattcctaacgtggatcgtgccaccttcaatcaaatgataaaagagttagaaagtagcctcatcgaaaataacgaaacccttcagtctgtttatgatgctaaacaaggagtttggggcattgtgaaatatgatgattctgtctctactatttccaaccaattccaagttttgaaacgtggagtctataccattcgaaaagaaggggatgaatataagattgcctactataatcctgaaacccaggaatcagctccagatcaggaagtctttaaaaagctagagcaagcagctcagccacaagtacagaattcaaaagaaaaggaaaaatctgaagaggaaaagaaccattcggatcaaaagaatctccctcagacaggagaaggtcagtcaatcttggcaagtctagggttcttgctacttggggcattttatctattccgtagaggaaagaacaactaa

SEQ ID NO: 63 - DNA, 3807 bp, Streptococcus pneumoniae
atgagaaaaacaccatctcacactgagaaaaaaatggtctacagcatccgttccctcaaaaatggaactggttctgtccttatcggcgcaagccttgtcttgcttgccatggctacaccaactatctcatccgacgaaagtacaccaaccactaacgaacccaacaacagaaatacaaccacccttgcccaacctcttactgatacagcagctggctctggtaagaacgaaagtgatatttcttcacctagaaatgcaaacgcttccctagagaaaacagaagaaaaacctgcaacagagccaactacttctactagtccagtaacaactgaaactaaggcagaagagcccatcgaagataactacttccgtatccatgtcaaaaaacttcctgaagaaaacaaggatgctcaaggactatggacttgggacgatgttgaaaaaccatctgaaaactggccaaacggagctttgtccttcaaggatgccaagaaagatgactacggctattacctagatgtcaaattaaagggagaacaagccaagaaaattagcttcctcatcaacaatacagctggaaaaaatctaaccggcgataaatctgtagaaaaactagttccaaaaatgaacgaggcttggttagaccaagattacaaggttttctcttacgagccacagcctgcaggaactgttcgcgtcaactactaccgcacagatggcaactatgacaagaaatctctctggtactggggagatgtaaaaaatccaagtagcgctcaatggcctgacggaacagactttacggctacaggcaaatatggccgctatatcgacattcctcttaatgaagccgcaagagaatttggatttttattactagatgagagcaaacaaggagtcgacgtgaaaatccgtaaagaaaattataagttcacagatttgaaaaatcatagccaaattttcctaaaagacgatgatgaatcgatttacacaaatccatactatgtccatgatatccgtatgacaggagcccaacacgtaggcacttctagcattgaaagtagcttttcaacacttgtcggtgctaaaaaagaagatatcctcaaacactccaacatcactaatcacctaggaaacaaggtaactattaccgatgttgcaatcgatgaagctggtaagaaagtgacctacagcggagatttctctgacacaaaacatccttatactgttagctacaattccgaccaattcactaccaaaacaagctggcgcctgaaagatgagacatacagctatgatggcaaactgggagctgacctaaaagaagaaggaaaacaagttgatttgaccctttggtcaccaagtgctgataaggtttctgttgttgtctacgacaagaatgaccctgacaaagtagttggaactgtcgctctcgaaaaaggggaaagaggaacttggaaacaaactctagacagcacaaacaaactcggaatcacagatttcactggctactattatcaataccaaatcgagcgtcaaggtaaaactgttcttgcactcgatccttacgctaaatctcttgctgcttggaatagcgacgatgccaaaattgacgatgtccataaagtggctaaagccgcctttgtagatccagctaaactcggacctcaagacttgacttatggtaagattcacaatttcaagactcgtgaagacgccgttatctacgaagctcatgtgcgtgacttcacttcagatcctgccattgcaaaagacttgaccaaaccatttgggacttttgaagccttcattgaaaaactagactatctcaaagacttgggtgtaacccatatccagctccttccagtcttgtcttactactttgtcaatgaattgaaaaaccatgaacgcttgtctgactacgcttcaagcaacagcaactacaactggggatatgaccctcaaaactacttctccttgactggtatgtactcaagcgatcctaagaatccagaaaaacgaatcgcagaatttaaaaacctcatcaacgaaatccacaaacgtggtatgggagctatcctagatgtcgtttataaccacacagccaaagtcgatatctttgaagacctagagccaaactactaccactttatggatgccgatggcacacctcgaactagctttggtggtggacgcttggggacaacccaccatatgaccaaacggctcctagttgactctatcaaatacctagttgatacctacaaagtggatggcttccgcttcgatatgatgggagaccatgacgccgcttctatcgaagaagcttacaaggctgcacgcgccctcaatccaaacctcatcatgcttggtgaaggttggagaacctatgccggtgatgaaaacatgcctactaaagctgctgaccaagattggatgaaacataccgatactgtcgctgtcttttcagatgacatccgtaacaacctcaaatctggttatccaaacgaaggtcaacctgcctttatcacaggtggcaagcgtgatgtcaacaccatctttaaaaatctcattgctcaaccaactaactttgaagctgacagccctggagatgtcatccaatacatcgcagcccatgataacttgaccctctttgacatcattgcccagtctatcaaaaaagacccaagcaaggctgagaactatgctgaaatccaccgtcgtttacgacttggaaatctcatggtcttgacagctcaaggaactccatttatccactccggtcaggaatatggacgtactaaacaattccgtgacccagcctacaagactccagtagcagaggataaggttccaaacaaatctcacttgttgcgtgataaggacggcaacccatttgactatccttacttcatccatgattcttacgattctagcgatgcagtcaacaagtttgactggactaaggctacagatggtaaagcttatcctgaaaatgtcaagagccgtgactatatgaaaggtttgattgcccttcgtcaatctacagatgccttccgacttaagagtcttcaagatatcaaagaccgtgtccacctcatcactgtcccaggccaaaatggtgtggaaaaagaggatgtagtgattggctaccaaatcactgctccaaacggcgatatctacgcagtctttgtcaatgcggatgaaaaagctcgcgaatttaatttgggaactgcctttgcacatctaagaaatgcggaagttttggcagatgaaaaccaagcaggatcagtcggaattgccaacccgaaaggacttgaatggactgaaaaaggcttgaaattgaatgcccttacagctactgttcttcgagtctctcaaaatggaactagccatgagtcaactgcagaagagaaaccagactcaaccccttccaagcctgaacatcaaaatgaagcttctcaccctgcacatcaagacccagctccagaagctagacctgattctactaaaccagatgccaaagtagctgatgcggaaaataaacctagccaagctacagctgattcacaagctgaacaaccagcacaagaagcacaagaagcacaagcatcatctgtaaaagaagcggttcgaaacgaatcggtagaaaactctagcaaggaaaatatacctgcaaccccagataaacaagctgaacttccaaatacaggaatcaaaaacgaaaacaaactcctatttgcaggaatcagcctccttgcgctccttggtctcggtttcttactaaaaaataaaaaagagaactaa

SEQ ID NO: 64 - DNA, 3939 bp, Streptococcus pneumoniae
atgaaacatgaaaaacaacagcgtttttctattcgtaaatacgctgtaggagcagcttctgttctaattggatttgccttccaagcacagactgttgcagccgatggagttactcctactactacagaaaaccaaccgaccatccatacggtttctgattcccctcaatcatccgaaaatcggactgaggaaacacctaaagcagtgcttcaaccagaagctccaaaaactgtagaaacagaaactccagctactgataaggtagctagtcttccaaaaacagaagaaaaaccacaagaggaagttagttcaactcctagtgataaagcagaagtggtaactccaacttctgctgaaaaagaaactgctaataaaaaggcagaagaagctagccctaaaaaggaagaagcgaaagaggttgattctaaagagtcaaatacagacaagactgacaaggataaaccagctaaaaaagatgaagcgaaagcagaggctgacaaaccggcaacagaggcaggaaaggaacgtgctgcaactgtaaatgaaaaactagcgaaaaagaaaattgtttctattgatgctggacgtaaatatttctcaccagaacagctcaaggaaatcatcgataaagcgaaacattatggctacactgatttacacctattagtcggaaatgatggactccgtttcatgttggacgatatgagcatcacagctaacggcaagacctatgccagtgacgatgtcaaacgcgccattgaaaaaggtacaaatgattattacaacgatccaaacggcaatcacttaacagaaagtcaaatgacagatctgattaactatgccaaagataaaggtatcggtctcattccgacagtaaatagtcctggacacatggatgcgattctcaatgccatgaaagaattgggaatccaaaaccctaactttagctattttgggaagaaatcagcccgtactgtcgatcttgacaacgaacaagctgtcgcttttacaaaagcccttatcgacaagtatgctgcttatttcgcgaaaaagactgaaatcttcaacatcggacttgatgaatatgccaatgatgcgacagatgctaaaggttggagtgtgcttcaagctgataaatactatccaaacgaaggctaccctgtaaaaggctatgaaaaatttattgcctacgccaatgacctcgctcgtattgtaaaatcgcacggtctcaaaccaatggcttttaacgacggtatctactacaatagcgacacaagctttggtagttttgacaaagacatcatcgtttctatgtggactggtggttggggaggctacgatgtcgcttcttctaaactactagctgaaaaaggtcaccaaatccttaataccaatgatgcttggtactacgttcttggacgaaacgctgatggccaaggctggtacaatctcgatcaggggctcaatggtattaaaaacacaccaatcacttctgtaccaaaaacagaaggagctgatatcccaatcatcggtggtatggtagctgcttgggctgacactccatctgcacgttattcaccatcacgcctcttcaaactcatgcgtcattttgcaaatgccaacgctgaatacttcgcagctgattatgaatctgcagagcaagcacttaacgaggtaccaaaagacctgaaccgttatactgcagaaagcgtcacggccgtaaaagaagctgaaaaagctattcgctctctcgatagcaaccttagccgtgcccaacaagatacgattgatcaagccattgctaaacttcaagaaactgtcaacaacttgaccctcacgcctgaagctcaaaaagaagaagaagctaaacgtgaggttgaaaaacttgccaaaaacaaggtaatctcaatcgatgctggacgcaaatactttactctgaaccagctcaaacgcatcgtagacaaggccagtgagctcggatattctgatgtccatctccttctaggaaatgacggacttcgctttctactcgatgatatgaccattactgccaacggaaaaacctatgctagtgatgacgttaaaaaagctattatcgaaggaactaaagcttactacgacgatccaaacggtactgcactaacacaggcagaagtaacagagctaattgaatacgctaaatctaaggacatcggtctcatcccagctattaacagtccaggtcacatggatgctatgctggttgccatggaaaaattaggtattaaaaatcctcaagcccactttgataaagtttcaaaaacaactatggacttgaaaaacgaagaagcgatgaactttgtaaaagccctcatcggtaaatacatggacttctttgcaggtaaaacaaagattttcaactttggtactgacgaatacgccaacgatgcgactagtgcccaaggctggtactacctcaagtggtatcaactctatggcaaatttgccgaatatgccaacaccctcgcagctatggccaaagaaagagggcttcaaccaatggccttcaacgatggcttctactatgaagacaaggacgatgttcagtttgacaaagatgtcttgatttcttactggtctaaaggctggtggggatataacctcgcatcacctcaatacctagcaagcaaaggctataaattcttgaataccaacggtgactggtactacattcttggtcaaaaaccagaagatggtggtggtttcctcaagaaagctattgagaatactggaaaaacaccattcaatcaactagcttctaccaaatatcctgaagtagatcttccaacagtcggaagtatgctttcaatctgggcagatagaccaagcgctgaatacaaggaagaggaaatctttgaactcatgactgcctttgcagaccacaacaaagactactttcgtgctaattataatgctctccgcgaagaattagctaaaattcctacaaacttagaaggatatagtaaagaaagtcttgaggcccttgacgcagctaaaacagctctaaattacaacctcaaccgtaataaacaagctgagcttgacacgcttgtagccaacctaaaagccgctcttcaaggcctcaaaccagctgtaactcattcaggaagcctagatgaaaatgaagtggctgccaatgttgaaaccagaccagaactcatcacaagaactgaagaaattccatttgaagttatcaagaaagaaaatcctaacctcccagccggtcaggaaaatattatcacagcaggagtcaaaggtgaacgaactcattacatctctgtactcactgaaaatggaaaaacaacagaaacagtccttgatagccaggtaaccaaagaagttataaaccaagtggttgaagttggcgctcctgtaactcacaagggtgatgaaagtggtcttgcaccaactactgaggtaaaacctagactggatatccaagaagaagaaattccatttaccacagtgacttgtgaaaatccactcttactcaaaggaaaaacacaagtcattactaagggcgtcaatggacatcgtagcaacttctactctgtgagcacttctgccgatggtaaggaagtgaaaacacttgtaaatagtgtcgtagcacaggaagccgttactcaaatagtcgaagtcggaactatggtaacacatgtaggcgatgaaaacggacaagccgctattgctgaagaaaaaccaaaactagaaatcccaagccaaccagctccatcaactgctcctgctgaggaaagcaaagttcttcctcaagatccagctcctgtggtaacagagaaaaaacttcctgaaacaggaactcacgattctgcaggactagtagtcgcaggactcatgtccacactagcagcctatggactcactaaaagaaaagaagactaa

SEQ ID NO: 65 - DNA, 4980 bp, Streptococcus pneumoniae
atgaagaatccattttttgaaagacgttgtcgttacagtattcgtaagttatcagtaggagcctgctcgctgatgattggtgctgttttatttgctggtccagccttggctgaagaaactgcagttcctgaaaatagcggagctaatacagagcttgtttcaggagagagtgagcattcgaccaatgaagctgataagcagaatgaaggggaacatgctagagaaaacaagctagaaaaggcagaaggagtagcgatagcatctgaaactgcttcgccagcaagcaatgaagctgcaactactgaaactgcagaagcagctagcgcagctaaaccagaggaaaaagcaagtgaggtggttgcagaaacaccatctgcagaagcaaaacctaagtctgacaaggaaacagaagcaaagcccgaagcaactaaccaaggggatgagtctaaaccagcagcagaagctaataagactgaaaaagaagtccagccagatgtccctaaaaatacagaaaaaacattaaaaccaaaggaaatcaaatttaattcttgggaagaattgttaaaatgggaaccaggtgctcgtgaagatgatgctattaaccgcggatctgttgtcctcgcttcacgtcggacaggtcatttagtcaatgaaaaagctagcaaggaagcaaaagttcaagccttatcaaacaccaattctaaagcaaaagaccatgcttctgttggtggagaagagttcaaggcctatgcttttgactattggcaatatctagattcaatggtcttctgggaaggtctcgtaccaactcctgacgttattgatgcaggtcaccgtaacggggttcctgtatacggtacactcttcttcaactggtctaatagtattgcagatcaagaaagatttgctgaagctttgaagcaagacgcagatggtagcttcccaattgcccgtaaattggtagacatggccaagtattatggctatgatggctatttcatcaaccaagaaacaactggagatttggttaaacctcttggagaaaagatgcgccagtttatgctctatagcaaggaatatgctgctaaggtaaaccatccaatcaagtattcttggtacgatgccatgacctataactatggacgttatcatcaagatggtttgggagaatacaactaccaattcatgcaaccagaaggagataaggttccggcagataacttctttgctaactttaactgggataaggctaaaaatgattacactattgcaactgccaactggattggtcgtaatccttatgatgtatttgcaggtttggaattgcaacagggtggttcctacaagacaaaggttaagtggaatgacattttagacgaaaatgggaaattgcgcctttctcttggtttatttgccccagataccattacaagtttaggaaaaactggtgaagattatcataaaaatgaagatatcttctttacaggttatcaaggagaccctactggccaaaaaccaggtgacaaagattggtatggtattgctaacctagttgcggaccgtacgccagcggtaggtaatacttttactacttcttttaatacaggtcatggtaaaaaatggttcgtagatggtaaggtttctaaggattctgagtggaattatcgttcagtatcaggtgttcttccaacatggcgctggtggcagacttcaacaggggaaaaacttcgtgcagaatatgattttacagatgcctataatggcggaaattcccttaaattctctggtgatgtagccggtaagacagatcaggatgtgagactttattctactaagttagaagtaactgagaagaccaaacttcgtgttgcccacaagggaggaaaaggttctaaagtttatatggcattctctacaactccagactacaaattcgatgatgcagatgcatggaaagagctaaccctttctgacaactggacaaatgaagaatttgatcttagctcactagcgggtaaaaccatctatgcagtcaaactatttttcgagcatgaaggtgctgtaaaagattatcagtttaacctaggacaattaactatctcggacaatcaccaagagccacaatcgccgacaagcttttctgtagtgaaacaatctcttaaaaatgcccaagaagcggaagcagttgtgcaatttaaaggcaacaaggatgcagatttctatgaagtttatgaaaaagatggagacagctggaaattactaactggctcatcttctacaactatttatctaccaaaagttagccgctcagcaagtgctcagggtacaactcaagaactgaaggttgtagcagtcggtaaaaatggagttcgttcagaagctgcaaccacaacctttgattggggtatgactgtaaaagataccagcctaccaaaaccactagctgaaaatatcgttccaggtgcaacagttattgatagtactttccctaagactgaaggtggagaaggtattgaaggtatgttgaacggtaccattactagcttgtcagataaatggtcttcagctcagttgagtggtagtgtggatattcgtttgaccaagccacgtaccgttgttagatgggtcatggatcatgcaggagctggtggtgagtctgttaacgatggcttgatgaacactaaagactttgacctttattataaagatgcagatggtgagtggaagctagctaaggaagtccgtggtaacaaagcacacgtgacagatatcactcttgataaaccaatcactgctcaagactggcgcttgaatgttgtcacttctgacaatggaactccatggaaggctattcgtatctataactggaaaatgtatgaaaagcttgatactgagagtgtcaatattccgatggccaaggctgcagcccgttctctaggcaataacaaggtacaagttggctttgcagatgtaccggctggagcaactattaccgtttatgataatccaaattctcaaactccgctcgcaaccttgaagagcgaagttggaggagacctagcaagtgcaccattggatttgacaaatcaatctggtcttctttattatcgtacccagttgccaggcaaggaaattagtaatgtcctagcagtttccgttccaaaagatgacagaagaatcaagtcagtcagcctagaaacaggacctaagaaaacaagctacgccgaaggggaggatttggaccttagaggtggtgttcttcgagttcagtatgaaggaggaactgaggacgaactcattcgcctaactcacgcaggtgtatcagtatcaggttttgatacgcatcataagggagaacagaatcttactctccaatatttgggacaaccggtaaatgctaatttgtcagtgactgtcactggccaagacgaagcaagtccgaaaactattttgggaattgaagtaagtcaggaaccgaaaaaagattacctagttggtgatagcttagacttgtctgaaggacgctttgcagtggcttatagcaatgacaccatggaagaacattcctttactgatgagggagttgaaatttctggttacgatgctcaaaagactggtcgtcaaaccttgacgcttcattaccaaggccatgaagttagctttgatgttttggtatctccaaaagcagcattgaacgatgagtacctcaaacaaaaattagcagaagttgaagctgctaagaacaaggtggtctataactttgcttcatcagaagtaaaagaagccttcttgaaagcaattgaagcggccgaacaagtgttgaaagaccatgaaactagcacccaagatcaagtcaatgaccgacttaataaattgacagaagctcataaagctctgaatggtcaagagaaatttacggaagaaaagacagagcttgatcgcttaacaggtgaggttcaagaactcttggctgccaaaccaaaccatccttcaggttctgccctagctccgcttcttgagaaaaacaaggccttggttgaaaaagtagatttgagtccagaagagcttacaacagcgaaacagagtctaaaagatctggttgctttattgaaagaagacaagccagcagtcttttctgatagtaaaacaggtgttgaagtacacttctcaaataaagagaagactgtcatcaagggtttgaaagtagagcgtgttcaagcaagtgctgaagagaagaaatactttgctggagaagatgctcatgtctttgaaatagaaggtttggatgaaaaaggtcaagatgttgatctctcttatgcttctattgtgaaaatcccaattgaaaaagataagaaagttaagaaagtatttttcttacctgaaggcaaagaggcagtagaattggcttttgaacaaacggatagtcatgttatctttacagcacctcactttactcattatgcctttgtttatgaatctgctgaaaaaccacaacctgctaaaccagcaccacaaaacacagtccttccaaaacctacttatcaaccgacttctgatcaacaaaaggctcctaaattggaagttcaagaggaaaaggttgcctttcatcgtcaagagcatgaaaatactgagatgctagttggggaacaacgagtcatcatacagggacgagatggactgttaagacatgtctttgaagttgatgaaaacggtcagcgtcgtcttcgttcaacagaagtcatccaagaagcgattccagaaattgttgaaattggaacaaaagtaaaaacagtaccagcagtagtagctacacaggaaaaaccagctcaaaatacagcagttaaatcagaagaagcaagcaaacaattgccaaatacaggaacagctgatgctaatgaagccctaatagcaggcttagccagccttggtcttgctagtttagccttgaccttgagacggaaaagagaagataaagattaa

SEQ ID NO: 66 - DNA, 5304 bp, Streptococcus pneumoniae
atgaataaaggattatttgaaaaacgttgtaaatatagtattcggaaattttcattaggtgttgcttctgttatgattggagctgcattctttgggacaagtccggttcttgcagatagcgtgcagtctggttccacggcgaacttaccagctgatttagctactgctcttgcaacagcaaaagagaatgatggacgtgattttgaagcgcctaaggtgggagaagaccaaggttctccagaagttacagatggacctaagacagaagaagaactattagcacttgaaaaagaaaaaccggctgaagaaaaaccaaaagaggataaacctgcagctgctaaacctgaaacacctaagacggtaacccctgaatggcaaacggtagagaaaaaagaacaaaagggaacagtcactatccgagaagaaaaaggtgtccgctacaaccaactatcctcaactgctcaaaatgataacgcaggaaaaccagccctgtttgaaaagaagggcttgaccgttgatgccaatggaaatgcaactgttgatttaaccttcaaagatgattctgaaaagggcaaatcacgctttggtgtcttcctgaaatttaaagataccaataataatgtttttgtcggttatgacaaggatggctggttctgggagtataaatctccaacaactagcacttggtatagaggtagtcgtgttgctgctcctgaaacaggatcaacaaaccgtctctctatcactctcaagtcagacggtcagctaaatgccagcaataacgatgtcaatctctttgacacagtgactctaccagctgcggtcaatgaccatcttaaaaatgagaagaagatccttctcaaggcgggctcttatggcaatgaccgtacggttgtcagcgttaaaacagacaatcaagaaggcgtaaaagcggatgatactcctgcccagaaagaaacaggtccggttgttgacgatagcaaggtgacttatgatacgatccagtctaaggtacttaaggcagtaattgaccaagccttccctcgtgtcaaggaatacagcttgaacgggcatactttgccaggacaggtgcaacagttcaaccaagtctttatcaataaccaccgaatcacccctgaagtcacttataagaaaatcaatgagacaacagcagagtacttgatgaagcttcgcgatgatgctcacttaatcaatgcggaaatgacagtacgcctgcaagttgtggacaatcaattgcactttgatgtgaccaagattgtcaaccacaatcaagtcactccaggtcaaaagattgatgacgaaagaaaactactttcttctattagtttcctcggcaatgctttagtctctgtttctagtgatcaaactggtgctaagtttgatggggcaaccatgtcaaacaatacgcatgtcagcggagatgatcatatcgatgtaaccaatccaatgaaagacctagccaagggttacatgtatggatttgtttctacagataagcttgctgctggtgtttggagtaactctcaaaacagctatggtggtggttcgaatgactggactcgtttgacagcttataaagaaacagtcggaaatgccaactatgtaggaatccacagctctgaatggcaatgggaaaaagcttataagggcattgttttcccagaatacacgaaggaacttccaagggctaaggttgttatcactgaagatgccaatgcagacaagaaagtcgattggcaggatggtgccattgcttatcgtagcattatgaacaaccctcaaggttgggaaaaagttaaggatatcacagcttaccgtatcgcgatgaactttggttctcaagcacaaaacccattccttatgaccttggatggtatcaagaaaatcaatctccacacagatggtcttgggcaaggtgttctccttaaaggatatggtagcgaaggccatgactctggtcacttgaactatgctgatattggtaagcgtatcggtggtgtcgaagacttcaagaccctaattgagaaggctaagaaatatggagctcatctaggtatccacgttaacgcttcagaaacttatcctgagtctaaatacttcaatgaaaaaattctccgtaagaatccagatggaagttatagctatggttggaactggctagatcaaggtatcaacattgatgctgcctatgacctagctcatggtcgtttggcacgttgggaagatttgaagaaaaaacttggtgacggtctcgactttatctatgtggacgtttggggtaatggtcaatcaggtgataacggtgcctgggctacccacgttcttgctaaagaaattaacaaacaaggctggcgctttgcgatcgagtggggccatggtggtgagtacgactctaccttccatcactgggcagctgacttgacctacggtggctacaccaataaaggtatcaacagtgccatcactcgctttatccgtaaccaccaaaaagatgcttgggttggggactacagaagttatggtggtgcagccaactatccactgctaggtggctacagcatgaaagactttgaaggctggcaaggaagaagtgactacaatggctacgtgactaacctctttgctcatgacgtcatgactaagtacttccaacacttcactgtaagtaaatgggaaaatggtacaccggtgactatgaccgataacggtagcacctataaatggactccagaaatgcgagtggaattggtagatactgacaataataaagtagttgtaactcgtaagtcaaatgatgtcaatagcccacaatatcgcgaacgtacagtaactctcaacggacgtgtcatacaagatggttcagcttacttgactccttggaactgggatgcaaatggtaagaaactttctactgataaggaaaagatgtactacttcaatacgcaggccggtgcaacaacttggacccttccaagcgattgggcaaagagcaaggtttacctttacaagctaactgaccaaggtaagacagaagagcaagaactaactgtaaaagatggtaaaattaccctagatcttctagcaaatcaaccatacgttctctatcgttcgaaacaaaccaatcctgaaatgtcatggagcgaaggcatgcacatctatgaccaaggatttaatagtggaaccttgaaacactggaccatttctggtgatgcttctaaggcagaaattgtcaagtctcaaggggcaaacgatatgcttcgtattcaaggaaacaaagaaaaagttagtctcactcagaaattaactggcttgaaaccaaataccaagtatgccgtttatgtcggtgtcgataaccgtagtaatgccaaggcgagcatcactgtaaatactggtgaaaaagaagtgactacttataccaataagtctctcgcgctcaactatgttaaggcttacgcccacaatacacgtcgtgacaatgctacagttaacgatacaagttacttccaaaacatgtacgccttctttacaacaggatcggacgtctcaaatgttactctgacattgagtcgtgaagctggtgatcaagcaacttactttgatgaaattcgtacctttgaaaacaattcaagcatgtatggagacaagcatgatacaggtaaaggcaccttcaagcaagactttgaaaatgttgctcagggtatcttcccatttgtagtgggtggtgttgaaggtgttgaagataaccgcactcacttgtctgaaaaacatgatccatatacacaacgtggttggaacggtaagaaagttgatgatgttatcgaaggaaattggtcactcaagacaaatggactagtgagccgtcgtaacttggtttaccaaactattccgcaaaacttccgttttgaagcaggtaagacctaccgtgtaacctttgaatacgaagcaggttcagacaatacctatgcttttgtagtcggtaagggagaattccagtcaggtcgtcgtggtactcaagcaagcaacttggaaatgcatgaattgccaaatacttggacagattctaagaaagccaagaaggcaaccttcctcgtgacaggtgcagaaacaggggatacttgggtaggtatctactcaactggaaatgcaagtaatactcgtggtgattctggtggaaatgccaacttccgtggttataacgacttcatgatggataatcttcaaatcgaagaaattaccctaacaggtaagatgttgacagaaaatgctctgaagaactacttgccaacggttgccatgactaactacaccaaagagtctatggatgctttgaaagaggcggtctttaacctcagtcaggccgatgatgatatcagtgtggaagaagcgcgtgcagagattgccaagattgaagccttgaagaatgctttggttcagaagaaaacggctttggtagcagaagactttgcaagtcttacagctcctgctcaggctcaagaaggtcttgcaaatgcctttgatggaaacttatctagtttatggcatacatcatggggcggaggagatgtaggcaagcctgcaaccatggtcttgaaagaaccaactgaaatcactggacttcgttatgttccacgtggatcaggttcaaatggtaacttgcgtgatgtgaaacttgttgtgacagatgagtctggcaaggagcatacctttactgcaactgattggccagataacaataagtcaaaagatattgactttggtaagacaatcaaggctaagaaaattgtccttactggtaccaagacatacggagatggtggcgataaataccaatctgcagcggaacttatctttactcgtccacaggtagcagaaacaccttttgacttgtcaggctatgaagcagctttggctaaggctcagaaattaacagacaaagacaatcaagaggaagtagctagcgttcaggcaagcatgaaatatgcgacggataaccatctcttgacggaaagaatggtggaatactttgcagattatctcaaccaattaaaagattctgctacgaaaccagatgctccaactgtagagaaacctgagtttaaacttagctctttagcttccgatcaaggtaagacgccagattataagcaagaaatagctagaccagaaacacctgaacaaatcttgccagcaacaggtgagagtcaatctgacacagccctcttcctagcaggtgttagcctagccctatctgctctctttgtagtaaaaacgaagaaagactag

SEQ ID NO: 67 - DNA, 6015 bp, Streptococcus pneumoniae
atggaaaagtattttggtgaaaaacaagagcgtttttcatttagaaaattatcagtaggacttgtatctgcaacgatttcaagtttattttttatgtctgtattagctagttcatctgtggatgctcaagaaactgcgggagttcactataaatatgtggcagattcagagctatcatcagaagaaaagaagcagcttgtctatgatattccgacatacgtggagaatgatgatgaaacttattatcttgtttataagttaaattctcaaaatcaactggcggaattgccaaatactggaagcaagaatgagaggcaagccctagttgctggtgctagcttagctgctatgggaattttaatttttgctgtttccaagaaaaaggttaagaataaaacggtattacatttagtattggttgcagggataggaaatggtgtcttagtttcagtccatgctttagaaaatcatcttttgctaaattacaatacggactatgaattgacctctggagaaaaattacctcttcctaaagagatttcaggttacacttatattggatatatcaaagagggaaaaacgacttctgagtctgaagtaagtaatcaaaagagttcagttgccactcctacaaaacaacaaaaggtggattataatgttacaccgaattttgtagaccatccatcaacagtacaagctattcaggaacaaacacctgtttcttcaactaagccgacagaagttcaagtagttgaaaaacctttctctactgaattaatcaatccaagaaaagaagagaaacaatcttcagattctcaagaacaattagccgaacataagaatctagaaacgaagaaagaggagaagatttctccaaaagaaaagactggggtaaatacattaaatccacaggatgaagttttatcaggtcaattgaacaaacctgaactcttatatcgtgaggaaactatggagacaaaaatagattttcaagaagaaattcaagaaaatcctgatttagctgaaggaactgtaagagtaaaacaagaaggtaaattaggtaagaaagttgaaatcgtcagaatattctctgtaaacaaggaagaagtttcgcgagaaattgtttcaacttcaacgactgcgcctagtccaagaatagtcgaaaaaggtactaaaaaaactcaagttataaaggaacaacctgagactggtgtagaacataaggacgtacagtctggagctattgttgaacccgcaattcagcctgagttgcccgaagctgtagtaagtgacaaaggcgaaccagaagttcaacctacattacccgaagcagttgtgaccgacaaaggtgagactgaggttcaaccagagtcgccagatactgtggtaagtgataaaggtgaaccagagcaggtagcaccgcttccagaatataagggtaatattgagcaagtaaaacctgaaactccggttgagaagaccaaagaacaaggtccagaaaaaactgaagaagttccagtaaaaccaacagaagaaacaccagtaaatccaaatgaaggtactacagaaggaacctcaattcaagaagcagaaaatccagttcaacctgcagaagaatcaacaacgaattcagagaaagtatcaccagatacatctagcaaaaatactggggaagtgtccagtaatcctagtgattcgacaacctcagttggagaatcaaataaaccagaacataatgactctaaaaatgaaaattcagaaaaaactgtagaagaagttccagtaaatccaaatgaaggcacagtagaaggtacctcaaatcaagaaacagaaaaaccagttcaacctgcagaagaaacacaaacaaactctgggaaaatagctaacgaaaatactggagaagtatccaataaacctagtgattcaaaaccaccagttgaagaatcaaatcaaccagaaaaaaacggaactgcaacaaaaccagaaaattcaggtaatacaacatcagagaatggacaaacagaaccagaaccatcaaacggaaattcaactgaggatgtttcaaccgaatcaaacacatccaattcaaatggaaacgaagaaattaaacaagaaaatgaactagaccctgataaaaaggtagaagaaccagagaaaacacttgaattaagaaatgtttccgacctagagttatacagtttgtcaaatggtacttataaacaacacatttcgttagagcaagttccaagcaatccaaatagctactttgttaaagtgaaatcttcttcattcaaagatgtatacctaccagtagcatcaatatcagaggaaagaaaaaatgataaaatcctttataaaatcacagcaaaagtagagaagcttcagcaggagatagaaagcagatataaagataattttaccttctatctagctaagaagggaacagaagaaacaacaaactttacttcctttagtaatctggtcaaagctataaaccaaaatccctctggaacctatcatttagcggccagcctgaatgctaacgaagtggagcttggtcctgatgaaagatcctatatcaaggacacctttactggtcgtttaatcggtgaaaaagatggcaagaattatgctatctataatttgaaaaaacctctgtttgaaaacttgagtggtgctacagtagaaaaactgagtctaaaaaatgttgctatttcagggaaagatgatatcggttcactggcaaatgaagctcagaataacacaaaaattaagcaagttcacgtcgatggtgttctggctggtgaacgtggtatcggtggtttgctggctaaggctgagcaatcaagcatcacagagagcagtttcaagggaagaattatcaacacttatgaaacgactgctgcctacaatatcggtggtatggtcggtcatttgacaggtgacaaggctttacttactaagtcaaaagcgacagtagccatttcatctaacacaaatacttcagatcagactgtgggtggacttgcaggcctagtagaccgagatgcacagatccaagatagctatgctgaaggtgatatcaacaatgtcaagcactttggtagagtcgctggagtggcaggcaatttgtgggatcgaacttctggtgatgttaggcatgctggaagtttgaccaatgttctcagcgatgttaatgtaaccaacggaaatgccatcactggttaccactataacgaaatgaaggtaaaggacacattcagcagcaaggccaacagagtctacaatgtcaccttggtcaaggatgaggtcgtcagcaaggaatcctttgaagaaagaggaacaatgctagatgcttctcaaattgcaagcaaaaaagcagaaatcaatcctctcattttaccaacagtggagccactttcaacaagtggcaaaaaagacagtgatttttctaaggtggcctattatcaagctaagcgcaacttgacttataaaaacattgaaaaattgctacctttctacaacaaggcaaccatcgtcaaatacggaaacctggtcaatgagaacagtcttttatatcaaaaagaactcttgtcagcagtcatgatgaaggacaaccaagtcatcacagacattgtttctaacaaacagactgcaaacaaactcttgcttcactacaaggatgatttatctgagaagctggatctcaaataccagaatgatttcgccaaattagcagaatatagtctgggcaatactggacttctctatacgccaaaccaattcctgtatgaccaaacctctatcatcaagcaagtcttacctgacttacaaaaggttgactatcattcagaagccatcagaaagacgctgggtatttctccaaacgtcaagcaaactgagctctatctagaagaccagttcgccaaaacaaaacaacaactggaagacagtttgaaaaaactcttgtcagcggatgctggactggctagtgctaaccccgtcactgaaggttatcttgtagataaaatcaaacgcaacaaggaagccttgctacttggcttgacctatctggaacggtggtataactttagctatggtcaggtgaatgtcaaagacctagttctgtaccatttggacttctttggtaaggggaatgcttcaccattagatactctgattgagttgggtaaatctggctttaacaatcttctagctaagaataatgtcgatacttatggtatcagtcttgccagtcaacatggaacgacagatttgtttagcacgctggaacattaccgaaaagtctttttaccaaatacaagcaataatgactggtttaaatcagagactaaggcttacattgtcgaagaaaaatccactatcgaagaggtgaaaacgaagcaagggttagctggcaccaagtattctatcggtgtttatgatcgtatcacgagtgccacatggaaataccgcaatatggtcttgcctctcctgaccttgccagagagatccgtatttgtcatctcgaccatgtctagtctaggatttggagcttatgatcgctaccgcagtagtgaccataaagcgggcaaggctctcaatgattttgttgaagaaaatgcgcgtgaaacagccaaacgtcagcgagatcactacgattattggtatcgtattttagacgacaatgcacgtgaaaaactttatagaaatattttgctttacgatgcttataaatttggcgatgataataccgtagggaaagctacagaagtggcagattttgataatccaaatcctgcaatgcaacatttctttggacctgttggaaataaagttgggcataatcaacacggtgcttatgctacaggtgatgcagtttattatatgggttatcgaatgttggataaggatggagctattacttatacgcatgagatgacacatgactcagatcaggacatttatcttggaggatatggtcgaagaagtggcttgggaccagagttctttgctaaaggattattacaagcaccagaccatccagatgatgcgaccattaccatcaactccatcttgaaacattcaaaatctgatagtacagaaagtcgacgattacaagtacttgatccaactacaagatttaataatgcagatgatttgaagcaatatgtccacaacatgtttgacgttgtttatatgttggaatatctcgaaggaaattcaattcttaaattggatacgaatcaaaaacaacaacttcttagaaaagttacaaatgagtaccatcctgatcctgatggaaataaggtctatgcaacaaatgttgtcagaaatctaacagtagaagaagttgaaagactacgttcattcaatgatttgattgataataatattctttcgtctagggaatatgcctcaggtaaatacgaaagaaatggctacttcactattaagttatttgcaccgatttatgctgcattaagtaatgatataggaacaccaggtgacctgatgggacgtcgtatagcctatgaactactagctgctaaaggctttaaagatggtatggtaccatatatctcaaaccaatacgaagaagaagccaaacaaaagggcaagacaatcaatctctacggtaaaacaagaggtttggttacagatgacttggttttggaaaaggtatttaataaccaatatcatacttggagtgagtttaagaaagctatgtatcaagaacgacaagatcagtttgatagattgaacaaagttacttttaatgatacaacacagccttggcaaacatttgccaagaaaactacaagcagtgtagatgaattacagaaattaatggacgttgctgttcgtaaggatgcagaacacaattactaccattggaataactacaatccagacatagatagtgaagtccacaagctcaagagagcaatctttaaagcctatcttgaccaaacaaatgattttagaagttcaatttttgagaataaaaaatag

SEQ ID NO: 68 - DNA, 6423 bp, Streptococcus pneumoniae
atgaaaaaaagtacagtattgtcattaaccacagctgcagttattttagcagcctatgcccctaatgaggtagtcttagcagacacatctagctctgaagatgctttaaacatctctgataaagaaaaagtagcagaaaataaagagaaacatgaaaatatccatagtgctatggaaacttcacaggattttaaagagaagaaaacagcagtcattaaggaaaaagaagttgttagtaaaaatcctgtgatagacaataacactagcaatgaagaagcaaaaatcaaagaagaaaattccaataaatcccaaggagattatacggactcatttgtgaataaaaacacagaaaatcccaaaaaagaagataaagttgtctatattgctgaatttaaagataaagaatctggagaaaaagcaatcaaggaactatccagtcttaagaatacaaaagttttatatacttatgatagaatttttaacggtagtgccatagaaacaactccagataacttggacaaaattaaacaaatagaaggtatttcatcggttgaaagggcacaaaaagtccaacccatgatgaatcatgccagaaaggaaattggagttgaggaagctattgattacctaaagtctatcaatgctccgtttgggaaaaattttgatggtagaggtatggtcatttcaaatatcgatactggaacagattatagacataaggctatgagaatcgatgatgatgccaaagcctcaatgagatttaaaaaagaagacttaaaaggcactgataaaaattattggttgagtgataaaatccctcatgcgttcaattattataatggtggcaaaatcactgtagaaaaatatgatgatggaagggattattttgacccacatgggatgcatattgcagggattcttgctggaaatgatactgaacaagacatcaaaaactttaacggcatagatggaattgcacctaatgcacaaattttctcttacaaaatgtattctgacgcaggatctgggtttgcgggtgatgaaacaatgtttcatgctattgaagattctatcaaacacaacgttgatgttgtttcggtatcatctggttttacaggaacaggtcttgtaggtgagaaatattggcaagctattcgggcattaagaaaagcaggcattccaatggttgtcgctacgggtaactatgcgacttctgcttcaagttcttcatgggatttagtagcaaataatcatctgaaaatgaccgacactggaaatgtaacacgaactgcagcacatgaagatgcgatagcggtcgcttctgctaaaaatcaaacagttgagtttgataaagttaacataggtggagaaagttttaaatacagaaatataggggcctttttcgataagagtaaaatcacaacaaatgaagatggaacaaaagctcctagtaaattaaaatttgtatatataggcaaggggcaagaccaagatttgataggtttggatcttaggggcaaaattgcagtaatggatagaatttatacaaaggatttaaaaaatgcttttaaaaaagctatggataagggtgcacgcgccattatggttgtaaatactgtaaattactacaatagagataattggacagagcttccagctatgggatatgaagcggatgaaggtactaaaagtcaagtgttttcaatttcaggagatgatggtgtaaagctatggaacatgattaatcctgataaaaaaactgaagtcaaaagaaataataaagaagattttaaagataaattggagcaatactatccaattgatatggaaagttttaattccaacaaaccgaatgtaggtgacgaaaaagagattgactttaagtttgcacctgacacagacaaagaactctataaagaagatatcatcgttccagcaggatctacatcttgggggccaagaatagatttacttttaaaacccgatgtttcagcacctggtaaaaatattaaatccacgcttaatgttattaatggcaaatcaacttatggctatatgtcaggaactagtatggcgactccaatcgtggcagcttctactgttttgattagaccgaaattaaaggaaatgcttgaaagacctgtattgaaaaatcttaagggagatgacaaaatagatcttacaagtcttacaaaaattgccctacaaaatactgcgcgacctatgatggatgcaacttcttggaaagaaaaaagtcaatactttgcatcacctagacaacagggagcaggcctaattaatgtggccaatgctttgagaaatgaagttgtagcaactttcaaaaacactgattctaaaggtttggtaaactcatatggttccatttctcttaaagaaataaaaggtgataaaaaatactttacaatcaagcttcacaatacatcaaacagacctttgacttttaaagtttcagcatcagcgataactacagattctctaactgacagattaaaacttgatgaaacatataaagatgaaaaatctccagatggtaagcaaattgttccagaaattcacccagaaaaagtcaaaggagcaaatatcacatttgagcatgatactttcactataggcgcaaattctagctttgatttgaatgcggttataaatgttggagaggccaaaaacaaaaataaatttgtagaatcatttattcattttgagtcagtggaagaaatggaagctctaaactccaacgggaagaaaataaacttccaaccttctttgtcgatgcctctaatgggatttgctgggaattggaaccacgaaccaatccttgataaatgggcttgggaagaagggtcaagatcaaaaacactgggaggttatgatgatgatggtaaaccgaaaattccaggaaccttaaataagggaattggtggagaacatggtatagataaatttaatccagcaggagttatacaaaatagaaaagataaaaatacaacatccctggatcaaaatccagaattatttgctttcaataacgaagggatcaacgctccatcatcaagtggttctaagattgctaacatttatcctttagattcaaatggaaatcctcaagatgctcaacttgaaagaggattaacaccttctccacttgtattaagaagtgcagaagaaggattgatttcaatagtaaatacaaataaagagggagaaaatcaaagagacttaaaagtcatttcgagagaacactttattagaggaattttaaattctaaaagcaatgatgcaaagggaatcaaatcatctaaactaaaagtttggggtgacttgaagtgggatggactcatctataatcctagaggtagagaagaaaatgcaccagaaagtaaggataatcaagatcctgctactaagataagaggtcaatttgaaccgattgcggaaggtcaatatttctataaatttaaatatagattaactaaagattacccatggcaggtttcctatattcctgtaaaaattgataacaccgcccctaagattgtttcggttgatttttcaaatcctgaaaaaattaagttgattacaaaggatacttatcataaggtaaaagatcagtataagaatgaaacgctatttgcgagagatcaaaaagaacatcctgaaaaatttgacgagattgcgaacgaagtttggtatgctggcgccgctcttgttaatgaagatggagaggttgaaaaaaatcttgaagtaacttacgcaggtgagggtcaaggaagaaatagaaaacttgataaagacggaaataccatttatgaaattaaaggtgcgggagatttaaggggaaaaatcattgaagtcattgcattagatggttctagcaatttcacaaagattcatagaattaaatttgctaatcaggctgatgaaaaggggatgatttcctattatctagtagatcctgatcaagattcatctaaatatcaaaagcttggcgagattgcagaatctaaatttaaaaatttaggaaatggaaaagagggtagtctaaaaaaagatacaactggggtagaacatcatcatcaagaaaatgaagagtctattaaagaaaaatctagttttactattgatagaaatatttcaacaattagagactttgaaaataaagacttaaagaaactcattaaaaagaaatttagagaagttgatgattttacaagtgaaactggtaagagaatggaggaatacgattataaatacgatgataaaggaaatataatagcctacgatgatgggactgatctagaatatgaaactgagaaacttgacgaaatcaaatcaaaaatttatggtgttctaagtccgtctaaagatggacactttgaaattcttggaaagataagtaatgtttctaaaaatgccaaggtatattatgggaataactataaatctatagaaatcaaagcgaccaagtatgatttccactcaaaaacgatgacatttgatctatacgctaatattaatgatattgtggatggattagcttttgcaggagatatgagattatttgttaaagataatgatcagaaaaaagctgaaattaaaattagaatgcctgaaaaaattaaggaaactaaatcagaatatccctatgtatcaagttatgggaatgtcatagaattaggggaaggagatctttcaaaaaacaaaccagacaatttaactaaaatggaatctggtaaaatctattctgattcagaaaaacaacaatatctgttaaaggataatatcattctaagaaaaggctatgcactaaaagtgactacctataatcctggaaaaacggatatgttagaaggaaatggagtctatagcaaggaagatatagcaaaaatacaaaaggccaatcctaatctaagagccctttcagaaacaacaatttatgctgatagtagaaatgttgaagatggaagaagtacccaatctgtattaatgtcggctttggacggctttaacattataaggtatcaagtgtttacatttaaaatgaacgataaaggggaagctatcgataaagacggaaatcttgtgacagattcttctaaacttgtattatttggtaaggatgataaagaatacactggagaggataagttcaatgtagaagctataaaagaagatggctccatgttatttattgataccaaaccagtaaacctttcaatggataagaactactttaatccatctaaatctaataaaatttatgtacgaaatccagaattttatttaagaggtaagatttctgataagggtggttttaactgggaattgagagttaatgaatcggttgtagataattatttaatctacggagatttacacattgataacactagagattttaatattaagctgaatgttaaagacggtgacatcatggactggggaatgaaagactataaagcaaacggatttccagataaggtaacagatatggatggaaatgtttatcttcaaactggctatagcgatttgaatgctaaagcagttggagtccactatcagtttttatatgataatgttaaacccgaagtaaacattgatcctaagggaaatactagtatcgaatatgctgatggaaaatctgtagtctttaacatcaatgataaaagaaataatggattcgatggtgagattcaagaacaacatatttatataaatggaaaagaatatacatcatttaatgatattaaacaaataatagacaagacactaaacattaagattgttgtaaaagattttgcaagaaatacaaccgtaaaagaattcattttaaataaagatacgggagaggtaagtgaattaaaacctcatagggtaactgtgaccattcaaaatggaaaagaaatgagttcaacgatagtgtcggaagaagattttattttacctgtttataagggtgaattagaaaaaggataccaatttgatggttgggaaatttctggtttcgaaggtaaaaaagacgctggctatgttattaatctatcaaaagatacctttataaaacctgtattcaagaaaatagaggagaaaaaggaggaagaaaataaacctacttttgatgtatcgaaaaagaaagataacccacaagtaaaccatagtcaattaaatgaaagtcacagaaaagaggatttacaaagagaagagcattcacaaaaatctgattcaactaaggatgttacagctacagttcttgataaaaacaatatcagtagtaaatcaactactaacaatcctaataagttgccaaaaactggaacagcaagcggagcccagacactattagctgccggaataatgtttatagtaggaatttttcttggattgaagaaaaaaaatcaagattaa

SEQ ID NO: 69 - DNA, 6702 bp, Streptococcus pneumoniae
atggggaaaggccattggaatcggaaaagagtttatagcattcgtaagtttgctgtgggagcttgctcagtaatgattgggacttgtgcagttttattaggaggaaatatagctggagaatctgtagtttatgcggatgaaacacttattactcatactgctgagaaacctaaagaggaaaaaatgatagtagaagaaaaggctgataaagctttggaaactaaaaatatagttgaaaggacagaacaaagtgaacctagttcaactgaggctattgcatctgagaagaaagaagatgaagccgtaactccaaaagaggaaaaagtgtctgctaaaccggaagaaaaagctccaaggatagaatcacaagcttcaaatcaagaaaaaccgctcaaggaagatgctaaagctgtaacaaatgaagaagtgaatcaaatgattgaagacaggaaagtggattttaatcaaaattggtactttaaactcaatgcaaattctaaggaagccattaaacctgatgcagacgtatctacgtggaaaaaattagatttaccgtatgactggagtatctttaacgatttcgatcatgaatctcctgcacaaaatgaaggtggacagctcaacggtggggaagcttggtatcgcaagactttcaaactagatgaaaaagacctcaagaaaaatgttcgccttacttttgatggcgtctacatggattctcaagtttatgtcaatggtcagttagtggggcattatccaaatggttataaccagttctcatatgatatcaccaaataccttcaaaaagatggtcgtgagaatgtgattgctgtccatgcagtcaacaaacagccaagtagccgttggtattcaggaagtggtatctatcgtgatgtgactttacaagtgacagataaggtgcatgttgagaaaaatgggacaactattttaacaccaaaacttgaagaacaacaacatggcaaggttgaaactcatgtgaccagcaaaatcgtcaatacggacgacaaagaccatgaacttgtagccgaatatcaaatcgttgaacgaggtggtcatgctgtaacaggcttagttcgtacagcgagtcgtaccttaaaagcacatgaatcaacaagcctagatgcgattttagaagttgaaagaccaaaactctggactgttttaaatgacaaacctgccttgtacgaattgattacgcgtgtttaccgtgacggtcaattggttgatgctaagaaggatttgtttggttaccgttactatcactggactccaaatgaaggtttctctttgaatggtgaacgtattaaattccatggagtatccttgcaccacgaccatggggcgcttggagcagaagaaaactataaagcagaatatcgccgtctcaaacaaatgaaggagatgggagttaactccatccgtacaacccacaaccctgctagtgagcaaaccttgcaaatcgcagcagaactaggtttactcgttcaggaagaggcctttgatacgtggtatggtggcaagaaaccttatgactatggacgtttctttgaaaaagatgccactcacccagaagctcgaaaaggtgaaaaatggtctgattttgacctacgtaccatggtcgaaagaggcaaaaacaaccctgctatcttcatgtggtcaattggtaatgaaataggtgaagctaatggtgatgcccactctttagcaactgttaaacgtttggttaaggttatcaaggatgttgataagactcgctatgttaccatgggagcagataaattccgtttcggtaatggtagcggagggcatgagaaaattgctgatgaactcgatgctgttggatttaactattctgaagataattacaaagcccttagagctaagcatccaaaatggttgatttatggatcagaaacatcttcagctacccgtacacgtggaagttactatcgccctgaacgtgaattgaaacatagcaatggacctgagcgtaattatgaacagtcagattatggaaatgatcgtgtgggttgggggaaaacagcaaccgcttcatggacttttgaccgtgacaacgctggctatgctggacagtttatctggacaggtacggactatattggtgaacctacaccatggcacaaccaaaatcaaactcctgttaagagctcttactttggtatcgtagatacagccggcattccaaaacatgacttctatctctaccaaagccaatgggtttctgttaagaagaaaccgatggtacaccttcttcctcactggaactgggaaaacaaagaattagcatccaaagtagctgactcagaaggtaagattccagttcgtgcttattcgaatgcttctagtgtagaattgttcttgaatggaaaatctcttggtcttaagactttcaataaaaaacaaaccagcgatgggcggacttaccaagaaggtgcaaatgctaatgaactttatcttgaatggaaagttgcctatcaaccaggtaccttggaagcaattgctcgtgatgaatctggcaaggaaattgctcgagataagattacgactgctggtaagccagcggcagttcgtcttattaaggaagaccatgcgattgcagcagatggaaaagacttgacttacatctactatgaaattgttgacagccaggggaatgtggttccaactgctaataatctggttcgcttccaattgcatggccaaggtcaactggtcggtgtagataacggagaacaagccagccgtgaacgctataaggcgcaagcagatggttcttggattcgtaaagcatttaatggtaaaggtgttgccattgtcaaatcaactgaacaagcagggaaattcaccctgactgcccactctgatctcttgaaatcgaaccaagtcactgtctttactggtaagaaagaaggacaagagaagactgttttggggacagaagtgccaaaagtacagaccattattggagaggcacctgaaatgcctaccactgttccgtttgtatacagtgatggtagccgtgcagaacgtcctgtaacctggtcttcagtagatgtgagcaagcctggtattgtaacggtgaaaggtatggctgacggacgagaagtagaagctcgtgtagaagtgattgctcttaaatcagagctaccagttgtgaaacgtattgctccaaatactgacttgaattctgtagacaaatctgtttcctatgttttgattgatggaagtgttgaagagtatgaagtggacaagtgggagattgccgaagaagataaagctaagttagcaattccaggttctcgtattcaagcgaccggttatttagaaggtcaaccaattcatgcaacccttgtggtagaagaaggcaatcctgcggcacctgcagtaccaactgtaacggttggtggtgaggcagtaacaggtcttactagtcaaaaaccaatgcaataccgcactcttgcttatggagctaagttgccagaagtcacagcaagtgctaaaaatgcagctgttacagttcttcaagcaagcgcagcaaacggcatgcgtgcgagcatctttattcagcctaaagatggtggccctcttcaaacctatgcaattcaattccttgaagaagcgccaaaaattgctcacttgagcttgcaagtggaaaaagctgacagtctcaaagaagaccaaactgtcaaattgtcggttcgagctcactatcaagatggaacgcaagctgtattaccagctgataaagtaaccttctctacaagtggtgaaggggaagtcgcaattcgtaaaggaatgcttgagttgcataagccaggagcagtcactctgaacgctgaatatgagggagctaaagaccaagttgaactcactatccaagccaatactgagaagaagattgcgcaatccatccgtcctgtaaatgtagtgacagatttgcatcaggaaccaagtcttccagcaacagtaacagttgagtatgacaaaggtttccctaaaactcataaagtcacttggcaagctattccgaaagaaaaactagactcctatcaaacatttgaagtactaggtaaagttgaaggaattgaccttgaagcgcgtgcaaaagtctctgtagaaggtatcgtttcagttgaagaagtcagtgtgacaactccaatcgcagaagcaccacaattaccagaaagtgttcggacatatgattcaaatggtcacgtttcatcagctaaggttgcatgggatgcgattcgtccagagcaatacgctaaggaaggtgtctttacagttaatggtcgcttagaaggtacgcaattaacaactaaacttcatgttcgcgtatctgctcaaactgagcaaggtgcaaacatttctgaccaatggaccggttcagaattgccacttgcctttgcttcagactcaaatccaagcgacccagtttcaaatgttaatgacaagctcatttcctacaataaccaaccagccaatcgttggacaaactggaatcgtactaatccagaagcttcagtcggtgttctgtttggagattcaggtatcttgagcaaacgctccgttgataatctaagtgtcggattccatgaagaccatggagttggtgtaccgaagtcttatgtgattgagtattatgttggtaagactgtcccaacagctcctaaaaaccctagttttgttggtaatgaggaccatgtctttaatgattctgccaactggaaaccagttactaatctaaaagcccctgctcaactcaaggctggagaaatgaaccactttagctttgataaagttgaaacctatgctgttcgtattcgcatggttaaagcagataacaagcgtggaacgtctatcacagaggtacaaatctttgcgaaacaagttgcggcagccaagcaaggacaaacaagaatccaagttgacggcaaagacttagcaaacttcaaccctgatttgacagactactaccttgagtctgtagatggaaaagttccggcagtcacagcaagtgttagcaacaatggtctcgctaccgtcgttccaagcgttcgtgaaggtgagccagttcgtgtcatcgcgaaagctgaaaatggcgacatcttaggagaataccgtctgcacttcactaaggataagagcttactttctcataaaccagttgctgcggttaaacaagctcgcttgctacaagtaggtcaagcacttgaattgccgactaaggttccagtttacttcacaggtaaagacggctacgaaacaaaagacctgacagttgaatgggaagaagttccagcggaaaatctgacaaaagcaggtcaatttactgttcgaggccgtgtccttggtagtaaccttgttgctgagatcactgtacgagtgacagacaaacttggtgagactctttcagataaccctaactatgatgaaaacagtaaccaggcctttgcttcagcaaccaatgatattgacaaaaactctcatgaccgcgttgactatctcaatgacggagatcattcagaaaatcgtcgttggacaaactggtcaccaacaccatcttctaatccagaagtatcagcgggtgtgattttccgtgaaaatggtaagattgtagaacggactgttacacaaggaaaagttcagttctttgcagatagtggtacggatgcaccatctaaactcgttttagaacgctatgtcggtccagagtttgaagtgccaacctactattcaaactaccaagcctacgacgcagaccatccattcaacaatccagaaaattgggaagctgttccttatcgtgcggataaagacattgcagctggtgatgaaatcaacgtaacatttaaagctatcaaagccaaagctatgagatggcgtatggagcgtaaagcagataagagcggtgttgcgatgattgagatgaccttccttgcaccaagtgaattgcctcaagaaagcactcaatcaaagattcttgtagatggaaaagaacttgctgatttcgctgaaaatcgtcaagactatcaaattacctataaaggtcaacggccaaaagtctcagttgaagaaaacaatcaagtagcttcaactgtggtagatagtggagaagatagctttccagtacttgttcgcctcgtttcagaaagtggaaaacaagtcaaggaataccgtatccacttgactaaggaaaaaccagtttctgagaagacagttgctgctgtacaagaagatcttccaaaaatcgaatttgttgaaaaagatttggcatacaagacagttgagaaaaaagattcaacactgtatctaggtgaaactcgtgtagaacaagaaggaaaagttggaaaagaacgtatctttacagcgattaatcctgatggaagtaaggaagaaaaactccgtgaagtggtagaagttccgacagaccgcatcgtcttggttggaaccaaaccagtagctcaagaagctaaaaaaccacaagtgtcagaaaaagcagatacaaaaccaattgattcaagtgaagctagtcaaactaataaagcccagttaccaagtacaggtagtgcggcaagccaagcagcagtagcagcaggtttaactcttctaggtttgagtgcaggattagtagttactaaaggtaaaaaagaagactag

SEQ ID NO: 70 - DNA, 14484 bp, Streptococcus pneumoniae
atgttttttagacgtcagaagggtgaatatagagaaacagaccgagtaactcggttcaaattaatcaaatcagggaagcattggctacgggcctcgacttctctttttggcttgtttaaggtcttgcgaggtggtgttgatactactcaggtcatgaccgaaacggtagaagataaagtaagtcattcaattactgggcttgatatcctcaaggggatagttgctgcgggagctgtcataagtggaaccgttgcaactcaaacgaaggtatttacaaatgagtcagcagtacttgaaaaaactgtagagaaaacggatgctttggcaacaaatgatacagtagttctaggtacgatatctacaagtaattcagcgagttcaactagtttgtcagcttcagagtcggcaagtacatctgcatctgagtcagcctcaaccagcgcttcgacctcagcaagtacaagtgcatcagaatcagcaagtacatcggcttcgacaagtatttctgcatcatctactgtggtaggttcacaaacagctgccgctacagaagcaactgctaagaaggtcgaagaagatcgtaagaaaccagctagtgattatgtagcatcagttacaaatgtcaatctccaatcttatgctaagcgacgcaagcgttcagtggattccatcgagcaattgctggcttctataaaaaatgctgctgttttttctggcaatacgattgtaaatggcgcccctgcaattaatgcaagtctaaacattgctaaaagtgagacaaaagtttatacaggtgaaggtgtagattcggtatatcgtgttccaatttactataaattgaaagtgacaaatgatggttcaaaattgacctttacctatacggttacgtatgtgaatcctaaaacaaatgatcttggtaatatatcaagtatgcgtcctggatattctatctataattcaggtacttcaacacaaacaatgttaacccttggcagtgatcttggtaaaccttcaggtgtaaagaactacattactgacaaaaatggtagacaggttctatcctataatacatctacaatgacgacgcagggtagtgggtatacttggggaaatggtgcccaaatgaatggtttctttgctaagaaaggatatggattaacatcatcttggactgtaccaattactggaacggatacatcctttacatttaccccttacgctgctagaacagatagaattggaattaactacttcaatggtggaggaaaggtagttgaatctagcacgaccagtcagtcactttcacagtctaagtcactctcagtaagtgctagtcaaagcgcctcagcttcagcatcaacaagtgcgtcggcttcagcatcaaccagtgcctcggcttcagcgtcaaccagtgcgtcagcttcagcaagtaccagtgcttcagtctcagcatcaacaagtgcttcagcctcagcatcgacaagtgcctcggcttcagcaagcacatcagcatctgaatcagcgtcaaccagtgcttcggcttcagcaagtaccagtgcttcagcttcagcatcaaccagcgcctcggcctcagcaagcacctcagcttctgaatcggcctcaaccagcgcctcggcctcagcaagcacctcagcttctgaatcggcctcaaccagcgcctcagcctcagcatcaacgagtgcttcggcttcagcaagcacaagcgcctcgggttcagcatcaacgagtacgtcagcttcagcgtcaaccagtgcttcagcctcagcatcaacaagtgcgtcagcctcagcaagtatctcagcgtctgaatcggcatcaacgagtgcgtctgagtcagcatcaacgagtacgtcagcctcagcaagcacctcagcttctgaatcggcctcaaccagtgcgtcagcctcagcatcgacaagcgcctcagcttcagcaagtaccagtgcttcagcctcagcgtcgacaagtgcgtcggcctcaaccagtgcatctgaatcggcatcaaccagtgcgtcagcctcagcaagtactagtgcatcggcttcagcatcaaccagtgcctcggcttcagcgtcaaccagtgcgtcagcttcagcaagtaccagtgcttcagtctcagcatcaacaagtgcttcagcctcagcatcgacaagtgcctcggcttcagcaagcacatcagcatctgaatcagcgtcgacaagcgcctcagcttcagcaagtaccagtgcgtcagcctcagcgtcgacaagtgcgtcagcctcagcaagtactagtgcatcagcttcagcatcaacgagtgcatcggcttcggcgtcaaccagtgcatcagagtcagcaagtaccagtgcgtcagcttccgcatcaacaagtgcctcggcttcagcaagcaccagtgcgtcggcttcagcaagtactagcgcctcagcctcagcctcaaccagtgcgtcagcctcagcaagtatctcagcgtctgaatcggcatcaacgagtgcgtccgcttcagcaagtactagcgcctcagcctcagcgtcaacaagtgcatcggcttcagcgtcaacgagtgcgtctgaatcggcatcaacgagtgcgtccgcttcagcaagtactagcgcctcagcctcagcgtcaacaagtgcatcggcttcagcatcaacgagtgcgtccgcttcagcaagtactagcgcctcagcctcagcgtcaacaagtgcatcggcttcagcgtcaacgagtgcgtctgagtcagcatcaacgagtgcgtcagcctcagcaagcacatcagcttctgaatctgcatcaaccagtgcgtcagcctcagcatcgacaagcgcctcagcttcagcaagtaccagtgcgtcagcctcagcgtcgacaagtgcgtcggcttcagcaagtaccagtgcgtcagcctcagcaagtaccagtgcgtcagcctcagcgtcgacaagtgcgtcggcctcaaccagtgcatctgaatcggcatcaaccagtgcgtcagcctcagcaagtactagtgcatcagcttcagcatcaacgagtgcatcggcttcagcatcaaccagtgcatcagagtcagcaagtaccagtgcgtcagcttccgcatcaacaagtgcctcggcttcagcaagtactagcgcctcagcctcagcgtcaacaagtgcttcagcttccgcgtcaaccagcgcctcggcctcagcaagtatctcagcgtctgaatcggcatcaacaagtgcctcggcttcagcatcaacgagtgcatcagtctcagcaagcaccagtgcgtcggcctcagcaagcaccagcgcgtctgaatccgcatcaaccagtgcctcagcttcagcaagtacctcagcatctgaatcagcatcaacaagtgcctcggcttcagcaagcacaagtgcttcagcctcagcaagtatctcagcgtctgaatcggcatcaacgagtgcgtccgcttcagcaagtactagcgcctcagcatcagcgtcaacaagtgcttcggcttcagcgtcaacgagtgcgtctgagtcagcatcaacgagtacgtcagcctcagcaagcacatcagcttctgaatctgcatcaaccagtgcgtcagcctcagcatcgacaagcgcctcagcttcagcaagtaccagtgcgtcagcctcagcaagtaccagtgcttcagcctcagcgtcgacaagtgcgtcggcctcaaccagtgcatctgaatcggcatcaaccagtgcgtcagcctcagcaagtactagcgcctcagcctcagcatcaacgagtgcgtccgcttcagcaagtactagtgcatcagcttcagcaagtactagcgcctcagcctcagcgtcgacaagcgcctcagcttcagcaagtaccagtgcgtcagcctcagcgtcgacaagtgcgtcggcttcagcaagtacctcagcgtctgaatcagcatcaacaagtgcgtcggcttcagcatcaacgagtgcatcagcttcagcatcaacaagtgcttcagcttcagcaagtaccagtgcgtcggcttcagcatcaacgagtgcttcagtctcagcgtcaaccagtgcctctgaatccgcatcaacaagtgcctcggcttcagcaagcaccagtgcttcggcttcagcgtcaacgagtgcgtctgagtcagcatcaacgagtgcgtcagcctcagcaagcacatcagcttctgaatctgcatcaaccagtgcgtcagcttccgcatcaacaagcgcctcggcctcagcaagtacaagtgcttcagcctcagcatcaaccagtgcatcagcttcagcctcaacaagtgcttcagcctcagcgtcaaccagtgcctcggcttcagcaagtaccagtgcgtcagcttcagcaagcacaagtgcgtcagcttcagcatcaaccagtgcttcggcttcggcatcaacaagtgcctcagcatcagcatcaacgagtgcgtcagcctcagcaagtactagtgcatcagcatcagcatcaaccagtgcatcagcctcagcaagtatctcagcgtctgaatcggcatcaacgagtgcatcagcatcagcatcaacgagtgcatcggcttcagcgtcaaccagtgcatcagtctcagcaagcaccagtgcgtcggcttcagcatcaacgagtgcctcagcctcagcaagtatctcagcgtctgaatcggcatcaacgagtgcgtcagcctcagcaagtactagtgcatcggcttcagcaagcaccagtgcgtcggcttcagcatcaaccagtgcctcagcctcagcaagtatctcagcgtctgaatcggcatcaacgagtgcgtcagcctcagcaagtactagtgcatcagcatcagcatcaacgagtgcatcggcttcagcaagtaccagcgcctcagcttcagcaagcaccagtgcgtcagcctcagcaagtaccagcgcctcagcctcagcaagcaccagtgcctcagcttcagcaagtaccagtgcgtcagcctcagcgtcgacaagtgcgtcggcttcagcaagtacctcagcgtctgaatcagcatcaacgagtgcatcagcttcagcatcaacaagtgcttcagcttcagcaagtaccagtgcgtcggcttcagcatcaacgagtgcttcagtctcagcgtcaaccagtgcctctgaatcagcatcaacaagtgcctcggcttcagcaagcaccagtgcgtcggcttcagcaagtactagtgcatcggcttcagcatcgacaagtgcgtctgaatcggcatcaacgagtgcttcggcttcagcatcaacgagtgcgtcagcctcagcaagcacatcagcttctgaatctgcatcaaccagtgcgtccgcttcagcgtcaaccagtgcgtcggcttcagcgtcgacaagtgcttcggcttcagcatcaacgagtgcgtcggcctcagcaagcgcaagtacctcagcgtcagcttccgcctcaaccagtgcgtcggcttcagcaagcacaagtgcgtcagcctcagcaagtatctcagcgtctgaatcggcatcaacgagtgcgtctgagtcagcatcaacgagtacgtcagcctcagcaagcacatcagcttctgaatctgcatcaaccagtgcgtcagcctcagcatcgacaagcgcctcagcttcagcaagtaccagtgcttcagcctcagcgtcgacaagtgcgtcggcctcaaccagtgcatctgaatcggcatcaaccagtgcgtcagcctcagcaagtactagtgcatcagcttcagcatcaacgagtgcatcggcttcagcatcaaccagtgcctcggcttcagcgtcaaccagtgcgtcagcttcagcaagtaccagtgcttcagtctcagcatcaacaagtgcttcagcctcagcatcgacaagtgcctcggcttcagcaagcacatcagcatctgaatcagcgtcgacaagcgcctcagcttcagcaagtaccagtgcgtcagcctcagcgtcgacaagtgcgtcagcctcagcaagtactagtgcatcagcttcagcatcaacgagtgcatcggcttcggcgtcaaccagtgcatcagagtcagcaagtaccagtgcgtcagcttccgcatcaacaagtgcctcggcttcagcaagcaccagtgcgtcggcttcagcaagtactagcgcctcagcctcagcctcaaccagtgcgtcagcctcagcaagtatctcagcgtctgaatcggcatcaacgagtgcgtccgcttcagcaagtactagcgcctcagcctcagcgtcaacaagtgcatcggcttcagcgtcaacgagtgcgtctgaatcggcatcaacgagtgcgtccgcttcagcaagtactagcgcctcagcctcagcgtcaacaagtgcatcggcttcagcatcaacgagtgcgtccgcttcagcaagtactagcgcctcagcctcagcgtcaacaagtgcatcggcttcagcgtcaacgagtgcgtctgagtcagcatcaacgagtgcgtcagcctcagcaagcacatcagcttctgaatctgcatcaaccagtgcgtcagcctcagcatcgacaagcgcctcagcttcagcaagtaccagtgcgtcagcctcagcgtcgacaagtgcgtcggcttcagcaagtaccagtgcgtcagcctcagcaagtaccagtgcgtcagcctcagcgtcgacaagtgcgtcggcctcaaccagtgcatctgaatcggcatcaaccagtgcgtcagcctcagcaagtactagtgcatcagcttcagcatcaacgagtgcatcggcttcagcatcaaccagtgcatcagagtcagcaagtaccagtgcgtcagcttccgcatcaacaagtgcctcggcttcagcaagtactagcgcctcagcctcagcgtcaacaagtgcttcagcttccgcgtcaaccagcgcctcggcctcagcaagtatctcagcgtctgaatcggcatcaacaagtgcctcggcttcagcatcaacgagtgcatcagtctcagcaagcaccagtgcgtcggcctcagcaagcaccagcgcgtctgaatccgcatcaaccagtgcctcagcttcagcaagtacctcagcatctgaatcagcatcaacaagtgcatcggcttcagcaagcacaagtgcttcagcctcagcaagtatctcagcgtctgaatcggcatcaacgagtgcgtccgcttcagcaagtactagcgcctcagcatcagcgtcaacaagtgcttcggcttcagcgtcaacgagtgcgtctgagtcagcatcaacgagtacgtcagcctcagcaagcacatcagcttctgaatctgcatcaaccagtgcgtcagcctcagcatcgacaagcgcctcagcttcagcaagtaccagtgcgtcagcctcagcaagtaccagtgcttcagcctcagcgtcgacaagtgcgtcggcctcaaccagtgcatctgaatcggcatcaaccagtgcgtcagcctcagcaagtactagcgcctcagcctcagcatcaacgagtgcgtccgcttcagcaagtactagtgcatcagcatcagcatcaacgagtgcatcggcttcagcaagtaccagcgcctcagcttcagcaagcaccagtgcgtcagcctcagcaagtaccagcgcctcagcctcagcaagcaccagtgcctcagcttcagcaagtaccagtgcgtcagcctcagcgtcgacaagtgcgtcggcttcagcaagtacctcagcgtctgaatcagcatcaacgagtgcatcagcttcagcatcaacaagtgcttcagcttcagcaagtaccagtgcgtcggcttcagcatcaacgagtgcttcagtctcagcgtcaaccagtgcctctgaatcagcatcaacaagtgcctcggcttcagcaagcaccagtgcgtcggcttcagcaagtactagtgcatcggcttcagcatcgacaagtgcgtctgaatcggcatcaacgagtgcttcggcttcagcatcaacgagtgcgtcagcctcagcaagcacatcagcttctgaatctgcatcaaccagtgcgtccgcttcagcgtcaaccagtgcgtcggcttcagcgtcgacaagtgcttcggcttcagcatcaacgagtgcgtcggcctcagcaagcgcaagtacctcagcgtcagcttccgcctcaaccagtgcgtccgcttcagcaagcacaagtgcgtcagcctcagcaagtatctcagcgtctgaatcggcatcaacgagtgcgtcggcctcagcaagcgcaagtacctcagcgtcagcttccgcctcaaccagtgcgtcggcttcagcaagcacaagtgcgtcagcctcagcaagtatctcagcgtctgaatcggcatcaacgagtgcgtctgagtcagcatcaacgagtacgtcagcctcagcaagcacatcagcttctgaatcggcatcaaccagtgcgtcagcctcagcatcgacaagcgcctcagcttcagcaagtaccagtgcttcagcctcagcgtcgacaagtgcgtcggcctcaaccagtgcatctgaatcggcatcaaccagtgcgtcagcctcagcaagtactagtgcatcagcttcagcatcaacgagtgcatcggcttcagcatcaaccagtgcctcggcttcagcgtcaaccagtgcgtcagcttcagcaagtaccagtgcttcagtctcagcatcaacaagtgcttcagcctcagcatcgacaagtgcctcggcttcagcaagcacatcagcatctgaatcagcgtcgacaagtgcgtcggcctcaaccagtgcatctgaatcggcatcaaccagtgcgtcagcctcagcaagtactagtgcatcagcttcagcatcaacgagtgcatcggcttcggcgtcaaccagtgcatcagagtcagcaagtaccagtgcgtcagcttccgcatcaacaagtgcctcggcttcagcaagcacatcagcatctgaatcagcgtcaaccagtgcttcggcttcagcaagtaccagtgcttcagcttcagcatcaaccagcgcctcggcctcagcaagcacctcagcttctgaatcggcctcaaccagcgcctcggcctcagcaagcacctcagcttctgaatcggcctcaaccagcgcctcagcctcagcatcaacgagtgcttcggcttcagcaagcacaagcgcctcgggttcagcatcaacgagtacgtcagcttcagcgtcaaccagtgcttcagcctcagcatcaacaagtgcgtcagcctcagcaagtatctcagcgtctgaatcggcatcaacgagtgcgtctgagtcagcatcaacgagtacgtcagcctcagcaagcacctcagcttctgaatcggcctcaaccagtgcgtcagcctcagcatcgacaagcgcctcagcttcagcaagtaccagtgcttcagcctcagcgtcgacaagtgcgtcggcctcaaccagtgcatctgaatcggcatcaaccagtgcgtcagcctcagcaagtactagtgcatcggcttcagcatcaaccagtgcctcggcttcagcgtcaaccagtgcgtcagcttcagcaagtaccagtgcttcagtctcagcatcaacaagtgcttcagcctcagcatcgacaagtgcctcggcttcagcaagcacatcagcatctgaatcagcgtcgacaagcgcctcagcttcagcaagtaccagtgcgtcagcctcagcgtcgacaagtgcgtcagcctcagcaagtactagtgcatcagcttcagcatcaacgagtgcatcggcttcggcgtcaaccagtgcatcagagtcagcaagtaccagtgcgtcagcttccgcatcaacaagtgcctcggcttcagcaagcaccagtgcgtcggcttcagcaagtactagcgcctcagcctcagcctcaaccagtgcgtcagcctcagcaagtatctcagcgtctgaatcggcatcaacgagtgcgtccgcttcagcaagtactagcgcctcagcctcagcgtcaacaagtgcatcggcttcagcgtcaacgagtgcgtctgaatcggcatcaacgagtgcgtccgcttcagcaagtactagcgcctcagcctcagcgtcaacaagtgcatcggcttcagcatcaacgagtgcgtccgcttcagcaagtactagcgcctcagcctcagcgtcaacaagtgcatcggcttcagcgtcaacgagtgcgtctgagtcagcatcaacgagtgcgtcagcctcagcaagcacatcagcttctgaatctgcatcaaccagtgcgtcagcctcagcatcgacaagcgcctcagcttcagcaagtaccagtgcgtcagcctcagcgtcgacaagtgcgtcggcttcagcaagtaccagtgcgtcagcctcagcaagtaccagtgcgtcagcctcagcgtcgacaagtgcgtcggcctcaaccagtgcatctgaatcggcatcaaccagtgcgtcagcctcagcaagtactagtgcatcagcttcagcatcaacgagtgcatcggcttcagcatcaaccagtgcatcagagtcagcaagtaccagtgcgtcagcttccgcatcaacaagtgcctcggcttcagcaagtactagcgcctcagcctcagcgtcaacaagtgcttcagcttccgcgtcaaccagcgcctcggcctcagcaagtatctcagcgtctgaatcggcatcaacaagtgcctcggcttcagcatcaacgagtgcatcagtctcagcaagcaccagtgcgtcggcctcagcaagcaccagcgcgtctgaatccgcatcaaccagtgcctcagcttcagcaagtacctcagcatctgaatcagcatcaacaagtgcctcggcttcagcaagcacaagtgcttcagcctcagcaagtatctcagcgtctgaatcggcatcaacgagtgcgtccgcttcagcaagtactagcgcctcagcatcagcgtcaacaagtgcttcggcttcagcgtcaacgagtgcgtctgagtcagcatcaacgagtacgtcagcctcagcaagcacatcagcttctgaatctgcatcaaccagtgcgtcagcctcagcatcgacaagcgcctcagcttcagcaagtaccagtgcgtcagcctcagcaagtaccagtgcttcagcctcagcgtcgacaagtgcgtcggcctcaaccagtgcatctgaatcggcatcaaccagtgcgtcagcctcagcaagtactagcgcctcagcctcagcatcaacgagtgcgtccgcttcagcaagtactagtgcatcagcttcagcaagtactagcgcctcagcctcagcgtcgacaagcgcctcagcttcagcaagtaccagtgcgtcagcctcagcgtcgacaagtgcgtcggcttcagcaagtacctcagcgtctgaatcagcatcaacaagtgcgtcggcttcagcatcaacgagtgcatcagcttcagcatcaacaagtgcttcagcttcagcaagtaccagtgcgtcggcttcagcatcaacgagtgcttcagtctcagcgtcaaccagtgcctctgaatccgcatcaacaagtgcctcggcttcagcaagcaccagtgcttcggcttcagcgtcaacgagtgcgtctgagtcagcatcaacgagtgcgtcagcctcagcaagcacatcagcttctgaatctgcatcaaccagtgcgtcagcttccgcatcaacaagcgcctcggcctcagcaagtacaagtgcttcagcctcagcatcaaccagtgcatcagcttcagcctcaacaagtgcttcagcctcagcgtcaaccagtgcctcggcttcagcaagtaccagtgcgtcagcttcagcaagcacaagtgcgtcagcttcagcatcaaccagtgcttcggcttcggcatcaacaagtgcctcagcatcagcatcaacgagtgcgtcagcctcagcaagtactagtgcatcagcatcagcatcaaccagtgcatcagcctcagcaagtatctcagcgtctgaatcggcatcaacgagtgcatcagcatcagcatcaacgagtgcatcggcttcagcgtcaaccagtgcatcagtctcagcaagcaccagtgcgtcggcttcagcatcaacgagtgcctcagcctcagcaagtatctcagcgtctgaatcggcatcaacgagtgcgtcagcctcagcaagtactagtgcatcggcttcagcaagcaccagtgcgtcggcttcagcatcaaccagtgcctcagcctcagcaagtatctcagcgtctgaatcggcatcaacgagtgcgtcagcctcagcaagtactagtgcatcagcatcagcatcaacgagtgcatcggcttcagcaagtaccagcgcctcagcttcagcaagcaccagtgcgtcagcctcagcaagtaccagcgcctcagcctcagcaagcaccagtgcctcagcttcagcaagtaccagtgcgtcagcctcagcgtcgacaagtgcgtcggcttcagcaagtacctcagcgtctgaatcagcatcaacgagtgcatcagcttcagcatcaacaagtgcttcagcttcagcaagtatctcagcgtctgaatcggcatcaacgagtgcgtccgcttcagcaagtactagcgcctcagcatcagcgtcaacaagtgcttcggcttcagcgtcaacgagtgcgtctgagtcagcatcaacgagtacgtcagcctcagcaagcacatcagcttctgaatctgcatcaaccagtgcgtcagcctcagcatcgacaagcgcctcagcttcagcaagtaccagtgcgtcagcctcagcaagtaccagtgcttcagcctcagcgtcgacaagtgcgtcggcctcaaccagtgcatctgaatcggcatcaaccagtgcgtcagcctcagcaagtactagcgcctcagcctcagcatcaacgagtgcgtccgcttcagcaagtactagtgcatcagcttcagcaagtactagcgcctcagcctcagcgtcgacaagcgcctcagcttcagcaagtaccagtgcgtcagcctcagcgtcgacaagtgcgtcggcttcagcaagtacctcagcgtctgaatcagcatcaacaagtgcgtcggcttcagcatcaacgagtgcatcagcttcagcatcaacaagtgcttcagcttcagcaagtaccagtgcgtcggcttcagcatcaacgagtgcttcagtctcagcgtcaaccagtgcctctgaatccgcatcaacaagtgcctcggcttcagcaagcaccagtgcttcggcttcagcgtcaacgagtgcgtctgagtcagcatcaacgagtgcgtcagcctcagcaagcacatcagcttctgaatctgcatcaaccagtgcgtcagcttccgcatcaacaagcgcctcggcctcagcaagtacaagtgcttcagcctcagcatcaaccagtgcatcagcttcagcctcaacaagtgcttcagcctcagcgtcaaccagtgcctcggcttcagcaagtaccagtgcgtcagcttcagcaagcacaagtgcgtcagcttcagcatcaaccagtgcttcggcttcggcatcaacaagtgcctcagcatcagcatcaacgagtgcgtcagcctcagcaagtactagtgcatcagcatcagcatcaaccagtgcatcagcctcagcaagtatctcagcgtctgaatcggcatcaacgagtgcatcagcatcagcatcaacgagtgcatcggcttcagcgtcaaccagtgcatcagtctcagcaagcaccagtgcgtcggcttcagcatcaacgagtgcctcagcctcagcaagtatctcagcgtctgaatcggcatcaacgagtgcgtcagcctcagcaagtactagtgcatcggcttcagcaagcaccagtgcgtcggcttcagcatcaaccagtgcctcagcctcagcaagtatctcagcgtctgaatcggcatcaacgagtgcgtcagcctcagcaagtactagtgcatcagcctcagcatcaacgagtgcatcggcttcagcaagtaccagcgcctcagcttcagcaagcaccagtgcgtcagcctcagcaagtaccagcgcctcagcctcagcaagcaccagtgcctcagcttcagcaagtaccagtgcgtcagcctcagcatcaacaagtgcttcagcttcggcctcaacaagtgcgtcagcttcagcatcaacgagtgcgtcggcttcagcaagcaccagtgcctcggcctcagcaagcaccagtgcttcagcttcagcatcaacaagtgcgtcagcttcagcaagtacatcagtttcaaattcagcaaaccattcgaactcacaagttggaaatacttctggatcgacaggtaaatcccaaaaagaattgcctaatacaggtactgagtcgtcaattggatctgtgttacttggagttctagcagctgttacaggtattggattggttgcgaaacgccgtaaacgtgatgaagaagagtaa

SEQ ID NO: 71 - RNA, 210 nt, Streptococcus pneumoniae
augaaaaucaaagggcaaacuagaaaacuagccgcagguugcucaaaacacuguuuugagguuguggauagaacugacgaagucagcucaaaauacuguuuugagguuguagauagaacugacgaagucagcucaaaacacuguuuugagguuguagauagaacugacgaagucaguaaccauaccuacggcaaggcgacguugacguga

SEQ ID NO: 72 - RNA, 651 nt, Streptococcus pneumoniae
auggaagcaauuaucgagaaaaucaaagaguauaaaaucaucgucaucuguacuggucugggcuugcuuguaggaggauuuuuccugcuaaaaccagcuccacaaacaccugucaaagagacgaauuugcaggcugaagucgcagcuguuuccaaggauuugguauccgaaaaggaagugaacaaggaagaaaaggaagaaccccuugaacaagaucuaaucacaguagaugucaaaggugcugucaaaucgccagggauuuaugacuugccuguagguagucgagucaaugaugcuguucagaaggcugguggcuugacagagcaagcagacagcaagucgcucaaucuagcucagaaaguuagugaugaggcucugguuuacguuccuacuaagggagaagaagcaguuagccaacagacugguuuggggacagcuucuucaauaagcaaggaaaagaaggucaaucucaacaaggccagucuggaagaacucaagcaggucaagggacugggaggaaaacgagcucaggacauuaucgaccaucgugaggcaaauggcaaguucaagucaguagacgagcucaagaaggucucuggcauugguggcaaaacaauagaaaagcuuaaagacuauguuacaguggauuaa

SEQ ID NO: 73 - RNA, 666 nt, Streptococcus pneumoniae
augacgauucuaggaaaagauacaguucaacaaucugcgaaaggugaaucuguaacucaagaagcuacaccagaguauaagcuagaaacuacaccagguggagauaagggaggcaauacuggaagcucagaugcuaaugcgaaugaaggcggugguagccaggcggguggaucagcucacacagguucacaaaacucagcucaaucacaagcuucuaagcaauuagcuacugaaaaagaaucagcuaaaaaugccauugaaaaagcagccaagaacaagcaggaugaaaucaaaggcgcaccgcuuucugauaaagaaaaagcagaacuuuuagcaagaguggaagcagaaaaacaagcagcucucaaagagauugaaaaugcgaaaacuauggaagaugugaaggaagcagaaacgauuggagugcaagccauugccaugguuacaguuccuaagagaccaguggcuccuaaugcugcuccuaagacaacaagugcaccgcaagcaacugcaggaacaaugcaagauguuaccuaccagucaccugcuggcaaacaauuaccuaacacagguucagcaucaagugcagcacuugcuagucuuggucuagugguggcaacaagugguuuugcuuugcuaggaagaaagacuagacguagaaaauag

SEQ ID NO: 74 - RNA, 942 nt, Streptococcus pneumoniae
augaagaaaaaauuauuggcaggugccaucacacuauuaucaguagcaacuuuagcagcuuguucgaaagggucagaaggugcagaccuuaucagcaugaaaggggaugucauuacagaacaucaauuuuaugagcaagugaaaagcaacccuucagcccaacaagucuuguuaaauaugaccauccaaaaaguuuuugaaaaacaauauggcucagagcuugaugauaaagagguugaugauacuauugccgaagaaaaaaaacaauauggcgaaaacuaccaacgugucuugucacaagcagguaugacucuugaaacacguaaagcucaaauucguacaaguaaauuaguugaguuggcaguuaagaagguagcagaagcugaauugacagaugaagccuauaagaaagccuuugaugaguacacuccagauguaacggcucaaaucauccgucuuaauaaugaagauaaggccaaagaaguucucgaaaaagccaaggcagaaggugcugauuuugcucaauuagccaaagauaauucaacugaugaaaaaacaaaagaaaaugguggagaaauuaccuuugauucugcuucaacagaaguaccugagcaagucaaaaaagccgcuuucgcuuuagauguggaugguguuucugaugugauuacagcaacuggcacacaagccuacaguagccaauauuacauuguaaaacucacuaagaaaacagaaaaaucaucuaauauugaugacuacaaagaaaaauuaaaaacuguuaucuugacucaaaaacaaaaugauucaacauuuguucaaagcauuaucggaaaagaauugcaagcagccaauaucaagguuaaggaccaagccuuccaaaauaucuuuacccaauauaucggugguggagauucaagcucaagcaguaguacaucaaacgaauag

SEQ ID NO: 75 - RNA, 942 nt, Streptococcus pneumoniae
auggagaacaaauuuguagaauuuaucgaaaacaauaaaaaaguaaucauuucaucaguugcaguugguguuguauugguauuaggguuuggaugguauucauauaaccaacaacaagcagaacaacaagcaaaaauuguacaauuagaaaaagauagcaaaucagacaaagaacaaguugauaaacuauuugaaucauuugaugcaucuucagaugaaucuauuucuaaauuaaaagaacuaucugaaacuucacuuaaaaccgaugcagguaaagacuaucuuaauaacaaagucaaagaaucaucuaaagcaauuguagauuuucauuugcaaaaagguuuggcuuaugauguuaaagauucagaugacaaauuuaaagauaaagcaacucuugaaacaaauguaaaagaaauuacaaaacaaauugauuuuaucaaaaaaguugaugaaacuuuuaaacaagagaauuuggaagaaacucuuaaaucucuaaaugaucuuguugauaaauaucaaaaacaaaucgaacuuuugaagaaagaagaagaaaaagcugcugaaaaagcugcugaaaaagcaaaggaaucuucuagucaaaguaauucuucugguagugcuucuaaugagucuuauaauggaucuuccaauucaaauguagauuauaguucaucugaacaaacuaauggauauucaaauaauuauggcggucaagauuauucugguucaggagauaguucaacaaaugguggaucaucagaacaauauucaucuagcaauucaaacagcggagcaaauaaugucuacagauauaaaggcacuggugcugacggcuaucaaagauacuacuacaaagaucauaauaauggagauguguaugaugacgauggaaauuaccuugggaacuuugguggcggcauugcagaaccuagucaacgcuaa

SEQ ID NO: 76 - RNA, 1113 nt, Streptococcus pneumoniae
augaagaaaagaauguuauuagcgucaacaguagccuugucauuugccccaguauuggcaacucaagcagaagaaguucuuuggacugcacguaguguugagcaaauccaaaacgauuugacuaaaacggacaacaaaacaaguuauaccguacaguauggugauacuuugagcaccauugcagaagccuuggguguagaugucacagugcuugcgaaucugaacaaaaucacuaauauggacuugauuuucccagaaacuguuuugacaacgacugucaaugaagcagaagaaguaacagaaguugaaauccaaacaccucaagcagacucuagugaagaagugacaacugcgacagcagauuugaccacuaaucaagugaccguugaugaucaaacuguucagguugcagaccuuucucaaccaauugcagaaguuacaaagacagugauugcuucugaagaaguggcaccaucuacgggcacuucugucccagaggagcaaacgaccgaaacaacucgcccaguugaagaagcaacuccucaggaaacgacuccagcugagaagcaggaaacacaagcaagcccucaagcugcaucagcaguggaaguaacuacaacaaguucagaagcaaaagaaguagcaucaucaaauggagcuacagcagcaguuucuacuuaucaaccagaagagacgaaaauaauuucaacaacuuacgaggcuccagcugcgcccgauuaugcuggacuugcaguagcaaaaucugaaaaugcaggucuucaaccacaaacagcugccuuuaaagaagaaauugcuaacuuguuuggcauuacauccuuuagugguuaucguccaggagacaguggagaucacggaaaagguuuggcuaucgacuuuaugguaccagaacguucagaauuaggggauaagauugcggaauaugcuauucaaaauauggccagccguggcauuaguuacaucaucuggaaacaacguuucuaugcuccauucgauagcaaauaugggccagcuaacacuuggaacccaaugccagaccgugguagugugacagaaaaucacuaugaucacguucacguuucaaugaauggauaa

SEQ ID NO: 77 - RNA, 1113 nt, Streptococcus pneumoniae
augaaugauaaguuaaaaaucuucuuguugcuaggaguauuuuuucuagccauaaccgguuucuauguucuauugauacgaaaugcagggcagacagaugccucgcaaauugaaaaggcggcaguuagccaaggaggaaaagcagugaaaaaaacagaaauuaguaaagacgcagacuugcacgaaauuuaucuagcuggagguuguuucuggggaguggaggaauauuucucacguguucccggggugacggaugccguuucaggcuaugcaaaugguagaggagaaacaaccaaguacgaauugauuaaccaaacaggucaugcagaaaccguccaugucaccuaugaugccaagcaaauuucucucaaggaaauccugcuucacuauuuccgcauuaucaauccaaccagcaaaaauaaacaaggaaaugauguggggacccaguaccguacugguguuuauuacacagaugacaaggauuuggaagugauuaaccaagucuuugaugagguggcuaagaaauacgaucaaccucuagcaguugaaaaggaaaacuugaagaauuuugugguggcugaggauuaccaucaagacuaucucaagaaaaauccaaauggcuacugccauaucaauguuaaucaggcggccuauccugucauugaugccagcaaauauccaaaaccaagugaugaggaauugaaaaagacccugucaccugaggaguaugcaguuacccaggaaaaucaaacagaacgagcuuucucaaaccguuacugggauaaauuugaauccgguaucuauguggauauagcaacuggggaaccucucuuuucaucaaaagacaaauuugagucugguuguggcuggccuaguuuuacccaacccaucaguccagauguugucaccuacaaggaagauaaguccuacaauaugacgcguauggaagugcggagccgaguaggagauucucaccuugggcaugucuuuacggaugguccacaggacaagggcggcuuacguuacuguaucaauagccucucuauccgcuuuauucccaaagaccaaauggaagaaaaaggcuacgcuuauuuacuagauuauguugauuaa

SEQ ID NO: 78 - RNA, 1179 nt, Streptococcus pneumoniae
augaagaaaaaaaucuuagcgucacuuuuauuaaguacaguaaugguuucucaaguagcuguuuuaacaacugcgcaugcagaaacgacugaugacaaaauugcugcucaagauaauaaaauuaguaacuuaacagcacaacaacaagaagcccaaaaacaaguugaccaaauucaggagcaaguaucagcuauucaagcugagcagucuaacuugcaagcugaaaaugauagauuacaagcagaaucuaagaaacucgagggugagauuacagaacuuucuaaaaacauuguuucucguaaccaaucguuggaaaaacaagcucguagugcucaaacaaauggagccguaacuagcuauaucaauaccauuguaaacucaaaaucaauuacagaagcuauuucacguguugcugcaaugagugaaaucguaucugcaaacaacaaaauguuagaacaacaaaaggcagauaaaaaagcuauuucugaaaaacaaguagcaaauaaugaugcuaucaauacuguaauugcuaaucaacaaaaauuggcugaugaugcucaagcauugacuacgaaacaggcagaacuaaaagcugcugaauuaagucuugcugcugagaaagcgacagcugaaggggaaaaagcaagucuauuagagcaaaaagcagcagcugaggcagaggcucgugcagcugcgguagcagaagcagcuuauaaagaaaaacgagcuagccaacaacaaucaguacuugcuucagcaaacacuaacuuaacagcucaagugcaagcaguaucugaaucugcagcagcaccuguccgugcaaaaguucguccaacauacaguacaaacgcuucaaguuauccaauuggagaauguacauggggaguaaaaacauuggcaccuugggcuggagacuacugggguaauggagcacagugggcuacaagugcagcagcagcagguuuccguacagguucaacaccucaaguuggagcaauugcauguuggaaugaugguggauauggucacguagcgguuguuacagcuguugaaucaacaacacguauccaaguaucagaaucaaauuaugcagguaaucguacaauuggaaaucaccguggaugguucaauccaacaacaacuucugaagguuuuguuacauauauuuaugcagauuaa

SEQ ID NO: 79 - RNA, 1200 nt, Streptococcus pneumoniae
augaagaaaaagaaugguaaagcuaaaaaguggcaacuguaugcagcaaucggugcugcgaguguaguuguauugggugcuggggggauuuuacucuuuagacaaccuucucagacugcucuaaaagaugagccuacucaucuuguuguugccaaggaaggaagcguggccuccucuguuuuauugucagggacaguaacagcaaaaaaugaacaauauguuuauuuugaugcuaguaagggugauuuagaugaaauccuuguuucugugggcgauaaggucagcgaagggcaggcuuuagucaaguacaguaguucagaagcgcaggcggccuaugauucagcuagucgagcaguagcuagggcagaucgucauaucaaugaacucaaucaagcacgaaaugaagccgcuucagcuccggcuccacaguuaccagcgccaguaggaggagaagaugcaacggugcaaagcccaacuccaguggcuggaaauucuguugcuucuauugacgcucaauugggugaugcccgugaugcgcgugcagaugcugcggcgcaauuaagcaaggcucaaagucaauuggaugcaacaacuguucucaguacccuagagggaacuguggucgaagucaauagcaauguuucuaaaucuccaacaggggcgagucaaguuaugguucauauugucagcaaugaaaauuuacaagucaagggagaauugucugaguacaaucuagccaaccuuucuguaggucaagaaguaagcuuuacuucuaaaguguauccugauaaaaaauggacugggaaauuaagcuauauuucugacuauccuaaaaacaauggugaagcagcuaguccagcagccgggaauaauacagguucuaaauacccuuauacuauugaugugacaggcgagguuggugauuugaaacaagguuuuucugucaacauugagguuaaaagcaaaacuaaggcuauucuuguuccuguuagcagucuaguaauggaugauaguaaaaauuaugucuggauuguggaugaacaacaaaaggcuaaaaaaguugagguuucauugggaaaugcugacgcagaaaaucaagaaaucacuucugguuuaacgaacggugcuaaggucaucaguaauccaacaucuuccuuggaagaaggaaaagaggugaaggcugaugaagcaacuaauuag

SEQ ID NO: 80 - RNA, 1272 nt, Streptococcus pneumoniae
augucaucuaaauuuaugaagagcgcugcggugcuuggaacugcuacacuugcuagcuugcuuuugguagcuugcggaagcaaaacugcugauaagccugcugauucugguucaucugaagucaaagaacucacuguauauguagacgagggauauaagagcuauauugaagagguugcuaaagcuuaugaaaaagaagcuggaguaaaagucacucuuaaaacuggugaugcucuaggaggucuugauaaacuuucucuugacaaccaaucugguaaugucccugauguuaugauggcuccauacgaccguguagguagccuugguucugacggacaacuuucagaagugaaauugagcgauggugcuaaaacagacgacacaacuaaaucucuuguaacagcugcuaaugguaaaguuuacggugcuccugccguuaucgagucacuuguuauguacuacaacaaagacuuggugaaagaugcuccaaaaacauuugcugacuuggaaaaccuugcuaaagauagcaaauacgcauucgcuggugaagaugguaaaacuacugccuuccuagcugacuggacaaacuucuacuauacauauggacuucuugccgguaacggugcuuacgucuuuggccaaaacgguaaagacgcuaaagacaucggucuugcaaacgacgguucuaucguagguaucaacuacgcuaaaucuugguacgaaaaauggccuaaagguaugcaagauacagaaggugcuggaaacuuaauccaaacucaauuccaagaagguaaaacagcugcuaucaucgacggaccuuggaaagcucaagccuuuaaagaugcuaaaguaaacuacggaguugcaacuaucccaacucuuccaaauggaaaagaauaugcugcauucgguggugguaaagcuugggucauuccucaagccguuaagaaccuugaagcuucucaaaaauuuguagacuuccuuguugcaacugaacaacaaaaaguauuauaugauaagacuaacgaaaucccagcuaauacugaggcucguucauacgcugaagguaaaaacgaugaguugacaacagcuguuaucaaacaguucaagaacacucaaccacugccaaacaucucucaaaugucugcaguuugggauccagcgaaaaauaugcucuuugaugcuguaaguggucaaaaagaugcuaaaacagcugcuaacgaugcuguaacauugaucaaagaaacaaucaaacaaaaauuuggugaauaa

SEQ ID NO: 81 - RNA, 1329 nt, Streptococcus pneumoniae
augaaauuuagaaaauuagcuuguacaguacuugcgggugcugcgguucuuggucuugcugcuuguggcaauucuggcggaaguaaagaugcugccaaaucagguggugacggugccaaaacagaaaucacuuggugggcauucccaguauuuacccaagaaaaaacuggugacgguguuggaacuuaugaaaaaucaaucaucgaagcguuugaaaaagcaaacccagauauaaaagugaaauuggaaaccaucgacuucaagucagguccugaaaaaaucacaacagccaucgaagcaggaacagcuccagacguacucuuugaugcaccaggacguaucauccaauacgguaaaaacgguaaauuggcugaguugaaugaccucuucacagaugaauuuguuaaagaugucaacaaugaaaacaucguacaagcaaguaaagcuggagacaaggcuuauauguauccgauuaguucugccccauucuacauggcaaugaacaagaaaauguuagaagaugcuggaguagcaaaccuuguaaaagaagguuggacaacugaugauuuugaaaaaguauugaaagcacuuaaagacaaggguuacacaccagguucauuguucaguucuggucaagggggagaccaaggaacacgugccuuuaucucuaaccuuuauagcgguucuguaacagaugaaaaaguuagcaaauauacaacugaugauccuaaauucgucaaaggucuugaaaaagcaacuagcuggauuaaagacaauuugaucaauaaugguucacaauuugacgguggggcagauauccaaaacuuugccaacggucaaacaucuuacacaauccuuugggcaccagcucaaaaugguauccaagcuaaacuuuuagaagcaaguaagguagaagugguagaaguaccauucccaucagacgaagguaagccagcucuugaguaccuuguaaacggguuugcaguauucaacaauaaagacgacaagaaagucgcugcaucuaagaaauucauccaguuuaucgcagaugacaaggaguggggaccuaaagacguaguucguacaggugcuuucccaguccguacuucauuuggaaaacuuuaugaagacaaacgcauggaaacaaucagcggcuggacucaauacuacucaccauacuacaacacuauugauggauuugcugaaaugagaacacuuugguucccaauguugcaaucuguaucaaauggugacgaaaaaccagcagaugcuuugaaagccuucacugaaaaagcgaacgaaacaaucaaaaaagcuaugaaacaauag

SEQ ID NO: 82 - RNA, 1395 nt, Streptococcus pneumoniae
augaguaaaaaaagacgaaaucgucauaaaaaagaaggucaagaaccgcaauuugauuuugaugaagcaaaagagcuaacaguuggucaagcuauucguaaaaaugaagaaguggaagcaggagucuugccugaggauuccauuuuggacaaguauguuaagcaacacagagaugaaauugaggcggauaaguuugcgacucgucaauacaaaaaagaggaguucguugaaacucagagucuggaugauuuaauucaagagaugcgugaggcuguagagaagucagaagcuucuucggaggaaguuccaucuucugaagacaucuuacuacccuugccucuggacgaugaggagcaaggcuuggauccucuauugcuagaugaugaaaauccaacagaaaugacugaagaaguggaagaggagcaaaaccuuucucgucuggaucaagaggacucagaaaagaaaaguaaaaaaggcuuuauuuugaccguuuuggcgcuuguaucaguaauuauuugugucagugcuuauuaugucuaccgucaaguggcucguucgaccaaggaaauugaaacuucucaaucaacuacagccaaucaaucagauguggaugauuuuaauacacuuuaugacgccuuuuacacagauagcaauaaaacggcuuugaaaaauagccaguuugauaaacugagucaacucaaaaccuugcuugauaaacuggaagguagucgugaacauacgcuugccaaaucuaaauaugauagucuagcaacgcaaaucaaggcuauucaagaugucaaugcacaauuugaaaaaccagcuauuguggaugguguguuggauaccaaugccaaagccaaaucggaugcuaaauuuacggauauuaaaacuggaaauacggagcuugauaaagugcuagauaaggcuaucagucuugguaagagccagcaaacaaguacuucuagcucaaguucaagucaaacuagcagcucaagcucuagucaagcaaguucaaauacgacuagugagccaaaaccaaguaguucaaaugagacuagaaguagucgcagugaagucaauaugggucucucgagugcagggguugcuguucaaagaagugccagucguguugccuauaaucagucugcuauugaugauaguaauaacucugccugggauuuugcggauggugucuuggaacaaauucuagcgacuucacguucacguggcuauaucacuggagaccaauauauccuugaacgugucaauaucguuaacggcaaugguuauuacaaccucuauaagccagauggaaccuaucucuuuacccuuaacuguaagacaggcuacuuugucggaaauggcgcuggucaugcggaugacuuggacuacuaa

SEQ ID NO: 83 - RNA, 1614 nt, Streptococcus pneumoniae
auggaaaucaugucgcuugcgauugcuguuuuugccgucaucauugguuuagucauuggauaugucagcaucucagcuaagaugaaaucaucucaggaagcugcagaguugaugcuuuuaaaugcugaacaagaagcaacuaauuuacguggacaagcugagcgugaagcggauuuacuuguuaaugaagccaaacgugaaagcaagucucuuaaaaaagaagcacuauuggaggccaaagaagaagccagaaaauaccgugaagaaguggacgcugaauucaaaucagaacgucaagaacucaaacaaaucgaaagucguuugacagagagagcuacuagccuugaccguaaggacgacaauuugacgaguaaagaacaaacacuugaacaaaaagaacaaaguauuucugauagagcgaaaaaccuugaugcgcgugaagagcaauuagaggaagucgaaagacaaaaagaagcagaacuagagcguauuggugcgcugucucaggcagaagcacgagauauuaucuuggcucagacagaggaaaacuugaccagggagauugccagucgcauucgcgaagcugagcaagaggucaaggaacguucugacaaaauggccaaggacaucuugguucaagcuaugcaacguaucgcuggugaauauguagcggagucaacaaacucaacaguucaucugccagacgauacuaugaagggacgcauuauuggucgugaaggucguaacauucguaccuuugaaaguuugacaggggucgaugugauuaucgacgauacaccagaaguggugaccuugucaggauuugauccgauucgucgugagauugcccguaugacuauggaaauguugcucaaagauggucguauacauccagcucguaucgaagaguugguugagaaaaaccgucaagagauugacaauaagauucgugaauacggugaggcugcugccuaugaaauuggugcgccaaaccuucauccagacuugaugaagauuaugggacguuugcaguuccguacuucauauggacaaaauguuuugcgccauucgauugagguugcuaaguuggcugguaucauggcgagcgaacuuggugaaaaugcggcucuugcccgucgugcuggauuccuucacgauaucgggaaagccauugaccaugagguugaagguagccacguugaaaucgguauggaauuggcccguaaguacaaggaacccccaguuguggugaauacgauugcuagucaccacggagauguugaagcugagagcgugauagcaguuaucgucgcugcagcagaugccuugagcgcagcccguccaggugcucguagugagucucuugaaagcuacaucaagcgucuccaugauuuggaagaaauugcuaacggcuuugaaggagugcaaacuagcuuugcccuucaagcaggacgugaaauucguaucauggucaauccaggaaaaaucaaggacgacaaagucacaaucuuggcucacaaaguucguaagaaaauugaaaacaaucucgauuauccaggaaauaucaagguaaccgugauucgcgagcuucgugcaguagauuaugcuaaauaa

SEQ ID NO: 84 - RNA, 1884 nt, Streptococcus pneumoniae
augaaaaagaaauuaacuaguuuagcacuuguaggcgcuuuuuuagguuugucaugguaugggaauguucaggcucaagaaaguucaggaaauaaaauccacuuuaucaauguucaagaagguggcagugaugcgauuauucuugaaagcaauggacauuuugccaugguggauacaggagaagauuaugauuucccagauggaagugauucucgcuauccauggagagaaggaauugaaacgucuuauaagcauguucuaacagaccgugucuuucgucguuugaaggaauuggguguccaaaaacuugauuuuauuuuggugacccauacccacagugaucauauuggaaauguugaugaauuacugucuaccuauccaguugaccgagucuaucuuaagaaauauagugauagucguauuacuaauucugaacgucuaugggauaaucuguauggcuaugauaagguuuuacagacugcugcagaaaaagguguuucaguuauucaaaauaucacacaaggggaugcucauuuucaguuuggggacauggauauucagcucuauaauuaugaaaaugaaacugauucaucgggugaauuaaagaaaauuugggaugacaauuccaauuccuugauuagcguggugaaagucaauggcaagaaaauuuaccuugggggcgauuuagauaauguucauggagcagaagacaaguaugguccucucauuggaaaaguugauuugaugaaguuuaaucaucaccaugauaccaacaaaucaaauaccaaggauuucauuaaaaauuugaguccgaguuugauuguucaaacuucggauagucuaccuuggaaaaaugguguugauagugaguauguuaauuggcucaaagaacgaggaauugagagaaucaacgcagccagcaaagacuaugaugcaacaguuuuugauauucgaaaagacgguuuugucaauauuucaacauccuacaagccgauuccaaguuuucaagcugguuggcauaagagugcauaugggaacuggugguaucaagcgccugauucuacaggagaguaugcugucgguuggaaugaaaucgaaggugaaugguauuacuuuaaccaaacggguaucuuguuacagaaucaauggaaaaaauggaacaaucauugguucuauuugacagacucuggugcuucugcuaaaaauuggaagaaaaucgcuggaaucugguauuauuuuaacaaagaaaaccagauggaaauugguuggauucaagauaaagagcagugguauuauuuggauguugaugguucuaugaagacaggauggcuucaauauauggggcaaugguauuacuuugcuccaucaggggaaaugaaaaugggcuggguaaaagauaaagaaaccugguacuauauggauucuacuggugucaugaagacaggugagauagaaguugcuggucaacauuauuaucuggaagauucaggagcuaugaagcaaggcuggcauaaaaaggcaaaugauugguauuucuacaagacagacgguucacgagcuguggguuggaucaaggacaaggauaaaugguacuucuugaaagaaaauggucaauuacuugugaacgguaagacaccagaagguuauacuguggauucaaguggugccugguuaguggauguuucgaucgagaaaucugcuacaauuaaaacuacaagucauucagaaauaaaagaauccaaagaaguagugaaaaaggaucuugaaaauaaagaaacgagucaacaugaaaguguuacaaauuuuucaacuagucaagauuugacauccucaacuucacaaagcucugaaacgaguguaaacaaaucggaaucagaacaguag

SEQ ID NO: 85 - RNA, 1980 nt, Streptococcus pneumoniae
augaaaaaaaaucguguauuugcuacagcaggucuuguuuuauuagcagcagguguacuugcagcaugcaguucuucaaaaucaucugauucaucagccccuaaagcuuauggcuauguuuauacagcagacccagaaaccuuggacuaccugauuucaaguaaaaauaguacaacaguagugacuucaaaugggauugaugguuuauucacuaacgauaauuacgguaaucuugcuccugcaguugcagaggauugggaagucucuaaggaugguuugaccuacacuuauaagauucguaaagggguuaaaugguuuaccucugauggagaagaauaugcagaggugacggcuaaagauuucgugaacgguuuaaaacacgcagcagauaaaaaaucagaagcuauguauuuagcugaaaauucgguuaaaggcuuggcagauuaucuaucaggaacuucaacagauuuuucaacaguuggugucaaggcgguugaugauuauacguuacaauacacuuugaaccagccugaaccguucuggaacucuaaguugaccuauucuauuuucuggccucugaaugaagaauucgaaacaucaaaaggaagcgauuuugcuaaaccaacagauccgacauccuugcuuuauaaugguccauucuuguugaaaggguugacugcaaaaucuucuguagaguuuguaaaaaaugagcaauauugggauaaagaaaauguccaccuagauacuaucaaucuagcuuacuaugauggaucagaucaggagucgcuagagcguaacuucacuaguggagcuuauaguuaugcccgucuuuacccuaccagcuccaacuauucuaagguugcagaagaauacaaggacaauaucuauuacacacaaucaggcucugggauugcuggucugggugugaauauugaucgccaaaguuacaacuauacuucuaaaacuacagauucagagaaaguagcuacuaagaaggcauugcuuaacaaagauuuccgucaagccuugaauuuugcucuugaucgcucagcuuacucagcucaaaucaaugguaaagauggagcagcuuuagcaguucguaauuuauuuguaaaaccagacuuuguuucagcuggugagaagaccuuuggugauuuagucgcugcucaacuuccugcuuauggugaugaguggaaaggugugaauuuagcugaugggcaggaugguuuauucaaugcugacaaggccaaggcagaguuugcgaaagcuaagaaagcuuuagaagcagacggcguucaguuuccuauucaucuggacguuccaguagaccaagcaucaaaaaacuacauaucucguauucaguccuuuaaacaaucuguagaaacaguucuugguguugaaaaugucguuguugauauucaacaaaugacaagugaugaauuccuuaauauuacuuacuaugcugccaaugcuucaucugaggauugggauguaucaggaggaguuucaugggggccagacuaucaagacccaucuacuuaccuggauauuuuaaaaacaacuagcagugaaacuacaaaaacauauuuaggauuugauaauccaaauagcccuucaguaguucaaguugguuugaaagaauacgauaaauuaguugaugaagcugccagagagacaagcgacuugaauguccguuaugaaaaauaugcagcggcucaagcaugguugacagauaguucacucuuuauuccugcuauggcuucuucuggugcagcaccagugcuuucacgaauuguuccauuuacuggagcuucugcgcaaacaggcucuaaggggucagauguuuacuucaaauauuugaaaucacaagauaaaguggugacuaaggaagaguaugaaaaagcucgugaaaaaugguugaaagaaaaagcugaaucaaaugagaaagcucaaaaagaauuggcaagucaugugaaguaa

SEQ ID NO: 86 - RNA, 1983 nt, Streptococcus pneumoniae
augaaaaguucaaaacuauuugcccuugcgggcgugacauuauuggcggcgacuacuuuagcugcaugcucuggaucagguucaagcacuaaaggugagaagacauucucauacauuuaugagacagacccugauaaccucaacuauuugacaacugcuaaggcugcgacagcaaauauuaccaguaacgugguugaugguuugcuagaaaaugaucgcuacgggaacuuugugccgucuauggcugaggauuggucuguauccaaggauggauugacuuacacuuauacuauccguaaggaugcaaaaugguauacuucugaaggugaagaauacgcggcagucaaagcucaagacuuuguaacaggauuaaaauaugcugcugauaaaaaaucagaugcucuuuaccuuguucaagaaucaaucaaaggguuggaugccuauguaaaaggggaaaucaaagauuucucacaaguaggaauuaaggcucuggaugaacagacaguucaguacacuuugaacaaaccagaaagcuucuggaauucuaagacaaccaugggugugcuugcgccaguuaaugaagaguuuuugaauucaaaaggagaugauuuugccaaagcuacggauccaaguagucucuuguauaacgguccuuauuuguugaaauccauugugaccaaauccucuguugaauuugcgaaaaauccgaacuacugggauaaggacaaugugcauguugacaaaguuaaauugucauucugggauggucaagauaccagcaaaccugcagaaaacuuuaaagaugguagccuuacagcagcucgucucuauccaacaagugcaaguuucgcagaacuugagaagaguaugaaggacaauauugucuauacucaacaagacucuauuacguaucuaguugguacaaauauugaccgucaguccuauaaauacacaucuaagaccagcgacgaacaaaaggcaucgacuaaaaaggcucucuuaaacaaggauuuccgucaggcuauugccuuuggauuugaccguacagccuaugccucucaguugaauggacaaacuggagcaaguaaaaucuugcguaaucucuuugugccaccaacauuuguucaagcagaugguaaaaacuuuggcgauauggucaaagagaaauuggucacuuauggggaugaauggaaggauguuaaucuugcagauucucaggauggucuuuacaauccagaaaaagccaaggcugaauuugcuaaagcuaaaucagccuuacaagcagaaggaguccaauucccaauucauuuggauaugccaguugaccaaacagcaacuacaaaaguucagcgcguccaaucuaugaaacaauccuuggaagcaacuuuaggagcugauaaugucauuauugauauucaacaacuacaaaaagacgaaguaaacaauauuacauauuuugcugaaaaugcugcuggcgaagacugggauuuaucagauaaugucgguugggguccagacuuugccgauccaucaaccuaccuugauauuaucaaaccuucuguaggagaaaguacuaaaacauauuuaggguuugacucaggggaagauaauguagcugcuaaaaaaguaggucuauaugacuacgaaaaauugguuacugaggcuggugaugagacuacagauguugcuaaacgcuaugauaaauacgcugcagcccaagcuugguugacagauagugcuuugauuauuccaacuacaucucguacagggcguccaaucuugucuaagaugguaccauuuacaauaccauuugcauugucaggaaauaaagguacaagugaaccagucuuguauaaauacuuggaacuucaagacaaggcagucacuguagaugaauaccaaaaagcucaggaaaaauggaugaaagaaaaagaagagucuaauaaaaaggcucaagaagaucucgcaaaacaugugaaauaa

SEQ ID NO: 87 - RNA, 1998 nt, Streptococcus pneumoniae
augaaaucaaucaacaaauuuuuaacaaugcuugcugccuuauuacugacagcgaguagccuguuuucagcugcaacaguuuuugcggcugggacgacaacaacaucuguuaccguucauaaacuauuggcaacagauggggauauggauaaaauugcaaaugaguuagaaacagguaacuaugcugguaauaaaguggguguucuaccugcaaaugcaaaagaaauugccgguguuauguucguuuggacaaauacuaauaaugaaauuauugaugaaaauggccaaacucuaggagugaauauugauccacaaacauuuaaacucucaggggcaaugccggcaacugcaaugaaaaaauuaacagaagcugaaggagcuaaauuuaacacggcaaauuuaccagcugcuaaguauaaaauuuaugaaauucacaguuuaucaacuuaugucggugaagauggagcaaccuuaacagguucuaaagcaguuccaauugaaauugaauuaccauugaacgauguuguggaugcgcauguguauccaaaaaauacagaagcaaagccaaaaauugauaaagauuucaaagguaaagcaaauccagauacaccacguguagauaaagauacaccugugaaccaccaaguuggagauguuguagaguacgaaauuguuacaaaaauuccagcacuugcuaauuaugcaacagcaaacuggagcgauagaaugacugaagguuuggcauucaacaaagguacagugaaaguaacuguugaugauguugcacuugaagcaggugauuaugcucuaacagaaguagcaacugguuuugauuugaaauuaacagaugcugguuuagcuaaagugaaugaccaaaacgcugaaaaaacugugaaaaucacuuauucggcaacauugaaugacaaagcaauuguagaaguaccagaaucuaaugauguaacauuuaacuaugguaauaauccagaucacgggaauacuccaaagccgaauaagccaaaugaaaacggcgauuugacauugaccaagacauggguugaugcuacaggugcaccaauuccggcuggagcugaagcaacguucgauuugguuaaugcucagacugguaaaguuguacaaacuguaacuuugacaacagacaaaaauacaguuacuguuaacggauuggauaaaaauacagaauauaaauucguugaacguaguauaaaaggguauucagcagauuaucaagaaaucacuacagcuggagaaauugcugucaagaacuggaaagacgaaaauccaaaaccacuugauccaacagagccaaaaguuguuacauaugguaaaaaguuugucaaaguuaaugauaaagauaaucguuuagcuggggcagaauuuguaauugcaaaugcugauaaugcuggucaauauuuagcacguaaagcagauaaagugagucaagaagagaagcaguugguuguuacaacaaaggaugcuuuagauagagcaguugcugcuuauaacgcucuuacugcacaacaacaaacucagcaagaaaaagagaaaguugacaaagcucaagcugcuuauaaugcugcugugauugcugccaacaaugcauuugaauggguggcagauaaggacaaugaaaauguugugaaauuaguuucugaugcacaaggucgcuuugaaauuacaggccuucuugcagguacauauuacuuagaagaaacaaaacagccugcugguuaugcauuacuaacuagccgucagaaauuugaagucacugcaacuucuuauucagcgacuggacaaggcauugaguauacugcugguucagguaaagaugacgcuacaaaaguagucaacaaaaaaaucacuaucccacaaacgggugguauugguacaauuaucuuugcuguagcgggggcugcgauuauggguauugcaguguacgcauauguuaaaaacaacaaagaugaggaucaacuugcuuaa

SEQ ID NO: 88 - RNA, 2223 nt, Streptococcus pneumoniae
augaaaaaaaauauuaaacaauauguaaccuuagguacuguagugguauuaucagcauuuguugcuaacucaguugcagcucaggagacugaaacuucugaaguaucaacaccaaaguuggugcaaccuguugcaccaacgacuccgauuucggaaguacaaccuacaucggauaacucuucggaaguuacuguacaaccucgaacaguugaaacuacuguuaaggauccaucuucuacagcggaagaaacuccugucuuagaaaaaaauaauguuacuuuaacagggggcggagaaaauguuacuaaagaguuaaaggauaaauuuacuagcggugacuuuacuguagugauuaaguacaaucagucaagugagaaaggcuuacaagcucuguuuggaauaucuaauuccaaacccggucaacaaaauaguuauguagauguguuccuuagagacaauggugaguuggggauggaagcgcgugauacuucuuccaauaaaaauaaccuaguauccagaccugcuucaguuugggguaaguacaaacaagaggcugugacuaacacuguugcaguaguagcagauucagucaaaaaaacauauucuuuauacgcaaaugguacaaaaguaguagaaaagaaaguggauaauuuccuaaacaucaaggauauuaaagguauugauuacuauaugcuugggggagugaaacgugcaggaaaaacggcguuugguuuuaacggaacacuagaaaauaucaaauucuuuaauagugcauuggaugaagaaacuguuaaaaagaugacaacaaacgcuguuacuggacauuuaauuuauacggcuaaugauacaacagguucuaacuauuuccguauuccaguucuguauacuuuuagcaauggucggguauuuucaagcauugacgcucguuacgguggaacucaugauuucuugaauaaaauuaauauugcuacaaguuauagugaugauaaugguaagacauggacuaaaccaaaauuaacauuggcauucgaugauuuugcgccaguaccauuagaauggccucgugaaguugguggacgugacuuacaaaucagcgguggugcaaccuauauugacucuguuauuguugaaaaaaagaacaaacaaguacucauguuugcugaugugaugccugcuggaguaaguuuuagagaagcaacuagaaaagauucagguuauaaacaaauugaugguaauuauuaccuuaaauuaaggaaacaaggugauacugauuacaauuauacuauucgugagaaugguacuguauacgacgaucguaccaacagaccaacugaauuuucaguagauaaaaauuucgguauuaaacaaaaugguaauuauuugacgguagagcaauauucgguuucauuugaaaauaauaaaaagacugaauaucguaaugggacuaaaguccauaugaauaucuucuacaaagaugcauuguuuaaaguagugccaacgaacuauauugcauauauuucuagcaaugaucauggcgaaucuuggucugcaccaacuuuauuaccuccuauaaugggacuuaaucggaaugcgccauauuuagguccuggacguggaaucauugaaagcucaacuggacguauucuuauuccgucuuacacugguaaagagucugcguucauuuauagugacgauaauggagcaucuuggaaaguuaaaguagugccacuuccuucuaguuggucagcagaagcacaauuuguagaauugaguccaggaguaauucaagcauauaugcguacaaauaaugguaaaauugcauauuuaacaaguaaagacgcagguacuacuuggagugcaccggaauauuugaaauuuguuucaaauccaaguuauggaacacaauuaucaaucaucaauuauagccaauugauugaugguaaaaaggcugucauuuuaaguacuccaaacuccacaaauggucguaaacacggacaaauuuggauuggucuaauuaaugaugauaauacaauugauuggcguuaucaucacgacguugauuauaguaacuauggauacucauauucaacauugacagaguuaccaaaucaugaaauuggauugauguuugaaaaauuugauucauggucucguaaugaacuucauaugaaaaauguuguaccauauauaacauuuaagauugaagaucugaaaaagaauuaa

SEQ ID NO: 89 - RNA, 2235 nt, Streptococcus pneumoniae
augaauaagaaaaaaaugauuuuaacaagucuagccagcgucgcuaucuuaggggcugguuuuguuacgucucagccuacuuuuguaagagcagaagaaucuccacaaguugucgaaaaaucuucauuagagaagaaauaugaggaagcaaaagcaaaagcugauacugccaagaaagauuacgaaacggcuaaaaagaaagcagaagacgcucagaaaaaguaugaagaugaucagaagagaacugaggagaaagcucgaaaagaagcagaagcaucucaaaaauugaaugauguggcgcuuguuguucaaaaugcauauaaagaguaccgagaaguucaaaaucaacguaguaaauauaaaucugacgcugaauaucagaaaaaauuaacagaggucgacucuaaaauagagaaggcuaggaaagagcaacaggacuugcaaaauaaauuuaaugaaguaagagcaguuguaguuccugaaccaaaugcguuggcugagacuaagaaaaaagcagaagaagcuaaagcagaagaaaaaguagcuaagagaaaauaugauuaugcaacucuaaagguagcacuagcgaagaaagaaguagaggcuaaggaacuugaaauugaaaaacuucaauaugaaauuucuacuuuggaacaagaaguugcuacugcucaacaucaaguagauaauuugaaaaaacuucuugcuggugcggauccugaugauggcacagaaguuauagaagcuaaauuaaaaaaaggagaagcugagcuaaacgcuaaacaagcugaguuagcaaaaaaacaaacagaacuugaaaaacuucuugacagccuugauccugaagguaagacucaggaugaauuagauaaagaagcagaagaagcugaguuggauaaaaaagcugaugaacuucaaaauaaaguugcugauuuagaaaaagaaauuaguaaccuugaaauauuacuuggaggggcugauccugaagaugauacugcugcucuucaaaauaaauuagcugcuaaaaaagcugaguuagcaaaaaaacaaacagaacuugaaaaacuucuugacagccuugauccugaagguaagacucaggaugaauuagauaaagaagcagaagaagcugaguuggauaaaaaagcugaugaacuucaaaauaaaguugcugauuuagaaaaagaaauuaguaaccuugaaauauuacuuggaggggcugauucugaagaugauacugcugcucuucaaaauaaauuagcuacuaaaaaagcugaauuggaaaaaacucaaaaagaauuagaugcagcucuuaaugaguuaggcccugauggagaugaagaagaaacuccagcgccggcuccucaaccagagcaaccagcuccugcaccaaaaccagagcaaccagcuccagcuccaaaaccagagcaaccagcuccugcaccaaaaccagagcaaccagcuccagcuccaaaaccagagcaaccagcuccagcuccaaaaccagagcaaccagcuaagccggagaaaccagcugaagagccuacucaaccagaaaaaccagccacuccaaaaacaggcuggaaacaagaaaacgguaugugguauuucuacaauacugaugguucaauggcaauagguuggcuccaaaacaacgguucaugguacuaccuaaacgcuaacggcgcuauggcaacagguugggugaaagauggagauaccugguacuaucuugaagcaucaggugcuaugaaagcaagccaaugguucaaaguaucagauaaaugguacuaugucaacagcaauggcgcuauggcgacaggcuggcuccaauacaauggcucaugguacuaccucaacgcuaauggugauauggcgacaggauggcuccaauacaacgguucaugguauuaccucaacgcuaauggugauauggcgacaggaugggcuaaagucaacgguucaugguacuaccuaaacgcuaacggugcuauggcuacagguugggcuaaagucaacgguucaugguacuaccuaaacgcuaacgguucaauggcaacagguugggugaaagauggagauaccugguacuaucuugaagcaucaggugcuaugaaagcaagccaaugguucaaaguaucagauaaaugguacuaugucaauggcuuaggugcccuugcagucaacacaacuguagauggcuauaaagucaaugccaauggugaauggguuuaa

SEQ ID NO: 90 - RNA, 2460 nt, Streptococcus pneumoniae
augaaaauuaauaaaaaauaucuagcagguucaguggcaguccuugcccuaaguguuuguuccuaugagcuuggacguuaccaagcuggucaggauaagaaagagucuaaucgaguugcuuauauagauggugaucaggcuggucaaaaggcagaaaacuugacaccagaugaagucaguaagagggaggggaucaacgccgaacaaauuguuaucaagauuacggaucaagguuaugugaccucucauggagaccauuaucauuacuauaauggcaagguuccuuaugaugccaucaucagugaagagcuccucaugaaagauccgaauuaucaguugaaggauucagacauugucaaugaaaucaagggugguuaugucauuaagguaaacgguaaauacuauguuuaccuuaaggaugcagcucaugcggauaauauucggacaaaagaagagauuaaacgucagaagcaggaacgcagucauaaucauaacucaagagcagauaaugcuguugcugcagccagagcccaaggacguuauacaacggaugauggguauaucuucaaugcaucugauaucauugaggacacgggugaugcuuauaucguuccucacggcgaccauuaccauuacauuccuaagaaugaguuaucagcuagcgaguuagcugcugcagaagccuauuggaaugggaagcagggaucucguccuucuucaaguucuaguuauaaugcaaauccagcucaaccaagauugucagagaaccacaaucugacugucacuccaacuuaucaucaaaaucaaggggaaaacauuucaagccuuuuacgugaauuguaugcuaaacccuuaucagaacgccauguggaaucugauggccuuauuuucgacccagcgcaaaucacaagucgaaccgccagagguguagcugucccucaugguaaccauuaccacuuuaucccuuaugaacaaaugucugaauuggaaaaacgaauugcucguauuauuccccuucguuaucguucaaaccauuggguaccagauucaagaccagaagaaccaaguccacaaccgacuccagaaccuaguccaaguccgcaaccagcuccaagcaauccaauugaugagaaauuggucaaagaagcuguucgaaaaguaggcgaugguuaugucuuugaggagaauggaguuucucguuauaucccagccaaggaucuuucagcagaaacagcagcaggcauugauagcaaacuggccaagcaggaaaguuuaucucauaagcuaggaacuaagaaaacugaccucccaucuagugaucgagaauuuuacaauaaggcuuaugacuuacuagcaagaauucaccaagauuuacuugauaauaaaggucgacaaguugauuuugaggcuuuggauaaccuguuggaacgacucaaggaugucucaagugauaaagucaaguuaguggaagauauucuugccuucuuagcuccgauucgucauccagaacguuuaggaaaaccaaaugcgcaaauuaccuacacugaugaugagauucaaguagccaaguuggcaggcaaguacacaacagaagacgguuauaucuuugauccucgugauauaaccagugaugagggggaugccuauguaacuccacauaugacccauagccacuggauuaaaaaagauaguuugucugaagcugagagagcggcagcccaggcuuaugcuaaagagaaagguuugaccccuccuucgacagaccaucaggauucaggaaauacugaggcaaaaggagcagaagcuaucuacaaccgcgugaaagcagcuaagaaggugccacuugaucguaugccuuacaaucuucaauauacuguagaagucaaaaacgguaguuuaaucauaccucauuaugaccauuaccauaacaucaaauuugagugguuugacgaaggccuuuaugaggcaccuaagggguauacucuugaggaucuuuuggcgacugucaaguacuaugucgaacauccaaacgaacguccgcauucagauaaugguuuugguaacgcuagcgaccauguucaaagaaacaaaaauggucaagcugauaccaaucaaacggaaaaaccaagcgaggagaaaccucagacagaaaaaccugaggaagaaaccccucgagaagagaaaccgcaaagcgagaaaccagagucuccaaaaccaacagaggaaccagaagaaucaccagaggaaucagaagaaccucaggucgagacugaaaagguugaagaaaaacugagagaggcugaagauuuacuuggaaaaauccaggauccaauuaucaaguccaaugccaaagagacucucacaggauuaaaaaauaauuuacuauuuggcacccaggacaacaauacuauuauggcagaagcugaaaaacuauuggcuuuauuaaaggagaguaaguaa

SEQ ID NO: 91 - RNA, 2520 nt, Streptococcus pneumoniae
augaaaauuaauaaaaaauaucuagcagguucaguggcaguccuugcccuaaguguuuguuccuaugaacuuggucgucaccaagcuggucagguuaagaaagagucuaaucgaguuucuuauauagauggugaucaggcuggucaaaaggcagaaaacuugacaccagaugaagucaguaagagggaggggaucaacgccgaacaaaucgucaucaagauuacggaucaagguuaugugaccucucauggagaccauuaucauuacuauaauggcaaggucccuuaugaugccaucaucagugaagagcuccucaugaaagauccgaauuaucaguugaaggauucagacauugucaaugaaaucaagggugguuauguuaucaagguagauggaaaauacuauguuuaccuuaaggaugcagcucaugcggauaauauucggacaaaagaagagauuaaacgucagaagcaggaacacagucauaaucacgggggugguucuaacgaucaagcaguaguugcagccagagcccaaggacgcuauacaacggaugaugguuauaucuucaaugcaucugauaucauugaggacacgggugaugcuuauaucguuccucacggcgaccauuaccauuacauuccuaagaaugaguuaucagcuagcgaguuagcugcugcagaagccuauuggaaugggaagcagggaucucguccuucuucaaguucuaguuauaaugcaaauccagcucaaccaagauugucagagaaccacaaucugacugucacuccaacuuaucaucaaaaucaaggggaaaacauuucaagccuuuuacgugaauuguaugcuaaacccuuaucagaacgccauguggaaucugauggccuuauuuucgacccagcgcaaaucacaagucgaaccgccagagguguagcugucccucaugguaaccauuaccacuuuaucccuuaugaacaaaugucugaauuggaaaaacgaauugcucguauuauuccccuucguuaucguucaaaccauuggguaccagauucaagaccagaacaaccaaguccacaaucgacuccggaaccuaguccaaguccgcaaccugcaccaaauccucaaccagcuccaagcaauccaauugaugagaaauuggucaaagaagcuguucgaaaaguaggcgaugguuaugucuuugaggagaauggaguuucucguuauaucccagccaaggaucuuucagcagaaacagcagcaggcauugauagcaaacuggccaagcaggaaaguuuaucucauaagcuaggagcuaagaaaacugaccucccaucuagugaucgagaauuuuacaauaaggcuuaugacuuacuagcaagaauucaccaagauuuacuugauaauaaaggucgacaaguugauuuugaggcuuuggauaaccuguuggaacgacucaaggaugucccaagugauaaagucaaguuaguggaugauauucuugccuucuuagcuccgauucgucauccagaacguuuaggaaaaccaaaugcgcaaauuaccuacacugaugaugagauucaaguagccaaguuggcaggcaaguacacaacagaagacgguuauaucuuugauccucgugauauaaccagugaugagggggaugccuauguaacuccacauaugacccauagccacuggauuaaaaaagauaguuugucugaagcugagagagcggcagcccaggcuuaugcuaaagagaaagguuugaccccuccuucgacagaccaucaggauucaggaaauacugaggcaaaaggagcagaagcuaucuacaaccgcgugaaagcagcuaagaaggugccacuugaucguaugccuuacaaucuucaauauacuguagaagucaaaaacgguaguuuaaucauaccucauuaugaccauuaccauaacaucaaauuugagugguuugacgaaggccuuuaugaggcaccuaagggguauacucuugaggaucuuuuggcgacugucaaguacuaugucgaacauccaaacgaacguccgcauucagauaaugguuuugguaacgcuagcgaccauguucguaaaaauaagguagaccaagacaguaaaccugaugaagauaaggaacaugaugaaguaagugagccaacucacccugaaucugaugaaaaagagaaucacgcugguuuaaauccuucagcagauaaucuuuauaaaccaagcacugauacggaagagacagaggaagaagcugaagauaccacagaugaggcugaaauuccucaaguagagaauucuguuauuaacgcuaagauagcagaugcggaggccuugcuagaaaaaguaacagauccuaguauuagacaaaaugcuauggagacauugacuggucuaaaaaguagucuucuucucggaacgaaagauaauaacacuauuucagcagaaguagauagucucuuggcuuuguuaaaagaaagucaaccggcuccuauacaguag

SEQ ID NO: 92 - RNA, 2574 nt, Streptococcus pneumoniae
augaaauucaauccaaaucaaagauauacucguuggucuauucgccgucucagugucgguguugccucaguuguuguggcuaguggcuucuuuguccuaguuggucagccaaguucuguacgugccgaugggcucaauccaaccccaggucaagucuuaccugaagagacaucgggaacgaaagagggugacuuaucagaaaaaccaggagacaccguucucacucaagcgaaaccugagggcguuacuggaaauacgaauucacuuccgacaccuacagaaagaacugaagugagcgaggaaacaagcccuucuagucuggauacacuuuuugaaaaagaugaagaagcucaaaaaaauccagagcuaacagaugucuuaaaagaaacuguagauacagcugauguggaugggacacaagcaaguccagcagaaacuacuccugaacaaguaaaagguggagugaaagaaaauacaaaagacagcaucgauguuccugcugcuuaucuugaaaaagcugaagggaaagguccuuucacugccgguguaaaccaaguaauuccuuaugaacuauucgcuggugaugguauguuaacucgucuauuacuaaaagcuucggauaaugcuccuuggucugacaaugguacugcuaaaaauccugcuuuaccuccucuugaaggauuaacaaaagggaaauacuucuaugaaguagacuuaaauggcaauacuguugguaaacaaggucaagcuuuaauugaucaacuucgcgcuaaugguacucaaacuuauaaagcuacuguuaaaguuuacggaaauaaagacgguaaagcugacuugacuaaucuaguugcuacuaaaaauguagacaucaacaucaauggauuaguugcuaaagaaacaguucaaaaagccguugcagacaacguuaaagacaguaucgauguuccagcagccuaccuagaaaaagccaagggugaagguccauucacagcaggugucaaccaugugauuccauacgaacucuucgcaggugauggcauguugacucgucucuugcucaaggcaucugacaaggcaccauggucagauaacggcgacgcuaaaaacccagcccuaucuccacuaggcgaaaacgugaagaccaaaggucaauacuucuaucaaguagccuuggacggaaauguagcuggcaaagaaaaacaagcgcucauugaccaguuccgagcaaaugguacucaaacuuacagcgcuacagucaaugucuaugguaacaaagacgguaaaccagacuuggacaacaucguagcaacuaaaaaagucacuauuaacauaaacgguuuaauuucuaaagaaacaguucaaaaagccguugcagacaacguuaaagacaguaucgauguuccagcagccuaccuagaaaaagccaagggugaagguccauucacagcaggugucaaccaugugauuccauacgaacucuucgcaggugaugguauguugacucgucucuugcucaaggcaucugacaaggcaccauggucagauaacggugacgcuaaaaacccagcccuaucuccacuaggugaaaacgugaagaccaaaggucaauacuucuaucaauuagccuuggacggaaauguagcuggcaaagaaaaacaagcgcucauugaccaguuccgagcaaacgguacucaaacuuacagcgcuacagucaaugucuaugguaacaaagacgguaaaccagacuuggacaacaucguagcaacuaaaaaagucacuauuaacauaaacgguuuaauuucuaaagaaacaguucaaaaagccguugcagacaacguuaaggacaguaucgauguuccagcagccuaccuagaaaaggccaagggugaagguccauucacagcaggugucaaccaugugauuccauacgaacucuucgcaggugauggcauguugacucgucucuugcucaaggcaucugacaaggcaccauggucagauaacggcgacgcuaaaaacccagcucuaucuccacuaggugaaaacgugaagaccaaaggucaauacuucuaucaaguagccuuggacggaaauguagcuggcaaagaaaaacaagcgcucauugaccaguuccgagcaaacgguacucaaacuuacagcgcuacagucaaugucuaugguaacaaagacgguaaaccagacuuggacaacaucguagcaacuaaaaaagucacuauuaagauaaauguuaaagaaacaucagacacagcaaaugguucauuaucaccuucuaacucugguucuggcgugacuccgaugaaucacaaucaugcuacagguacuacagauagcaugccugcugacaccaugacaaguucuaccaacacgauggcaggugaaaacauggcugcuucugcuaacaagaugucugauacgaugaugucagaggauaaagcuaugcuaccaaauacuggugagacucaaacaucaauggcaaguauugguuuccuugggcuugcgcuugcagguuuacucgguggucuagguuugaaaaacaaaaaagaagaaaacuaa

SEQ ID NO: 93 - RNA, 2661 nt, Streptococcus pneumoniae
augaaaaaaguaagaaagauauuucagaaggcaguugcaggacugugcuguauaucucaguugacagcuuuuucuucgauaguugcuuuagcagaaacgccugaaaccaguccagcgauaggaaaaguagugauuaaggagacaggcgaaggaggagcgcuucuaggagaugccgucuuugaguugaaaaacaauacggauggcacaacuguuucgcaaaggacagaggcgcaaacaggagaagcgauauuuucaaacauaaaaccugggacauacaccuugacagaagcccaaccuccaguugguuauaaacccucuacuaaacaauggacuguugaaguugagaagaauggucggacgacuguccaaggugaacagguagaaaaucgagaagaggcucuaucugaccaguauccacaaacagggacuuauccagauguucaaacaccuuaucagauuauuaagguagaugguucggaaaaaaacggacagcacaaggcguugaauccgaauccauaugaacgugugauuccagaagguacacuuucaaagagaauuuaucaagugaauaauuuggaugauaaccaauauggaaucgaauugacgguuagugggaaaacaguguaugaacaaaaagauaagucugugccgcuggaugucguuaucuugcucgauaacucaaauaguaugaguaacauucgaaacaagaaugcucgacgugcggaaagagcuggugaggcgacacguucucuuauugauaaaauuacaucugauucagaaaauaggguagcgcuugugacuuaugcuuccacuaucuuugaugggaccgaguuuacaguagaaaaagggguagcagauaaaaacggaaagcgauugaaugauucucuuuuuuggaauuaugaucagacgaguuuuacaaccaauaccaaagauuauaguuauuuaaagcugacuaaugauaagaaugacauuguagaauuaaaaaauaagguaccuaccgaggcagaagaccaugauggaaauagauugauguaccaauucggugccacuuuuacucagaaagcuuugaugaaggcagaugagauuuugacacaacaagcgagacaaaauagucaaaaagucauuuuccauauuacggauggugucccaacuaugucguauccgauuaauuuuaaucaugcuacguuugcuccaucauaucaaaaucaacuaaaugcauuuuuuaguaaaucuccuaauaaagauggaauacuauuaagugauuuuauuacgcaagcaacuaguggagaacauacaauuguacgcggagaugggcaaaguuaccagauguuuacagauaagacaguuuaugaaaaaggugcuccugcagcuuucccaguuaaaccugaaaaauauucugaaaugaaggcggcugguuaugcaguuauaggcgauccaauuaaugguggauauauuuggcuuaauuggagagagaguauucuggcuuauccguuuaauucuaauacugcuaaaauuaccaaucauggugacccuacaagaugguacuauaacgggaauauugcuccugauggguaugaugucuuuacgguagguauugguauuaacggagauccugguacggaugaagcaacggcuacuaguuuuaugcaaaguauuucuaguaaaccugaaaacuauaccaauguuacugacacgacaaaaauauuggaacaguugaaucguuauuuccacaccaucguaacugaaaagaaaucaauugagaaugguacgauuacagauccgaugggugaguuaauugauuugcaauugggcacagauggaagauuugauccagcagauuacacuuuaacugcaaacgaugguagucgcuuggagaauggacaagcuguaggugguccacaaaaugauggugguuuguuaaaaaaugcaaaagugcucuaugauacgacugagaaaaggauucguguaacaggucuguaccuuggaacggaugaaaaaguuacguugaccuacaauguucguuugaaugaugaguuuguaagcaauaaauuuuaugauaccaauggucgaacaaccuuacauccuaaggaaguagaacagaacacagugcgcgacuucccgauuccuaagauucgugaugugcggaaguauccagaaaucacaauuucaaaagagaaaaaacuuggugacauugaguuuauuaaggucaauaaaaaugauaaaaaaccacugagaggugcggucuuuagucuucaaaaacaacauccggauuauccagauauuuauggagcuauugaucaaaauggcacuuaucaaaaugugagaacaggugaagaugguaaguugaccuuuaaaaaucugucagaugggaaauaucgauuauuugaaaauucugaaccagcugguuauaaacccguucaaaauaagccuaucguugccuuccaaauaguaaauggagaagucagagaugugacuucaaucguuccacaagauauaccagcggguuacgaguuuacgaaugauaagcacuauauuaccaaugaaccuauuccuccaaagagagaauauccucgaacuggugguaucggaauguugccauucuaucugauagguugcaugaugaugggaggaguucuauuauacacacggaaacauccguaa

SEQ ID NO: 94 - RNA, 2877 nt, Streptococcus pneumoniae
augucuaagaaaagauuguacgaaaucgcaaaagaacuuggaaaagaaaguaaagaaguuguagcgcgugcaaaagaguugggcuuggaugugaaaagccacucaucaaguguggaagaagcugucgcugcaaaaauugcugccagcuuuaagccugcagcugcuccgaaaguagaagcaaaaccugcagccccaaaaguaagugcagaaaagaaagccgaaaaaucugagccagcuaaaccagcuguagcuaaggaagaggcaaaaccugcagccccaaaagcaagugcagaaaagaaagccgaaaagucugaaccaguaaaaccagcuguagccaaggaagaggcaaaaccagcugagccagucacuccgaaaacagaaaaaguagcggcuaaaccgcaaagucguaauuucaaggcugagcgugaagcacgugcuaaagagcaggcagagcgacgcaagcaaaauaagggcaauaaccgugaccaacaacaaaacggaaaccgucagaaaaacgacggccguaaugguggaaaacaaggucaaagcaaccgcgacaaucgucgcuuuaaugaccaagcuaagaagcagcaaggucagcaaaaacguagaaaugagcgccgucagcaagaggauaaacguucaaaucaagcggcuccacguauugacuuuaaagcccgugcagcagcccuaaaagcagagcaaaaugcagaguacgcucguucaagugaggaacgcuucaagcaguaucaggcugcuaaagaagccuuggcucaagcuaacaaacgcaaggaaccagaggaaaucuuugaagaagcggcuaaguuagcugaacaagcacagcaaguucaagcagugguugaagucgucccugagaaaaaagaaccugcaguggauacacgucguaaaaaacaagcucgaccagacaaaaaucgugacgauuaugaucaugaagaagaugguccuagaaaacaacaaaagaaucgaaguagucaaaaucaagugagaaaucaaaagaauaguaacuggaauaacaacaaaaagaacaaaaaaggcaauaacaagaacaaccguaaucagacuccaaaaccuguuacggagcguaaauuccaugaauugccaacagaauuugaauauacagaugguaugaccguugcggaaaucgcaaaacguaucaaacgugaaccagcugaaauuguuaagaaacuuuucaugaugggugucauggccacacaaaaccaauccuuggauggggaaacaauugaacuccucaugguggauuacgguaucgaagccaaacaaaagguugaaguggauaaugcugacaucgaacguuucuuugucgaagaugguuaucucaaugaagaugaauugguugagcguccaccaguuguuacuaucaugggacacguugaccacgguaaaacaacccuuuuggauacucuucguaacucacguguugcgacaggugaagcaggugguauuacucagcauaucggugccuaccaaaucguggaaaaugguaagaagauuaccuuccuugauacaccaggacacgcggccuuuacaucaaugcgugcgcguggugcuucuguuaccgauauuacgaucuuggucguagcggcagaugacgggguuaugccucagacuauugaagccaucaaccacucaaaagcagcuaacguuccaaucaucguagcuauuaacaagauugauaaaccaggugcuaacccagaacgcguuaucggugaauuggcagagcauggugugaugucaacugcuugggguggagauucugaauuuguugaaauuucggcuaaauucaaccaaaauaucgaagaauuguuggaaacaguccuucuuguggcugaaauccaagaacucaaagcagacccaacaguucgugcgaucgguacgguuaucgaagcgcgcuuggauaaaggaaaaggugcggucgcaacccuucuuguacaacaagguaccuugaauguucaagacccaaucguugucggaaauaccuucggucguguccgugcuaugaccaacgaccuuggucgucguguuaaaguugcuggaccaucaacaccagucucuaucacagguuugaacgaagcaccgauggcgggugaccacuuugccguuuacgaggaugaaaaaucugcgcgugcagcaggugaagagcgugccaaacgugcccucaugaaacaacgucaagcuacccaacguguuagccuugaaaaccucuuugauacccuuaaagcuggggaacucaaaucuguuaauguuaucaucaaggcugauguacaagguucuguugaagcccuuucugccucacuucaaaagauugacguggaaggugucaaagugacuaucguccacucagcggucggugcuaucaacgaaucagacgugacccuugccgaagcuucaaaugccuuuaucguugguuucaacguacgcccuacaccacaagcucgucaacaagcagaagcugacgauguggaaauccgucuucacagcauuaucuacaagguuaucgaagagauggaagaagcuaugaaagggaugcuugauccagaauuugaagaaaaaguuauuggugaagcgguuauccgugaaaccuucaaggugucuaaagugggaacuaucgguggauuuaugguuaucaacgguaagguugcccgugacucuaaaguccguguuauccgugauggugucguuaucuaugauggugaacucgcaagcuugaaacacuauaaagacgacgugaaagaagugacaaacggucgugaagguggauugaugaucgacggcuacaaugauauuaagauggaugaugugauugaggcguaugucauggaagaaaucaagagauaa

SEQ ID NO: 95 - RNA, 3117 nt, Streptococcus pneumoniae
augaauaaagaaaaaauaaaaagaaaauuaaucacaauacuguuuguauguauugggauguuauguuuuggauuguuagcaggaguuaaggcugauaaucguguucaaaugagaacgacgauuaauaaugaaucgccauuguugcuuucuccguuguauggcaaugauaaugguaacggauuauggugggggaacacauugaagggagcaugggaagcuauuccugaagauguaaaaccauaugcagcgauugaacuucauccugcaaaagucuguaaaccaacaaguuguauuccacgagauacgaaagaauugagagaaugguaugucaagauguuggaggaagcucaaagucuaaacauuccaguuuucuugguuauuaugucggcuggagagcguaauacaguuccuccagagugguuagaugaacaauuccaaaaguauaguguguuaaaagguguuuuaaauauugagaauuauuggauuuacaauaaccaguuagcuccgcauagugcuaaauauuuggaaguuugugccaaauauggagcgcauuuuaucuggcaugaucaugaaaaaugguucugggaaacuauuaugaaugauccgacauucuuugaagcgagucaaaaauaucauaaaaauuugguguuggcaacuaaaaauacgccaauaagagaugaugcggguacagauucuaucguuaguggauuuugguugaguggcuuaugugauaacuggggcucaucaacagauacauggaaauggugggaaaaacauuauacaaacacauuugaaacuggaagagcuagggauaugagauccuaugcaucggaaccagaaucaaugauugcuauggaaaugaugaauguauauacugggggaggcacaguuuauaauuucgaaugugccgcguauacauuuaugacaaaugauguaccaacuccagcauuuacuaaagguauuauuccuuucuuuagacaugcuauacaaaauccagcuccaaguaaggaagaaguuguaaauagaacaaaagcuguauuuuggaauggagaagguaggauuaguucauuaaacggauuuuaucaaggacuuuauucgaaugaugaaacaaugccuuuauauaauaaugggagauaucauauucuuccuguaauacaugagaaaauugauaaggaaaagauuucaucuauauucccuaaugcaaaaauuuugacuaaaaauagugaggaauugucuaguaaagucaacuauuuaaacucgcuuuauccaaaacuuuaugaaggagauggguaugcucagcguguagguaauuccugguauauuuauaauaguaaugcuaauaucaauaaaaaucagcaaguaauguugccuauguauacuaauaauacaaagucguuaucguuagauuugacgccacauacuuacgcuguuguuaaagaaaauccaaauaauuuacauauuuuauugaauaauuacaggacagauaagacagcuaugugggcauuaucaggaaauuuugaugcaucaaaaaguuggaagaaagaagaauuagaguuagcgaacuggauaagcaaaaauuauuccaucaauccuguagauaaugacuuuaggacaacaacacuuacauuaaaagggcauacuggucauaaaccucagauaaauauaaguggcgauaaaaaucauuauacuuauacagaaaauugggaugagaauacccauguuuauaccauuacgguuaaucauaauggaaugguagagaugucuauaaauacugaggggacagguccagucucuuucccaacaccagauaaauuuaaugaugguaauuugaauauagcauaugcaaaaccaacaacacaaaguucuguagauuacaauggagacccuaauagagcuguggaugguaacagaaaugguaauuuuaacucugguucgguaacacacacuagggcagauaaucccucuuggugggaagucgauuugaaaaaaauggauaaaguugggcuuguuaaaauuuauaaucgcacagaugcugagacucaacgucuaucuaauuuugaugugauucuauaugacaauaauagaaacgaaguugcuaagaaacauguuaauaauuugucgggugaaucuguuagucuagauuucaaagaaaaaggagcaagguauauuaaaguuaaauuacuaacgaguggagugccuuugaguuuagcagaaguagagguuuuuagagaaucagaugguaagcaaucugaagaggauauagauaaaauaacagaagauaaaguagucucuacaaauaagguagcuacucaaaguucaaccaauuaugaggguguagcugcuuuagcaguugaugguaauaaagauggagauuacggacaucauucggugacucauacuaaggcagauucuaacgcuugguggcaggucgaucugggagaagaguuuacgguuucuaaaguugauauuuauaauagaacagaugccgaaccucagcguuuaucuaauuuugauguuauuuuucuaucuucaucaggagaagaaguuuuuagaagacauuuugauaaaguaguugaugguuuguuaucuuuaaaaguaccuucuguaggggcuaagcuagucaaaauagaauuaaaaucagcagcuauuccguuaaguuuagcggaaguugaagucuaugguucaaagagaacuccgaagaaacuuucuaauauugcauuaaccaaagaaacucgacagaguucaacggauuacaaugguuuuucucgucuagcaguugauggaaauaaaaacggagauuauggucaucauucagugacucauaccaaaggagauucuccuucauggugggagauagauuuagcacaaaccgaagaauuagaaaaguuaauuauuuauaauagaacagaugcugaaauucagagauuaucaaauuuugauauuauuauauaugauucaaauaaucaugaaguuuuuaaacaacauauugacaguuuagaaagcaauaaucuauccauagacuuaaaagggcugaagggaaaaaagguuagaauuucuuugagaaacgcaggaauuccuuuaaguuuggcagagguagagguuuauacuuauaaguaa

SEQ ID NO: 96 - RNA, 3120 nt, Streptococcus pneumoniae
augaaauuuaguaaaaaauauauagcagcuggaucagcuguuaucguauccuugagucuaugugccuaugcacuaaaccagcaucguucgcaggaaaauaaggacaauaaucgugucucuuauguggauggcagccagucaagucagaaaagugaaaacuugacaccagaccagguuagccagaaagaaggaauucaggcugagcaaauuguaaucaaaauuacagaucagggcuauguaacgucacacggugaccacuaucauuacuauaaugggaaaguuccuuaugaugcccucuuuagugaagaacucuugaugaaggauccaaacuaucaacuuaaagacgcugauauugucaaugaagucaagggugguuauaucaucaaggucgauggaaaauauuaugucuaccugaaagaugcagcucaugcugauaauguucgaacuaaagaugaaaucaaucgucaaaaacaagaacaugucaaagauaaugagaagguuaacucuaauguugcuguagcaaggucucagggacgauauacgacaaaugaugguuaugucuuuaauccagcugauauuaucgaagauacggguaaugcuuauaucguuccucauggaggucacuaucacuacauucccaaaagcgauuuaucugcuagugaauuagcagcagcuaaagcacaucuggcuggaaaaaauaugcaaccgagucaguuaagcuauucuucaacagcuagugacaauaacacgcaaucuguagcaaaaggaucaacuagcaagccagcaaauaaaucugaaaaucuccagagucuuuugaaggaacucuaugauucaccuagcgcccaacguuacagugaaucagauggccuggucuuugacccugcuaagauuaucagucguacaccaaauggaguugcgauuccgcauggcgaccauuaccacuuuauuccuuacagcaagcuuucugccuuagaagaaaagauugccagaauggugccuaucaguggaacugguucuacaguuucuacaaaugcaaaaccuaaugaaguagugucuagucuaggcagucuuucaagcaauccuucuucuuuaacgacaaguaaggagcucucuucagcaucugaugguuauauuuuuaauccaaaagauaucguugaagaaacggcuacagcuuauauuguaagacauggugaucauuuccauuacauuccaaaaucaaaucaaauugggcaaccgacucuuccaaacaauagucuagcaacaccuucuccaucucuuccaaucaauccaggaacuucacaugagaaacaugaagaagauggauacggauuugaugcuaaucguauuaucgcugaagaugaaucagguuuugucaugagucacggagaccacaaucauuauuucuucaagaaggacuugacagaagagcaaauuaaggcugcgcaaaaacauuuagaggaaguuaaaacuagucauaauggauuagauucuuugucaucucaugaacaggauuauccaaguaaugccaaagaaaugaaagauuuagauaaaaaaaucgaagaaaaaauugcuggcauuaugaaacaauauggugucaaacgugaaaguauugucgugaauaaagaaaaaaaugcgauuauuuauccgcauggagaucaccaucaugcagauccgauugaugaacauaaaccgguuggaauuggucauucucacaguaacuaugaacuguuuaaacccgaagaaggaguugcuaaaaaagaagggaauaaaguuuauacuggagaagaauuaacgaauguuguuaauuuguuaaaaaauaguacguuuaauaaucaaaacuuuacucuagccaauggucaaaaacgcguuucuuuuaguuuuccgccugaauuggagaaaaaauuagguaucaauaugcuaguaaaauuaauaacaccagauggaaaaguauuggagaaaguaucugguaaaguauuuggagaaggaguagggaauauugcaaacuuugaauuagaucaaccuuauuuaccaggacaaacauuuaaguauacuaucgcuucaaaagauuauccagaaguaaguuaugaugguacauuuacaguuccaaccucuuuagcuuacaaaauggccagucaaacgauuuucuauccuuuccaugcaggggauacuuauuuaagagugaacccucaauuugcagugccuaaaggaacugaugcuuuagucagaguguuugaugaauuucauggaaaugcuuauuuagaaaauaacuauaaaguuggugaaaucaaauuaccgauuccgaaauuaaaccaaggaacaaccagaacggccggaaauaaaauuccuguaaccuucauggcaaaugcuuauuuggacaaucaaucgacuuauauuguggaaguaccuaucuuggaaaaagaaaaucaaacugauaaaccaaguauucuaccacaauuuaaaaggaauaaagcacaagaaaacuuaaaacuugaugaaaagguagaagaaccaaagacuagugagaagguagaaaaagaaaaacuuucugaaacugggaauaguacuaguaauucaacguuagaagaaguuccuacaguggauccuguacaagaaaaaguagcaaaauuugcugaaaguuaugggaugaagcuagaaaaugucuuguuuaauauggacggaacaauugaauuauauuuaccaucgggagaagucauuaaaaagaauauggcagauuuuacaggagaagcaccucaaggaaauggugaaaauaaaccaucugaaaauggaaaaguaucuacuggaacaguugagaaccaaccaacagaaaauaaaccagcagauucuuuaccagaggcaccaaacgaaaaaccuguaaaaccagaaaacucaacggauaauggaauguugaauccagaagggaauguggggagugacccuauguuagauccagcauuagaggaagcuccagcaguagauccuguacaagaaaaauuagaaaaauuuacagcuaguuacggauuaggcuuagauaguguuauauucaauauggauggaacgauugaauuaagauugccaaguggagaagugauaaaaaagaauuuaucugaucucauagcguaa

SEQ ID NO: 97 - RNA, 3234 nt, Streptococcus pneumoniae
augauuuuacaauauguuuauuggaguguauacaugcaaacaaaaacaaagaagcucauugugaguuugucuucacuuguuuuaucaggauuuuuauuaaaccauuauaugacaauuggagcggaagaaacgacuacgaauaccauucagcaaagccagaaggaaguucaguaucagcaaagggauacaaaaaauuuaguugaaaauggugauuuuggucagacggaggacggaagcaguccguggacaggaagcaaagcucagggguggucagcuuggguagaccagaagaauagugcagaugccucaacucgagucauugaggcuaaggauggggcuaucacuaucucaagccaugagaaauuaagggcagcgcuucaccguaugguuccuauugaagcuaagaaaaaguauaaacugcguuucaagauuaaaacagauaauaaaaucgggauugccaaaguucguaucauugaggaaagugguaaggacaagcgauuguggaauucugcaacgacgucaggaacaaaggacuggcagaccauugaagcagacuauagcccgacuuuagauguugauaaaaucaagcuggaguuauucuaugaaacaggaacugggacuguuuccuuuaaggauauugagcugguagagguagcagaccagcuuucugaggauucucaaacagauaaacagcuugaggaaaagauugauuuaccaauuggaaaaaaacauguuuuuucucuugcggacuauacuuauaagguagaaaauccugacguugcuucagucaaaaauggaauuuuagaaccucuuaaggaagggacaaccaaugucauugucaguaaagauggcaaggaagugaaaaagauuccuuugaagauucuggccucuguuaaggaugcauacacagaccguuuggaugacuggaauggcaucaucgcugggaaucaauacuaugauucuaaaaaugaacagauggccaaauuaaaccaggaauuggaaggaaagguagcugauagccuauccaguauuucaagucaggcggaccgcaccuauuugugggaaaaauuuucaaauuauaaaacgucugcaaaucugacugccacuuaucggaaauuggaggagauggccaagcaagugaccaauccuucuucucguuauuaucaagaugaaacugucguucgaacagucagggauuccauggaauggaugcauaaacaugucuacaauagugaaaagagcauuguugggaacuggugggauuaugaaaucgguacaccucgugccaucaacaauaccuugucucugaugaaagaauacuucucugaugaggaaauuaaaaaauauacagaugugauugaaaaauuuguaccagaucccgaacauuuccgaaagacgacugauaacccauucaaggcucuagguggaaacuuaguugauaugggaaggguaaaaguaauagcugguuuacugcguaaggaugaucaagaaauuucuucuaccauucgcucgauugagcaaguguucaaguugguagaccaaggugaagguuuuuaucaagauggauccuauaucgaccacaccaauguugccuauacgggugcuuaugggaauguuuugauugauggccugucucaacuguugccagucauucaaaagaccaagaauccaaucgauaaagauaaaaugcaaaccauguaccacuggauugauaaaucguuugcuccuuugcuggugaauggagaguugauggauaugagucguggacgcucgaucagucgugcaaauagcgaggggcacguggccgcaguagaaguacuaagagggauucaccgaauagcggauaugucugaaggagaaaccaaacaauguuugcagagucuugugaagaccauuguucaaucggauaguuauuaugaugucuuuaagaauuugaagacuuauaaggauaucaguuugaugcaauccuuguuaagugaugcaggagucgcaaguguuccaagaccaaguuaccuaucugccuuuaacaagauggauaaaacagccauguacaaugcagagaaaggguuuggauuuggcuugucacucuuuuccagucguaccuugaauuacgaacacaugaacaaggaaaauaaacgugguugguauacgagugaugggauguucuaucuuuacaauggcgauuugagucacuauagcgauggcuacuggccaacaguuaauccauauaagaugccugguacaacagagacggaugcuaagagagcggauagcgauacagguaaaguuuuaccgucugcuuucguuggaacgagcaaacuagaugaugccaaugcgacagcaaccauggauuucaccaacuggaaucaaacauugacugcucauaagagcugguuuaugcuaaaggauaagaucgccuuuuuaggaagcaauauccaaaacacuucaacagauacugcugcaacuacaauugaccagagaaaacuggaaucagguaauccauauaaagucuaugucaaugauaaagaagccucccuuacagaacaagaaaaggauuauccugaaacccaaagugucuuuuuagaaucguucgauucgaaaaagaauauugguuacuuuuucuuuaagaagaguucaaucaguaugaguaaggcuuugcaaaagggagccuggaaggauaucaaugaaggacagucagacaaggaaguugaaaaugaauuucuuacgauuagucaggcucauaagcaaaauagagauucuuauggcuauaugcucauuccuaacguggaucgugccaccuucaaucaaaugauaaaagaguuagaaaguagccucaucgaaaauaacgaaacccuucagucuguuuaugaugcuaaacaaggaguuuggggcauugugaaauaugaugauucugucucuacuauuuccaaccaauuccaaguuuugaaacguggagucuauaccauucgaaaagaaggggaugaauauaagauugccuacuauaauccugaaacccaggaaucagcuccagaucaggaagucuuuaaaaagcuagagcaagcagcucagccacaaguacagaauucaaaagaaaaggaaaaaucugaagaggaaaagaaccauucggaucaaaagaaucucccucagacaggagaaggucagucaaucuuggcaagucuaggguucuugcuacuuggggcauuuuaucuauuccguagaggaaagaacaacuaa

SEQ ID NO: 98 - RNA, 3807 nt, Streptococcus pneumoniae
augagaaaaacaccaucucacacugagaaaaaaauggucuacagcauccguucccucaaaaauggaacugguucuguccuuaucggcgcaagccuugucuugcuugccauggcuacaccaacuaucucauccgacgaaaguacaccaaccacuaacgaacccaacaacagaaauacaaccacccuugcccaaccucuuacugauacagcagcuggcucugguaagaacgaaagugauauuucuucaccuagaaaugcaaacgcuucccuagagaaaacagaagaaaaaccugcaacagagccaacuacuucuacuaguccaguaacaacugaaacuaaggcagaagagcccaucgaagauaacuacuuccguauccaugucaaaaaacuuccugaagaaaacaaggaugcucaaggacuauggacuugggacgauguugaaaaaccaucugaaaacuggccaaacggagcuuuguccuucaaggaugccaagaaagaugacuacggcuauuaccuagaugucaaauuaaagggagaacaagccaagaaaauuagcuuccucaucaacaauacagcuggaaaaaaucuaaccggcgauaaaucuguagaaaaacuaguuccaaaaaugaacgaggcuugguuagaccaagauuacaagguuuucucuuacgagccacagccugcaggaacuguucgcgucaacuacuaccgcacagauggcaacuaugacaagaaaucucucugguacuggggagauguaaaaaauccaaguagcgcucaauggccugacggaacagacuuuacggcuacaggcaaauauggccgcuauaucgacauuccucuuaaugaagccgcaagagaauuuggauuuuuauuacuagaugagagcaaacaaggagucgacgugaaaauccguaaagaaaauuauaaguucacagauuugaaaaaucauagccaaauuuuccuaaaagacgaugaugaaucgauuuacacaaauccauacuauguccaugauauccguaugacaggagcccaacacguaggcacuucuagcauugaaaguagcuuuucaacacuugucggugcuaaaaaagaagauauccucaaacacuccaacaucacuaaucaccuaggaaacaagguaacuauuaccgauguugcaaucgaugaagcugguaagaaagugaccuacagcggagauuucucugacacaaaacauccuuauacuguuagcuacaauuccgaccaauucacuaccaaaacaagcuggcgccugaaagaugagacauacagcuaugauggcaaacugggagcugaccuaaaagaagaaggaaaacaaguugauuugacccuuuggucaccaagugcugauaagguuucuguuguugucuacgacaagaaugacccugacaaaguaguuggaacugucgcucucgaaaaaggggaaagaggaacuuggaaacaaacucuagacagcacaaacaaacucggaaucacagauuucacuggcuacuauuaucaauaccaaaucgagcgucaagguaaaacuguucuugcacucgauccuuacgcuaaaucucuugcugcuuggaauagcgacgaugccaaaauugacgauguccauaaaguggcuaaagccgccuuuguagauccagcuaaacucggaccucaagacuugacuuaugguaagauucacaauuucaagacucgugaagacgccguuaucuacgaagcucaugugcgugacuucacuucagauccugccauugcaaaagacuugaccaaaccauuugggacuuuugaagccuucauugaaaaacuagacuaucucaaagacuuggguguaacccauauccagcuccuuccagucuugucuuacuacuuugucaaugaauugaaaaaccaugaacgcuugucugacuacgcuucaagcaacagcaacuacaacuggggauaugacccucaaaacuacuucuccuugacugguauguacucaagcgauccuaagaauccagaaaaacgaaucgcagaauuuaaaaaccucaucaacgaaauccacaaacgugguaugggagcuauccuagaugucguuuauaaccacacagccaaagucgauaucuuugaagaccuagagccaaacuacuaccacuuuauggaugccgauggcacaccucgaacuagcuuuggugguggacgcuuggggacaacccaccauaugaccaaacggcuccuaguugacucuaucaaauaccuaguugauaccuacaaaguggauggcuuccgcuucgauaugaugggagaccaugacgccgcuucuaucgaagaagcuuacaaggcugcacgcgcccucaauccaaaccucaucaugcuuggugaagguuggagaaccuaugccggugaugaaaacaugccuacuaaagcugcugaccaagauuggaugaaacauaccgauacugucgcugucuuuucagaugacauccguaacaaccucaaaucugguuauccaaacgaaggucaaccugccuuuaucacagguggcaagcgugaugucaacaccaucuuuaaaaaucucauugcucaaccaacuaacuuugaagcugacagcccuggagaugucauccaauacaucgcagcccaugauaacuugacccucuuugacaucauugcccagucuaucaaaaaagacccaagcaaggcugagaacuaugcugaaauccaccgucguuuacgacuuggaaaucucauggucuugacagcucaaggaacuccauuuauccacuccggucaggaauauggacguacuaaacaauuccgugacccagccuacaagacuccaguagcagaggauaagguuccaaacaaaucucacuuguugcgugauaaggacggcaacccauuugacuauccuuacuucauccaugauucuuacgauucuagcgaugcagucaacaaguuugacuggacuaaggcuacagaugguaaagcuuauccugaaaaugucaagagccgugacuauaugaaagguuugauugcccuucgucaaucuacagaugccuuccgacuuaagagucuucaagauaucaaagaccguguccaccucaucacugucccaggccaaaaugguguggaaaaagaggauguagugauuggcuaccaaaucacugcuccaaacggcgauaucuacgcagucuuugucaaugcggaugaaaaagcucgcgaauuuaauuugggaacugccuuugcacaucuaagaaaugcggaaguuuuggcagaugaaaaccaagcaggaucagucggaauugccaacccgaaaggacuugaauggacugaaaaaggcuugaaauugaaugcccuuacagcuacuguucuucgagucucucaaaauggaacuagccaugagucaacugcagaagagaaaccagacucaaccccuuccaagccugaacaucaaaaugaagcuucucacccugcacaucaagacccagcuccagaagcuagaccugauucuacuaaaccagaugccaaaguagcugaugcggaaaauaaaccuagccaagcuacagcugauucacaagcugaacaaccagcacaagaagcacaagaagcacaagcaucaucuguaaaagaagcgguucgaaacgaaucgguagaaaacucuagcaaggaaaauauaccugcaaccccagauaaacaagcugaacuuccaaauacaggaaucaaaaacgaaaacaaacuccuauuugcaggaaucagccuccuugcgcuccuuggucucgguuucuuacuaaaaaauaaaaaagagaacuaa

SEQ ID NO: 99 - RNA, 3939 nt, Streptococcus pneumoniae
augaaacaugaaaaacaacagcguuuuucuauucguaaauacgcuguaggagcagcuucuguucuaauuggauuugccuuccaagcacagacuguugcagccgauggaguuacuccuacuacuacagaaaaccaaccgaccauccauacgguuucugauuccccucaaucauccgaaaaucggacugaggaaacaccuaaagcagugcuucaaccagaagcuccaaaaacuguagaaacagaaacuccagcuacugauaagguagcuagucuuccaaaaacagaagaaaaaccacaagaggaaguuaguucaacuccuagugauaaagcagaagugguaacuccaacuucugcugaaaaagaaacugcuaauaaaaaggcagaagaagcuagcccuaaaaaggaagaagcgaaagagguugauucuaaagagucaaauacagacaagacugacaaggauaaaccagcuaaaaaagaugaagcgaaagcagaggcugacaaaccggcaacagaggcaggaaaggaacgugcugcaacuguaaaugaaaaacuagcgaaaaagaaaauuguuucuauugaugcuggacguaaauauuucucaccagaacagcucaaggaaaucaucgauaaagcgaaacauuauggcuacacugauuuacaccuauuagucggaaaugauggacuccguuucauguuggacgauaugagcaucacagcuaacggcaagaccuaugccagugacgaugucaaacgcgccauugaaaaagguacaaaugauuauuacaacgauccaaacggcaaucacuuaacagaaagucaaaugacagaucugauuaacuaugccaaagauaaagguaucggucucauuccgacaguaaauaguccuggacacauggaugcgauucucaaugccaugaaagaauugggaauccaaaacccuaacuuuagcuauuuugggaagaaaucagcccguacugucgaucuugacaacgaacaagcugucgcuuuuacaaaagcccuuaucgacaaguaugcugcuuauuucgcgaaaaagacugaaaucuucaacaucggacuugaugaauaugccaaugaugcgacagaugcuaaagguuggagugugcuucaagcugauaaauacuauccaaacgaaggcuacccuguaaaaggcuaugaaaaauuuauugccuacgccaaugaccucgcucguauuguaaaaucgcacggucucaaaccaauggcuuuuaacgacgguaucuacuacaauagcgacacaagcuuugguaguuuugacaaagacaucaucguuucuauguggacuggugguuggggaggcuacgaugucgcuucuucuaaacuacuagcugaaaaaggucaccaaauccuuaauaccaaugaugcuugguacuacguucuuggacgaaacgcugauggccaaggcugguacaaucucgaucaggggcucaaugguauuaaaaacacaccaaucacuucuguaccaaaaacagaaggagcugauaucccaaucaucggugguaugguagcugcuugggcugacacuccaucugcacguuauucaccaucacgccucuucaaacucaugcgucauuuugcaaaugccaacgcugaauacuucgcagcugauuaugaaucugcagagcaagcacuuaacgagguaccaaaagaccugaaccguuauacugcagaaagcgucacggccguaaaagaagcugaaaaagcuauucgcucucucgauagcaaccuuagccgugcccaacaagauacgauugaucaagccauugcuaaacuucaagaaacugucaacaacuugacccucacgccugaagcucaaaaagaagaagaagcuaaacgugagguugaaaaacuugccaaaaacaagguaaucucaaucgaugcuggacgcaaauacuuuacucugaaccagcucaaacgcaucguagacaaggccagugagcucggauauucugauguccaucuccuucuaggaaaugacggacuucgcuuucuacucgaugauaugaccauuacugccaacggaaaaaccuaugcuagugaugacguuaaaaaagcuauuaucgaaggaacuaaagcuuacuacgacgauccaaacgguacugcacuaacacaggcagaaguaacagagcuaauugaauacgcuaaaucuaaggacaucggucucaucccagcuauuaacaguccaggucacauggaugcuaugcugguugccauggaaaaauuagguauuaaaaauccucaagcccacuuugauaaaguuucaaaaacaacuauggacuugaaaaacgaagaagcgaugaacuuuguaaaagcccucaucgguaaauacauggacuucuuugcagguaaaacaaagauuuucaacuuugguacugacgaauacgccaacgaugcgacuagugcccaaggcugguacuaccucaagugguaucaacucuauggcaaauuugccgaauaugccaacacccucgcagcuauggccaaagaaagagggcuucaaccaauggccuucaacgauggcuucuacuaugaagacaaggacgauguucaguuugacaaagaugucuugauuucuuacuggucuaaaggcugguggggauauaaccucgcaucaccucaauaccuagcaagcaaaggcuauaaauucuugaauaccaacggugacugguacuacauucuuggucaaaaaccagaagaugguggugguuuccucaagaaagcuauugagaauacuggaaaaacaccauucaaucaacuagcuucuaccaaauauccugaaguagaucuuccaacagucggaaguaugcuuucaaucugggcagauagaccaagcgcugaauacaaggaagaggaaaucuuugaacucaugacugccuuugcagaccacaacaaagacuacuuucgugcuaauuauaaugcucuccgcgaagaauuagcuaaaauuccuacaaacuuagaaggauauaguaaagaaagucuugaggcccuugacgcagcuaaaacagcucuaaauuacaaccucaaccguaauaaacaagcugagcuugacacgcuuguagccaaccuaaaagccgcucuucaaggccucaaaccagcuguaacucauucaggaagccuagaugaaaaugaaguggcugccaauguugaaaccagaccagaacucaucacaagaacugaagaaauuccauuugaaguuaucaagaaagaaaauccuaaccucccagccggucaggaaaauauuaucacagcaggagucaaaggugaacgaacucauuacaucucuguacucacugaaaauggaaaaacaacagaaacaguccuugauagccagguaaccaaagaaguuauaaaccaagugguugaaguuggcgcuccuguaacucacaagggugaugaaaguggucuugcaccaacuacugagguaaaaccuagacuggauauccaagaagaagaaauuccauuuaccacagugacuugugaaaauccacucuuacucaaaggaaaaacacaagucauuacuaagggcgucaauggacaucguagcaacuucuacucugugagcacuucugccgaugguaaggaagugaaaacacuuguaaauagugucguagcacaggaagccguuacucaaauagucgaagucggaacuaugguaacacauguaggcgaugaaaacggacaagccgcuauugcugaagaaaaaccaaaacuagaaaucccaagccaaccagcuccaucaacugcuccugcugaggaaagcaaaguucuuccucaagauccagcuccugugguaacagagaaaaaacuuccugaaacaggaacucacgauucugcaggacuaguagucgcaggacucauguccacacuagcagccuauggacucacuaaaagaaaagaagacuaa

SEQ ID NO: 100 - RNA, 4980 nt, Streptococcus pneumoniae
augaagaauccauuuuuugaaagacguugucguuacaguauucguaaguuaucaguaggagccugcucgcugaugauuggugcuguuuuauuugcugguccagccuuggcugaagaaacugcaguuccugaaaauagcggagcuaauacagagcuuguuucaggagagagugagcauucgaccaaugaagcugauaagcagaaugaaggggaacaugcuagagaaaacaagcuagaaaaggcagaaggaguagcgauagcaucugaaacugcuucgccagcaagcaaugaagcugcaacuacugaaacugcagaagcagcuagcgcagcuaaaccagaggaaaaagcaagugaggugguugcagaaacaccaucugcagaagcaaaaccuaagucugacaaggaaacagaagcaaagcccgaagcaacuaaccaaggggaugagucuaaaccagcagcagaagcuaauaagacugaaaaagaaguccagccagaugucccuaaaaauacagaaaaaacauuaaaaccaaaggaaaucaaauuuaauucuugggaagaauuguuaaaaugggaaccaggugcucgugaagaugaugcuauuaaccgcggaucuguuguccucgcuucacgucggacaggucauuuagucaaugaaaaagcuagcaaggaagcaaaaguucaagccuuaucaaacaccaauucuaaagcaaaagaccaugcuucuguugguggagaagaguucaaggccuaugcuuuugacuauuggcaauaucuagauucaauggucuucugggaaggucucguaccaacuccugacguuauugaugcaggucaccguaacgggguuccuguauacgguacacucuucuucaacuggucuaauaguauugcagaucaagaaagauuugcugaagcuuugaagcaagacgcagaugguagcuucccaauugcccguaaauugguagacauggccaaguauuauggcuaugauggcuauuucaucaaccaagaaacaacuggagauuugguuaaaccucuuggagaaaagaugcgccaguuuaugcucuauagcaaggaauaugcugcuaagguaaaccauccaaucaaguauucuugguacgaugccaugaccuauaacuauggacguuaucaucaagaugguuugggagaauacaacuaccaauucaugcaaccagaaggagauaagguuccggcagauaacuucuuugcuaacuuuaacugggauaaggcuaaaaaugauuacacuauugcaacugccaacuggauuggucguaauccuuaugauguauuugcagguuuggaauugcaacagggugguuccuacaagacaaagguuaaguggaaugacauuuuagacgaaaaugggaaauugcgccuuucucuugguuuauuugccccagauaccauuacaaguuuaggaaaaacuggugaagauuaucauaaaaaugaagauaucuucuuuacagguuaucaaggagacccuacuggccaaaaaccaggugacaaagauugguaugguauugcuaaccuaguugcggaccguacgccagcgguagguaauacuuuuacuacuucuuuuaauacaggucaugguaaaaaaugguucguagaugguaagguuucuaaggauucugaguggaauuaucguucaguaucagguguucuuccaacauggcgcugguggcagacuucaacaggggaaaaacuucgugcagaauaugauuuuacagaugccuauaauggcggaaauucccuuaaauucucuggugauguagccgguaagacagaucaggaugugagacuuuauucuacuaaguuagaaguaacugagaagaccaaacuucguguugcccacaagggaggaaaagguucuaaaguuuauauggcauucucuacaacuccagacuacaaauucgaugaugcagaugcauggaaagagcuaacccuuucugacaacuggacaaaugaagaauuugaucuuagcucacuagcggguaaaaccaucuaugcagucaaacuauuuuucgagcaugaaggugcuguaaaagauuaucaguuuaaccuaggacaauuaacuaucucggacaaucaccaagagccacaaucgccgacaagcuuuucuguagugaaacaaucucuuaaaaaugcccaagaagcggaagcaguugugcaauuuaaaggcaacaaggaugcagauuucuaugaaguuuaugaaaaagauggagacagcuggaaauuacuaacuggcucaucuucuacaacuauuuaucuaccaaaaguuagccgcucagcaagugcucaggguacaacucaagaacugaagguuguagcagucgguaaaaauggaguucguucagaagcugcaaccacaaccuuugauugggguaugacuguaaaagauaccagccuaccaaaaccacuagcugaaaauaucguuccaggugcaacaguuauugauaguacuuucccuaagacugaagguggagaagguauugaagguauguugaacgguaccauuacuagcuugucagauaaauggucuucagcucaguugagugguaguguggauauucguuugaccaagccacguaccguuguuagaugggucauggaucaugcaggagcugguggugagucuguuaacgauggcuugaugaacacuaaagacuuugaccuuuauuauaaagaugcagauggugaguggaagcuagcuaaggaaguccgugguaacaaagcacacgugacagauaucacucuugauaaaccaaucacugcucaagacuggcgcuugaauguugucacuucugacaauggaacuccauggaaggcuauucguaucuauaacuggaaaauguaugaaaagcuugauacugagagugucaauauuccgauggccaaggcugcagcccguucucuaggcaauaacaagguacaaguuggcuuugcagauguaccggcuggagcaacuauuaccguuuaugauaauccaaauucucaaacuccgcucgcaaccuugaagagcgaaguuggaggagaccuagcaagugcaccauuggauuugacaaaucaaucuggucuucuuuauuaucguacccaguugccaggcaaggaaauuaguaauguccuagcaguuuccguuccaaaagaugacagaagaaucaagucagucagccuagaaacaggaccuaagaaaacaagcuacgccgaaggggaggauuuggaccuuagaggugguguucuucgaguucaguaugaaggaggaacugaggacgaacucauucgccuaacucacgcagguguaucaguaucagguuuugauacgcaucauaagggagaacagaaucuuacucuccaauauuugggacaaccgguaaaugcuaauuugucagugacugucacuggccaagacgaagcaaguccgaaaacuauuuugggaauugaaguaagucaggaaccgaaaaaagauuaccuaguuggugauagcuuagacuugucugaaggacgcuuugcaguggcuuauagcaaugacaccauggaagaacauuccuuuacugaugagggaguugaaauuucugguuacgaugcucaaaagacuggucgucaaaccuugacgcuucauuaccaaggccaugaaguuagcuuugauguuuugguaucuccaaaagcagcauugaacgaugaguaccucaaacaaaaauuagcagaaguugaagcugcuaagaacaagguggucuauaacuuugcuucaucagaaguaaaagaagccuucuugaaagcaauugaagcggccgaacaaguguugaaagaccaugaaacuagcacccaagaucaagucaaugaccgacuuaauaaauugacagaagcucauaaagcucugaauggucaagagaaauuuacggaagaaaagacagagcuugaucgcuuaacaggugagguucaagaacucuuggcugccaaaccaaaccauccuucagguucugcccuagcuccgcuucuugagaaaaacaaggccuugguugaaaaaguagauuugaguccagaagagcuuacaacagcgaaacagagucuaaaagaucugguugcuuuauugaaagaagacaagccagcagucuuuucugauaguaaaacagguguugaaguacacuucucaaauaaagagaagacugucaucaaggguuugaaaguagagcguguucaagcaagugcugaagagaagaaauacuuugcuggagaagaugcucaugucuuugaaauagaagguuuggaugaaaaaggucaagauguugaucucucuuaugcuucuauugugaaaaucccaauugaaaaagauaagaaaguuaagaaaguauuuuucuuaccugaaggcaaagaggcaguagaauuggcuuuugaacaaacggauagucauguuaucuuuacagcaccucacuuuacucauuaugccuuuguuuaugaaucugcugaaaaaccacaaccugcuaaaccagcaccacaaaacacaguccuuccaaaaccuacuuaucaaccgacuucugaucaacaaaaggcuccuaaauuggaaguucaagaggaaaagguugccuuucaucgucaagagcaugaaaauacugagaugcuaguuggggaacaacgagucaucauacagggacgagauggacuguuaagacaugucuuugaaguugaugaaaacggucagcgucgucuucguucaacagaagucauccaagaagcgauuccagaaauuguugaaauuggaacaaaaguaaaaacaguaccagcaguaguagcuacacaggaaaaaccagcucaaaauacagcaguuaaaucagaagaagcaagcaaacaauugccaaauacaggaacagcugaugcuaaugaagcccuaauagcaggcuuagccagccuuggucuugcuaguuuagccuugaccuugagacggaaaagagaagauaaagauuaa

SEQ ID NO: 101 - RNA, 5304 nt, Streptococcus pneumoniae
augaauaaaggauuauuugaaaaacguuguaaauauaguauucggaaauuuucauuagguguugcuucuguuaugauuggagcugcauucuuugggacaaguccgguucuugcagauagcgugcagucugguuccacggcgaacuuaccagcugauuuagcuacugcucuugcaacagcaaaagagaaugauggacgugauuuugaagcgccuaaggugggagaagaccaagguucuccagaaguuacagauggaccuaagacagaagaagaacuauuagcacuugaaaaagaaaaaccggcugaagaaaaaccaaaagaggauaaaccugcagcugcuaaaccugaaacaccuaagacgguaaccccugaauggcaaacgguagagaaaaaagaacaaaagggaacagucacuauccgagaagaaaaagguguccgcuacaaccaacuauccucaacugcucaaaaugauaacgcaggaaaaccagcccuguuugaaaagaagggcuugaccguugaugccaauggaaaugcaacuguugauuuaaccuucaaagaugauucugaaaagggcaaaucacgcuuuggugucuuccugaaauuuaaagauaccaauaauaauguuuuugucgguuaugacaaggauggcugguucugggaguauaaaucuccaacaacuagcacuugguauagagguagucguguugcugcuccugaaacaggaucaacaaaccgucucucuaucacucucaagucagacggucagcuaaaugccagcaauaacgaugucaaucucuuugacacagugacucuaccagcugcggucaaugaccaucuuaaaaaugagaagaagauccuucucaaggcgggcucuuauggcaaugaccguacgguugucagcguuaaaacagacaaucaagaaggcguaaaagcggaugauacuccugcccagaaagaaacagguccgguuguugacgauagcaaggugacuuaugauacgauccagucuaagguacuuaaggcaguaauugaccaagccuucccucgugucaaggaauacagcuugaacgggcauacuuugccaggacaggugcaacaguucaaccaagucuuuaucaauaaccaccgaaucaccccugaagucacuuauaagaaaaucaaugagacaacagcagaguacuugaugaagcuucgcgaugaugcucacuuaaucaaugcggaaaugacaguacgccugcaaguuguggacaaucaauugcacuuugaugugaccaagauugucaaccacaaucaagucacuccaggucaaaagauugaugacgaaagaaaacuacuuucuucuauuaguuuccucggcaaugcuuuagucucuguuucuagugaucaaacuggugcuaaguuugauggggcaaccaugucaaacaauacgcaugucagcggagaugaucauaucgauguaaccaauccaaugaaagaccuagccaaggguuacauguauggauuuguuucuacagauaagcuugcugcugguguuuggaguaacucucaaaacagcuaugguggugguucgaaugacuggacucguuugacagcuuauaaagaaacagucggaaaugccaacuauguaggaauccacagcucugaauggcaaugggaaaaagcuuauaagggcauuguuuucccagaauacacgaaggaacuuccaagggcuaagguuguuaucacugaagaugccaaugcagacaagaaagucgauuggcaggauggugccauugcuuaucguagcauuaugaacaacccucaagguugggaaaaaguuaaggauaucacagcuuaccguaucgcgaugaacuuugguucucaagcacaaaacccauuccuuaugaccuuggaugguaucaagaaaaucaaucuccacacagauggucuugggcaagguguucuccuuaaaggauaugguagcgaaggccaugacucuggucacuugaacuaugcugauauugguaagcguaucgguggugucgaagacuucaagacccuaauugagaaggcuaagaaauauggagcucaucuagguauccacguuaacgcuucagaaacuuauccugagucuaaauacuucaaugaaaaaauucuccguaagaauccagauggaaguuauagcuaugguuggaacuggcuagaucaagguaucaacauugaugcugccuaugaccuagcucauggucguuuggcacguugggaagauuugaagaaaaaacuuggugacggucucgacuuuaucuauguggacguuugggguaauggucaaucaggugauaacggugccugggcuacccacguucuugcuaaagaaauuaacaaacaaggcuggcgcuuugcgaucgaguggggccaugguggugaguacgacucuaccuuccaucacugggcagcugacuugaccuacgguggcuacaccaauaaagguaucaacagugccaucacucgcuuuauccguaaccaccaaaaagaugcuuggguuggggacuacagaaguuaugguggugcagccaacuauccacugcuagguggcuacagcaugaaagacuuugaaggcuggcaaggaagaagugacuacaauggcuacgugacuaaccucuuugcucaugacgucaugacuaaguacuuccaacacuucacuguaaguaaaugggaaaaugguacaccggugacuaugaccgauaacgguagcaccuauaaauggacuccagaaaugcgaguggaauugguagauacugacaauaauaaaguaguuguaacucguaagucaaaugaugucaauagcccacaauaucgcgaacguacaguaacucucaacggacgugucauacaagaugguucagcuuacuugacuccuuggaacugggaugcaaaugguaagaaacuuucuacugauaaggaaaagauguacuacuucaauacgcaggccggugcaacaacuuggacccuuccaagcgauugggcaaagagcaagguuuaccuuuacaagcuaacugaccaagguaagacagaagagcaagaacuaacuguaaaagaugguaaaauuacccuagaucuucuagcaaaucaaccauacguucucuaucguucgaaacaaaccaauccugaaaugucauggagcgaaggcaugcacaucuaugaccaaggauuuaauaguggaaccuugaaacacuggaccauuucuggugaugcuucuaaggcagaaauugucaagucucaaggggcaaacgauaugcuucguauucaaggaaacaaagaaaaaguuagucucacucagaaauuaacuggcuugaaaccaaauaccaaguaugccguuuaugucggugucgauaaccguaguaaugccaaggcgagcaucacuguaaauacuggugaaaaagaagugacuacuuauaccaauaagucucucgcgcucaacuauguuaaggcuuacgcccacaauacacgucgugacaaugcuacaguuaacgauacaaguuacuuccaaaacauguacgccuucuuuacaacaggaucggacgucucaaauguuacucugacauugagucgugaagcuggugaucaagcaacuuacuuugaugaaauucguaccuuugaaaacaauucaagcauguauggagacaagcaugauacagguaaaggcaccuucaagcaagacuuugaaaauguugcucaggguaucuucccauuuguagugggugguguugaagguguugaagauaaccgcacucacuugucugaaaaacaugauccauauacacaacgugguuggaacgguaagaaaguugaugauguuaucgaaggaaauuggucacucaagacaaauggacuagugagccgucguaacuugguuuaccaaacuauuccgcaaaacuuccguuuugaagcagguaagaccuaccguguaaccuuugaauacgaagcagguucagacaauaccuaugcuuuuguagucgguaagggagaauuccagucaggucgucgugguacucaagcaagcaacuuggaaaugcaugaauugccaaauacuuggacagauucuaagaaagccaagaaggcaaccuuccucgugacaggugcagaaacaggggauacuuggguagguaucuacucaacuggaaaugcaaguaauacucguggugauucugguggaaaugccaacuuccgugguuauaacgacuucaugauggauaaucuucaaaucgaagaaauuacccuaacagguaagauguugacagaaaaugcucugaagaacuacuugccaacgguugccaugacuaacuacaccaaagagucuauggaugcuuugaaagaggcggucuuuaaccucagucaggccgaugaugauaucaguguggaagaagcgcgugcagagauugccaagauugaagccuugaagaaugcuuugguucagaagaaaacggcuuugguagcagaagacuuugcaagucuuacagcuccugcucaggcucaagaaggucuugcaaaugccuuugauggaaacuuaucuaguuuauggcauacaucauggggcggaggagauguaggcaagccugcaaccauggucuugaaagaaccaacugaaaucacuggacuucguuauguuccacguggaucagguucaaaugguaacuugcgugaugugaaacuuguugugacagaugagucuggcaaggagcauaccuuuacugcaacugauuggccagauaacaauaagucaaaagauauugacuuugguaagacaaucaaggcuaagaaaauuguccuuacugguaccaagacauacggagaugguggcgauaaauaccaaucugcagcggaacuuaucuuuacucguccacagguagcagaaacaccuuuugacuugucaggcuaugaagcagcuuuggcuaaggcucagaaauuaacagacaaagacaaucaagaggaaguagcuagcguucaggcaagcaugaaauaugcgacggauaaccaucucuugacggaaagaaugguggaauacuuugcagauuaucucaaccaauuaaaagauucugcuacgaaaccagaugcuccaacuguagagaaaccugaguuuaaacuuagcucuuuagcuuccgaucaagguaagacgccagauuauaagcaagaaauagcuagaccagaaacaccugaacaaaucuugccagcaacaggugagagucaaucugacacagcccucuuccuagcagguguuagccuagcccuaucugcucucuuuguaguaaaaacgaagaaagacuag

SEQ ID NO: 102 - RNA, 6015 nt, Streptococcus pneumoniae
auggaaaaguauuuuggugaaaaacaagagcguuuuucauuuagaaaauuaucaguaggacuuguaucugcaacgauuucaaguuuauuuuuuaugucuguauuagcuaguucaucuguggaugcucaagaaacugcgggaguucacuauaaauauguggcagauucagagcuaucaucagaagaaaagaagcagcuugucuaugauauuccgacauacguggagaaugaugaugaaacuuauuaucuuguuuauaaguuaaauucucaaaaucaacuggcggaauugccaaauacuggaagcaagaaugagaggcaagcccuaguugcuggugcuagcuuagcugcuaugggaauuuuaauuuuugcuguuuccaagaaaaagguuaagaauaaaacgguauuacauuuaguauugguugcagggauaggaaauggugucuuaguuucaguccaugcuuuagaaaaucaucuuuugcuaaauuacaauacggacuaugaauugaccucuggagaaaaauuaccucuuccuaaagagauuucagguuacacuuauauuggauauaucaaagagggaaaaacgacuucugagucugaaguaaguaaucaaaagaguucaguugccacuccuacaaaacaacaaaagguggauuauaauguuacaccgaauuuuguagaccauccaucaacaguacaagcuauucaggaacaaacaccuguuucuucaacuaagccgacagaaguucaaguaguugaaaaaccuuucucuacugaauuaaucaauccaagaaaagaagagaaacaaucuucagauucucaagaacaauuagccgaacauaagaaucuagaaacgaagaaagaggagaagauuucuccaaaagaaaagacugggguaaauacauuaaauccacaggaugaaguuuuaucaggucaauugaacaaaccugaacucuuauaucgugaggaaacuauggagacaaaaauagauuuucaagaagaaauucaagaaaauccugauuuagcugaaggaacuguaagaguaaaacaagaagguaaauuagguaagaaaguugaaaucgucagaauauucucuguaaacaaggaagaaguuucgcgagaaauuguuucaacuucaacgacugcgccuaguccaagaauagucgaaaaagguacuaaaaaaacucaaguuauaaaggaacaaccugagacugguguagaacauaaggacguacagucuggagcuauuguugaacccgcaauucagccugaguugcccgaagcuguaguaagugacaaaggcgaaccagaaguucaaccuacauuacccgaagcaguugugaccgacaaaggugagacugagguucaaccagagucgccagauacugugguaagugauaaaggugaaccagagcagguagcaccgcuuccagaauauaaggguaauauugagcaaguaaaaccugaaacuccgguugagaagaccaaagaacaagguccagaaaaaacugaagaaguuccaguaaaaccaacagaagaaacaccaguaaauccaaaugaagguacuacagaaggaaccucaauucaagaagcagaaaauccaguucaaccugcagaagaaucaacaacgaauucagagaaaguaucaccagauacaucuagcaaaaauacuggggaaguguccaguaauccuagugauucgacaaccucaguuggagaaucaaauaaaccagaacauaaugacucuaaaaaugaaaauucagaaaaaacuguagaagaaguuccaguaaauccaaaugaaggcacaguagaagguaccucaaaucaagaaacagaaaaaccaguucaaccugcagaagaaacacaaacaaacucugggaaaauagcuaacgaaaauacuggagaaguauccaauaaaccuagugauucaaaaccaccaguugaagaaucaaaucaaccagaaaaaaacggaacugcaacaaaaccagaaaauucagguaauacaacaucagagaauggacaaacagaaccagaaccaucaaacggaaauucaacugaggauguuucaaccgaaucaaacacauccaauucaaauggaaacgaagaaauuaaacaagaaaaugaacuagacccugauaaaaagguagaagaaccagagaaaacacuugaauuaagaaauguuuccgaccuagaguuauacaguuugucaaaugguacuuauaaacaacacauuucguuagagcaaguuccaagcaauccaaauagcuacuuuguuaaagugaaaucuucuucauucaaagauguauaccuaccaguagcaucaauaucagaggaaagaaaaaaugauaaaauccuuuauaaaaucacagcaaaaguagagaagcuucagcaggagauagaaagcagauauaaagauaauuuuaccuucuaucuagcuaagaagggaacagaagaaacaacaaacuuuacuuccuuuaguaaucuggucaaagcuauaaaccaaaaucccucuggaaccuaucauuuagcggccagccugaaugcuaacgaaguggagcuugguccugaugaaagauccuauaucaaggacaccuuuacuggucguuuaaucggugaaaaagauggcaagaauuaugcuaucuauaauuugaaaaaaccucuguuugaaaacuugaguggugcuacaguagaaaaacugagucuaaaaaauguugcuauuucagggaaagaugauaucgguucacuggcaaaugaagcucagaauaacacaaaaauuaagcaaguucacgucgaugguguucuggcuggugaacgugguaucggugguuugcuggcuaaggcugagcaaucaagcaucacagagagcaguuucaagggaagaauuaucaacacuuaugaaacgacugcugccuacaauaucggugguauggucggucauuugacaggugacaaggcuuuacuuacuaagucaaaagcgacaguagccauuucaucuaacacaaauacuucagaucagacuguggguggacuugcaggccuaguagaccgagaugcacagauccaagauagcuaugcugaaggugauaucaacaaugucaagcacuuugguagagucgcuggaguggcaggcaauuugugggaucgaacuucuggugauguuaggcaugcuggaaguuugaccaauguucucagcgauguuaauguaaccaacggaaaugccaucacugguuaccacuauaacgaaaugaagguaaaggacacauucagcagcaaggccaacagagucuacaaugucaccuuggucaaggaugaggucgucagcaaggaauccuuugaagaaagaggaacaaugcuagaugcuucucaaauugcaagcaaaaaagcagaaaucaauccucucauuuuaccaacaguggagccacuuucaacaaguggcaaaaaagacagugauuuuucuaagguggccuauuaucaagcuaagcgcaacuugacuuauaaaaacauugaaaaauugcuaccuuucuacaacaaggcaaccaucgucaaauacggaaaccuggucaaugagaacagucuuuuauaucaaaaagaacucuugucagcagucaugaugaaggacaaccaagucaucacagacauuguuucuaacaaacagacugcaaacaaacucuugcuucacuacaaggaugauuuaucugagaagcuggaucucaaauaccagaaugauuucgccaaauuagcagaauauagucugggcaauacuggacuucucuauacgccaaaccaauuccuguaugaccaaaccucuaucaucaagcaagucuuaccugacuuacaaaagguugacuaucauucagaagccaucagaaagacgcuggguauuucuccaaacgucaagcaaacugagcucuaucuagaagaccaguucgccaaaacaaaacaacaacuggaagacaguuugaaaaaacucuugucagcggaugcuggacuggcuagugcuaaccccgucacugaagguuaucuuguagauaaaaucaaacgcaacaaggaagccuugcuacuuggcuugaccuaucuggaacggugguauaacuuuagcuauggucaggugaaugucaaagaccuaguucuguaccauuuggacuucuuugguaaggggaaugcuucaccauuagauacucugauugaguuggguaaaucuggcuuuaacaaucuucuagcuaagaauaaugucgauacuuaugguaucagucuugccagucaacauggaacgacagauuuguuuagcacgcuggaacauuaccgaaaagucuuuuuaccaaauacaagcaauaaugacugguuuaaaucagagacuaaggcuuacauugucgaagaaaaauccacuaucgaagaggugaaaacgaagcaaggguuagcuggcaccaaguauucuaucgguguuuaugaucguaucacgagugccacauggaaauaccgcaauauggucuugccucuccugaccuugccagagagauccguauuugucaucucgaccaugucuagucuaggauuuggagcuuaugaucgcuaccgcaguagugaccauaaagcgggcaaggcucucaaugauuuuguugaagaaaaugcgcgugaaacagccaaacgucagcgagaucacuacgauuauugguaucguauuuuagacgacaaugcacgugaaaaacuuuauagaaauauuuugcuuuacgaugcuuauaaauuuggcgaugauaauaccguagggaaagcuacagaaguggcagauuuugauaauccaaauccugcaaugcaacauuucuuuggaccuguuggaaauaaaguugggcauaaucaacacggugcuuaugcuacaggugaugcaguuuauuauauggguuaucgaauguuggauaaggauggagcuauuacuuauacgcaugagaugacacaugacucagaucaggacauuuaucuuggaggauauggucgaagaaguggcuugggaccagaguucuuugcuaaaggauuauuacaagcaccagaccauccagaugaugcgaccauuaccaucaacuccaucuugaaacauucaaaaucugauaguacagaaagucgacgauuacaaguacuugauccaacuacaagauuuaauaaugcagaugauuugaagcaauauguccacaacauguuugacguuguuuauauguuggaauaucucgaaggaaauucaauucuuaaauuggauacgaaucaaaaacaacaacuucuuagaaaaguuacaaaugaguaccauccugauccugauggaaauaaggucuaugcaacaaauguugucagaaaucuaacaguagaagaaguugaaagacuacguucauucaaugauuugauugauaauaauauucuuucgucuagggaauaugccucagguaaauacgaaagaaauggcuacuucacuauuaaguuauuugcaccgauuuaugcugcauuaaguaaugauauaggaacaccaggugaccugaugggacgucguauagccuaugaacuacuagcugcuaaaggcuuuaaagaugguaugguaccauauaucucaaaccaauacgaagaagaagccaaacaaaagggcaagacaaucaaucucuacgguaaaacaagagguuugguuacagaugacuugguuuuggaaaagguauuuaauaaccaauaucauacuuggagugaguuuaagaaagcuauguaucaagaacgacaagaucaguuugauagauugaacaaaguuacuuuuaaugauacaacacagccuuggcaaacauuugccaagaaaacuacaagcaguguagaugaauuacagaaauuaauggacguugcuguucguaaggaugcagaacacaauuacuaccauuggaauaacuacaauccagacauagauagugaaguccacaagcucaagagagcaaucuuuaaagccuaucuugaccaaacaaaugauuuuagaaguucaauuuuugagaauaaaaaauag

SEQ ID NO: 103 - RNA, 6423 nt, Streptococcus pneumoniae
augaaaaaaaguacaguauugucauuaaccacagcugcaguuauuuuagcagccuaugccccuaaugagguagucuuagcagacacaucuagcucugaagaugcuuuaaacaucucugauaaagaaaaaguagcagaaaauaaagagaaacaugaaaauauccauagugcuauggaaacuucacaggauuuuaaagagaagaaaacagcagucauuaaggaaaaagaaguuguuaguaaaaauccugugauagacaauaacacuagcaaugaagaagcaaaaaucaaagaagaaaauuccaauaaaucccaaggagauuauacggacucauuugugaauaaaaacacagaaaaucccaaaaaagaagauaaaguugucuauauugcugaauuuaaagauaaagaaucuggagaaaaagcaaucaaggaacuauccagucuuaagaauacaaaaguuuuauauacuuaugauagaauuuuuaacgguagugccauagaaacaacuccagauaacuuggacaaaauuaaacaaauagaagguauuucaucgguugaaagggcacaaaaaguccaacccaugaugaaucaugccagaaaggaaauuggaguugaggaagcuauugauuaccuaaagucuaucaaugcuccguuugggaaaaauuuugaugguagagguauggucauuucaaauaucgauacuggaacagauuauagacauaaggcuaugagaaucgaugaugaugccaaagccucaaugagauuuaaaaaagaagacuuaaaaggcacugauaaaaauuauugguugagugauaaaaucccucaugcguucaauuauuauaaugguggcaaaaucacuguagaaaaauaugaugauggaagggauuauuuugacccacaugggaugcauauugcagggauucuugcuggaaaugauacugaacaagacaucaaaaacuuuaacggcauagauggaauugcaccuaaugcacaaauuuucucuuacaaaauguauucugacgcaggaucuggguuugcgggugaugaaacaauguuucaugcuauugaagauucuaucaaacacaacguugauguuguuucgguaucaucugguuuuacaggaacaggucuuguaggugagaaauauuggcaagcuauucgggcauuaagaaaagcaggcauuccaaugguugucgcuacggguaacuaugcgacuucugcuucaaguucuucaugggauuuaguagcaaauaaucaucugaaaaugaccgacacuggaaauguaacacgaacugcagcacaugaagaugcgauagcggucgcuucugcuaaaaaucaaacaguugaguuugauaaaguuaacauagguggagaaaguuuuaaauacagaaauauaggggccuuuuucgauaagaguaaaaucacaacaaaugaagauggaacaaaagcuccuaguaaauuaaaauuuguauauauaggcaaggggcaagaccaagauuugauagguuuggaucuuaggggcaaaauugcaguaauggauagaauuuauacaaaggauuuaaaaaaugcuuuuaaaaaagcuauggauaagggugcacgcgccauuaugguuguaaauacuguaaauuacuacaauagagauaauuggacagagcuuccagcuaugggauaugaagcggaugaagguacuaaaagucaaguguuuucaauuucaggagaugaugguguaaagcuauggaacaugauuaauccugauaaaaaaacugaagucaaaagaaauaauaaagaagauuuuaaagauaaauuggagcaauacuauccaauugauauggaaaguuuuaauuccaacaaaccgaauguaggugacgaaaaagagauugacuuuaaguuugcaccugacacagacaaagaacucuauaaagaagauaucaucguuccagcaggaucuacaucuugggggccaagaauagauuuacuuuuaaaacccgauguuucagcaccugguaaaaauauuaaauccacgcuuaauguuauuaauggcaaaucaacuuauggcuauaugucaggaacuaguauggcgacuccaaucguggcagcuucuacuguuuugauuagaccgaaauuaaaggaaaugcuugaaagaccuguauugaaaaaucuuaagggagaugacaaaauagaucuuacaagucuuacaaaaauugcccuacaaaauacugcgcgaccuaugauggaugcaacuucuuggaaagaaaaaagucaauacuuugcaucaccuagacaacagggagcaggccuaauuaauguggccaaugcuuugagaaaugaaguuguagcaacuuucaaaaacacugauucuaaagguuugguaaacucauaugguuccauuucucuuaaagaaauaaaaggugauaaaaaauacuuuacaaucaagcuucacaauacaucaaacagaccuuugacuuuuaaaguuucagcaucagcgauaacuacagauucucuaacugacagauuaaaacuugaugaaacauauaaagaugaaaaaucuccagaugguaagcaaauuguuccagaaauucacccagaaaaagucaaaggagcaaauaucacauuugagcaugauacuuucacuauaggcgcaaauucuagcuuugauuugaaugcgguuauaaauguuggagaggccaaaaacaaaaauaaauuuguagaaucauuuauucauuuugagucaguggaagaaauggaagcucuaaacuccaacgggaagaaaauaaacuuccaaccuucuuugucgaugccucuaaugggauuugcugggaauuggaaccacgaaccaauccuugauaaaugggcuugggaagaagggucaagaucaaaaacacugggagguuaugaugaugaugguaaaccgaaaauuccaggaaccuuaaauaagggaauugguggagaacaugguauagauaaauuuaauccagcaggaguuauacaaaauagaaaagauaaaaauacaacaucccuggaucaaaauccagaauuauuugcuuucaauaacgaagggaucaacgcuccaucaucaagugguucuaagauugcuaacauuuauccuuuagauucaaauggaaauccucaagaugcucaacuugaaagaggauuaacaccuucuccacuuguauuaagaagugcagaagaaggauugauuucaauaguaaauacaaauaaagagggagaaaaucaaagagacuuaaaagucauuucgagagaacacuuuauuagaggaauuuuaaauucuaaaagcaaugaugcaaagggaaucaaaucaucuaaacuaaaaguuuggggugacuugaagugggauggacucaucuauaauccuagagguagagaagaaaaugcaccagaaaguaaggauaaucaagauccugcuacuaagauaagaggucaauuugaaccgauugcggaaggucaauauuucuauaaauuuaaauauagauuaacuaaagauuacccauggcagguuuccuauauuccuguaaaaauugauaacaccgccccuaagauuguuucgguugauuuuucaaauccugaaaaaauuaaguugauuacaaaggauacuuaucauaagguaaaagaucaguauaagaaugaaacgcuauuugcgagagaucaaaaagaacauccugaaaaauuugacgagauugcgaacgaaguuugguaugcuggcgccgcucuuguuaaugaagauggagagguugaaaaaaaucuugaaguaacuuacgcaggugagggucaaggaagaaauagaaaacuugauaaagacggaaauaccauuuaugaaauuaaaggugcgggagauuuaaggggaaaaaucauugaagucauugcauuagaugguucuagcaauuucacaaagauucauagaauuaaauuugcuaaucaggcugaugaaaaggggaugauuuccuauuaucuaguagauccugaucaagauucaucuaaauaucaaaagcuuggcgagauugcagaaucuaaauuuaaaaauuuaggaaauggaaaagaggguagucuaaaaaaagauacaacugggguagaacaucaucaucaagaaaaugaagagucuauuaaagaaaaaucuaguuuuacuauugauagaaauauuucaacaauuagagacuuugaaaauaaagacuuaaagaaacucauuaaaaagaaauuuagagaaguugaugauuuuacaagugaaacugguaagagaauggaggaauacgauuauaaauacgaugauaaaggaaauauaauagccuacgaugaugggacugaucuagaauaugaaacugagaaacuugacgaaaucaaaucaaaaauuuaugguguucuaaguccgucuaaagauggacacuuugaaauucuuggaaagauaaguaauguuucuaaaaaugccaagguauauuaugggaauaacuauaaaucuauagaaaucaaagcgaccaaguaugauuuccacucaaaaacgaugacauuugaucuauacgcuaauauuaaugauauuguggauggauuagcuuuugcaggagauaugagauuauuuguuaaagauaaugaucagaaaaaagcugaaauuaaaauuagaaugccugaaaaaauuaaggaaacuaaaucagaauaucccuauguaucaaguuaugggaaugucauagaauuaggggaaggagaucuuucaaaaaacaaaccagacaauuuaacuaaaauggaaucugguaaaaucuauucugauucagaaaaacaacaauaucuguuaaaggauaauaucauucuaagaaaaggcuaugcacuaaaagugacuaccuauaauccuggaaaaacggauauguuagaaggaaauggagucuauagcaaggaagauauagcaaaaauacaaaaggccaauccuaaucuaagagcccuuucagaaacaacaauuuaugcugauaguagaaauguugaagauggaagaaguacccaaucuguauuaaugucggcuuuggacggcuuuaacauuauaagguaucaaguguuuacauuuaaaaugaacgauaaaggggaagcuaucgauaaagacggaaaucuugugacagauucuucuaaacuuguauuauuugguaaggaugauaaagaauacacuggagaggauaaguucaauguagaagcuauaaaagaagauggcuccauguuauuuauugauaccaaaccaguaaaccuuucaauggauaagaacuacuuuaauccaucuaaaucuaauaaaauuuauguacgaaauccagaauuuuauuuaagagguaagauuucugauaagggugguuuuaacugggaauugagaguuaaugaaucgguuguagauaauuauuuaaucuacggagauuuacacauugauaacacuagagauuuuaauauuaagcugaauguuaaagacggugacaucauggacuggggaaugaaagacuauaaagcaaacggauuuccagauaagguaacagauauggauggaaauguuuaucuucaaacuggcuauagcgauuugaaugcuaaagcaguuggaguccacuaucaguuuuuauaugauaauguuaaacccgaaguaaacauugauccuaagggaaauacuaguaucgaauaugcugauggaaaaucuguagucuuuaacaucaaugauaaaagaaauaauggauucgauggugagauucaagaacaacauauuuauauaaauggaaaagaauauacaucauuuaaugauauuaaacaaauaauagacaagacacuaaacauuaagauuguuguaaaagauuuugcaagaaauacaaccguaaaagaauucauuuuaaauaaagauacgggagagguaagugaauuaaaaccucauaggguaacugugaccauucaaaauggaaaagaaaugaguucaacgauagugucggaagaagauuuuauuuuaccuguuuauaagggugaauuagaaaaaggauaccaauuugaugguugggaaauuucugguuucgaagguaaaaaagacgcuggcuauguuauuaaucuaucaaaagauaccuuuauaaaaccuguauucaagaaaauagaggagaaaaaggaggaagaaaauaaaccuacuuuugauguaucgaaaaagaaagauaacccacaaguaaaccauagucaauuaaaugaaagucacagaaaagaggauuuacaaagagaagagcauucacaaaaaucugauucaacuaaggauguuacagcuacaguucuugauaaaaacaauaucaguaguaaaucaacuacuaacaauccuaauaaguugccaaaaacuggaacagcaagcggagcccagacacuauuagcugccggaauaauguuuauaguaggaauuuuucuuggauugaagaaaaaaaaucaagauuaa

SEQ ID NO: 104 - RNA, 6702 nt, Streptococcus pneumoniae
auggggaaaggccauuggaaucggaaaagaguuuauagcauucguaaguuugcugugggagcuugcucaguaaugauugggacuugugcaguuuuauuaggaggaaauauagcuggagaaucuguaguuuaugcggaugaaacacuuauuacucauacugcugagaaaccuaaagaggaaaaaaugauaguagaagaaaaggcugauaaagcuuuggaaacuaaaaauauaguugaaaggacagaacaaagugaaccuaguucaacugaggcuauugcaucugagaagaaagaagaugaagccguaacuccaaaagaggaaaaagugucugcuaaaccggaagaaaaagcuccaaggauagaaucacaagcuucaaaucaagaaaaaccgcucaaggaagaugcuaaagcuguaacaaaugaagaagugaaucaaaugauugaagacaggaaaguggauuuuaaucaaaauugguacuuuaaacucaaugcaaauucuaaggaagccauuaaaccugaugcagacguaucuacguggaaaaaauuagauuuaccguaugacuggaguaucuuuaacgauuucgaucaugaaucuccugcacaaaaugaagguggacagcucaacgguggggaagcuugguaucgcaagacuuucaaacuagaugaaaaagaccucaagaaaaauguucgccuuacuuuugauggcgucuacauggauucucaaguuuaugucaauggucaguuaguggggcauuauccaaaugguuauaaccaguucucauaugauaucaccaaauaccuucaaaaagauggucgugagaaugugauugcuguccaugcagucaacaaacagccaaguagccguugguauucaggaagugguaucuaucgugaugugacuuuacaagugacagauaaggugcauguugagaaaaaugggacaacuauuuuaacaccaaaacuugaagaacaacaacauggcaagguugaaacucaugugaccagcaaaaucgucaauacggacgacaaagaccaugaacuuguagccgaauaucaaaucguugaacgagguggucaugcuguaacaggcuuaguucguacagcgagucguaccuuaaaagcacaugaaucaacaagccuagaugcgauuuuagaaguugaaagaccaaaacucuggacuguuuuaaaugacaaaccugccuuguacgaauugauuacgcguguuuaccgugacggucaauugguugaugcuaagaaggauuuguuugguuaccguuacuaucacuggacuccaaaugaagguuucucuuugaauggugaacguauuaaauuccauggaguauccuugcaccacgaccauggggcgcuuggagcagaagaaaacuauaaagcagaauaucgccgucucaaacaaaugaaggagaugggaguuaacuccauccguacaacccacaacccugcuagugagcaaaccuugcaaaucgcagcagaacuagguuuacucguucaggaagaggccuuugauacgugguaugguggcaagaaaccuuaugacuauggacguuucuuugaaaaagaugccacucacccagaagcucgaaaaggugaaaaauggucugauuuugaccuacguaccauggucgaaagaggcaaaaacaacccugcuaucuucauguggucaauugguaaugaaauaggugaagcuaauggugaugcccacucuuuagcaacuguuaaacguuugguuaagguuaucaaggauguugauaagacucgcuauguuaccaugggagcagauaaauuccguuucgguaaugguagcggagggcaugagaaaauugcugaugaacucgaugcuguuggauuuaacuauucugaagauaauuacaaagcccuuagagcuaagcauccaaaaugguugauuuauggaucagaaacaucuucagcuacccguacacguggaaguuacuaucgcccugaacgugaauugaaacauagcaauggaccugagcguaauuaugaacagucagauuauggaaaugaucguguggguugggggaaaacagcaaccgcuucauggacuuuugaccgugacaacgcuggcuaugcuggacaguuuaucuggacagguacggacuauauuggugaaccuacaccauggcacaaccaaaaucaaacuccuguuaagagcucuuacuuugguaucguagauacagccggcauuccaaaacaugacuucuaucucuaccaaagccaauggguuucuguuaagaagaaaccgaugguacaccuucuuccucacuggaacugggaaaacaaagaauuagcauccaaaguagcugacucagaagguaagauuccaguucgugcuuauucgaaugcuucuaguguagaauuguucuugaauggaaaaucucuuggucuuaagacuuucaauaaaaaacaaaccagcgaugggcggacuuaccaagaaggugcaaaugcuaaugaacuuuaucuugaauggaaaguugccuaucaaccagguaccuuggaagcaauugcucgugaugaaucuggcaaggaaauugcucgagauaagauuacgacugcugguaagccagcggcaguucgucuuauuaaggaagaccaugcgauugcagcagauggaaaagacuugacuuacaucuacuaugaaauuguugacagccaggggaaugugguuccaacugcuaauaaucugguucgcuuccaauugcauggccaaggucaacuggucgguguagauaacggagaacaagccagccgugaacgcuauaaggcgcaagcagaugguucuuggauucguaaagcauuuaaugguaaagguguugccauugucaaaucaacugaacaagcagggaaauucacccugacugcccacucugaucucuugaaaucgaaccaagucacugucuuuacugguaagaaagaaggacaagagaagacuguuuuggggacagaagugccaaaaguacagaccauuauuggagaggcaccugaaaugccuaccacuguuccguuuguauacagugaugguagccgugcagaacguccuguaaccuggucuucaguagaugugagcaagccugguauuguaacggugaaagguauggcugacggacgagaaguagaagcucguguagaagugauugcucuuaaaucagagcuaccaguugugaaacguauugcuccaaauacugacuugaauucuguagacaaaucuguuuccuauguuuugauugauggaaguguugaagaguaugaaguggacaagugggagauugccgaagaagauaaagcuaaguuagcaauuccagguucucguauucaagcgaccgguuauuuagaaggucaaccaauucaugcaacccuugugguagaagaaggcaauccugcggcaccugcaguaccaacuguaacgguugguggugaggcaguaacaggucuuacuagucaaaaaccaaugcaauaccgcacucuugcuuauggagcuaaguugccagaagucacagcaagugcuaaaaaugcagcuguuacaguucuucaagcaagcgcagcaaacggcaugcgugcgagcaucuuuauucagccuaaagaugguggcccucuucaaaccuaugcaauucaauuccuugaagaagcgccaaaaauugcucacuugagcuugcaaguggaaaaagcugacagucucaaagaagaccaaacugucaaauugucgguucgagcucacuaucaagauggaacgcaagcuguauuaccagcugauaaaguaaccuucucuacaaguggugaaggggaagucgcaauucguaaaggaaugcuugaguugcauaagccaggagcagucacucugaacgcugaauaugagggagcuaaagaccaaguugaacucacuauccaagccaauacugagaagaagauugcgcaauccauccguccuguaaauguagugacagauuugcaucaggaaccaagucuuccagcaacaguaacaguugaguaugacaaagguuucccuaaaacucauaaagucacuuggcaagcuauuccgaaagaaaaacuagacuccuaucaaacauuugaaguacuagguaaaguugaaggaauugaccuugaagcgcgugcaaaagucucuguagaagguaucguuucaguugaagaagucagugugacaacuccaaucgcagaagcaccacaauuaccagaaaguguucggacauaugauucaaauggucacguuucaucagcuaagguugcaugggaugcgauucguccagagcaauacgcuaaggaaggugucuuuacaguuaauggucgcuuagaagguacgcaauuaacaacuaaacuucauguucgcguaucugcucaaacugagcaaggugcaaacauuucugaccaauggaccgguucagaauugccacuugccuuugcuucagacucaaauccaagcgacccaguuucaaauguuaaugacaagcucauuuccuacaauaaccaaccagccaaucguuggacaaacuggaaucguacuaauccagaagcuucagucgguguucuguuuggagauucagguaucuugagcaaacgcuccguugauaaucuaagugucggauuccaugaagaccauggaguugguguaccgaagucuuaugugauugaguauuauguugguaagacugucccaacagcuccuaaaaacccuaguuuuguugguaaugaggaccaugucuuuaaugauucugccaacuggaaaccaguuacuaaucuaaaagccccugcucaacucaaggcuggagaaaugaaccacuuuagcuuugauaaaguugaaaccuaugcuguucguauucgcaugguuaaagcagauaacaagcguggaacgucuaucacagagguacaaaucuuugcgaaacaaguugcggcagccaagcaaggacaaacaagaauccaaguugacggcaaagacuuagcaaacuucaacccugauuugacagacuacuaccuugagucuguagauggaaaaguuccggcagucacagcaaguguuagcaacaauggucucgcuaccgucguuccaagcguucgugaaggugagccaguucgugucaucgcgaaagcugaaaauggcgacaucuuaggagaauaccgucugcacuucacuaaggauaagagcuuacuuucucauaaaccaguugcugcgguuaaacaagcucgcuugcuacaaguaggucaagcacuugaauugccgacuaagguuccaguuuacuucacagguaaagacggcuacgaaacaaaagaccugacaguugaaugggaagaaguuccagcggaaaaucugacaaaagcaggucaauuuacuguucgaggccguguccuugguaguaaccuuguugcugagaucacuguacgagugacagacaaacuuggugagacucuuucagauaacccuaacuaugaugaaaacaguaaccaggccuuugcuucagcaaccaaugauauugacaaaaacucucaugaccgcguugacuaucucaaugacggagaucauucagaaaaucgucguuggacaaacuggucaccaacaccaucuucuaauccagaaguaucagcgggugugauuuuccgugaaaaugguaagauuguagaacggacuguuacacaaggaaaaguucaguucuuugcagauagugguacggaugcaccaucuaaacucguuuuagaacgcuaugucgguccagaguuugaagugccaaccuacuauucaaacuaccaagccuacgacgcagaccauccauucaacaauccagaaaauugggaagcuguuccuuaucgugcggauaaagacauugcagcuggugaugaaaucaacguaacauuuaaagcuaucaaagccaaagcuaugagauggcguauggagcguaaagcagauaagagcgguguugcgaugauugagaugaccuuccuugcaccaagugaauugccucaagaaagcacucaaucaaagauucuuguagauggaaaagaacuugcugauuucgcugaaaaucgucaagacuaucaaauuaccuauaaaggucaacggccaaaagucucaguugaagaaaacaaucaaguagcuucaacugugguagauaguggagaagauagcuuuccaguacuuguucgccucguuucagaaaguggaaaacaagucaaggaauaccguauccacuugacuaaggaaaaaccaguuucugagaagacaguugcugcuguacaagaagaucuuccaaaaaucgaauuuguugaaaaagauuuggcauacaagacaguugagaaaaaagauucaacacuguaucuaggugaaacucguguagaacaagaaggaaaaguuggaaaagaacguaucuuuacagcgauuaauccugauggaaguaaggaagaaaaacuccgugaagugguagaaguuccgacagaccgcaucgucuugguuggaaccaaaccaguagcucaagaagcuaaaaaaccacaagugucagaaaaagcagauacaaaaccaauugauucaagugaagcuagucaaacuaauaaagcccaguuaccaaguacagguagugcggcaagccaagcagcaguagcagcagguuuaacucuucuagguuugagugcaggauuaguaguuacuaaagguaaaaaagaagacuag

SEQ ID NO: 105 - RNA, 14484 nt, Streptococcus pneumoniae
auguuuuuuagacgucagaagggugaauauagagaaacagaccgaguaacucgguucaaauuaaucaaaucagggaagcauuggcuacgggccucgacuucucuuuuuggcuuguuuaaggucuugcgaggugguguugauacuacucaggucaugaccgaaacgguagaagauaaaguaagucauucaauuacugggcuugauauccucaaggggauaguugcugcgggagcugucauaaguggaaccguugcaacucaaacgaagguauuuacaaaugagucagcaguacuugaaaaaacuguagagaaaacggaugcuuuggcaacaaaugauacaguaguucuagguacgauaucuacaaguaauucagcgaguucaacuaguuugucagcuucagagucggcaaguacaucugcaucugagucagccucaaccagcgcuucgaccucagcaaguacaagugcaucagaaucagcaaguacaucggcuucgacaaguauuucugcaucaucuacugugguagguucacaaacagcugccgcuacagaagcaacugcuaagaaggucgaagaagaucguaagaaaccagcuagugauuauguagcaucaguuacaaaugucaaucuccaaucuuaugcuaagcgacgcaagcguucaguggauuccaucgagcaauugcuggcuucuauaaaaaaugcugcuguuuuuucuggcaauacgauuguaaauggcgccccugcaauuaaugcaagucuaaacauugcuaaaagugagacaaaaguuuauacaggugaagguguagauucgguauaucguguuccaauuuacuauaaauugaaagugacaaaugaugguucaaaauugaccuuuaccuauacgguuacguaugugaauccuaaaacaaaugaucuugguaauauaucaaguaugcguccuggauauucuaucuauaauucagguacuucaacacaaacaauguuaacccuuggcagugaucuugguaaaccuucagguguaaagaacuacauuacugacaaaaaugguagacagguucuauccuauaauacaucuacaaugacgacgcaggguaguggguauacuuggggaaauggugcccaaaugaaugguuucuuugcuaagaaaggauauggauuaacaucaucuuggacuguaccaauuacuggaacggauacauccuuuacauuuaccccuuacgcugcuagaacagauagaauuggaauuaacuacuucaaugguggaggaaagguaguugaaucuagcacgaccagucagucacuuucacagucuaagucacucucaguaagugcuagucaaagcgccucagcuucagcaucaacaagugcgucggcuucagcaucaaccagugccucggcuucagcgucaaccagugcgucagcuucagcaaguaccagugcuucagucucagcaucaacaagugcuucagccucagcaucgacaagugccucggcuucagcaagcacaucagcaucugaaucagcgucaaccagugcuucggcuucagcaaguaccagugcuucagcuucagcaucaaccagcgccucggccucagcaagcaccucagcuucugaaucggccucaaccagcgccucggccucagcaagcaccucagcuucugaaucggccucaaccagcgccucagccucagcaucaacgagugcuucggcuucagcaagcacaagcgccucggguucagcaucaacgaguacgucagcuucagcgucaaccagugcuucagccucagcaucaacaagugcgucagccucagcaaguaucucagcgucugaaucggcaucaacgagugcgucugagucagcaucaacgaguacgucagccucagcaagcaccucagcuucugaaucggccucaaccagugcgucagccucagcaucgacaagcgccucagcuucagcaaguaccagugcuucagccucagcgucgacaagugcgucggccucaaccagugcaucugaaucggcaucaaccagugcgucagccucagcaaguacuagugcaucggcuucagcaucaaccagugccucggcuucagcgucaaccagugcgucagcuucagcaaguaccagugcuucagucucagcaucaacaagugcuucagccucagcaucgacaagugccucggcuucagcaagcacaucagcaucugaaucagcgucgacaagcgccucagcuucagcaaguaccagugcgucagccucagcgucgacaagugcgucagccucagcaaguacuagugcaucagcuucagcaucaacgagugcaucggcuucggcgucaaccagugcaucagagucagcaaguaccagugcgucagcuuccgcaucaacaagugccucggcuucagcaagcaccagugcgucggcuucagcaaguacuagcgccucagccucagccucaaccagugcgucagccucagcaaguaucucagcgucugaaucggcaucaacgagugcguccgcuucagcaaguacuagcgccucagccucagcgucaacaagugcaucggcuucagcgucaacgagugcgucugaaucggcaucaacgagugcguccgcuucagcaaguacuagcgccucagccucagcgucaacaagugcaucggcuucagcaucaacgagugcguccgcuucagcaaguacuagcgccucagccucagcgucaacaagugcaucggcuucagcgucaacgagugcgucugagucagcaucaacgagugcgucagccucagcaagcacaucagcuucugaaucugcaucaaccagugcgucagccucagcaucgacaagcgccucagcuucagcaaguaccagugcgucagccucagcgucgacaagugcgucggcuucagcaaguaccagugcgucagccucagcaaguaccagugcgucagccucagcgucgacaagugcgucggccucaaccagugcaucugaaucggcaucaaccagugcgucagccucagcaaguacuagugcaucagcuucagcaucaacgagugcaucggcuucagcaucaaccagugcaucagagucagcaaguaccagugcgucagcuuccgcaucaacaagugccucggcuucagcaaguacuagcgccucagccucagcgucaacaagugcuucagcuuccgcgucaaccagcgccucggccucagcaaguaucucagcgucugaaucggcaucaacaagugccucggcuucagcaucaacgagugcaucagucucagcaagcaccagugcgucggccucagcaagcaccagcgcgucugaauccgcaucaaccagugccucagcuucagcaaguaccucagcaucugaaucagcaucaacaagugccucggcuucagcaagcacaagugcuucagccucagcaaguaucucagcgucugaaucggcaucaacgagugcguccgcuucagcaaguacuagcgccucagcaucagcgucaacaagugcuucggcuucagcgucaacgagugcgucugagucagcaucaacgaguacgucagccucagcaagcacaucagcuucugaaucugcaucaaccagugcgucagccucagcaucgacaagcgccucagcuucagcaaguaccagugcgucagccucagcaaguaccagugcuucagccucagcgucgacaagugcgucggccucaaccagugcaucugaaucggcaucaaccagugcgucagccucagcaaguacuagcgccucagccucagcaucaacgagugcguccgcuucagcaaguacuagugcaucagcuucagcaaguacuagcgccucagccucagcgucgacaagcgccucagcuucagcaaguaccagugcgucagccucagcgucgacaagugcgucggcuucagcaaguaccucagcgucugaaucagcaucaacaagugcgucggcuucagcaucaacgagugcaucagcuucagcaucaacaagugcuucagcuucagcaaguaccagugcgucggcuucagcaucaacgagugcuucagucucagcgucaaccagugccucugaauccgcaucaacaagugccucggcuucagcaagcaccagugcuucggcuucagcgucaacgagugcgucugagucagcaucaacgagugcgucagccucagcaagcacaucagcuucugaaucugcaucaaccagugcgucagcuuccgcaucaacaagcgccucggccucagcaaguacaagugcuucagccucagcaucaaccagugcaucagcuucagccucaacaagugcuucagccucagcgucaaccagugccucggcuucagcaaguaccagugcgucagcuucagcaagcacaagugcgucagcuucagcaucaaccagugcuucggcuucggcaucaacaagugccucagcaucagcaucaacgagugcgucagccucagcaaguacuagugcaucagcaucagcaucaaccagugcaucagccucagcaaguaucucagcgucugaaucggcaucaacgagugcaucagcaucagcaucaacgagugcaucggcuucagcgucaaccagugcaucagucucagcaagcaccagugcgucggcuucagcaucaacgagugccucagccucagcaaguaucucagcgucugaaucggcaucaacgagugcgucagccucagcaaguacuagugcaucggcuucagcaagcaccagugcgucggcuucagcaucaaccagugccucagccucagcaaguaucucagcgucugaaucggcaucaacgagugcgucagccucagcaaguacuagugcaucagcaucagcaucaacgagugcaucggcuucagcaaguaccagcgccucagcuucagcaagcaccagugcgucagccucagcaaguaccagcgccucagccucagcaagcaccagugccucagcuucagcaaguaccagugcgucagccucagcgucgacaagugcgucggcuucagcaaguaccucagcgucugaaucagcaucaacgagugcaucagcuucagcaucaacaagugcuucagcuucagcaaguaccagugcgucggcuucagcaucaacgagugcuucagucucagcgucaaccagugccucugaaucagcaucaacaagugccucggcuucagcaagcaccagugcgucggcuucagcaaguacuagugcaucggcuucagcaucgacaagugcgucugaaucggcaucaacgagugcuucggcuucagcaucaacgagugcgucagccucagcaagcacaucagcuucugaaucugcaucaaccagugcguccgcuucagcgucaaccagugcgucggcuucagcgucgacaagugcuucggcuucagcaucaacgagugcgucggccucagcaagcgcaaguaccucagcgucagcuuccgccucaaccagugcgucggcuucagcaagcacaagugcgucagccucagcaaguaucucagcgucugaaucggcaucaacgagugcgucugagucagcaucaacgaguacgucagccucagcaagcacaucagcuucugaaucugcaucaaccagugcgucagccucagcaucgacaagcgccucagcuucagcaaguaccagugcuucagccucagcgucgacaagugcgucggccucaaccagugcaucugaaucggcaucaaccagugcgucagccucagcaaguacuagugcaucagcuucagcaucaacgagugcaucggcuucagcaucaaccagugccucggcuucagcgucaaccagugcgucagcuucagcaaguaccagugcuucagucucagcaucaacaagugcuucagccucagcaucgacaagugccucggcuucagcaagcacaucagcaucugaaucagcgucgacaagcgccucagcuucagcaaguaccagugcgucagccucagcgucgacaagugcgucagccucagcaaguacuagugcaucagcuucagcaucaacgagugcaucggcuucggcgucaaccagugcaucagagucagcaaguaccagugcgucagcuuccgcaucaacaagugccucggcuucagcaagcaccagugcgucggcuucagcaaguacuagcgccucagccucagccucaaccagugcgucagccucagcaaguaucucagcgucugaaucggcaucaacgagugcguccgcuucagcaaguacuagcgccucagccucagcgucaacaagugcaucggcuucagcgucaacgagugcgucugaaucggcaucaacgagugcguccgcuucagcaaguacuagcgccucagccucagcgucaacaagugcaucggcuucagcaucaacgagugcguccgcuucagcaaguacuagcgccucagccucagcgucaacaagugcaucggcuucagcgucaacgagugcgucugagucagcaucaacgagugcgucagccucagcaagcacaucagcuucugaaucugcaucaaccagugcgucagccucagcaucgacaagcgccucagcuucagcaaguaccagugcgucagccucagcgucgacaagugcgucggcuucagcaaguaccagugcgucagccucagcaaguaccagugcgucagccucagcgucgacaagugcgucggccucaaccagugcaucugaaucggcaucaaccagugcgucagccucagcaaguacuagugcaucagcuucagcaucaacgagugcaucggcuucagcaucaaccagugcaucagagucagcaaguaccagugcgucagcuuccgcaucaacaagugccucggcuucagcaaguacuagcgccucagccucagcgucaacaagugcuucagcuuccgcgucaaccagcgccucggccucagcaaguaucucagcgucugaaucggcaucaacaagugccucggcuucagcaucaacgagugcaucagucucagcaagcaccagugcgucggccucagcaagcaccagcgcgucugaauccgcaucaaccagugccucagcuucagcaaguaccucagcaucugaaucagcaucaacaagugcaucggcuucagcaagcacaagugcuucagccucagcaaguaucucagcgucugaaucggcaucaacgagugcguccgcuucagcaaguacuagcgccucagcaucagcgucaacaagugcuucggcuucagcgucaacgagugcgucugagucagcaucaacgaguacgucagccucagcaagcacaucagcuucugaaucugcaucaaccagugcgucagccucagcaucgacaagcgccucagcuucagcaaguaccagugcgucagccucagcaaguaccagugcuucagccucagcgucgacaagugcgucggccucaaccagugcaucugaaucggcaucaaccagugcgucagccucagcaaguacuagcgccucagccucagcaucaacgagugcguccgcuucagcaaguacuagugcaucagcaucagcaucaacgagugcaucggcuucagcaaguaccagcgccucagcuucagcaagcaccagugcgucagccucagcaaguaccagcgccucagccucagcaagcaccagugccucagcuucagcaaguaccagugcgucagccucagcgucgacaagugcgucggcuucagcaaguaccucagcgucugaaucagcaucaacgagugcaucagcuucagcaucaacaagugcuucagcuucagcaaguaccagugcgucggcuucagcaucaacgagugcuucagucucagcgucaaccagugccucugaaucagcaucaacaagugccucggcuucagcaagcaccagugcgucggcuucagcaaguacuagugcaucggcuucagcaucgacaagugcgucugaaucggcaucaacgagugcuucggcuucagcaucaacgagugcgucagccucagcaagcacaucagcuucugaaucugcaucaaccagugcguccgcuucagcgucaaccagugcgucggcuucagcgucgacaagugcuucggcuucagcaucaacgagugcgucggccucagcaagcgcaaguaccucagcgucagcuuccgccucaaccagugcguccgcuucagcaagcacaagugcgucagccucagcaaguaucucagcgucugaaucggcaucaacgagugcgucggccucagcaagcgcaaguaccucagcgucagcuuccgccucaaccagugcgucggcuucagcaagcacaagugcgucagccucagcaaguaucucagcgucugaaucggcaucaacgagugcgucugagucagcaucaacgaguacgucagccucagcaagcacaucagcuucugaaucggcaucaaccagugcgucagccucagcaucgacaagcgccucagcuucagcaaguaccagugcuucagccucagcgucgacaagugcgucggccucaaccagugcaucugaaucggcaucaaccagugcgucagccucagcaaguacuagugcaucagcuucagcaucaacgagugcaucggcuucagcaucaaccagugccucggcuucagcgucaaccagugcgucagcuucagcaaguaccagugcuucagucucagcaucaacaagugcuucagccucagcaucgacaagugccucggcuucagcaagcacaucagcaucugaaucagcgucgacaagugcgucggccucaaccagugcaucugaaucggcaucaaccagugcgucagccucagcaaguacuagugcaucagcuucagcaucaacgagugcaucggcuucggcgucaaccagugcaucagagucagcaaguaccagugcgucagcuuccgcaucaacaagugccucggcuucagcaagcacaucagcaucugaaucagcgucaaccagugcuucggcuucagcaaguaccagugcuucagcuucagcaucaaccagcgccucggccucagcaagcaccucagcuucugaaucggccucaaccagcgccucggccucagcaagcaccucagcuucugaaucggccucaaccagcgccucagccucagcaucaacgagugcuucggcuucagcaagcacaagcgccucggguucagcaucaacgaguacgucagcuucagcgucaaccagugcuucagccucagcaucaacaagugcgucagccucagcaaguaucucagcgucugaaucggcaucaacgagugcgucugagucagcaucaacgaguacgucagccucagcaagcaccucagcuucugaaucggccucaaccagugcgucagccucagcaucgacaagcgccucagcuucagcaaguaccagugcuucagccucagcgucgacaagugcgucggccucaaccagugcaucugaaucggcaucaaccagugcgucagccucagcaaguacuagugcaucggcuucagcaucaaccagugccucggcuucagcgucaaccagugcgucagcuucagcaaguaccagugcuucagucucagcaucaacaagugcuucagccucagcaucgacaagugccucggcuucagcaagcacaucagcaucugaaucagcgucgacaagcgccucagcuucagcaaguaccagugcgucagccucagcgucgacaagugcgucagccucagcaaguacuagugcaucagcuucagcaucaacgagugcaucggcuucggcgucaaccagugcaucagagucagcaaguaccagugcgucagcuuccgcaucaacaagugccucggcuucagcaagcaccagugcgucggcuucagcaaguacuagcgccucagccucagccucaaccagugcgucagccucagcaaguaucucagcgucugaaucggcaucaacgagugcguccgcuucagcaaguacuagcgccucagccucagcgucaacaagugcaucggcuucagcgucaacgagugcgucugaaucggcaucaacgagugcguccgcuucagcaaguacuagcgccucagccucagcgucaacaagugcaucggcuucagcaucaacgagugcguccgcuucagcaaguacuagcgccucagccucagcgucaacaagugcaucggcuucagcgucaacgagugcgucugagucagcaucaacgagugcgucagccucagcaagcacaucagcuucugaaucugcaucaaccagugcgucagccucagcaucgacaagcgccucagcuucagcaaguaccagugcgucagccucagcgucgacaagugcgucggcuucagcaaguaccagugcgucagccucagcaaguaccagugcgucagccucagcgucgacaagugcgucggccucaaccagugcaucugaaucggcaucaaccagugcgucagccucagcaaguacuagugcaucagcuucagcaucaacgagugcaucggcuucagcaucaaccagugcaucagagucagcaaguaccagugcgucagcuuccgcaucaacaagugccucggcuucagcaaguacuagcgccucagccucagcgucaacaagugcuucagcuuccgcgucaaccagcgccucggccucagcaaguaucucagcgucugaaucggcaucaacaagugccucggcuucagcaucaacgagugcaucagucucagcaagcaccagugcgucggccucagcaagcaccagcgcgucugaauccgcaucaaccagugccucagcuucagcaaguaccucagcaucugaaucagcaucaacaagugccucggcuucagcaagcacaagugcuucagccucagcaaguaucucagcgucugaaucggcaucaacgagugcguccgcuucagcaaguacuagcgccucagcaucagcgucaacaagugcuucggcuucagcgucaacgagugcgucugagucagcaucaacgaguacgucagccucagcaagcacaucagcuucugaaucugcaucaaccagugcgucagccucagcaucgacaagcgccucagcuucagcaaguaccagugcgucagccucagcaaguaccagugcuucagccucagcgucgacaagugcgucggccucaaccagugcaucugaaucggcaucaaccagugcgucagccucagcaaguacuagcgccucagccucagcaucaacgagugcguccgcuucagcaaguacuagugcaucagcuucagcaaguacuagcgccucagccucagcgucgacaagcgccucagcuucagcaaguaccagugcgucagccucagcgucgacaagugcgucggcuucagcaaguaccucagcgucugaaucagcaucaacaagugcgucggcuucagcaucaacgagugcaucagcuucagcaucaacaagugcuucagcuucagcaaguaccagugcgucggcuucagcaucaacgagugcuucagucucagcgucaaccagugccucugaauccgcaucaacaagugccucggcuucagcaagcaccagugcuucggcuucagcgucaacgagugcgucugagucagcaucaacgagugcgucagccucagcaagcacaucagcuucugaaucugcaucaaccagugcgucagcuuccgcaucaacaagcgccucggccucagcaaguacaagugcuucagccucagcaucaaccagugcaucagcuucagccucaacaagugcuucagccucagcgucaaccagugccucggcuucagcaaguaccagugcgucagcuucagcaagcacaagugcgucagcuucagcaucaaccagugcuucggcuucggcaucaacaagugccucagcaucagcaucaacgagugcgucagccucagcaaguacuagugcaucagcaucagcaucaaccagugcaucagccucagcaaguaucucagcgucugaaucggcaucaacgagugcaucagcaucagcaucaacgagugcaucggcuucagcgucaaccagugcaucagucucagcaagcaccagugcgucggcuucagcaucaacgagugccucagccucagcaaguaucucagcgucugaaucggcaucaacgagugcgucagccucagcaaguacuagugcaucggcuucagcaagcaccagugcgucggcuucagcaucaaccagugccucagccucagcaaguaucucagcgucugaaucggcaucaacgagugcgucagccucagcaaguacuagugcaucagcaucagcaucaacgagugcaucggcuucagcaaguaccagcgccucagcuucagcaagcaccagugcgucagccucagcaaguaccagcgccucagccucagcaagcaccagugccucagcuucagcaaguaccagugcgucagccucagcgucgacaagugcgucggcuucagcaaguaccucagcgucugaaucagcaucaacgagugcaucagcuucagcaucaacaagugcuucagcuucagcaaguaucucagcgucugaaucggcaucaacgagugcguccgcuucagcaaguacuagcgccucagcaucagcgucaacaagugcuucggcuucagcgucaacgagugcgucugagucagcaucaacgaguacgucagccucagcaagcacaucagcuucugaaucugcaucaaccagugcgucagccucagcaucgacaagcgccucagcuucagcaaguaccagugcgucagccucagcaaguaccagugcuucagccucagcgucgacaagugcgucggccucaaccagugcaucugaaucggcaucaaccagugcgucagccucagcaaguacuagcgccucagccucagcaucaacgagugcguccgcuucagcaaguacuagugcaucagcuucagcaaguacuagcgccucagccucagcgucgacaagcgccucagcuucagcaaguaccagugcgucagccucagcgucgacaagugcgucggcuucagcaaguaccucagcgucugaaucagcaucaacaagugcgucggcuucagcaucaacgagugcaucagcuucagcaucaacaagugcuucagcuucagcaaguaccagugcgucggcuucagcaucaacgagugcuucagucucagcgucaaccagugccucugaauccgcaucaacaagugccucggcuucagcaagcaccagugcuucggcuucagcgucaacgagugcgucugagucagcaucaacgagugcgucagccucagcaagcacaucagcuucugaaucugcaucaaccagugcgucagcuuccgcaucaacaagcgccucggccucagcaaguacaagugcuucagccucagcaucaaccagugcaucagcuucagccucaacaagugcuucagccucagcgucaaccagugccucggcuucagcaaguaccagugcgucagcuucagcaagcacaagugcgucagcuucagcaucaaccagugcuucggcuucggcaucaacaagugccucagcaucagcaucaacgagugcgucagccucagcaaguacuagugcaucagcaucagcaucaaccagugcaucagccucagcaaguaucucagcgucugaaucggcaucaacgagugcaucagcaucagcaucaacgagugcaucggcuucagcgucaaccagugcaucagucucagcaagcaccagugcgucggcuucagcaucaacgagugccucagccucagcaaguaucucagcgucugaaucggcaucaacgagugcgucagccucagcaaguacuagugcaucggcuucagcaagcaccagugcgucggcuucagcaucaaccagugccucagccucagcaaguaucucagcgucugaaucggcaucaacgagugcgucagccucagcaaguacuagugcaucagccucagcaucaacgagugcaucggcuucagcaaguaccagcgccucagcuucagcaagcaccagugcgucagccucagcaaguaccagcgccucagccucagcaagcaccagugccucagcuucagcaaguaccagugcgucagccucagcaucaacaagugcuucagcuucggccucaacaagugcgucagcuucagcaucaacgagugcgucggcuucagcaagcaccagugccucggccucagcaagcaccagugcuucagcuucagcaucaacaagugcgucagcuucagcaaguacaucaguuucaaauucagcaaaccauucgaacucacaaguuggaaauacuucuggaucgacagguaaaucccaaaaagaauugccuaauacagguacugagucgucaauuggaucuguguuacuuggaguucuagcagcuguuacagguauuggauugguugcgaaacgccguaaacgugaugaagaagaguaa

SEQ ID NO: 106 - protein, 6 aa, Artificial sequence
  Synthetic linker
GSGGGA

SEQ ID NO: 107 - protein, 12 aa, Artificial sequence
  Synthetic linker
GSGGGAGSGGGA

SEQ ID NO: 108 - protein, 18 aa, Artificial sequence
  Synthetic linker
GSGGGAGSGGGAGSGGGA

SEQ ID NO: 109 - protein, 24 aa, Artificial sequence
  synthetic linker
GSGGGAGSGGGAGSGGGAGSGGGA

SEQ ID NO: 110 - protein, 10 aa, Artificial sequence
  Synthetic linker
GENLYFQSGG

SEQ ID NO: 111 - protein, 12 aa, Artificial sequence
  Synthetic linker
KPEPKPAPAPKP

SEQ ID NO: 112 - protein, 12 aa, Artificial sequence
  Synthetic linker
AEAAAKEAAAKA

SEQ ID NO: 113 - protein, 8 aa, Artificial sequence
  Synthetic linker
SACYCELS

(19) United States
(12) Patent Application Publication
(10) Pub. No.: US 2023/0050225 A1
(43) Pub. Date: Feb. 16, 2023

(54) Vaccines targeting Neisseria gonorrhoeae

(71) Applicant: Evaxion Biotech A/S, Hørsholm (DK)

(72) Inventors: Andreas Holm Mattsson, Hørsholm (DK); Christian Skjødt Steenmans, Hørsholm (DK)

(21) Appl. No.: 17/791,038

(22) PCT Filed: Jan. 6, 2021
(86) PCT No.: PCT/EP2021/050134

§ 371 Date: Jul. 6, 2022

(30) Foreign Application Priority Data
Jan. 6, 2020 (EP) 20150369.5

(51) Int. Cl. A61K 39/095; A61P 31/04

(52) U.S. Cl. CPC A61K 39/095; A61P 31/04; A61K 2039/575

(57) ABSTRACT

Proteins, protein fragments, nucleic acids and vectors derived from Neisseria gonorrhoeae, as well as to methods of inducing immunity against N. gonorrhoeae. Also disclosed are antibodies binding to the proteins and protein fragments.

### FIELD OF THE INVENTION

The present invention relates to the field of antimicrobial prophylaxis and therapy. In particular the present invention relates to novel proteins and polynucleotides derived from Neisseria Gonorrhoeae (NeGo). The invention further relates to vectors comprising the polynucleotides, transformed host organisms expressing the polynucleotides, antibodies (mono- or polyclonal) specific for the polypeptides as well as diagnostic, prophylactic and therapeutic uses and methods. Finally, also methods of preparation are part of the invention

### BACKGROUND OF THE INVENTION

Neisseria gonorrhoeae is a bacterial pathogen (a Gram-negative diplococcus), which i.a. causes the sexually transmitted disease gonorrhoea. There is currently no effective vaccine against Nego infection.

Gonorrhea poses a worldwide risk as one of the most commonly reported communicable diseases. Although NeGo primarily infects mucous membranes, it is capable of invading tissues and evading host defenses. It is the causative agent of a spectrum of sequelae, ranging from asymptomatic mucosal infection to significant disease syndromes in both men and women. These include disseminated gonococcal infection (“DGI”) in men and women, as well as salpingitis or pelvic inflammatory disease (“PID”) in women. Either salpingitis or PID may themselves lead to long-term sequelae, including ectopic pregnancy and infertility. Other important sequelae, sometimes requiring surgical intervention, include recurrent infection, chronic pelvic pain, dyspareunia, pelvic adhesions and other inflammatory residua.

### OBJECT OF THE INVENTION

It is an object of embodiments of the invention to provide NeGo derived antigenic polypeptides that may serve as constituents in vaccines against NeGo infections and in diagnosis of NeGo infections. It is also an object to provide nucleic acids, vectors, transformed cells, vaccine compositions, and other useful means for molecular cloning as well as for therapy and diagnosis with relevance for NeGo.

### SUMMARY OF THE INVENTION

It has been found by the present inventor(s) that NeGo expresses a number of proteins, which are candidates as vaccine targets as well as candidates as immunizing agents for preparation of antibodies that target NeGo.

So, in a 1^{st} aspect the present invention relates to a polypeptide comprising

a) an amino acid sequence selected from the group consisting of any one of SEQ ID NOs: 1-35, or

b) an amino acid sequence consisting of at least or exactly 5 contiguous amino acid residues from any one of SEQ ID NOs: 1-35, or

c) an amino acid sequence having a sequence identity of at least 60% with the amino acid sequence of a),

d) an amino acid sequence having a sequence identity of at least 60% with the amino acid sequence of b), or

e) an assembly of amino acids derived from any one of SEQ ID NOs: 1-35, which has essentially the same 3D conformation as in the protein from which said assembly is derived so as to constitute a B-cell epitope, said polypeptide being antigenic in a mammal.

In a 2^{nd} aspect, the invention relates to an isolated nucleic acid fragment, which comprises

i) a nucleotide sequence encoding a polypeptide of the 1^{st} aspect of the invention and of any embodiment of the 1^{st} aspect disclosed herein, or

ii) a nucleotide sequence consisting of the part of any one of SEQ ID NOs: 31-90 that encodes any one of SEQ ID NOs: 1-35,

iii) a nucleotide sequence consisting of a fragment of at least 12 consecutive nucleotides of the nucleotide sequence defined in ii and in same reading frame,

iv) a nucleotide sequence having a sequence identity of at least 60% with the nucleotide sequence in i) or ii),

v) a nucleotide sequence having a sequence identity of at least 60% with the nucleotide sequence in iii),

vi) a nucleotide sequence complementary to the nucleotide sequence in any one of i)-v), or

vii) a nucleotide sequence which hybridizes under highly stringent conditions with the nucleotide sequence in i)-vi).

In a 3^{rd} aspect, the invention relates to a vector comprising the nucleic acid of the 2^{nd} aspect of the invention and of any embodiment of said 2^{nd} aspect, such as a cloning vector or an expression vector.

In a 4^{th} aspect, the invention relates to a transformed cell, which carries the vector of the 3^{rd} aspect of the invention and of any embodiment of the 3^{rd} aspect disclosed herein. Also included in this aspect is a cell line derived from a transformed cell of the invention.

In a 5^{th} aspect, the invention relates to a pharmaceutical composition comprising
a polypeptide of the 1^{st} aspect of the invention and of any embodiment of the 1^{st} aspect disclosed herein,
a nucleic acid fragment of to the 2^{nd} aspect of the invention and of any embodiment of the 2^{nd} aspect disclosed herein,
a vector of the 3^{rd} aspect of the invention and of any embodiment of the 3^{rd} aspect disclosed herein, or
a cell of the 4^{th} aspect of the invention and of any embodiment of the 4^{th} aspect disclosed herein; and
   a pharmaceutically acceptable carrier, vehicle or diluent.

In a 6^{th} aspect, the invention relates to a method for inducing immunity in an animal by administering at least once an immunogenically effective amount of
a polypeptide of the 1^{st} aspect of the invention and of any embodiment of the 1^{st} aspect disclosed herein,
a nucleic acid fragment of to the 2^{nd} aspect of the invention and of any embodiment of the 2^{nd} aspect disclosed herein,
a vector of the 3^{rd} aspect of the invention and of any embodiment of the 3^{rd} aspect disclosed herein,
a cell of the 4^{th} aspect of the invention and of any embodiment of the 4^{th} aspect disclosed herein, or
a pharmaceutical composition of the 5^{th} aspect of the invention or of any embodiment of the 5^{th} aspect disclosed herein
   so as to induce adaptive immunity against NeGo in the animal.

In a 7^{th} aspect, the invention relates to a polyclonal antibody in which the antibodies specifically bind to at least one polypeptide of the 1^{st} aspect of the invention and of any embodiment of the 1^{st} aspect disclosed herein, and which is essentially free from antibodies binding specifically to other NeGo polypeptides; or a an isolated monoclonal antibody or antibody analogue which binds specifically to a polypeptide according to the 1^{st} aspect of the invention and of any embodiment of the 1^{st} aspect disclosed herein.

In an 8^{th} aspect, the invention relates to a pharmaceutical composition comprising an antibody of the 7^{th} aspect of the invention and of any embodiment of the 7^{th} aspect disclosed herein and a pharmaceutically acceptable carrier, vehicle or diluent.

In an 9^{th} aspect, the invention relates to a method for prophylaxis, treatment or amelioration of infection with NeGo, comprising administering a therapeutically effective amount of 1) an antibody of the 7^{th} aspect of the invention and of any embodiment of the 7^{th} aspect disclosed herein or 2) a pharmaceutical composition of the 8^{th} aspect of the invention and of any embodiment of the 8^{th} aspect disclosed herein, to an individual in need thereof.

In a 10^{th} aspect, the invention relates to a method for determining, quantitatively or qualitatively, the presence of NeGo, in a sample, the method comprising contacting the sample with an antibody of the 7^{th} aspect of the invention and of any embodiment of the 7^{th} aspect disclosed herein and detecting the presence of antibody bound to material in the sample.

In an 11^{th} aspect, the invention relates to a method for determining, quantitatively or qualitatively, the presence of antibodies specific for NeGo, in a sample, the method comprising contacting the sample with a polypeptide of the 1^{st} aspect of the invention and of any embodiment of the 1^{st} aspect disclosed herein, and detecting the presence of antibody said polypeptide.

In a 12^{th} aspect, the invention relates to a method for determining, quantitatively or qualitatively, the presence of a nucleic acid characteristic of NeGo in a sample, the method comprising contacting the sample with a nucleic acid fragment of the 2^{nd} aspect of the invention and of any embodiment of the 2^{nd} aspect disclosed herein, and detecting the presence of nucleic acid in the sample that hybridized to said nucleic acid fragment.

In a 13^{th} aspect, the invention relates to a method for the preparation of the polypeptide of the 1^{st} aspect of the invention and of any embodiment thereof, comprising
culturing a transformed cell of the 4^{th} aspect of the invention and of any embodiment of the 4^{th} aspect disclosed herein, insofar as these relate to a cell capable of expressing the polypeptide of the invention, under conditions that facilitate that the transformed cell expresses the nucleic acid fragment of the 2^{nd} aspect of the invention, option i), and of any embodiment thereof, and subsequently recovering said polypeptide, or
preparing said polypeptide by means of solid or liquid phase peptide synthesis.

In a 14^{th} aspect, the invention relates to a method for determining whether a substance, such as an antibody, is potentially useful for treating infection with NeGo, the method comprising contacting the polypeptide of the 1^{st} aspect of the invention and of any embodiment thereof with the substance and subsequently establishing whether the substance has at least one of the following characteristics:

1) the ability to bind specifically to said polypeptide,

2) the ability to compeed with said polypeptide for specific binding to a ligand/receptor,

3) the ability to specifically inactivate said polypeptide.

In a 15^{th} aspect, the invention relates to a method for determining whether a substance, such as a nucleic acid, is potentially useful for treating infection with NeGo, the method comprising contacting the substance with the nucleic acid fragment of the 2^{nd} aspect of the invention and of any embodiment thereof, and subsequently establishing whether the substance has either the ability to

1) bind specifically to the nucleic acid fragment, or

2) bind specifically to a nucleic acid that hybridizes specifically with the nucleic acid fragment.

In a 16^{th} aspect, the invention relates to the polypeptide of the 1^{st} aspect of the invention and of any embodiment of the 1^{st} aspect disclosed herein, for use as a pharmaceutical, notably for use as a pharmaceutical in the treatment, prophylaxis or amelioration of infection with NeGo.

In a 17^{th} aspect, the invention relates to a nucleic acid fragment of the 2^{nd} aspect of the invention and of any embodiment of the 1^{st} aspect disclosed herein, or a vector of the 3^{rd} aspect of the invention and of any embodiment of the 2^{nd} aspect disclosed herein, for use as a pharmaceutical, notably for use as a pharmaceutical in the treatment, prophylaxis or amelioration of infection with NeGo.

In an 18^{th} aspect of the invention, the invention relates to a cell of the 4^{th} aspect of the invention and of any embodiment of the 4^{th} aspect disclosed herein for use as a pharmaceutical, notably for use as a pharmaceutical in the treatment, prophylaxis or amelioration of infection with NeGo.

Finally, in a 19^{th} aspect, the invention relates to an antibody, antibody fragment or antibody analogue of the 7^{th} aspect of the invention and of any embodiment of the 7^{th} aspect disclosed herein, use as a pharmaceutical, notably use as a pharmaceutical in the treatment, prophylaxis or amelioration of infection with NeGo.

### LEGENDS TO THE FIGURES

FIG. 1: Kaplan-Meyer plots showing infection rates post challenge infection in mice immunized with vaccine (-□-) (group 1) and mice receiving adjuvant only (-●-) as described in Example 1.

A: After infection with N. gonorrhoeae MS11.

B: After infection with N. gonorrhoeae H041.

FIG. 2: Kaplan-Meyer plots showing infection rates post challenge infection in mice immunized with vaccine (-□-) (group 2) and mice receiving adjuvant only (-●-) as described in Example 1.

A: After infection with N. gonorrhoeae MS11.

B: After infection with N. gonorrhoeae H041.

FIG. 3: Kaplan-Meyer plots showing infection rates post challenge infection in mice immunized with vaccine (-□-) (group 3) and mice receiving adjuvant only (-●-) as described in Example 1.

A: After infection with N. gonorrhoeae MS11.

B: After infection with N. gonorrhoeae H041.

FIG. 4: Kaplan-Meyer plots showing infection rates post challenge infection in mice immunized with vaccine (-□-) (group 4) and mice receiving adjuvant only (-●-) as described in Example 1.

A: After infection with N. gonorrhoeae MS11.

B: After infection with N. gonorrhoeae H041.

FIG. 5: Kaplan-Meyer plots showing infection rates post challenge infection in mice immunized with vaccine (-□-) (group 5) and mice receiving adjuvant only (-●-) as described in Example 1.

A: After infection with N. gonorrhoeae MS11.

B: After infection with N. gonorrhoeae H041.

FIG. 6: Kaplan-Meyer plots showing infection rates post challenge infection in mice immunized with vaccine (-□-) (group 6) and mice receiving adjuvant only (-●-) as described in Example 1.

A: After infection with N. gonorrhoeae MS11.

B: After infection with N. gonorrhoeae H041.

FIG. 7: Kaplan-Meyer plots showing infection rates post challenge infection in mice immunized with vaccine (-□-) (group 7) and mice receiving adjuvant only (-●-) as described in Example 1.

A: After infection with N. gonorrhoeae MS11.

B: After infection with N. gonorrhoeae H041.

FIG. 8: Kaplan-Meyer plots showing infection rates post challenge infection in mice immunized with vaccine (-□-) (group 8) and mice receiving adjuvant only (-●-) as described in Example 1.

A: After infection with N. gonorrhoeae MS11.

B: After infection with N. gonorrhoeae H041.

FIG. 9: Kaplan-Meyer plots showing infection rates post challenge infection in mice immunized with vaccine (-□-) (group 9) and mice receiving adjuvant only (-●-) as described in Example 1.

A: After infection with N. gonorrhoeae MS11.

B: After infection with N. gonorrhoeae H041.

FIG. 10: Kaplan-Meyer plots showing infection rates post challenge infection in mice immunized with vaccine (-□-) (group 10) and mice receiving adjuvant only (-●-) as described in Example 1.

A: After infection with N. gonorrhoeae MS11.

B: After infection with N. gonorrhoeae H041.

FIG. 11: Kaplan-Meyer plots showing infection rates post challenge infection in mice immunized with vaccine (-□-) (group 11) and mice receiving adjuvant only (-●-) as described in Example 1.

A: After infection with N. gonorrhoeae MS11.

B: After infection with N. gonorrhoeae H041.

FIG. 12: Kaplan-Meyer plots showing infection rates post challenge infection in mice immunized with vaccine (-□-) (group 12) and mice receiving adjuvant only (-●-) as described in Example 1.

A: After infection with N. gonorrhoeae MS11.

B: After infection with N. gonorrhoeae H041.

FIG. 13: Kaplan-Meyer plots showing infection rates post challenge infection in mice vaccinated with construct NGO1549-35-289 or receiving adjuvant only.

A: After infection with N. gonorrhoeae MS11.

B: After infection with N. gonorrhoeae FA1090.

FIG. 14: Kaplan-Meyer plots showing infection rates post challenge infection in mice vaccinated with construct NGO264-44-346 or receiving adjuvant only.

A: After infection with N. gonorrhoeae MS11.

B: After infection with N. gonorrhoeae FA1090.

FIG. 15: Kaplan-Meyer plots showing infection rates post challenge infection in mice vaccinated with composition of constructs NGO1549-35-289 and NGO264-44-346 or receiving adjuvant only.

A: After infection with N. gonorrhoeae MS11.

B: After infection with N. gonorrhoeae FA1090.

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively.

The term “amino acid sequence” is the order in which amino acid residues, connected by peptide bonds, lie in the chain in peptides and proteins in the direction from the free N-terminus to the free C-terminus.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAAC-3′ and 5′-ATACGGGAC-3′ will provide the sequence identity 77.8% (N_{ref}=9 and N_{dif}=2). It will be understood that such a sequence identity determination requires that the two aligned sequences are aligned so that there are no overhangs between the two sequences: each amino acid in each sequence will have to be matched with a counterpart in the other sequence.

An “assembly of amino acids” means two or more amino acids bound together by physical or chemical means.

The “3D conformation” is the 3 dimensional structure of a biomolecule such as a protein. In monomeric polypeptides/proteins, the 3D conformation is also termed “the tertiary structure” and denotes the relative locations in 3 dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “linker” is an amino acid sequence, which is introduced between two other amino acid sequences in order to separate them spatially. A linker may be “rigid”, meaning that it does substantially not allow the two amino acid sequences that it connects to move freely relative to each other. Likewise, a “flexible” linker allows the two sequences connected via the linker to move substantially freely relative to each other. In the fusion proteins, which are part of the present invention, both types of linkers are useful. However, one particular interesting linker useful in the present invention has the 12 amino acid residue sequence AEAAAKEAAAKA (SEQ ID NO: 112).

Other linkers of interest are listed in the following table:

| Type | Name | Sequence |
|---|---|---|
| Flexible | FS | GSGGGA (SEQ ID NO: 106) |
| Flexible | FL | GSGGGAGSGGGA (SEQ ID NO: 107) |
| Flexible | FV1 | GSGGGAGSGGGAGSGGGA (SEQ ID |
| | | NO: 108) |
| Flexible | FV2 | GSGGGAGSGGGAGSGGGAGSGGGA (SEQ |
| | | ID NO: 109) |
| Flexible | FM | GENLYFQSGG (SEQ ID NO: 110) |
| Rigid | RL1 | KPEPKPAPAPKP (SEQ ID NO: 111) |
| Rigid | RL2 | AEAAAKEAAAKA (SEQ ID NO: 112) |
| Rigid | RM | SACYCELS (SEQ ID NO: 113) |

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce nor elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Hybridization under “stringent conditions” is herein defined as hybridization performed under conditions by which a probe will hybridize to its target sequence, to a detectably greater degree than to other sequences. Stringent conditions are target-sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Specificity is typically the function of post-hybridization washes, the critical factors being the ionic strength and temperature of the final wash solution. Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature. Formulas are available in the art for the determination of melting temperatures.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method disclosed herein substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

### SPECIFIC EMBODIMENTS OF THE INVENTION

The Polypeptides of the Invention

In some embodiments the at least 5 contiguous amino acids referred to in option b) in the definition of the 1^{st} aspect of the invention constitute at least or exactly or at most 6, such as at least or exactly or at most 7, at least or exactly or at most 8, at least or exactly or at most 9, at least or exactly or at most 10, at least or exactly or at most 11, at least or exactly or at most 12, at least or exactly or at most 13, at least or exactly or at most 14, at least or exactly or at most 15, at least or exactly or at most 16, at least or exactly or at most 17, at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27 at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, or at least or exactly or at most 52 contiguous amino acid residues.

The number of contiguous amino acids in option b) can be higher, for all of SEQ ID NOs. 2-35. Another way to phrase this is that for each of SEQ ID NOs: 1-35, the number of the contiguous amino acid residues is at least or exactly or at most N-n, where N is the length of the sequence ID in question and n is any integer between 1 and N-5; that is, the at least or exactly 5 contiguous amino acids can be at least any number between 5 and the length of the reference sequence minus one, in increments of one.

Insofar as embodiment b relates to SEQ ID NOs: 2-35, the at least 5 contiguous amino acids referred to in option b) in the definition of the 1^{st} aspect of the invention may also constitute at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, or at least or exactly or at most 101 contiguous amino acid residues.

Insofar as embodiment b relates to SEQ ID NOs: 3-35, the at least 5 contiguous amino acids referred to in option b) in the definition of the 1^{st} aspect of the invention may also constitute at least or exactly or at most 102, at least or exactly or at most 103, at least or exactly or at most 104, at least or exactly or at most 105, at least or exactly or at most 106, at least or exactly or at most 107, or at least or exactly or at most 108 contiguous amino acid residues.

Insofar as embodiment b relates to SEQ ID NOs: 4-35, the at least 5 contiguous amino acids referred to in option b) in the definition of the 1^{st} aspect of the invention may also constitute at least or exactly or at most 109, at least or exactly or at most 110, at least or exactly or at most 111, at least or exactly or at most 112, or at least or exactly or at most 113 contiguous amino acid residues.

Insofar as embodiment b relates to SEQ ID NOs: 5-35, the at least 5 contiguous amino acids referred to in option b) in the definition of the 1^{st} aspect of the invention may also constitute at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, at least or exactly or at most 192, at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, or at least or exactly or at most 215 contiguous amino acid residues.

Insofar as embodiment b relates to SEQ ID NOs: 6-35, the at least 5 contiguous amino acids referred to in option b) in the definition of the 1^{st} aspect of the invention may also constitute at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, or at least or exactly or at most 227 contiguous amino acid residues.

Insofar as embodiment b relates to SEQ ID NOs: 7-35, the at least 5 contiguous amino acids referred to in option b) in the definition of the 1^{st} aspect of the invention may also constitute at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, at least or exactly or at most 263, at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, or at least or exactly or at most 282 contiguous amino acid residues.

Insofar as embodiment b relates to SEQ ID NOs: 8-35, the at least 5 contiguous amino acids referred to in option b) in the definition of the 1^{st} aspect of the invention may also constitute at least or exactly or at most 283, at least or exactly or at most 284, at least or exactly or at most 285, at least or exactly or at most 286, at least or exactly or at most 287, or at least or exactly or at most 288 contiguous amino acid residues.

Insofar as embodiment b relates to SEQ ID NOs: 9-35, the at least 5 contiguous amino acids referred to in option b) in the definition of the 1^{st} aspect of the invention may also constitute at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, at least or exactly or at most 326, at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, or at least or exactly or at most 336 contiguous amino acid residues.

Insofar as embodiment b relates to SEQ ID NOs: 10-35, the at least 5 contiguous amino acids referred to in option b) in the definition of the 1^{st} aspect of the invention may also constitute at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, or at least or exactly or at most 345 contiguous amino acid residues.

Insofar as embodiment b relates to SEQ ID NOs: 11-35, the at least 5 contiguous amino acids referred to in option b) in the definition of the 1^{st} aspect of the invention may also constitute at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356, at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, or at least or exactly or at most 376 contiguous amino acid residues.

Insofar as embodiment b relates to SEQ ID NOs: 12-35, the at least 5 contiguous amino acids referred to in option b) in the definition of the 1^{st} aspect of the invention may also constitute at least or exactly or at most 377, at least or exactly or at most 378, at least or exactly or at most 379, at least or exactly or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, or at least or exactly or at most 397 contiguous amino acid residues.

Insofar as embodiment b relates to SEQ ID NOs: 13-35, the at least 5 contiguous amino acids referred to in option b) in the definition of the 1^{st} aspect of the invention may also constitute at least or exactly or at most 398, at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, at least or exactly or at most 407, at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420, or at least or exactly or at most 421 contiguous amino acid residues.

Insofar as embodiment b relates to SEQ ID NOs: 14-35, the at least 5 contiguous amino acids referred to in option b) in the definition of the 1^{st} aspect of the invention may also constitute at least or exactly or at most 422, at least or exactly or at most 423, at least or exactly or at most 424, or at least or exactly or at most 425 contiguous amino acid residues.

Insofar as embodiment b relates to SEQ ID NOs: 15-35, the at least 5 contiguous amino acids referred to in option b) in the definition of the 1^{st} aspect of the invention may also constitute at least or exactly or at most 426, at least or exactly or at most 427, at least or exactly or at most 428, at least or exactly or at most 429, at least or exactly or at most 430, at least or exactly or at most 431, at least or exactly or at most 432, at least or exactly or at most 433, at least or exactly or at most 434, at least or exactly or at most 435, at least or exactly or at most 436, at least or exactly or at most 437, or at least or exactly or at most 438 contiguous amino acid residues.

Insofar as embodiment b relates to SEQ ID NOs: 16-35, the at least 5 contiguous amino acids referred to in option b) in the definition of the 1^{st} aspect of the invention may also constitute at least or exactly or at most 439, at least or exactly or at most 440, at least or exactly or at most 441, at least or exactly or at most 442, at least or exactly or at most 443, at least or exactly or at most 444, at least or exactly or at most 445, at least or exactly or at most 446, at least or exactly or at most 447, at least or exactly or at most 448, at least or exactly or at most 449, at least or exactly or at most 450, at least or exactly or at most 451, at least or exactly or at most 452, at least or exactly or at most 453, at least or exactly or at most 454, at least or exactly or at most 455, at least or exactly or at most 456, at least or exactly or at most 457, at least or exactly or at most 458, at least or exactly or at most 459, at least or exactly or at most 460, at least or exactly or at most 461, at least or exactly or at most 462, at least or exactly or at most 463, at least or exactly or at most 464, at least or exactly or at most 465, at least or exactly or at most 466, or at least or exactly or at most 467 contiguous amino acid residues.

Insofar as embodiment b relates to SEQ ID NOs: 17-35, the at least 5 contiguous amino acids referred to in option b) in the definition of the 1^{st} aspect of the invention may also constitute at least or exactly or at most 468, at least or exactly or at most 469, at least or exactly or at most 470, at least or exactly or at most 471, at least or exactly or at most 472, at least or exactly or at most 473, at least or exactly or at most 474, at least or exactly or at most 475, at least or exactly or at most 476, at least or exactly or at most 477, at least or exactly or at most 478, at least or exactly or at most 479, at least or exactly or at most 480, at least or exactly or at most 481, at least or exactly or at most 482, at least or exactly or at most 483, at least or exactly or at most 484, at least or exactly or at most 485, at least or exactly or at most 486, at least or exactly or at most 487, at least or exactly or at most 488, at least or exactly or at most 489, at least or exactly or at most 490, at least or exactly or at most 491, at least or exactly or at most 492, at least or exactly or at most 493, at least or exactly or at most 494, at least or exactly or at most 495, at least or exactly or at most 496, or at least or exactly or at most 497 contiguous amino acid residues.

Insofar as embodiment b relates to SEQ ID NOs: 18-35, the at least 5 contiguous amino acids referred to in option b) in the definition of the 1^{st} aspect of the invention may also constitute at least or exactly or at most 498, at least or exactly or at most 499, at least or exactly or at most 500, at least or exactly or at most 501, at least or exactly or at most 502, at least or exactly or at most 503, at least or exactly or at most 504, at least or exactly or at most 505, at least or exactly or at most 506, at least or exactly or at most 507, at least or exactly or at most 508, at least or exactly or at most 509, at least or exactly or at most 510, at least or exactly or at most 511, at least or exactly or at most 512, at least or exactly or at most 513, at least or exactly or at most 514, at least or exactly or at most 515, at least or exactly or at most 516, at least or exactly or at most 517, at least or exactly or at most 518, at least or exactly or at most 519, at least or exactly or at most 520, or at least or exactly or at most 521 contiguous amino acid residues.

Insofar as embodiment b relates to SEQ ID NOs: 19-35, the at least 5 contiguous amino acids referred to in option b) in the definition of the 1^{st} aspect of the invention may also constitute at least or exactly or at most 522, at least or exactly or at most 523, at least or exactly or at most 524, at least or exactly or at most 525, or at least or exactly or at most 526 contiguous amino acid residues.

Insofar as embodiment b relates to SEQ ID NOs: 20-35, the at least 5 contiguous amino acids referred to in option b) in the definition of the 1^{st} aspect of the invention may also constitute at least or exactly or at most 527, at least or exactly or at most 528, at least or exactly or at most 529, at least or exactly or at most 530, at least or exactly or at most 531, at least or exactly or at most 532, at least or exactly or at most 533, at least or exactly or at most 534, at least or exactly or at most 535, at least or exactly or at most 536, at least or exactly or at most 537, at least or exactly or at most 538, at least or exactly or at most 539, at least or exactly or at most 540, at least or exactly or at most 541, at least or exactly or at most 542, at least or exactly or at most 543, at least or exactly or at most 544, at least or exactly or at most 545, at least or exactly or at most 546, at least or exactly or at most 547, at least or exactly or at most 548, at least or exactly or at most 549, at least or exactly or at most 550, at least or exactly or at most 551, at least or exactly or at most 552, at least or exactly or at most 553, at least or exactly or at most 554, at least or exactly or at most 555, at least or exactly or at most 556, at least or exactly or at most 557, at least or exactly or at most 558, at least or exactly or at most 559, at least or exactly or at most 560, at least or exactly or at most 561, at least or exactly or at most 562, at least or exactly or at most 563, at least or exactly or at most 564, at least or exactly or at most 565, at least or exactly or at most 566, at least or exactly or at most 567, at least or exactly or at most 568, at least or exactly or at most 569, at least or exactly or at most 570, at least or exactly or at most 571, at least or exactly or at most 572, at least or exactly or at most 573, at least or exactly or at most 574, or at least or exactly or at most 575 contiguous amino acid residues.

Insofar as embodiment b relates to SEQ ID NOs: 21-35, the at least 5 contiguous amino acids referred to in option b) in the definition of the 1^{st} aspect of the invention may also constitute at least or exactly or at most 576, at least or exactly or at most 577, at least or exactly or at most 578, at least or exactly or at most 579, at least or exactly or at most 580, at least or exactly or at most 581, at least or exactly or at most 582, at least or exactly or at most 583, at least or exactly or at most 584, at least or exactly or at most 585, at least or exactly or at most 586, at least or exactly or at most 587, at least or exactly or at most 588, at least or exactly or at most 589, at least or exactly or at most 590, at least or exactly or at most 591, at least or exactly or at most 592, at least or exactly or at most 593, at least or exactly or at most 594, at least or exactly or at most 595, at least or exactly or at most 596, or at least or exactly or at most 597 contiguous amino acid residues.

Insofar as embodiment b relates to SEQ ID NOs: 22-35, the at least 5 contiguous amino acids referred to in option b) in the definition of the 1^{st} aspect of the invention may also constitute at least or exactly or at most 598, at least or exactly or at most 599, at least or exactly or at most 600, at least or exactly or at most 601, at least or exactly or at most 602, at least or exactly or at most 603, at least or exactly or at most 604, at least or exactly or at most 605, at least or exactly or at most 606, at least or exactly or at most 607, at least or exactly or at most 608, at least or exactly or at most 609, at least or exactly or at most 610, at least or exactly or at most 611, at least or exactly or at most 612, at least or exactly or at most 613, at least or exactly or at most 614, at least or exactly or at most 615, at least or exactly or at most 616, at least or exactly or at most 617, at least or exactly or at most 618, at least or exactly or at most 619, at least or exactly or at most 620, at least or exactly or at most 621, at least or exactly or at most 622, at least or exactly or at most 623, at least or exactly or at most 624, at least or exactly or at most 625, at least or exactly or at most 626, or at least or exactly or at most 627 contiguous amino acid residues.

Insofar as embodiment b relates to SEQ ID NOs: 23-35, the at least 5 contiguous amino acids referred to in option b) in the definition of the 1^{st} aspect of the invention may also constitute at least or exactly or at most 628, at least or exactly or at most 629, at least or exactly or at most 630, at least or exactly or at most 631, at least or exactly or at most 632, at least or exactly or at most 633, at least or exactly or at most 634, at least or exactly or at most 635, at least or exactly or at most 636, at least or exactly or at most 637, at least or exactly or at most 638, at least or exactly or at most 639, at least or exactly or at most 640, at least or exactly or at most 641, at least or exactly or at most 642, at least or exactly or at most 643, at least or exactly or at most 644, at least or exactly or at most 645, at least or exactly or at most 646, at least or exactly or at most 647, at least or exactly or at most 648, at least or exactly or at most 649, at least or exactly or at most 650, at least or exactly or at most 651, at least or exactly or at most 652, at least or exactly or at most 653, at least or exactly or at most 654, at least or exactly or at most 655, at least or exactly or at most 656, at least or exactly or at most 657, at least or exactly or at most 658, at least or exactly or at most 659, at least or exactly or at most 660, at least or exactly or at most 661, at least or exactly or at most 662, at least or exactly or at most 663, at least or exactly or at most 664, at least or exactly or at most 665, at least or exactly or at most 666, at least or exactly or at most 667, at least or exactly or at most 668, at least or exactly or at most 669, at least or exactly or at most 670, at least or exactly or at most 671, at least or exactly or at most 672, at least or exactly or at most 673, at least or exactly or at most 674, at least or exactly or at most 675, at least or exactly or at most 676, at least or exactly or at most 677, at least or exactly or at most 678, at least or exactly or at most 679, at least or exactly or at most 680, at least or exactly or at most 681, at least or exactly or at most 682, at least or exactly or at most 683, at least or exactly or at most 684, at least or exactly or at most 685, at least or exactly or at most 686, at least or exactly or at most 687, at least or exactly or at most 688, at least or exactly or at most 689, at least or exactly or at most 690, at least or exactly or at most 691, or at least or exactly or at most 692 contiguous amino acid residues.

Insofar as embodiment b relates to SEQ ID NOs: 24-35, the at least 5 contiguous amino acids referred to in option b) in the definition of the 1^{st} aspect of the invention may also constitute at least or exactly or at most 693, at least or exactly or at most 694, at least or exactly or at most 695, at least or exactly or at most 696, at least or exactly or at most 697, at least or exactly or at most 698, at least or exactly or at most 699, at least or exactly or at most 700, at least or exactly or at most 701, at least or exactly or at most 702, at least or exactly or at most 703, at least or exactly or at most 704, at least or exactly or at most 705, at least or exactly or at most 706, at least or exactly or at most 707, at least or exactly or at most 708, at least or exactly or at most 709, at least or exactly or at most 710, at least or exactly or at most 711, at least or exactly or at most 712, at least or exactly or at most 713, at least or exactly or at most 714, at least or exactly or at most 715, at least or exactly or at most 716, at least or exactly or at most 717, at least or exactly or at most 718, or at least or exactly or at most 719 contiguous amino acid residues.

Insofar as embodiment b relates to SEQ ID NOs: 25-35, the at least 5 contiguous amino acids referred to in option b) in the definition of the 1^{st} aspect of the invention may also constitute at at least or exactly or at most 720, at least or exactly or at most 721, at least or exactly or at most 722, at least or exactly or at most 723, at least or exactly or at most 724, at least or exactly or at most 725, at least or exactly or at most 726, at least or exactly or at most 727, at least or exactly or at most 728, at least or exactly or at most 729, at least or exactly or at most 730, at least or exactly or at most 731, at least or exactly or at most 732, at least or exactly or at most 733, at least or exactly or at most 734, at least or exactly or at most 735, at least or exactly or at most 736, at least or exactly or at most 737, at least or exactly or at most 738, at least or exactly or at most 739, at least or exactly or at most 740, at least or exactly or at most 741, at least or exactly or at most 742, at least or exactly or at most 743, at least or exactly or at most 744, at least or exactly or at most 745, at least or exactly or at most 746, at least or exactly or at most 747, at least or exactly or at most 748, at least or exactly or at most 749, at least or exactly or at most 750, at least or exactly or at most 751, at least or exactly or at most 752, at least or exactly or at most 753, at least or exactly or at most 754, at least or exactly or at most 755, at least or exactly or at most 756, at least or exactly or at most 757, at least or exactly or at most 758, at least or exactly or at most 759, at least or exactly or at most 760, at least or exactly or at most 761, at least or exactly or at most 762, at least or exactly or at most 763, at least or exactly or at most 764, at least or exactly or at most 765, at least or exactly or at most 766, at least or exactly or at most 767, at least or exactly or at most 768, at least or exactly or at most 769, at least or exactly or at most 770, at least or exactly or at most 771, at least or exactly or at most 772, at least or exactly or at most 773, at least or exactly or at most 774, at least or exactly or at most 775, at least or exactly or at most 776, at least or exactly or at most 777, at least or exactly or at most 778, at least or exactly or at most 779, at least or exactly or at most 780, at least or exactly or at most 781, at least or exactly or at most 782, at least or exactly or at most 783, at least or exactly or at most 784, at least or exactly or at most 785, at least or exactly or at most 786, at least or exactly or at most 787, at least or exactly or at most 788, at least or exactly or at most 789, at least or exactly or at most 790, or at least or exactly or at most 791 contiguous amino acid residues.

Insofar as embodiment b relates to SEQ ID NOs: 26-35, the at least 5 contiguous amino acids referred to in option b) in the definition of the 1^{st} aspect of the invention may also constitute at least or exactly or at most 792, at least or exactly or at most 793, at least or exactly or at most 794, at least or exactly or at most 795, at least or exactly or at most 796, at least or exactly or at most 797, at least or exactly or at most 798, at least or exactly or at most 799, or at least or exactly or at most 800 contiguous amino acid residues.

Insofar as embodiment b relates to SEQ ID NOs: 27-35, the at least 5 contiguous amino acids referred to in option b) in the definition of the 1^{st} aspect of the invention may also constitute at least or exactly or at most 801, at least or exactly or at most 802, at least or exactly or at most 803, at least or exactly or at most 804, at least or exactly or at most 805, at least or exactly or at most 806, at least or exactly or at most 807, or at least or exactly or at most 808 contiguous amino acid residues.

Insofar as embodiment b relates to SEQ ID NOs: 28-35, the at least 5 contiguous amino acids referred to in option b) in the definition of the 1^{st} aspect of the invention may also constitute at least or exactly or at most 809, at least or exactly or at most 810, at least or exactly or at most 811, at least or exactly or at most 812, at least or exactly or at most 813, at least or exactly or at most 814, at least or exactly or at most 815, at least or exactly or at most 816, at least or exactly or at most 817, at least or exactly or at most 818, at least or exactly or at most 819, at least or exactly or at most 820, at least or exactly or at most 821, at least or exactly or at most 822, at least or exactly or at most 823, at least or exactly or at most 824, at least or exactly or at most 825, at least or exactly or at most 826, at least or exactly or at most 827, at least or exactly or at most 828, at least or exactly or at most 829, at least or exactly or at most 830, at least or exactly or at most 831, at least or exactly or at most 832, at least or exactly or at most 833, at least or exactly or at most 834, at least or exactly or at most 835, at least or exactly or at most 836, at least or exactly or at most 837, at least or exactly or at most 838, at least or exactly or at most 839, at least or exactly or at most 840, at least or exactly or at most 841, at least or exactly or at most 842, at least or exactly or at most 843, at least or exactly or at most 844, at least or exactly or at most 845, at least or exactly or at most 846, at least or exactly or at most 847, at least or exactly or at most 848, at least or exactly or at most 849, at least or exactly or at most 850, at least or exactly or at most 851, at least or exactly or at most 852, at least or exactly or at most 853, at least or exactly or at most 854, at least or exactly or at most 855, at least or exactly or at most 856, at least or exactly or at most 857, at least or exactly or at most 858, at least or exactly or at most 859, at least or exactly or at most 860, at least or exactly or at most 861, at least or exactly or at most 862, at least or exactly or at most 863, at least or exactly or at most 864, at least or exactly or at most 865, at least or exactly or at most 866, at least or exactly or at most 867, at least or exactly or at most 868, at least or exactly or at most 869, at least or exactly or at most 870, at least or exactly or at most 871, at least or exactly or at most 872, at least or exactly or at most 873, at least or exactly or at most 874, at least or exactly or at most 875, at least or exactly or at most 876, at least or exactly or at most 877, at least or exactly or at most 878, at least or exactly or at most 879, at least or exactly or at most 880, at least or exactly or at most 881, at least or exactly or at most 882, at least or exactly or at most 883, at least or exactly or at most 884, at least or exactly or at most 885, at least or exactly or at most 886, at least or exactly or at most 887, at least or exactly or at most 888, at least or exactly or at most 889, at least or exactly or at most 890, at least or exactly or at most 891, at least or exactly or at most 892, at least or exactly or at most 893, at least or exactly or at most 894, at least or exactly or at most 895, at least or exactly or at most 896, at least or exactly or at most 897, at least or exactly or at most 898, at least or exactly or at most 899, at least or exactly or at most 900, at least or exactly or at most 901, at least or exactly or at most 902, at least or exactly or at most 903, at least or exactly or at most 904, at least or exactly or at most 905, at least or exactly or at most 906, at least or exactly or at most 907, at least or exactly or at most 908, at least or exactly or at most 909, at least or exactly or at most 910, or at least or exactly or at most 911 contiguous amino acid residues.

Insofar as embodiment b relates to SEQ ID NOs: 29-35, the at least 5 contiguous amino acids referred to in option b) in the definition of the 1^{st} aspect of the invention may also constitute at least or exactly or at most 912, at least or exactly or at most 913, at least or exactly or at most 914, at least or exactly or at most 915, at least or exactly or at most 916, at least or exactly or at most 917, or at least or exactly or at most 918 contiguous amino acid residues.

Insofar as embodiment b relates to SEQ ID NOs: 30-35, the at least 5 contiguous amino acids referred to in option b) in the definition of the 1^{st} aspect of the invention may also constitute at least or exactly or at most 919, at least or exactly or at most 920, or at least or exactly or at most 921 contiguous amino acid residues.

Insofar as embodiment b relates to SEQ ID NOs: 31-35, the at least 5 contiguous amino acids referred to in option b) in the definition of the 1^{st} aspect of the invention may also constitute at least or exactly or at most 922, at least or exactly or at most 923, at least or exactly or at most 924, at least or exactly or at most 925, at least or exactly or at most 926, at least or exactly or at most 927, at least or exactly or at most 928, at least or exactly or at most 929, at least or exactly or at most 930, at least or exactly or at most 931, at least or exactly or at most 932, at least or exactly or at most 933, at least or exactly or at most 934, at least or exactly or at most 935, at least or exactly or at most 936, at least or exactly or at most 937, at least or exactly or at most 938, at least or exactly or at most 939, at least or exactly or at most 940, at least or exactly or at most 941, or at least or exactly or at most 942 contiguous amino acid residues.

Insofar as embodiment b relates to SEQ ID NOs: 32-35, the at least 5 contiguous amino acids referred to in option b) in the definition of the 1^{st} aspect of the invention may also constitute at least or exactly or at most 943, at least or exactly or at most 944, at least or exactly or at most 945, at least or exactly or at most 946, at least or exactly or at most 947, at least or exactly or at most 948, at least or exactly or at most 949, at least or exactly or at most 950, at least or exactly or at most 951, at least or exactly or at most 952, at least or exactly or at most 953, at least or exactly or at most 954, at least or exactly or at most 955, at least or exactly or at most 956, at least or exactly or at most 957, at least or exactly or at most 958, at least or exactly or at most 959, at least or exactly or at most 960, at least or exactly or at most 961, at least or exactly or at most 962, at least or exactly or at most 963, at least or exactly or at most 964, at least or exactly or at most 965, at least or exactly or at most 966, at least or exactly or at most 967, at least or exactly or at most 968, at least or exactly or at most 969, at least or exactly or at most 970, at least or exactly or at most 971, at least or exactly or at most 972, at least or exactly or at most 973, at least or exactly or at most 974, at least or exactly or at most 975, at least or exactly or at most 976, at least or exactly or at most 977, at least or exactly or at most 978, at least or exactly or at most 979, at least or exactly or at most 980, at least or exactly or at most 981, at least or exactly or at most 982, at least or exactly or at most 983, at least or exactly or at most 984, at least or exactly or at most 985, at least or exactly or at most 986, at least or exactly or at most 987, at least or exactly or at most 988, at least or exactly or at most 989, at least or exactly or at most 990, at least or exactly or at most 991, at least or exactly or at most 992, at least or exactly or at most 993, at least or exactly or at most 994, at least or exactly or at most 995, at least or exactly or at most 996, at least or exactly or at most 997, at least or exactly or at most 998, at least or exactly or at most 999, at least or exactly or at most 1000, at least or exactly or at most 1001, at least or exactly or at most 1002, at least or exactly or at most 1003, at least or exactly or at most 1004, at least or exactly or at most 1005, at least or exactly or at most 1006, at least or exactly or at most 1007, at least or exactly or at most 1008, at least or exactly or at most 1009, at least or exactly or at most 1010, at least or exactly or at most 1011, at least or exactly or at most 1012, or at least or exactly or at most 1013 contiguous amino acid residues.

Insofar as embodiment b relates to SEQ ID NOs: 33-35, the at least 5 contiguous amino acids referred to in option b) in the definition of the 1^{st} aspect of the invention may also constitute at least or exactly or at most 1014, at least or exactly or at most 1015, at least or exactly or at most 1016, at least or exactly or at most 1017, at least or exactly or at most 1018, at least or exactly or at most 1019, at least or exactly or at most 1020, at least or exactly or at most 1021, at least or exactly or at most 1022, at least or exactly or at most 1023, at least or exactly or at most 1024, at least or exactly or at most 1025, at least or exactly or at most 1026, at least or exactly or at most 1027, at least or exactly or at most 1028, at least or exactly or at most 1029, at least or exactly or at most 1030, at least or exactly or at most 1031, at least or exactly or at most 1032, at least or exactly or at most 1033, at least or exactly or at most 1034, at least or exactly or at most 1035, at least or exactly or at most 1036, at least or exactly or at most 1037, at least or exactly or at most 1038, at least or exactly or at most 1039, at least or exactly or at most 1040, at least or exactly or at most 1041, at least or exactly or at most 1042, at least or exactly or at most 1043, at least or exactly or at most 1044, at least or exactly or at most 1045, at least or exactly or at most 1046, at least or exactly or at most 1047, at least or exactly or at most 1048, at least or exactly or at most 1049, at least or exactly or at most 1050, at least or exactly or at most 1051, at least or exactly or at most 1052, at least or exactly or at most 1053, at least or exactly or at most 1054, at least or exactly or at most 1055, at least or exactly or at most 1056, at least or exactly or at most 1057, at least or exactly or at most 1058, at least or exactly or at most 1059, at least or exactly or at most 1060, at least or exactly or at most 1061, at least or exactly or at most 1062, at least or exactly or at most 1063, at least or exactly or at most 1064, at least or exactly or at most 1065, at least or exactly or at most 1066, at least or exactly or at most 1067, at least or exactly or at most 1068, at least or exactly or at most 1069, at least or exactly or at most 1070, at least or exactly or at most 1071, at least or exactly or at most 1072, at least or exactly or at most 1073, or at least or exactly or at most 1074 contiguous amino acid residues.

Insofar as embodiment b relates to SEQ ID NO: 34 or 35, the at least 5 contiguous amino acids referred to in option b) in the definition of the 1^{st} aspect of the invention may also constitute at least or exactly or at most 1075, at least or exactly or at most 1076, at least or exactly or at most 1077, at least or exactly or at most 1078, at least or exactly or at most 1079, at least or exactly or at most 1080, at least or exactly or at most 1081, at least or exactly or at most 1082, at least or exactly or at most 1083, at least or exactly or at most 1084, at least or exactly or at most 1085, at least or exactly or at most 1086, at least or exactly or at most 1087, at least or exactly or at most 1088, at least or exactly or at most 1089, at least or exactly or at most 1090, at least or exactly or at most 1091, at least or exactly or at most 1092, at least or exactly or at most 1093, at least or exactly or at most 1094, at least or exactly or at most 1095, at least or exactly or at most 1096, at least or exactly or at most 1097, at least or exactly or at most 1098, at least or exactly or at most 1099, at least or exactly or at most 1100, at least or exactly or at most 1101, at least or exactly or at most 1102, at least or exactly or at most 1103, at least or exactly or at most 1104, at least or exactly or at most 1105, at least or exactly or at most 1106, at least or exactly or at most 1107, at least or exactly or at most 1108, at least or exactly or at most 1109, at least or exactly or at most 1110, at least or exactly or at most 1111, at least or exactly or at most 1112, at least or exactly or at most 1113, at least or exactly or at most 1114, at least or exactly or at most 1115, at least or exactly or at most 1116, at least or exactly or at most 1117, at least or exactly or at most 1118, at least or exactly or at most 1119, at least or exactly or at most 1120, at least or exactly or at most 1121, at least or exactly or at most 1122, at least or exactly or at most 1123, at least or exactly or at most 1124, at least or exactly or at most 1125, at least or exactly or at most 1126, at least or exactly or at most 1127, at least or exactly or at most 1128, at least or exactly or at most 1129, at least or exactly or at most 1130, at least or exactly or at most 1131, at least or exactly or at most 1132, at least or exactly or at most 1133, at least or exactly or at most 1134, at least or exactly or at most 1135, at least or exactly or at most 1136, at least or exactly or at most 1137, at least or exactly or at most 1138, at least or exactly or at most 1139, at least or exactly or at most 1140, at least or exactly or at most 1141, at least or exactly or at most 1142, at least or exactly or at most 1143, at least or exactly or at most 1144, at least or exactly or at most 1145, at least or exactly or at most 1146, at least or exactly or at most 1147, at least or exactly or at most 1148, at least or exactly or at most 1149, at least or exactly or at most 1150, at least or exactly or at most 1151, at least or exactly or at most 1152, at least or exactly or at most 1153, at least or exactly or at most 1154, at least or exactly or at most 1155, at least or exactly or at most 1156, at least or exactly or at most 1157, at least or exactly or at most 1158, at least or exactly or at most 1159, at least or exactly or at most 1160, at least or exactly or at most 1161, at least or exactly or at most 1162, at least or exactly or at most 1163, at least or exactly or at most 1164, at least or exactly or at most 1165, at least or exactly or at most 1166, at least or exactly or at most 1167, at least or exactly or at most 1168, at least or exactly or at most 1169, at least or exactly or at most 1170, at least or exactly or at most 1171, at least or exactly or at most 1172, at least or exactly or at most 1173, at least or exactly or at most 1174, at least or exactly or at most 1175, at least or exactly or at most 1176, at least or exactly or at most 1177, at least or exactly or at most 1178, at least or exactly or at most 1179, at least or exactly or at most 1180, at least or exactly or at most 1181, at least or exactly or at most 1182, at least or exactly or at most 1183, at least or exactly or at most 1184, at least or exactly or at most 1185, at least or exactly or at most 1186, at least or exactly or at most 1187, at least or exactly or at most 1188, at least or exactly or at most 1189, at least or exactly or at most 1190, at least or exactly or at most 1191, at least or exactly or at most 1192, at least or exactly or at most 1193, at least or exactly or at most 1194, at least or exactly or at most 1195, at least or exactly or at most 1196, at least or exactly or at most 1197, at least or exactly or at most 1198, at least or exactly or at most 1199, at least or exactly or at most 1200, at least or exactly or at most 1201, at least or exactly or at most 1202, at least or exactly or at most 1203, at least or exactly or at most 1204, at least or exactly or at most 1205, at least or exactly or at most 1206, at least or exactly or at most 1207, at least or exactly or at most 1208, at least or exactly or at most 1209, at least or exactly or at most 1210, at least or exactly or at most 1211, at least or exactly or at most 1212, at least or exactly or at most 1213, at least or exactly or at most 1214, at least or exactly or at most 1215, at least or exactly or at most 1216, at least or exactly or at most 1217, at least or exactly or at most 1218, at least or exactly or at most 1219, at least or exactly or at most 1220, at least or exactly or at most 1221, at least or exactly or at most 1222, at least or exactly or at most 1223, at least or exactly or at most 1224, at least or exactly or at most 1225, at least or exactly or at most 1226, at least or exactly or at most 1227, at least or exactly or at most 1228, at least or exactly or at most 1229, at least or exactly or at most 1230, at least or exactly or at most 1231, at least or exactly or at most 1232, at least or exactly or at most 1233, at least or exactly or at most 1234, at least or exactly or at most 1235, at least or exactly or at most 1236, at least or exactly or at most 1237, at least or exactly or at most 1238, at least or exactly or at most 1239, at least or exactly or at most 1240, at least or exactly or at most 1241, at least or exactly or at most 1242, at least or exactly or at most 1243, at least or exactly or at most 1244, at least or exactly or at most 1245, at least or exactly or at most 1246, at least or exactly or at most 1247, at least or exactly or at most 1248, at least or exactly or at most 1249, at least or exactly or at most 1250, at least or exactly or at most 1251, at least or exactly or at most 1252, at least or exactly or at most 1253, at least or exactly or at most 1254, at least or exactly or at most 1255, at least or exactly or at most 1256, at least or exactly or at most 1257, at least or exactly or at most 1258, at least or exactly or at most 1259, at least or exactly or at most 1260, at least or exactly or at most 1261, at least or exactly or at most 1262, at least or exactly or at most 1263, at least or exactly or at most 1264, at least or exactly or at most 1265, at least or exactly or at most 1266, at least or exactly or at most 1267, at least or exactly or at most 1268, at least or exactly or at most 1269, at least or exactly or at most 1270, at least or exactly or at most 1271, at least or exactly or at most 1272, at least or exactly or at most 1273, at least or exactly or at most 1274, at least or exactly or at most 1275, at least or exactly or at most 1276, at least or exactly or at most 1277, at least or exactly or at most 1278, at least or exactly or at most 1279, at least or exactly or at most 1280, at least or exactly or at most 1281, at least or exactly or at most 1282, at least or exactly or at most 1283, at least or exactly or at most 1284, at least or exactly or at most 1285, at least or exactly or at most 1286, at least or exactly or at most 1287, at least or exactly or at most 1288, at least or exactly or at most 1289, at least or exactly or at most 1290, at least or exactly or at most 1291, at least or exactly or at most 1292, at least or exactly or at most 1293, at least or exactly or at most 1294, at least or exactly or at most 1295, at least or exactly or at most 1296, at least or exactly or at most 1297, at least or exactly or at most 1298, at least or exactly or at most 1299, at least or exactly or at most 1300, at least or exactly or at most 1301, at least or exactly or at most 1302, at least or exactly or at most 1303, at least or exactly or at most 1304, at least or exactly or at most 1305, at least or exactly or at most 1306, at least or exactly or at most 1307, at least or exactly or at most 1308, at least or exactly or at most 1309, at least or exactly or at most 1310, at least or exactly or at most 1311, at least or exactly or at most 1312, at least or exactly or at most 1313, at least or exactly or at most 1314, at least or exactly or at most 1315, at least or exactly or at most 1316, at least or exactly or at most 1317, at least or exactly or at most 1318, at least or exactly or at most 1319, at least or exactly or at most 1320, at least or exactly or at most 1321, at least or exactly or at most 1322, at least or exactly or at most 1323, at least or exactly or at most 1324, at least or exactly or at most 1325, at least or exactly or at most 1326, at least or exactly or at most 1327, at least or exactly or at most 1328, at least or exactly or at most 1329, at least or exactly or at most 1330, at least or exactly or at most 1331, at least or exactly or at most 1332, at least or exactly or at most 1333, at least or exactly or at most 1334, at least or exactly or at most 1335, at least or exactly or at most 1336, at least or exactly or at most 1337, at least or exactly or at most 1338, at least or exactly or at most 1339, at least or exactly or at most 1340, at least or exactly or at most 1341, at least or exactly or at most 1342, at least or exactly or at most 1343, at least or exactly or at most 1344, at least or exactly or at most 1345, at least or exactly or at most 1346, at least or exactly or at most 1347, at least or exactly or at most 1348, at least or exactly or at most 1349, at least or exactly or at most 1350, at least or exactly or at most 1351, at least or exactly or at most 1352, at least or exactly or at most 1353, at least or exactly or at most 1354, at least or exactly or at most 1355, at least or exactly or at most 1356, at least or exactly or at most 1357, at least or exactly or at most 1358, at least or exactly or at most 1359, at least or exactly or at most 1360, at least or exactly or at most 1361, at least or exactly or at most 1362, at least or exactly or at most 1363, at least or exactly or at most 1364, at least or exactly or at most 1365, at least or exactly or at most 1366, at least or exactly or at most 1367, at least or exactly or at most 1368, at least or exactly or at most 1369, at least or exactly or at most 1370, at least or exactly or at most 1371, at least or exactly or at most 1372, at least or exactly or at most 1373, at least or exactly or at most 1374, at least or exactly or at most 1375, at least or exactly or at most 1376, at least or exactly or at most 1377, at least or exactly or at most 1378, at least or exactly or at most 1379, at least or exactly or at most 1380, at least or exactly or at most 1381, at least or exactly or at most 1382, at least or exactly or at most 1383, at least or exactly or at most 1384, at least or exactly or at most 1385, at least or exactly or at most 1386, at least or exactly or at most 1387, at least or exactly or at most 1388, at least or exactly or at most 1389, at least or exactly or at most 1390, at least or exactly or at most 1391, at least or exactly or at most 1392, at least or exactly or at most 1393, at least or exactly or at most 1394, at least or exactly or at most 1395, at least or exactly or at most 1396, at least or exactly or at most 1397, at least or exactly or at most 1398, at least or exactly or at most 1399, at least or exactly or at most 1400, at least or exactly or at most 1401, at least or exactly or at most 1402, at least or exactly or at most 1403, at least or exactly or at most 1404, at least or exactly or at most 1405, at least or exactly or at most 1406, at least or exactly or at most 1407, at least or exactly or at most 1408, at least or exactly or at most 1409, at least or exactly or at most 1410, at least or exactly or at most 1411, at least or exactly or at most 1412, at least or exactly or at most 1413, at least or exactly or at most 1414, at least or exactly or at most 1415, at least or exactly or at most 1416, at least or exactly or at most 1417, at least or exactly or at most 1418, at least or exactly or at most 1419, at least or exactly or at most 1420, at least or exactly or at most 1421, at least or exactly or at most 1422, at least or exactly or at most 1423, at least or exactly or at most 1424, at least or exactly or at most 1425, at least or exactly or at most 1426, at least or exactly or at most 1427, at least or exactly or at most 1428, at least or exactly or at most 1429, at least or exactly or at most 1430, at least or exactly or at most 1431, at least or exactly or at most 1432, at least or exactly or at most 1433, at least or exactly or at most 1434, at least or exactly or at most 1435, at least or exactly or at most 1436, at least or exactly or at most 1437, at least or exactly or at most 1438, at least or exactly or at most 1439, at least or exactly or at most 1440, at least or exactly or at most 1441, at least or exactly or at most 1442, at least or exactly or at most 1443, at least or exactly or at most 1444, at least or exactly or at most 1445, at least or exactly or at most 1446, at least or exactly or at most 1447, at least or exactly or at most 1448, at least or exactly or at most 1449, at least or exactly or at most 1450, at least or exactly or at most 1451, at least or exactly or at most 1452, at least or exactly or at most 1453, at least or exactly or at most 1454, at least or exactly or at most 1455, at least or exactly or at most 1456, at least or exactly or at most 1457, at least or exactly or at most 1458, at least or exactly or at most 1459, at least or exactly or at most 1460, at least or exactly or at most 1461, at least or exactly or at most 1462, at least or exactly or at most 1463, at least or exactly or at most 1464, at least or exactly or at most 1465, at least or exactly or at most 1466, or at least or exactly or at most 1467 contiguous amino acid residues.

Insofar as embodiment b relates to SEQ ID NO: 35, the at least 5 contiguous amino acids referred to in option b) in the definition of the 1^{st} aspect of the invention may also constitute contiguous at least or exactly or at most 1469, at least or exactly or at most 1470, at least or exactly or at most 1471, at least or exactly or at most 1472, at least or exactly or at most 1473, at least or exactly or at most 1474, at least or exactly or at most 1475, at least or exactly or at most 1476, at least or exactly or at most 1477, at least or exactly or at most 1478, at least or exactly or at most 1479, at least or exactly or at most 1480, at least or exactly or at most 1481, at least or exactly or at most 1482, at least or exactly or at most 1483, at least or exactly or at most 1484, at least or exactly or at most 1485, at least or exactly or at most 1486, at least or exactly or at most 1487, at least or exactly or at most 1488, at least or exactly or at most 1489, at least or exactly or at most 1490, at least or exactly or at most 1491, at least or exactly or at most 1492, at least or exactly or at most 1493, at least or exactly or at most 1494, at least or exactly or at most 1495, at least or exactly or at most 1496, at least or exactly or at most 1497, at least or exactly or at most 1498, at least or exactly or at most 1499, at least or exactly or at most 1500, at least or exactly or at most 1501, at least or exactly or at most 1502, at least or exactly or at most 1503, at least or exactly or at most 1504, at least or exactly or at most 1505, at least or exactly or at most 1506, at least or exactly or at most 1507, at least or exactly or at most 1508, at least or exactly or at most 1509, at least or exactly or at most 1510, at least or exactly or at most 1511, at least or exactly or at most 1512, at least or exactly or at most 1513, at least or exactly or at most 1514, at least or exactly or at most 1515, at least or exactly or at most 1516, at least or exactly or at most 1517, at least or exactly or at most 1518, at least or exactly or at most 1519, at least or exactly or at most 1520, at least or exactly or at most 1521, at least or exactly or at most 1522, at least or exactly or at most 1523, at least or exactly or at most 1524, at least or exactly or at most 1525, at least or exactly or at most 1526, at least or exactly or at most 1527, at least or exactly or at most 1528, at least or exactly or at most 1529, at least or exactly or at most 1530, at least or exactly or at most 1531, at least or exactly or at most 1532, at least or exactly or at most 1533, at least or exactly or at most 1534, at least or exactly or at most 1535, at least or exactly or at most 1536, at least or exactly or at most 1537, at least or exactly or at most 1538, at least or exactly or at most 1539, at least or exactly or at most 1540, at least or exactly or at most 1541, at least or exactly or at most 1542, at least or exactly or at most 1543, at least or exactly or at most 1544, at least or exactly or at most 1545, at least or exactly or at most 1546, at least or exactly or at most 1547, at least or exactly or at most 1548, at least or exactly or at most 1549, at least or exactly or at most 1550, at least or exactly or at most 1551, at least or exactly or at most 1552, at least or exactly or at most 1553, at least or exactly or at most 1554, at least or exactly or at most 1555, at least or exactly or at most 1556, at least or exactly or at most 1557, at least or exactly or at most 1558, at least or exactly or at most 1559, at least or exactly or at most 1560, at least or exactly or at most 1561, at least or exactly or at most 1562, at least or exactly or at most 1563, at least or exactly or at most 1564, at least or exactly or at most 1565, at least or exactly or at most 1566, at least or exactly or at most 1567, at least or exactly or at most 1568, at least or exactly or at most 1569, at least or exactly or at most 1570, at least or exactly or at most 1571, at least or exactly or at most 1572, at least or exactly or at most 1573, at least or exactly or at most 1574, at least or exactly or at most 1575, at least or exactly or at most 1576, at least or exactly or at most 1577, at least or exactly or at most 1578, at least or exactly or at most 1579, at least or exactly or at most 1580, at least or exactly or at most 1581, at least or exactly or at most 1582, at least or exactly or at most 1583, at least or exactly or at most 1584, at least or exactly or at most 1585, at least or exactly or at most 1586, at least or exactly or at most 1587, at least or exactly or at most 1588, at least or exactly or at most 1589, at least or exactly or at most 1590, at least or exactly or at most 1591, at least or exactly or at most 1592, at least or exactly or at most 1593, at least or exactly or at most 1594, at least or exactly or at most 1595, at least or exactly or at most 1596, at least or exactly or at most 1597, at least or exactly or at most 1598, at least or exactly or at most 1599, at least or exactly or at most 1600, at least or exactly or at most 1601, at least or exactly or at most 1602, at least or exactly or at most 1603, at least or exactly or at most 1604, at least or exactly or at most 1605, at least or exactly or at most 1606, at least or exactly or at most 1607, at least or exactly or at most 1608, at least or exactly or at most 1609, at least or exactly or at most 1610, at least or exactly or at most 1611, at least or exactly or at most 1612, at least or exactly or at most 1613, at least or exactly or at most 1614, at least or exactly or at most 1615, at least or exactly or at most 1616, at least or exactly or at most 1617, at least or exactly or at most 1618, at least or exactly or at most 1619, at least or exactly or at most 1620, at least or exactly or at most 1621, at least or exactly or at most 1622, at least or exactly or at most 1623, at least or exactly or at most 1624, at least or exactly or at most 1625, at least or exactly or at most 1626, at least or exactly or at most 1627, at least or exactly or at most 1628, at least or exactly or at most 1629, at least or exactly or at most 1630, at least or exactly or at most 1631, at least or exactly or at most 1632, at least or exactly or at most 1633, at least or exactly or at most 1634, at least or exactly or at most 1635, at least or exactly or at most 1636, at least or exactly or at most 1637, at least or exactly or at most 1638, at least or exactly or at most 1639, at least or exactly or at most 1640, at least or exactly or at most 1641, at least or exactly or at most 1642, at least or exactly or at most 1643, at least or exactly or at most 1644, at least or exactly or at most 1645, at least or exactly or at most 1646, at least or exactly or at most 1647, at least or exactly or at most 1648, at least or exactly or at most 1649, at least or exactly or at most 1650, at least or exactly or at most 1651, at least or exactly or at most 1652, at least or exactly or at most 1653, at least or exactly or at most 1654, at least or exactly or at most 1655, at least or exactly or at most 1656, at least or exactly or at most 1657, at least or exactly or at most 1658, at least or exactly or at most 1659, at least or exactly or at most 1660, at least or exactly or at most 1661, at least or exactly or at most 1662, at least or exactly or at most 1663, at least or exactly or at most 1664, at least or exactly or at most 1665, at least or exactly or at most 1666, at least or exactly or at most 1667, at least or exactly or at most 1668, at least or exactly or at most 1669, at least or exactly or at most 1670, at least or exactly or at most 1671, at least or exactly or at most 1672, at least or exactly or at most 1673, at least or exactly or at most 1674, at least or exactly or at most 1675, at least or exactly or at most 1676, at least or exactly or at most 1677, at least or exactly or at most 1678, at least or exactly or at most 1679, at least or exactly or at most 1680, at least or exactly or at most 1681, at least or exactly or at most 1682, at least or exactly or at most 1683, at least or exactly or at most 1684, at least or exactly or at most 1685, at least or exactly or at most 1686, at least or exactly or at most 1687, at least or exactly or at most 1688, at least or exactly or at most 1689, at least or exactly or at most 1690, at least or exactly or at most 1691, at least or exactly or at most 1692, at least or exactly or at most 1693, at least or exactly or at most 1694, at least or exactly or at most 1695, at least or exactly or at most 1696, at least or exactly or at most 1697, at least or exactly or at most 1698, at least or exactly or at most 1699, at least or exactly or at most 1700, at least or exactly or at most 1701, at least or exactly or at most 1702, at least or exactly or at most 1703, at least or exactly or at most 1704, at least or exactly or at most 1705, at least or exactly or at most 1706, at least or exactly or at most 1707, at least or exactly or at most 1708, at least or exactly or at most 1709, at least or exactly or at most 1710, at least or exactly or at most 1711, at least or exactly or at most 1712, at least or exactly or at most 1713, at least or exactly or at most 1714, at least or exactly or at most 1715, at least or exactly or at most 1716, at least or exactly or at most 1717, at least or exactly or at most 1718, at least or exactly or at most 1719, at least or exactly or at most 1720, at least or exactly or at most 1721, at least or exactly or at most 1722, at least or exactly or at most 1723, at least or exactly or at most 1724, at least or exactly or at most 1725, at least or exactly or at most 1726, at least or exactly or at most 1727, at least or exactly or at most 1728, at least or exactly or at most 1729, at least or exactly or at most 1730, at least or exactly or at most 1731, at least or exactly or at most 1732, at least or exactly or at most 1733, at least or exactly or at most 1734, at least or exactly or at most 1735, at least or exactly or at most 1736, at least or exactly or at most 1737, at least or exactly or at most 1738, at least or exactly or at most 1739, at least or exactly or at most 1740, at least or exactly or at most 1741, at least or exactly or at most 1742, at least or exactly or at most 1743, at least or exactly or at most 1744, at least or exactly or at most 1745, at least or exactly or at most 1746, at least or exactly or at most 1747, at least or exactly or at most 1748, at least or exactly or at most 1749, at least or exactly or at most 1750, at least or exactly or at most 1751, at least or exactly or at most 1752, at least or exactly or at most 1753, at least or exactly or at most 1754, at least or exactly or at most 1755, at least or exactly or at most 1756, at least or exactly or at most 1757, at least or exactly or at most 1758, at least or exactly or at most 1759, at least or exactly or at most 1760, at least or exactly or at most 1761, at least or exactly or at most 1762, at least or exactly or at most 1763, at least or exactly or at most 1764, at least or exactly or at most 1765, at least or exactly or at most 1766, at least or exactly or at most 1767, at least or exactly or at most 1768, at least or exactly or at most 1769, at least or exactly or at most 1770, at least or exactly or at most 1771, at least or exactly or at most 1772, at least or exactly or at most 1773, at least or exactly or at most 1774, at least or exactly or at most 1775, at least or exactly or at most 1776, at least or exactly or at most 1777, at least or exactly or at most 1778, at least or exactly or at most 1779, at least or exactly or at most 1780, at least or exactly or at most 1781, at least or exactly or at most 1782, at least or exactly or at most 1783, at least or exactly or at most 1784, at least or exactly or at most 1785, at least or exactly or at most 1786, at least or exactly or at most 1787, at least or exactly or at most 1788, at least or exactly or at most 1789, at least or exactly or at most 1790, at least or exactly or at most 1791, at least or exactly or at most 1792, at least or exactly or at most 1793, at least or exactly or at most 1794, at least or exactly or at most 1795, at least or exactly or at most 1796, at least or exactly or at most 1797, at least or exactly or at most 1798, at least or exactly or at most 1799, at least or exactly or at most 1800, at least or exactly or at most 1801, at least or exactly or at most 1802, at least or exactly or at most 1803, at least or exactly or at most 1804, at least or exactly or at most 1805, at least or exactly or at most 1806, at least or exactly or at most 1807, at least or exactly or at most 1808, at least or exactly or at most 1809, at least or exactly or at most 1810, at least or exactly or at most 1811, at least or exactly or at most 1812, at least or exactly or at most 1813, at least or exactly or at most 1814, at least or exactly or at most 1815, at least or exactly or at most 1816, at least or exactly or at most 1817, at least or exactly or at most 1818, at least or exactly or at most 1819, at least or exactly or at most 1820, at least or exactly or at most 1821, at least or exactly or at most 1822, at least or exactly or at most 1823, at least or exactly or at most 1824, at least or exactly or at most 1825, at least or exactly or at most 1826, at least or exactly or at most 1827, at least or exactly or at most 1828, at least or exactly or at most 1829, at least or exactly or at most 1830, at least or exactly or at most 1831, at least or exactly or at most 1832, at least or exactly or at most 1833, at least or exactly or at most 1834, at least or exactly or at most 1835, at least or exactly or at most 1836, at least or exactly or at most 1837, at least or exactly or at most 1838, at least or exactly or at most 1839, at least or exactly or at most 1840, at least or exactly or at most 1841, at least or exactly or at most 1842, at least or exactly or at most 1843, at least or exactly or at most 1844, at least or exactly or at most 1845, at least or exactly or at most 1846, at least or exactly or at most 1847, at least or exactly or at most 1848, at least or exactly or at most 1849, at least or exactly or at most 1850, at least or exactly or at most 1851, at least or exactly or at most 1852, at least or exactly or at most 1853, at least or exactly or at most 1854, at least or exactly or at most 1855, at least or exactly or at most 1856, at least or exactly or at most 1857, at least or exactly or at most 1858, at least or exactly or at most 1859, at least or exactly or at most 1860, at least or exactly or at most 1861, at least or exactly or at most 1862, at least or exactly or at most 1863, at least or exactly or at most 1864, at least or exactly or at most 1865, at least or exactly or at most 1866, at least or exactly or at most 1867, at least or exactly or at most 1868, at least or exactly or at most 1869, at least or exactly or at most 1870, at least or exactly or at most 1871, at least or exactly or at most 1872, at least or exactly or at most 1873, at least or exactly or at most 1874, at least or exactly or at most 1875, at least or exactly or at most 1876, at least or exactly or at most 1877, at least or exactly or at most 1878, at least or exactly or at most 1879, at least or exactly or at most 1880, at least or exactly or at most 1881, at least or exactly or at most 1882, at least or exactly or at most 1883, at least or exactly or at most 1884, at least or exactly or at most 1885, at least or exactly or at most 1886, at least or exactly or at most 1887, at least or exactly or at most 1888, at least or exactly or at most 1889, at least or exactly or at most 1890, at least or exactly or at most 1891, at least or exactly or at most 1892, at least or exactly or at most 1893, at least or exactly or at most 1894, at least or exactly or at most 1895, at least or exactly or at most 1896, at least or exactly or at most 1897, at least or exactly or at most 1898, at least or exactly or at most 1899, at least or exactly or at most 1900, at least or exactly or at most 1901, at least or exactly or at most 1902, at least or exactly or at most 1903, at least or exactly or at most 1904, at least or exactly or at most 1905, at least or exactly or at most 1906, at least or exactly or at most 1907, at least or exactly or at most 1908, at least or exactly or at most 1909, at least or exactly or at most 1910, at least or exactly or at most 1911, at least or exactly or at most 1912, at least or exactly or at most 1913, at least or exactly or at most 1914, at least or exactly or at most 1915, at least or exactly or at most 1916, at least or exactly or at most 1917, at least or exactly or at most 1918, at least or exactly or at most 1919, at least or exactly or at most 1920, at least or exactly or at most 1921, at least or exactly or at most 1922, at least or exactly or at most 1923, at least or exactly or at most 1924, at least or exactly or at most 1925, at least or exactly or at most 1926, at least or exactly or at most 1927, at least or exactly or at most 1928, at least or exactly or at most 1929, at least or exactly or at most 1930, at least or exactly or at most 1931, at least or exactly or at most 1932, at least or exactly or at most 1933, at least or exactly or at most 1934, at least or exactly or at most 1935, at least or exactly or at most 1936, at least or exactly or at most 1937, at least or exactly or at most 1938, at least or exactly or at most 1939, at least or exactly or at most 1940, at least or exactly or at most 1941, at least or exactly or at most 1942, at least or exactly or at most 1943, at least or exactly or at most 1944, at least or exactly or at most 1945, at least or exactly or at most 1946, at least or exactly or at most 1947, at least or exactly or at most 1948, at least or exactly or at most 1949, at least or exactly or at most 1950, at least or exactly or at most 1951, at least or exactly or at most 1952, at least or exactly or at most 1953, at least or exactly or at most 1954, at least or exactly or at most 1955, at least or exactly or at most 1956, at least or exactly or at most 1957, at least or exactly or at most 1958, at least or exactly or at most 1959, at least or exactly or at most 1960, at least or exactly or at most 1961, at least or exactly or at most 1962, at least or exactly or at most 1963, at least or exactly or at most 1964, at least or exactly or at most 1965, at least or exactly or at most 1966, at least or exactly or at most 1967, at least or exactly or at most 1968, at least or exactly or at most 1969, at least or exactly or at most 1970, at least or exactly or at most 1971, at least or exactly or at most 1972, at least or exactly or at most 1973, at least or exactly or at most 1974, at least or exactly or at most 1975, or exactly or at most 1976 contiguous amino acid residues.

In some embodiments, the polypeptide of the invention also has a sequence identity with the amino acid sequence of a) defined above for all embodiments of at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%. Similarly, the polypeptide of the invention in some embodiments also has a sequence identity with the amino acid sequence of b) defined above for all embodiments of at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%.

In any of the embodiments defined by option b) above, the polypeptide of the invention is also one that has at least 5 contiguous amino acid residues defined for option b) above and also has its N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49 in any one of SEQ ID NOs: 1-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, and 98 in any one of SEQ ID NOs: 2-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1

In any of the embodiments defined by option b) above, the polypeptide of the invention is also one that has at least 5 contiguous amino acid residues defined for option b) above and also has its N-terminal amino acid residue corresponding to any one of amino acid residues 99, 100, 101, 102, 103, 104, and 105 in any one of SEQ ID NOs: 3-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 106, 107, 108, 109, and 110 in any one of SEQ ID NOs: 4-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, and 212 in any one of SEQ ID NOs: 5-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 213, 214, 215, 216, 217, 218, 219, 220, 221, 222, 223, and 224 in any one of SEQ ID NOs: 6-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, and 279 in any one of SEQ ID NOs: 7-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 280, 281, 282, 283, 284, and 285 in any one of SEQ ID NOs: 8-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, and 333 in any one of SEQ ID NOs: 9-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 334, 335, 336, 337, 338, 339, 340, 341, and 342 in any one of SEQ ID NOs: 10-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, and 373 in any one of SEQ ID NOs: 11-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 374, 375, 376, 377, 378, 379, 380, 381, 382, 383, 384, 385, 386, 387, 388, 389, 390, 391, 392, 393, and 394 in any one of SEQ ID NOs: 12-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 395, 396, 397, 398, 399, 400, 401, 402, 403, 404, 405, 406, 407, 408, 409, 410, 411, 412, 413, 414, 415, 416, 417, and 418 in any one of SEQ ID NOs: 13-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 419, 420, 421, and 422 in any one of SEQ ID NOs: 14-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 423, 424, 425, 426, 427, 428, 429, 430, 431, 432, 433, 434, and 435 in any one of SEQ ID NOs: 15-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 436, 437, 438, 439, 440, 441, 442, 443, 444, 445, 446, 447, 448, 449, 450, 451, 452, 453, 454, 455, 456, 457, 458, 459, 460, 461, 462, 463, and 464 in any one of SEQ ID NOs: 16-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 465, 466, 467, 468, 469, 470, 471, 472, 473, 474, 475, 476, 477, 478, 479, 480, 481, 482, 483, 484, 485, 486, 487, 488, 489, 490, 491, 492, 493, and 494 in any one of SEQ ID NOs: 17-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 495, 496, 497, 498, 499, 500, 501, 502, 503, 504, 505, 506, 507, 508, 509, 510, 511, 512, 513, 514, 515, 516, 517, and 518 in any one of SEQ ID NOs: 18-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 519, 520, 521, 522, and 523 in any one of SEQ ID NOs: 19-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, and 572 in any one of SEQ ID NOs: 20-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 573, 574, 575, 576, 577, 578, 579, 580, 581, 582, 583, 584, 585, 586, 587, 588, 589, 590, 591, 592, 593, and 594 in any one of SEQ ID NOs: 21-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 595, 596, 597, 598, 599, 600, 601, 602, 603, 604, 605, 606, 607, 608, 609, 610, 611, 612, 613, 614, 615, 616, 617, 618, 619, 620, 621, 622, 623, and 624 in any one of SEQ ID NOs: 22-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, and 689 in any one of SEQ ID NOs: 23-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 690, 691, 692, 693, 694, 695, 696, 697, 698, 699, 700, 701, 702, 703, 704, 705, 706, 707, 708, 709, 710, 711, 712, 713, 714, 715, and 716 in any one of SEQ ID NOs: 24-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, and 788 in any one of SEQ ID NOs: 25-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 789, 790, 791, 792, 793, 794, 795, 796, and 797 in any one of SEQ ID NOs: 26-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 798, 799, 800, 801, 802, 803, 804, and 805 in any one of SEQ ID NOs: 27-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, and 908 in any one of SEQ ID NOs: 28-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 909, 910, 911, 912, 913, 914, and 915 in any one of SEQ ID NOs: 29-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 916, 917, and 918 in any one of SEQ ID NOs: 30-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 919, 920, 921, 922, 923, 924, 925, 926, 927, 928, 929, 930, 931, 932, 933, 934, 935, 936, 937, 938, and 939 in any one of SEQ ID NOs: 31-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, and 1010 in any one of SEQ ID NOs: 32-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, and 1071 in any one of SEQ ID NOs: 33-35,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, and 1464 in SEQ ID NO: 35 or 35,

with the proviso that the selected amino acid residue satisfies the formula N≤L−n+1, where N is the number of the selected residue, L is the number of amino acid residues in SEQ ID NO: 34 or 35, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, and 1973 in SEQ ID NO: 35,

with the proviso that the selected amino acid residue satisfies the formula N≤L−n+1, where N is the number of the selected residue, L is the number of amino acid residues in SEQ ID NO: 34 or 35, and n is the number of consecutive amino acid residues defined for option b, that is, if the length of the at least 5 amino acids is higher than 5, then the N-terminal first residue will not be higher numbered than 1974-n+1.

The polypeptide of the invention is in certain embodiments also fused or conjugated to an immunogenic carrier molecule; or, phrased otherwise, the polypeptide of the invention also includes such an immunogenic carrier molecule in addition to the material derived from SEQ ID NOs: 1-35. The immunogenic carrier molecule is a typically polypeptide that induces T-helper lymphocyte responses in a majority of humans, such as immunogenic carrier proteins selected from the group consisting of keyhole limpet hemocyanino or a fragment thereof, tetanus toxoid or a fragment thereof, dipththeria toxoid or a fragment thereof. Other suitable carrier molecules are discussed infra.

Also, the polypeptide of the invention can comprise a fusion polypeptide between two distinct sequences from any one of SEQ ID NOs: 1-35, where these two fused sequences do not appear naturally fused directly to each other. Thus, such fusions may include two subsequences of the same of SEQ ID NOs: 1-35, but in an arrangement not found naturally, or the fusions may include two sequences derived from two of SEQ ID NOs: 1-35. Also, fusions of more sequences from a plurality of SEQ ID NOs: 1-35 are also possible. Any of these constructs may include an immunogenic carrier as discussed above, and the individual sequences derived from SEQ ID NOs: 1-35 may also be connected directly or via rigid or flexible linkers, such as the linker with the amino acid sequence set forth in any one of SEQ ID NOs: 106-113.

In preferred embodiments, the polypeptide of the invention detailed above is capable of inducing an adaptive immune response against the polypeptide in a mammal, in particular in a human being. Preferably, the adaptive immune response is a protective adaptive immune response against infection with NeGo. The polypeptide may in these cases induce a humoral and/or a cellular immune response.

Regions (i.e. fragments defined by N and C-terminal amino acid residues) of particular interest in SEQ ID NOs: 1-35 are set forth in the following table using the nomenclature disclosed below. Interesting polypeptides of the invention typically include or consist of amino acids from these particular regions: cNGO1947-24-102; cNGO0725-1-109; NGO1043-22-114; cNGO1984-59-216; NGO0182-26-228; NGO1379-28-283; NGO1549-35-289; NGO0721-22-337; NGO0265-44-346; cNGO1094-1-398; NGO1158-27-422; cNGO1958-20-426; cNGO1392-28-439; cNGO1068-27-468; cNGO1971-27-498; NGO2059-22-522; cNGO1585-28-576; cNGO0571-21-598; NGO0225-25-628; cNGO1496-1-693; cNGO2093-23-720; cNGO1801-22-792; cNGO1715-25-801; cNGO2109-23-809; cNGO1495-25-912; NGO1785-1-919; cNGO0952-26-922; NGO0851-25-1014; cNGO0275-28-1075; NGO2105-44-1468; cNGO1286-1-943; NGO1125-1-53; NGO1092-1-649; NGO1092-650-1610; NGO1092-650-1977; RS11935-1-377; RS10860-23-527; and RS10860-23-300.

Also fragments of these fragments are particularly preferred, that is, any of the fragments in the above list can serve as starting point for a defined fragment of a given length and a give N-terminal amino acid residue as specified above.

Epitopes

SEQ ID NOs: 1-35 include antigenic determinants (epitopes) that are as such recognized by antibodies and/or when bound to MHC molecules by T-cell receptors. For the purposes of the present invention, B-cell epitopes (i.e. antibody binding epitopes) are of particular relevance.

It is relatively uncomplicated to identify linear B-cell epitopes—one very simple approach entails that antibodies raised against NeGo or NeGo derived proteins disclosed herein are tested for binding to overlapping oligomeric peptides derived from any one of SEQ ID NO: 1-35. Thereby, the regions of the NeGo polypeptide which are responsible for or contribute to binding to the antibodies can be identified.

Alternatively, or additionally, one can produce mutated versions of the polypeptides disclosed herein, e.g. versions where each single non-alanine residue in SEQ ID NOs.: 1-35 are point mutated to alanine—this method also assists in identifying complex assembled B-cell epitopes; this is the case when binding of the same antibody is modified by exchanging amino acids in different areas of the full-length polypeptide.

Also, in silico methods for B-cell epitope prediction can be employed: useful state-of-the-art systems for β-turn prediction is provided in Petersen B et al. (November 2010), Plos One 5(11): e15079; prediction of linear B-cell epitopes, cf: Larsen J E P et al. (April 2006), Immunome Research, 2:2; prediction of solvent exposed amino acids: Petersen B et al (July 2009), BMC Structural Biology, 9:51.

The Nucleic Acid Fragments of the Invention

The nucleic acid fragment of the invention referred to above is preferably is a DNA fragment (such as SEQ ID NOs: 31-60) or an RNA fragment (such as SEQ ID NOs 61-90).

The nucleic acid fragment of the invention typically

1) consists of at least 15, such as at least 18, at least 21, at least 24, at least 27, at least 30, at least 33, at least 36, at least 39, at least 42, at least 45, at least 48, at least 51, at least 54, at least 57, at least 60, at least 63, at least 66, at least 69, at least 72, at least 75, at least 78, at least 81, at least 84, least 87, at least 90, at least 93, at least 96, at least 99, at least 102, at least 105, at least 108, at least 111, at least 114, at least 117, at least 120, at least 123, at least 126, at least 129, at least 132, at least 135, at least 138, at least 141, at least 144, at least 147, at least 150, at least 153, at least 156, or at least 159 consecutive nucleotides of the part of any one of SEQ ID NOs: 36-105 that encodes any one of SEQ ID NOs: 1-35, and

2) is in same reading frame as the part of any one of SEQ ID NOs: 35-105 that encodes any one of SEQ ID NOs: 1-35.

Longer fragments are contemplated, i.e. fragments having at least 300, at least 420, at least 520, at least 600, at least 720, at least 810, at least 900, at least 1020, at least 1500, at least 2010, at least 2510, at least 3000, at least 3510, and at least 4020 nucleotides from those of SEQ ID NOs: 36-105 that encompass fragments of such lengths.

The nucleic acid fragment of the 2^{nd} aspect of the invention is typically one wherein the sequence identity defined in iii) is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the 2^{nd} aspect of the invention is also typically one wherein the sequence identity defined in iv) is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

In embodiments of the 2^{nd} aspect of the invention, the nucleic acid sequences are codon optimized for expression in a host cell or host organism. Technologies for devising such codon optimized sequences for a given host cell or organism are well-known to the person skilled in molecular biology.

The Vectors of the Invention

Vectors disclosed herein fall into several categories discussed infra. One preferred vector disclosed herein comprises in operable linkage and in the 5′-3′ direction, an expression control region comprising an enhancer/promoter for driving expression of the nucleic acid fragment defined for option i) above, optionally a signal peptide coding sequence, a nucleotide sequence defined for option i), and optionally a terminator. Hence, such a vector constitutes an expression vector useful for effecting production in cells of the polypeptide of the invention. Since the polypeptides of the invention are bacterial of origin, recombinant production is conveniently effected in bacterial host cells, so here it is preferred that the expression control region drives expression in prokaryotic cell such as a bacterium, e.g. in E coii. However, if the vector is to drive expression of nucleic acids in mammalian cell (as would be the case for a DNA or an RNA vaccine vector), the expression control region should be adapted to suit this particular use.

The vector may as indicated further comprises a sequence encoding a signal peptide, which may provide for secretion or membrane integration of the expression product from said vector. For the purposes of nucleic acid vaccination, the signal peptides encoded are typically selected from those described in Williams J. A. Vaccines (Basel). 2013 September; 1(3): 225-249 as well as in the references cited therein.

At any rate, certain vectors disclosed herein are capable of autonomous replication.

Also, the vector disclosed herein may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in e.g. nucleic acid vaccination.

Typically, the vector disclosed herein is selected from the group consisting of a virus, such as a attenuated virus (which may in itself be useful as a vaccine agent), a bacteriophage, a plasmid, a minichromosome, and a cosmid.

A more detailed discussion of vectors disclosed herein is provided in the following:

Polypeptides disclosed herein may be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al, 2001; Ausubel et al, 1996, both incorporated herein by reference). In addition to encoding the polypeptides of this invention, a vector of the present invention may encode polypeptide sequences such as a tag or immunogenicity enhancing peptide (e.g. an immunogenic carrier or a fusion partner that stimulates the immune system, such as a cytokine or active fragment thereof). Useful vectors encoding such fusion proteins include pIN vectors, vectors encoding a stretch of histidines, and pGEX vectors, for use in generating glutathione S-transferase (GST) soluble fusion proteins for later purification and separation or cleavage.

Vectors disclosed herein may be used in a host cell to produce a polypeptide disclosed herein that may subsequently be purified for administration to a subject or the vector may be purified for direct administration to a subject for expression of the protein in the subject (as is the case when administering a nucleic acid vaccine).

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous”. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain, Immunoglobulin Light Chain, T Cell Receptor, HLA DQα and/or DQβ, β-Interferon, Interleukin-2, Interleukin-2 Receptor, MHC Class II 5, MHC Class II HLA-DRα, β-Actin, Muscle Creatine Kinase (MCK), Prealbumin (Transthyretin), Elastase I, Metallothionein (MTII), Collagenase, Albumin, α-Fetoprotein, γ-Globin, β-Globin, c-fos, c-HA-ras, Insulin, Neural Cell Adhesion Molecule (NCAM), al-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV) IE, and Gibbon Ape Leukemia Virus.

Inducible Elements include MT II—Phorbol Ester (TFA)/Heavy metals; MMTV (mouse mammary tumor virus)—Glucocorticoids; β-Interferon—poly(rl)x/poly(rc); Adenovirus 5 E2—EIA; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; α-2-Macroglobulin—IL-6; Vimentin—Serum; MHC Class I Gene H-2κb—Interferon; HSP70—E1A/SV40 Large T Antigen; Proliferin—Phorbol Ester/TPA; Tumor Necrosis Factor—PMA; and Thyroid Stimulating Hormonea Gene—Thyroid Hormone.

Also contemplated as useful in the present invention are the dectin-1 and dectin-2 promoters. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide disclosed herein is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments disclosed herein, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

2. Multiple Cloning Sites

Vectors can include a multiple cloning site (MCS), which is a nucleic acid region that contains multiple restriction enzyme sites, any of which can be used in conjunction with standard recombinant technology to digest the vector. Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression.

4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport. Consequently, the corresponding encoded RNA fragment preferably comprises a poly(A) tail.

6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host cell is yeast.

7. Selectable and Screenable Markers

In certain embodiments disclosed herein, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein disclosed herein. Further examples of selectable and screenable markers are well known to one of skill in the art.

The Transformed Cells of the Invention

Transformed cells disclosed herein are useful as organisms for producing the polypeptide of the invention, but also as simple “containers” of nucleic acids and vectors disclosed herein.

Certain transformed cells disclosed herein are capable of replicating the nucleic acid fragment defined for option i) of the second aspect of the invention. Preferred transformed cells disclosed herein are capable of expressing the nucleic acid fragment defined for option i).

For recombinant production it is convenient, but not a prerequisite that the transformed cell according is prokaryotic, such as a bacterium, but generally both prokaryotic cells and eukaryotic cells may be used.

Suitable prokaryotic cells are bacterial cells selected from the group consisting of Escherichia (such as E. coli.), Bacillus [e.g. Bacillus subtilis], Salmonella, and Mycobacterium [preferably non-pathogenic, e.g. M. bovis BCG]. Generally, and in particular for live vaccination purposes, prokaryotic cells used in the invention are non-pathogenic.

Eukaryotic cells can be in the form of yeasts (such as Saccharomyces cerevisiae) and protozoans. Alternatively, the transformed eukaryotic cells are derived from a multicellular organism such as a fungus, an insect cell, a plant cell, or a mammalian cell.

For production purposes, it is advantageous that the transformed cell disclosed herein is stably transformed by having the nucleic acid defined above for option i) stably integrated into its genome, and in certain embodiments it is also preferred that the transformed cell secretes or carries on its surface the polypeptide disclosed herein, since this facilitates recovery of the polypeptides produced. A particular version of this embodiment is one where the transformed cell is a bacterium and secretion of the polypeptide disclosed herein is into the periplasmic space.

An interesting production system is the use of plants. For instance, proteins can be produced at low cost in plants using an Agrobacterium transfection system to genetically modify plants to express genes that encode the protein of interest. One commercially available platform are those provided by iBio CMO LLC (8800 HSC Pkwy, Bryan, Tex. 77807, USA) and iBio, Inc (9 Innovatiin Way, Suite 100, Newark, Del. 19711, USA) and disclosed in e.g. EP 2 853 599, EP 1 769 068, and EP 2 192 172. Hence, in such systems the vector is an Agrobacterium vector or other vector suitable for transfection of plants.

As noted above, stably transformed cells are preferred—these i.a. allows that cell lines comprised of transformed cells as defined herein may be established—such cell lines are particularly preferred aspects of the invention.

Further details on cells and cell lines are presented in the following:

Suitable cells for recombinant nucleic acid expression of the nucleic acid fragments of the present invention are prokaryotes and eukaryotes. Examples of prokaryotic cells include E. coli; members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe). As mentioned above, the nucleic acid sequence of the present invention can be appropriately codon optimized to facilitate effective expression from each of the transformed cells disclosed herein.

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2 nd Edition, Cold Spring Harbor Laboratory Press, 1989.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials or from other depository institutions such as Deutsche Sammlung vor Micrroorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5α, JMI 09, and KC8, as well as a number of commercially available bacterial hosts such as SURE® Competent Cells and SOLOP ACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems disclosed herein, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Amplification of Nucleic Acids

Nucleic acids used as a template for amplification may be isolated from cells, tissues or other samples according to standard methodologies (Sambrook et al, 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to sequences of genes identified herein are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Methods of Gene Transfer

Suitable methods for nucleic acid delivery to effect expression of compositions of the present invention are believed to include virtually any method by which a nucleic acid (e.g., DNA, including viral and nonviral vectors, as well as RNA) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859), including microinjection (U.S. Pat. No. 5,789,215); by electroporation (U.S. Pat. No. 5,384,253); by calcium phosphate precipitation; by using DEAE dextran followed by polyethylene glycol; by direct sonic loading; by liposome mediated transfection; by microprojectile bombardment (PCT Application Nos. WO 94/09699 and 95/06128; U.S. Pat. Nos. 5,610,042; 5,322,783 5,563,055, 5,550,318, 5,538,877 and 5,538,880); by agitation with silicon carbide fibers (U.S. Pat. Nos. 5,302,523 and 5,464,765); by Agrobacterium mediated transformation (U.S. Pat. Nos. 5,591,616 and 5,563,055); or by PEG mediated transformation of protoplasts (U.S. Pat. Nos. 4,684,611 and 4,952,500); by desiccation/inhibition mediated DNA uptake. Through the application of techniques such as these, organelle(s), cell(s), tissue(s) or organism(s) may be stably or transiently transformed.

Recently, the development of RNA vaccines has shown great promise. Hence technology for RNA vaccine delivery and expression are within the ambit of the present application. Generally the teachings provided in Deering R. P. et al., Expert Opin Drug Deliv. 2014 June; 11(6):885-99 can be followed in order to effect vaccination with RNA.

The Antibodies of the Invention—and their Production/Isolation

Antibodies directed against the proteins disclosed herein are useful for affinity chromatography, immunoassays, and for distinguishing/identifying Pseudomonas proteins as well as for passive immunisation and therapy.

Antibodies to the proteins disclosed herein, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 10-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antiserum is obtained by bleeding the immunized animal into a glass or plastic container, incubating the blood at 25 C for one hour, followed by incubating at 4° C. for 2-18 hours. The serum is recovered by centrifugation (eg. 1,000 g for 10 minutes). About 20-50 ml per bleed may be obtained from rabbits.

Monoclonal antibodies are prepared using the standard method of Köhler & Milstein [Nature (1975) 256: 495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective I aedium (elg. hypexanthine, aminopterin, thymidine medium, “HAT”). The resulting hybridomas are plated by limiting dilution, and are assayed for production of antibodies, which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly 32p and 125I), electron-dense reagents, enzymes, and ligands having specific binding partners. Enzymes are typically detected by their activity. For example, horseradish peroxidase is usually detected by its ability to convert 3,3′, 5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, 1151 may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labelled with, 125I, or with an anti-biotin MAb labeled with HRP. Other permutations and possibilities will be readily apparent to those of ordinary skill in the art, and are considered as equivalents within the scope of the instant invention.

According to the invention, the isolated monoclonal antibody or antibody analogue is preferably a monoclonal antibody selected from a multi-domain antibody such as a murine antibody, a chimeric antibody such as a humanized antibody, a fully human antibody, and single-domain antibody of a llama or a camel, or which is an antibody analogue selected from a fragment of an antibody such as an Fab or an F(ab′)₂, an scFV; cf. also the definition of the term “antibody” presented above.

Compositions of the Invention; Vaccines

Pharmaceutical compositions, in particular vaccines, according to the invention may either be prophylactic (i.e. suited to prevent infection) or therapeutic (i.e. to treat disease after infection).

In some embodiments disclosed herein, the pharmaceutical compositions such as vaccines include merely one single antigen, immunogen, polypeptide, protein, nucleic acid or vector of the invention, but in other embodiments, the pharmaceutical compositions comprise “cocktails” of the antigens or of the immunogens or of the polypeptides or of the protein or of the nucleic acids or of the vectors disclosed herein.

In particularly interesting embodiments, the pharmaceutical composition is an MVA vector mentioned herein, which encodes and can effect expression of at least 2 nucleic acid fragments disclosed herein.

An embodiment of a pharmaceutical composition disclosed herein comprises exactly Y or at least Y distinct (i.e. having non-identical primary structure) polypeptides disclosed herein, where each of said Y or at least Y distinct polypeptides comprises an immunogenic amino acid sequence present in or derived from any one of SEQ ID NOs: 1-35 and wherein said Y or at least Y distinct polypeptides together comprise immunogenic amino acid sequences present in or derived from Y or at least Y of SEQ ID NOs: 1-35, wherein Y is an integer selected from 2, 3, 4, 5, 6, 7, 8, 9, 10, 11, 12, 13, 15, 16, 17, 18, 19, 20, 21, 22, 23, 24, 25, 26, 27, 28, 29, and 30.

Another embodiment of a pharmaceutical composition disclosed herein comprises a peptide/polypeptide comprising or consisting of an immunogenic amino acid sequence present in or derived from SEQ ID NO: 1 in combination with at least one NeGo peptide/polypeptide, in particular with at least one peptide/polypeptide comprising or consisting of an immunogenic amino acid sequence present in or derived from any one of SEQ ID NOs: 2-35. Another embodiment of a pharmaceutical composition disclosed herein comprises a peptide/polypeptide comprising or consisting of an immunogenic amino acid sequence present in or derived from SEQ ID NO: 2 in combination with at least one NeGo peptide/polypeptide, in particular with at least one peptide/polypeptide comprising or consisting of an immunogenic amino acid sequence present in or derived from any one of SEQ ID NOs: 1, and 3-35. Another embodiment of a pharmaceutical composition disclosed herein comprises a peptide/polypeptide comprising or consisting of an immunogenic amino acid sequence present in or derived from SEQ ID NO: 3 in combination with at least one NeGo peptide/polypeptide, in particular with at least one peptide/polypeptide comprising or consisting of an immunogenic amino acid sequence present in or derived from any one of SEQ ID NOs: 1, 2, and 4-35. Another embodiment of a pharmaceutical composition disclosed herein comprises a peptide/polypeptide comprising or consisting of an immunogenic amino acid sequence present in or derived from SEQ ID NO: 4 in combination with at least one NeGo peptide/polypeptide, in particular with at least one peptide/polypeptide comprising or consisting of an immunogenic amino acid sequence present in or derived from any one of SEQ ID NOs: 1-3, and 5-35. Another embodiment of a pharmaceutical composition disclosed herein comprises a peptide/polypeptide comprising or consisting of an immunogenic amino acid sequence present in or derived from SEQ ID NO: 5 in combination with at least one NeGo peptide/polypeptide, in particular with at least one peptide/polypeptide comprising or consisting of an immunogenic amino acid sequence present in or derived from any one of SEQ ID NOs: 1-4, and 6-35. Another embodiment of a pharmaceutical composition disclosed herein comprises a peptide/polypeptide comprising or consisting of an immunogenic amino acid sequence present in or derived from SEQ ID NO: 6 in combination with at least one NeGo peptide/polypeptide, in particular with at least one peptide/polypeptide comprising or consisting of an immunogenic amino acid sequence present in or derived from any one of SEQ ID NOs: 1-5, and 7-35. Another embodiment of a pharmaceutical composition disclosed herein comprises a peptide/polypeptide comprising or consisting of an immunogenic amino acid sequence present in or derived from SEQ ID NO: 7 in combination with at least one NeGo peptide/polypeptide, in particular with at least one peptide/polypeptide comprising or consisting of an immunogenic amino acid sequence present in or derived from any one of SEQ ID NOs: 1-6, and 8-35. Another embodiment of a pharmaceutical composition disclosed herein comprises a peptide/polypeptide comprising or consisting of an immunogenic amino acid sequence present in or derived from SEQ ID NO: 8 in combination with at least one NeGo peptide/polypeptide, in particular with at least one peptide/polypeptide comprising or consisting of an immunogenic amino acid sequence present in or derived from any one of SEQ ID NOs: 1-7, and 9-35. Another embodiment of a pharmaceutical composition disclosed herein comprises a peptide/polypeptide comprising or consisting of an immunogenic amino acid sequence present in or derived from SEQ ID NO: 9 in combination with at least one NeGo peptide/polypeptide, in particular with at least one peptide/polypeptide comprising or consisting of an immunogenic amino acid sequence present in or derived from any one of SEQ ID NOs: 1-8, and 10-35. Another embodiment of a pharmaceutical composition disclosed herein comprises a peptide/polypeptide comprising or consisting of an immunogenic amino acid sequence present in or derived from SEQ ID NO: 10 in combination with at least one NeGo peptide/polypeptide, in particular with at least one peptide/polypeptide comprising or consisting of an immunogenic amino acid sequence present in or derived from any one of SEQ ID NOs: 1-9, and 11-35. Another embodiment of a pharmaceutical composition disclosed herein comprises a peptide/polypeptide comprising or consisting of an immunogenic amino acid sequence present in or derived from SEQ ID NO: 11 in combination with at least one NeGo peptide/polypeptide, in particular with at least one peptide/polypeptide comprising or consisting of an immunogenic amino acid sequence present in or derived from any one of SEQ ID NOs: 1-10, and 12-35. Another embodiment of a pharmaceutical composition disclosed herein comprises a peptide/polypeptide comprising or consisting of an immunogenic amino acid sequence present in or derived from SEQ ID NO: 12 in combination with at least one NeGo peptide/polypeptide, in particular with at least one peptide/polypeptide comprising or consisting of an immunogenic amino acid sequence present in or derived from any one of SEQ ID NOs: 1-11, and 13-35. Another embodiment of a pharmaceutical composition disclosed herein comprises a peptide/polypeptide comprising or consisting of an immunogenic amino acid sequence present in or derived from SEQ ID NO: 13 in combination with at least one NeGo peptide/polypeptide, in particular with at least one peptide/polypeptide comprising or consisting of an immunogenic amino acid sequence present in or derived from any one of SEQ ID NOs: 1-12, and 14-35. Another embodiment of a pharmaceutical composition disclosed herein comprises a peptide/polypeptide comprising or consisting of an immunogenic amino acid sequence present in or derived from SEQ ID NO: 14 in combination with at least one NeGo peptide/polypeptide, in particular with at least one peptide/polypeptide comprising or consisting of an immunogenic amino acid sequence present in or derived from any one of SEQ ID NOs: 1-13, and 15-35. Another embodiment of a pharmaceutical composition disclosed herein comprises a peptide/polypeptide comprising or consisting of an immunogenic amino acid sequence present in or derived from SEQ ID NO: 15 in combination with at least one NeGo peptide/polypeptide, in particular with at least one peptide/polypeptide comprising or consisting of an immunogenic amino acid sequence present in or derived from any one of SEQ ID NOs: 1-14, and 16-35. Another embodiment of a pharmaceutical composition disclosed herein comprises a peptide/polypeptide comprising or consisting of an immunogenic amino acid sequence present in or derived from SEQ ID NO: 16 in combination with at least one NeGo peptide/polypeptide, in particular with at least one peptide/polypeptide comprising or consisting of an immunogenic amino acid sequence present in or derived from any one of SEQ ID NOs: 1-15, and 17-35. Another embodiment of a pharmaceutical composition disclosed herein comprises a peptide/polypeptide comprising or consisting of an immunogenic amino acid sequence present in or derived from SEQ ID NO: 17 in combination with at least one NeGo peptide/polypeptide, in particular with at least one peptide/polypeptide comprising or consisting of an immunogenic amino acid sequence present in or derived from any one of SEQ ID NOs: 1-16, and 18-35. Another embodiment of a pharmaceutical composition disclosed herein comprises a peptide/polypeptide comprising or consisting of an immunogenic amino acid sequence present in or derived from SEQ ID NO: 18 in combination with at least one NeGo peptide/polypeptide, in particular with at least one peptide/polypeptide comprising or consisting of an immunogenic amino acid sequence present in or derived from any one of SEQ ID NOs: 1-17, and 19-35. Another embodiment of a pharmaceutical composition disclosed herein comprises a peptide/polypeptide comprising or consisting of an immunogenic amino acid sequence present in or derived from SEQ ID NO: 19 in combination with at least one NeGo peptide/polypeptide, in particular with at least one peptide/polypeptide comprising or consisting of an immunogenic amino acid sequence present in or derived from any one of SEQ ID NOs: 1-18, and 20-35. Another embodiment of a pharmaceutical composition disclosed herein comprises a peptide/polypeptide comprising or consisting of an immunogenic amino acid sequence present in or derived from SEQ ID NO: 20 in combination with at least one NeGo peptide/polypeptide, in particular with at least one peptide/polypeptide comprising or consisting of an immunogenic amino acid sequence present in or derived from any one of SEQ ID NOs: 1-19, and 21-35. Another embodiment of a pharmaceutical composition disclosed herein comprises a peptide/polypeptide comprising or consisting of an immunogenic amino acid sequence present in or derived from SEQ ID NO: 21 in combination with at least one NeGo peptide/polypeptide, in particular with at least one peptide/polypeptide comprising or consisting of an immunogenic amino acid sequence present in or derived from any one of SEQ ID NOs: 1-20, and 22-35. Another embodiment of a pharmaceutical composition disclosed herein comprises a peptide/polypeptide comprising or consisting of an immunogenic amino acid sequence present in or derived from SEQ ID NO: 22 in combination with at least one NeGo peptide/polypeptide, in particular with at least one peptide/polypeptide comprising or consisting of an immunogenic amino acid sequence present in or derived from any one of SEQ ID NOs: 1-21, and 23-35. Another embodiment of a pharmaceutical composition disclosed herein comprises a peptide/polypeptide comprising or consisting of an immunogenic amino acid sequence present in or derived from SEQ ID NO: 23 in combination with at least one NeGo peptide/polypeptide, in particular with at least one peptide/polypeptide comprising or consisting of an immunogenic amino acid sequence present in or derived from any one of SEQ ID NOs: 1-22, and 24-35. Another embodiment of a pharmaceutical composition disclosed herein comprises a peptide/polypeptide comprising or consisting of an immunogenic amino acid sequence present in or derived from SEQ ID NO: 24 in combination with at least one NeGo peptide/polypeptide, in particular with at least one peptide/polypeptide comprising or consisting of an immunogenic amino acid sequence present in or derived from any one of SEQ ID NOs: 1-23, and 25-35. Another embodiment of a pharmaceutical composition disclosed herein comprises a peptide/polypeptide comprising or consisting of an immunogenic amino acid sequence present in or derived from SEQ ID NO: 25 in combination with at least one NeGo peptide/polypeptide, in particular with at least one peptide/polypeptide comprising or consisting of an immunogenic amino acid sequence present in or derived from any one of SEQ ID NOs: 1-24, and 26-35. Another embodiment of a pharmaceutical composition disclosed herein comprises a peptide/polypeptide comprising or consisting of an immunogenic amino acid sequence present in or derived from SEQ ID NO: 26 in combination with at least one NeGo peptide/polypeptide, in particular with at least one peptide/polypeptide comprising or consisting of an immunogenic amino acid sequence present in or derived from any one of SEQ ID NOs: 1-25, and 27-35. Another embodiment of a pharmaceutical composition disclosed herein comprises a peptide/polypeptide comprising or consisting of an immunogenic amino acid sequence present in or derived from SEQ ID NO: 27 in combination with at least one NeGo peptide/polypeptide, in particular with at least one peptide/polypeptide comprising or consisting of an immunogenic amino acid sequence present in or derived from any one of SEQ ID NOs: 1-26, and 28-35. Another embodiment of a pharmaceutical composition disclosed herein comprises a peptide/polypeptide comprising or consisting of an immunogenic amino acid sequence present in or derived from SEQ ID NO: 28 in combination with at least one NeGo peptide/polypeptide, in particular with at least one peptide/polypeptide comprising or consisting of an immunogenic amino acid sequence present in or derived from any one of SEQ ID NOs: 1-27, and 29-30. Another embodiment of a pharmaceutical composition disclosed herein comprises a peptide/polypeptide comprising or consisting of an immunogenic amino acid sequence present in or derived from SEQ ID NO: 29 in combination with at least one NeGo peptide/polypeptide, in particular with at least one peptide/polypeptide comprising or consisting of an immunogenic amino acid sequence present in or derived from any one of SEQ ID NOs: 1-28, and 30-35. Another embodiment of a pharmaceutical composition disclosed herein comprises a peptide/polypeptide comprising or consisting of an immunogenic amino acid sequence present in or derived from SEQ ID NO: 30 in combination with at least one NeGo peptide/polypeptide, in particular with at least one peptide/polypeptide comprising or consisting of an immunogenic amino acid sequence present in or derived from any one of SEQ ID NOs: 1-29 and 31-35. Another embodiment of a pharmaceutical composition disclosed herein comprises a peptide/polypeptide comprising or consisting of an immunogenic amino acid sequence present in or derived from SEQ ID NO: 31 in combination with at least one NeGo peptide/polypeptide, in particular with at least one peptide/polypeptide comprising or consisting of an immunogenic amino acid sequence present in or derived from any one of SEQ ID NOs: 1-30, and 32-35. Another embodiment of a pharmaceutical composition disclosed herein comprises a peptide/polypeptide comprising or consisting of an immunogenic amino acid sequence present in or derived from SEQ ID NO: 32 in combination with at least one NeGo peptide/polypeptide, in particular with at least one peptide/polypeptide comprising or consisting of an immunogenic amino acid sequence present in or derived from any one of SEQ ID NOs: 1-31, and 33-35. Another embodiment of a pharmaceutical composition disclosed herein comprises a peptide/polypeptide comprising or consisting of an immunogenic amino acid sequence present in or derived from SEQ ID NO: 33 in combination with at least one NeGo peptide/polypeptide, in particular with at least one peptide/polypeptide comprising or consisting of an immunogenic amino acid sequence present in or derived from any one of SEQ ID NOs: 1-32, 34, and 35. Another embodiment of a pharmaceutical composition disclosed herein comprises a peptide/polypeptide comprising or consisting of an immunogenic amino acid sequence present in or derived from SEQ ID NO: 34 in combination with at least one NeGo peptide/polypeptide, in particular with at least one peptide/polypeptide comprising or consisting of an immunogenic amino acid sequence present in or derived from any one of SEQ ID NOs: 1-23 and 35. Another embodiment of a pharmaceutical composition disclosed herein comprises a peptide/polypeptide comprising or consisting of an immunogenic amino acid sequence present in or derived from SEQ ID NO: 35 in combination with at least one NeGo peptide/polypeptide, in particular with at least one peptide/polypeptide comprising or consisting of an immunogenic amino acid sequence present in or derived from any one of SEQ ID NOs: 1-34.

In this context, “derived from” is intended to denote that the amino acid sequence is a fragment or sequence variant of any one of SEQ ID NOs: 1-35 disclosed above.

These embodiments entail combinations of peptides/polypeptides which are admixed with each other. Alternatively, the same combinations of peptides/polypeptides can be constructed as fusion polypeptides, optionally connected via a linker as described above. Another alternative entails compositions where the immunogens are nucleic acids (DNA or RNA) encoding the peptide combinations or encoding such fusion polypeptides. In particular RNA vaccines have attracted attention recently, with the Covid-19 RNA vaccines from Pfizer/BioNTech and Moderne being the first examples used in larger scale in humans.

Another embodiment of the pharmaceutical composition disclosed herein comprises Z or at least Z distinct nucleic acid molecules each encoding a polypeptide disclosed herein, where each of said Z or at least Z distinct nucleic acid molecules encodes an immunogenic amino acid sequence present in or derived from any one of SEQ ID NOs: 1-35, and wherein said at Z or least Z distinct nucleic acid molecules together encode immunogenic amino acid sequences present in or derived from at Z or least Z of SEQ ID NOs.: 1-35, wherein Z is an integer selected from 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, and 35. Also, such a pharmaceutical composition may include nucleic acids that encode several immunogenic amino acid sequences disclosed herein, either as separate encoded species or as peptides fused to each other. So one variation of this embodiment is one single nucleic acid molecule, which encodes one or more of the polypeptides disclosed above or one or more of the combinations of peptides disclosed above.

Vaccines disclosed herein typically comprise immunising antigen(s), immunogen(s), polypeptide(s), protein(s) or nucleic acid(s), usually in combination with “pharmaceutically acceptable carriers”, which include any carrier that does not itself induce the production of antibodies harmful to the individual receiving the composition or targeting the protein/pathogen. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra.

The pharmaceutical compositions disclosed herein thus typically contain an immunological adjuvant, which is commonly an aluminium based adjuvant or one of the other adjuvants described in the following:

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi adjuvant system (RAS), (Ribi Immunochem, Hamilton, Mont.) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition. Alum and MF59™ adjuvants are preferred.

Muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

As indicated in the examples, the glucopyranosyl lipid adjuvant-stable emulsion (GLA-SE; developed by the Infectious Disease Research Institute, Seattle, Wash.) is one interesting adjuvant useful in the present invention.

The immunogenic compositions (eg. the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (eg. WO 98/20734). Additional formulations suitable for other modes of administration include oral, pulmonary and nasal formulations, suppositories, and transdermal applications. In the case of nucleic acid vaccination and antibody treatment, also the intravenous or intraarterial routes may be applicable.

Dosage treatment may be a single dose schedule or a multiple dose schedule. The vaccine may be administered in conjunction with other immunoregulatory agents.

As an alternative to protein-based vaccines, DNA vaccination (also termed nucleic acid vaccination or gene vaccination) may be used [eg. Robinson & Torres (1997) Seminars in Immunol 9: 271-283; Donnelly et al. (1997) Annu Rev Immunol 15: 617-648; later herein]. Also and as also pointed out herein, vaccination with RNA (mRNA) is an interesting and highly promising technology, cf. the above-mentioned reference by Deering R. P. et al.

Treatment Methods Disclosed Herein

The method of the sixth aspect disclosed herein generally relates to induction of immunity and as such also entails method that relate to treatment, prophylaxis and amelioration of disease.

When immunization methods entail that a polypeptide disclosed herein or a composition comprising such a polypeptide is administered the animal (e.g. the human) typically receives between 0.5 and 5,000 μg of the polypeptide disclosed herein per administration.

In preferred embodiments of this aspect, the immunization scheme includes that the animal (e.g. the human) receives a priming administration and one or more booster administrations.

Preferred embodiments of this aspect disclosed herein comprise that the administration is for the purpose of inducing protective immunity against NeGo. In turn this means that the administration is a prophylactic or therapeutic treatment of gonorrhoea.

As mentioned herein, the preferred vaccines disclosed herein induce humoral immunity, so it is preferred that the administration is for the purpose of inducing antibodies specific for NeGo and wherein said antibodies or B-lymphocytes producing said antibodies are subsequently recovered from the animal.

But, as also mentioned the method of this aspect may also be useful in antibody production, so in other embodiments the administration is for the purpose of inducing antibodies specific for NeGo and wherein B-lymphocytes producing said antibodies are subsequently recovered from the animal and used for preparation of monoclonal antibodies.

Pharmaceutical compositions can as mentioned above comprise polypeptides, antibodies, or nucleic acids disclosed herein. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance.

Reference is however made to the ranges for dosages of immunologically effective amounts of polypeptides, cf. above.

However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can as described herein also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

As is apparent from the claims, the invention also relates to related aspect and embodiments to the treatment and prophylaxis disclosed herein: the invention also includes aspects and embodiments where
the polypeptide disclosed herein is for use as a pharmaceutical, in particular for use as a pharmaceutical in the treatment, prophylaxis or amelioration of infection with NeGo;
the nucleic acid fragment disclosed herein or the vector disclosed herein is for use as a pharmaceutical, in particular for use as a pharmaceutical in the treatment, prophylaxis or amelioration of infection with NeGo;
the transformed cell disclosed herein is for use as a pharmaceutical, in particular for use as a pharmaceutical in the treatment, prophylaxis or amelioration of infection with NeGo.
the antibody, antibody fragment or antibody analogue disclosed herein is for use as a pharmaceutical, in particular for use as a pharmaceutical in the treatment, prophylaxis or amelioration of infection with NeGo.

Sequence Information

The proteins having the amino acid sequences numbered 1-35 in the sequence listing are named according to the following table:

| | SEQ ID |
|---|---|
| Name | NO: |
| NGO1125 | 1 |
| cNGO1947 | 2 |
| cNGO0725 | 3 |
| NGO1043 | 4 |
| cNGO1984 | 5 |
| NGO0182 | 6 |
| NGO1379 | 7 |
| NGO1549 | 8 |
| NGO0721 | 9 |
| NGO0265 | 10 |
| RS11935 | 11 |
| cNGO1094 | 12 |
| NGO1158 | 13 |
| cNGO1958 | 14 |
| cNGO1392 | 15 |
| cNGO1068 | 16 |
| cNGO1971 | 17 |
| NGO2059 | 18 |
| RS10860 | 19 |
| cNGO1585 | 20 |
| cNGO0571 | 21 |
| NGO0225 | 22 |
| cNGO1496 | 23 |
| cNGO2093 | 24 |
| cNGO1801 | 25 |
| cNGO1715 | 26 |
| cNGO2109 | 27 |
| cNGO1495 | 28 |
| NGO1785 | 29 |
| cNGO0952 | 30 |
| cNGO1286 | 31 |
| NGO0851 | 32 |
| cNGO0275 | 33 |
| NGO2105 | 34 |
| NGO1092 | 35 |

A number of the polypeptides of the invention are fragments of the full-length, native polypeptides. Such fragments are named as follows: NGOXXXX_Y-Z or cNGOXXXX_Y-Z (or sometimes NGOXXXX-Y-Z or cNGOXXXX-Y-Z), where XXXX is the 4 digit number in the polypeptide designation, Y is the number of the N-terminal amino acid residue in the fragment and Z is the number of the C-terminal amino acid residue in the fragment. For instance, NGO0952_100-400 (NGO0952-100-400) would be the polypeptide having the amino acid sequence SEQ ID NO: 30, residues 100-400, and cNGO0275_150-350 (or cNGO0275-150-350) would be the polypeptide having the amino acid sequence SEQ ID NO: 33, residues 150-350.

A corresponding naming convention is used in respect of SEQ ID NO: 11 and 19: RS11935_20-100 is the polypeptide having amino acid residues 20-100 in SEQ ID NO: 11.

The amino acid sequences of the polypeptides disclosed herein are derived from the following SEQ ID NOs:

SEQ ID NO: 1:
MSFHPETAYN GGGETEPYGP SPEEIKYRQS PETAETRRMT EKQAEGHIKS HR
SEQ ID NO: 2:
MNKNIAAALA GALSLSLAAG AVAAHKPASN ATGVQKSAQG SCGASKSAEG SCGAAASKAG
EGKCGEGKCG ATVKKAHKHT KASKAKAKSA EGKCGEGKCG SK
SEQ ID NO: 3:
MPRQSCHSSP FPRKRESGTQ TRQESIGKNN PTAVIPAQAG IQTHNVKAVY QKKPKPNALD
FRLRGNDEEL GSDERREQPR KKPPTPSDGI GNKNRTAETA RERIAGRAR
SEQ ID NO: 4:
MKKLLIAAMM AAALAACSQE AKQEVKEAAQ AVESDVKDTA ASAAESAASA VEEAKGQVKD
AAADAKASAE EAVTEAKDAA AETKEAVSEA AKDTLNKAAD AAQEAADKMK DAAK
SEQ ID NO: 5:
MPFEPPSDGI ARHPKSTIKM AKKPNKPFRL TPKLLIRAVL LICITAIGAL AVGIVSTFNP
NGDKTLQTEP QHTDSPRETE FWLPNGAVGQ DAAQPEHHHA ASSEPAQPDG TEESGSGLPP
PAAPKKNRVK PRPSDAARAA DSLTGTGTQA ENTLKETPVL PTNAPHPEPR KETPEKQAQP
KETPKEKETP KENHTKPDTP KNTPAKPHKE ILDNLF
SEQ ID NO: 6:
MFDFGLGELI FVGIIALIVL GPERLPEAAR TAGRLIGRLQ RFVGSVKQEL DTQIELEELR
KVKQAFEAAA AQVRDSLKET DTDMQNSLHD ISDGLKPWEK LPEQRTPADF GVDENGNPLP
DTANTVSDGI SDVMPSERSD TSAETLGDDR QTGSTAEPAE TDKDRAWREY LTASAAAPVV
QAVEVSYIDT AVETPVPHTT SLRKQAINRK RDFRPKHRAK PKLRVRKS
SEQ ID NO: 7:
MDKERILTPA VVFSVALLHL AIVALLWQAH KLPVIESGNV IEFVDLGDFG GGGGAPEGAG
APAAPEPQPA PDPPKPVEPP KPVLKPAVTK KADADIQQPK EKPKPEEKPK PEPEPEAKPA
PKPAEKPAEK PSEKPAEHSG NASAKAGSEQ GNGEGKGTGT KGDGTGRGEG SGKGSGGAKG
EHGEGAGGSG GGTGVGSSKG NPLRANGSIP RPAYPALSME NDEQGMVVLS VLVSPGGHVE
SVKVVKSSGF SRLDNAARKA AQNGHFQANA WTEFKVPVKF ELN
SEQ ID NO: 8:
MFMNKFSQSG KGLSGFFFGL ILATVIIAGI LLYLNQGGQN AFKIPAPSKQ PAETEILKLK
NQPKEDIQPE PADQNALSEP DVAKEAEQSD AEKAADKQPV ADKADEVEEK AGEPEREEPD
GQAVRKKALT EEREQTVREK AQKKDAETVK KQAVKPSKET EKKASKEEKK AAKEKVAPKP
TPEQILNSGS IEKARSAAAK EVQKMKTFGK AEATHYLQMG AYADRRSAEG QRAKLAILGI
SSEVVGYQAG HKTLYRVQSG NMSADAVKKM QDELKKHGVA SLIRAIEGK
SEQ ID NO: 9:
MKKNLPALAL ASMLILSGCD RLGIGNPFSG KEISCGSEET KEILVKLVRD NVEGETVKTF
DDDAFKDQAF ADIGISHIRR MVERLGITVD EVRTTEKTDT SSKLKCEAAL KLDVPDDWD
YAVAANQSIG NSHKKTPDFF EPYYRKEGAY YVKTISYSVQ PTDDKSKIFA ELSQAHDIIH
PLSELVSMAL IKEPLDKAKQ RNEKLEAAEA TAQEAREAEE AAAQEALGRE QEAARVSEWE
ERYKLSRSEF EQFWKGLPQT VQNKLQASQK TWKSGMDKIC ANNAKAEGET PNGIKVSELA
CKTAETEARL EELHNRKKAL IDEMVREEDK KELPKRL
SEQ ID NO: 10:
MSENKQNEVL TGYEQLKRRN RRRLVTASSL VAASCILLAA ALSSDPADSN PAPQAGETGA
TESQTANTAQ TPALKSAAEN GETAADKPQD LAGEDKPSAA DSEISEPENV GAPLVLINDR
LEDSNIKGLE ESEKLQQAET AKTEPKQAKQ RAAEKVSATA DSTDTVAVEK PKRTAEPKPQ
KAERTAEAKP KAKETKTAEK VADKPKTAAE KTKPDTAKSD SAVKEAKKAD KAEGKKTAEK
DRSDGKKHET AQKTDKADKT KTAEKEKSGK AGKKAAIQAG YAEKERALSL QRKMKAAGID
STITEIMTDN GKVYRVKSSN YKNARDAERD LNKLRVHGIA GQVTNE
SEQ ID NO: 11:
MAGLSNRQRR TKLSQWYNQC QTSQHLHNLR RTAKPTNHPS SSQSSPSESN SSRRQNYPYC
RRCGEQSNIN ITGSGVSGRA GTGLIADKQI HLQSAEQSNT ERSQNKSAGW NAGAAVSFGQ
GGWSLGVAAG GNVGKGYGNG DSVTHRHSHI GDKGSQTLIQ SGGDTIIKGA QVRGKGVQVN
AKNLSIQSVQ DRETYQSKQQ NAGAQVTVGY GFSASGDYSQ SKIRADHASV TEQSGIYAGE
DSYQIKVGNH TGLKGGIITS SQSAKDKGKN RFSTGTLAGS DIQNYSQYEG KSFGLGASVA
VSGKTLGQGA KNKPQDKHLT SIADKNGASS SVGYGSDSDS QSSITKSGIN TQKHSNHRRS
RTNQADRQNS GTNQSRY
SEQ ID NO: 12:
MGLFEPSAGD FWEMKEKEKK EKARKGAEER ERAAAQAHRA DAVRRTVANY EAGPARYRNV
MDLSRNNIED GARRLRRAGA FERGADAGLG FSGGDKALSP DARAGADFAR RDTRPTDAGG
RTPPPLGFDG NVYRDGKPVR DFDAQRPLVS AGPDALSPEE RELYRRATTP YAGALNGQLT
AAQLNAARGI VAEHNKNAAV RELGRERLAA AAAENAANRE AVLQKGRFDA AVKANEGALN
REMAQRNADR AFDVQQAELG MKRQGFEMKR EADALELEDR KRIADLTRAY GFAKSDGQRG
EIARQIDALN GKFERQGEKG FDPNVFKTIS YEVADPDTGL TAKREGIVDL RTGKPLDVEF
AGEREKRYAA LGFKPNGQKT AGGKIIYENE KGEKRVEQ
SEQ ID NO: 13:
MKQKKTVQCI LLGFAAASMH AQGAAAANSG TIEKTDKYTL VLAKQGQENN YTLNGGTEVK
PLNSLIIAAN GGTNNITIKG KLADGPADAP PTIDNNSIER NINKNGYTYA WQNWSGAVML
VDQSYEGENK VTFENVTIAA HNAPAGILSD DRHKSSSLAP AMLAFKGRNT INMDADSNAN
SSNEGILLLN NGEKMGEYRL VSEEGSTLNI NIKSGKDKGQ GITANHYGNS DINFNKASPN
ITTMEFKGDV NIKIDRNGQE EAESNGFGFY SSRKLGNKKQ IPEGSKMEAI FRGNVDIVAT
PVYDEQGRPK SIGSAFAIDG KYSKVEVVGG EGKVVKIKGD IFAYNGGSVS VNLANKDSYF
EGEAHIGKRS FAKGKDMFAL TVDADGYELT PDTKSIEKKK KELNVRGCTR LSLNSTPIPQ
DF
SEQ ID NO: 14:
MFKRSVIAMA CIFPLSACGG GGGGSPDVKS ADTPSKPAAP VVAENAGEGV LPKEKKDEEA
AGGAPQADTQ DATAGEGSQD MAAVSAENTG NGGAATTDNP KNEDAGAQND MPQNAAESAN
QTGNNQPAGS SDSAPASNPA PANGGSDFGR TNVGNSVVID GPSQNITLTH CKGDPCNGDN
LLDEEAPPKS EFESLSDEEK IKKYKKDGEK FTGLVAIKVE NNGLNKYTII YQAQPTRSAR
SRRSLPAEIP LIPVNQADTL IVDGEAVSLT GHSGNIFAPE GNYRYLTYGA EKLPGGSYAL
RVQGEPAKGE MLVGTAVYNG EVLHFHMENG RPYPSGGRFA AKVDFGSKSV DGIIDSGDDL
HMGTQKFKAA IDGNGFKGTW TENGGGDVSG RFYGPAGEEV AGKYSYRPTD AEKGGFGVFA
GKKDRD
SEQ ID NO: 15:
MKPLRRLTNL LAACAVAAAA LIQPALAADL AQDPFITDNT QRQHYEPGGK YHLFGDPRGS
VSDRTGKINV IQDYTHQMGN LLIQQAAIQG NLGYTVRFSG HGHEEHAPFD NHAADSASEE
KGNVDDGFTV YRLNWEGHEH HPADAYDGPK GGNYPKPTGA RDEYTYHVNG TARSIKLNPT
DTRSIRQRIS DNYNNLGSNF SDRADEANRK MFEHNAKLDR RGNSMEFVNG VAAGALNPFI
SAGEALGIGD ILYGTGYAID KAAMRNIAPL PAEGKFAVIG GLGSAAGFEK NTREAVDRWI
QENPNAAETV EAVFNVAAAA KVAKLAKAAK PGKAAVSGDF SKSYTCSFHG STLVRTADGY
KAIAHIQAGD RVLSKDEASG ETGYKPVTAR YGNPYQETVY IKVSDGIGNS QTLISNRIHP
FYSDGKWIKA EDLKAGSRL
SEQ ID NO: 16:
MNLPIQKFMM LFAAAISLLQ IPISHANGLD ARLRDDMQAK HYEPGGKYHL FGNGRGSVKN
RVCAVQTFDA TAVGPILPIT HERTGFEGII GYETHFSGHG HEVHSPFDNH DSKSTSDFSG
GVDGGFTVYQ LHRTGSEIHP EDGYDGPQGS DYPPPGGARD IYSYYVKGTS TKTKINTVPQ
APFSDRWLKE NAGAASGFLS RADEAGKLIW ENDPDKNWRA NRMDDIRGIV QGAVNPFLTG
FQGLGVGAIT DSAVNPVTDT AAQQTLQGIN DLGNLSPEAQ LAAASLLQDS AFAVKDGINS
ARQWADAHPN ITATAQTALA VAEAAGTVWR GKKVELNPAK WDWVKNTGYK KPAARHMQTV
DGEMAGGNRP LESKNTVTTN NFFENTGYTE KVLRQASNGD YHGFPQSVDA FSENGTVIQI
VGGDNIVRHK LYIPGSYKGK DGNFEYIREA DGKINHRLFV PNQQLPEK
SEQ ID NO: 17:
MNLPIQKFMM LFAAAISLLQ IPISHANGLD ARLRDDMQAK HYEPGGKYHL FGNGRGSVKN
RVCAVQTFDA TAVGPILPIT HERTGFEGII GYETHFSGHG HEVHSPFDNH DSKSTSDFSG
SVDGGFTVYQ LHRTGSEIHP ADGYDGPQGG GYPEPQGARD IYSYHIKGTS TKTKINTVPQ
APFSDRWLKE NAGAASGFLS RADEAGKLIW ENDPDKNWRA NRMDDIRGIV QGAVNPFLTG
FQGLGVGAIT DSAVNPVTYA AARKTLQGIH NLGNLSPEAQ LAAASLLQDS AFAVKDGINS
ARQWADAHPN ITATAQTALA VAEAAGTVWG GKKVELNPAK WDWVKNTGYK KPAARHMQTV
DGEMAGGNRP PKSITSEGKA NAATYPKLVN QLNEQNLNNI AAQDPRLSLA IHEGKKNFPI
GTATYEEADR LGKIWVGEGA RQTSGGGWLS RDGTRQYRPP TEKKSQFATT GIQANFETYT
IDSNEKRNKI KNGHLNIR
SEQ ID NO: 18:
MKHRTFFSLC AKFGCLLALG ACSPKIVDAG TATVPHTLST LKTADNRPAS VYLKKDKPTL
IKFWASWCPL CLSELGQAEK WAQDAKFSSA NLITVASPGF LHEKKDGEFQ KWYAGLNYPK
LPVVTDNGGT IAQNLNISVY PSWALIGKDG DVQRIVKGSI NEAQALALIR NPNADLGSLK
HSFYKPDTQK KDSAIMNTRT IYLAGGCFWG LEAYFQRIDG VVDAVSGYAN GNTENPSYED
VSYRHTGHAE TVKVTYDADK LSLDDILQYY FRVVDPTSLN KQGNDTGTQY RSGVYYTDPA
EKAVIAAALK REQQKYQLPL VVENEPLKNF YDAEEYHQDY LIKNPNGYCH IDIRKADEPL
PGKTKAAPQG KGFDAATYKK PSDAELKRTL TEEQYQVTQN SATEYAFSHE YDHLFKPGIY
VDVVSGEPLF SSADKYDSGC GWPSFTRPID AKSVTEHDDF SFNMRRTEVR SRAADSHLGH
VFPDGPRDKG RLRYCINGAS LKFIPLEQMD AAGYGALKGK VK
SEQ ID NO: 19:
MRAITSLLVM ICHFMLITSA SAAALRESAA CTRTSSVCVD GPSTKNINGV DVTKDCWEYK
EEYQCLEKDS ADYCAPLKDP SAKCEVQGQT CLEQSNEGEC LRYTHKYSCD VDLRTLHQGR
LPTKVEEMEH THLISSQWDE SSCQVQGKKC KAVATECLEP GSTKTINGVP VTRDCWKERR
TVQCTDGSDS ETCSAYTSSD QCRLIGDKCT HQLPDGTCQA REKQFECTEK GETTKEVSGC
QDRDFAKTMT TMEFARETQR FYDPEKQRFF NGEAGQCSIK LDGALDSVFG GDCCRTKADP
GKFVDFAVQT GTTMATTYFM ASVASHYTFT TMFVSSAAQA MGTTLSAAGG ITGTSQIGAL
GFSAAGQQGM GVIVGFNPAV FAAAIAVIAI QQWLKCPQSE ILVAMKRKAD LCHYVGSYCG
SKILGACVTM IESQCCFISK LAKIVNVGGK EQLKRGWGTP ENPKCEGFTA QELEQLDFSK
LDLSGFYEEI YANMDNVAKQ GQKVSQKIRE ASVNGKNLEV KNYYEYQ
SEQ ID NO: 20:
MKPLRRLIKL LAACAVAAAA LIQPALAADL AQDPFITDNT QRQHYEPGGK YHLFGDPRGS
VSDRTGKINV IQDYTHQMGN LLIQQANING TIGYHTRFSG HGHEEHAPFD NHAADSASEE
KGNVDDGFTV YRLNWEGHEH HPADAYDGPK GGNYPKPTGA RDEYTYHVNG TARSIKLNPT
DTRSIRQRIS DNYNNLGSNF SDRADEANRK MFEHNAKLDR RGNSMEFVNG VAAGALNPFI
SAGEALGIGD ILYGTRYAID KAAMRNIAPL PAEGKFAAIG GLGSVAGFEK NTREAVDRWI
QENPNAAETV EAVFNVAAAA KVAKLAKAAK PGKAAVSGDF SKSYTCSFHG STLVRTADGY
KAIAHIQAGD RVLSKDEASG ETGYKPVTAR YGNPYQETVY IKVSDGIGNS QTLISNRIHP
FYSDGKWIKA EDLKAGSRLF AENGAEQTVQ SVTVKPEPLK AYNLTVADWH TYFVKGSQAE
TEGVWVHNDC PPKPKPTNHA QQRKEEAKND SHRSVGDSNR VVREGKQYLD SDTGNHVYVK
GDKVVILTPD GRQVTQFKNS KANTSKRVKN GKWTPK
SEQ ID NO: 21:
MQYKPLLLAL MLVFSAPAVA AHDAAHNRSA EVKKQAKNKK EQPEAAEGKK EKGKNAAVKD
KKTGGKEAAK EFKKTAKNRK EAEKEATSRQ SARKGREGDK ESKAEHKKAH GKPVSGSKEK
NAKTQPENKQ GKKGAKGQGN PRKGGKAEKD TVSANKKARS DKNGKAVKQD KKYREEKNAK
TDSDELKAAV AAATNDVENK KALLKQSEGM LLHVSNSLKQ LQEERIRQER IRQERIRQAR
GNLASVNRKQ REAWDKFQKL NTELNRLKTE IAATKAQISR FVSGNYKNSQ PNAVALFLKN
AEPGQKNRFL RYTRYVNASN REVVKDLEKQ QKALAVQEQK INNELARLKK IQANVQSLLK
KQGVTDAAEQ TESRRQNAKI SKDARKLLEQ KGNEQQLNKL LSNLEKKKAE HRIQDAEAKR
KLAEARLAAA EKARKEAAQQ KAEARRAEMS NLTAEDRNIQ APSVMGIGSA DGFSRMQGRL
KKPVDGVPTG LFGQNRSGGD VWKGVFYSTA PATVESIAPG TVSYADELDG YGKVWIDHG
ENYISIYAGL SEISAGKGYT VAAGSKIGTS GSLPDGEEGL YLQIRYQGQV LNPSGWIR
SEQ ID NO: 22:
MGISRKISLI LSILAVCLPM HAHASDLAND PFIRQVLDRQ HFEPDGKYHL FGSRGELAER
SGHIGLGNIQ SHQLGNLMIQ QAAIKGNIGY IVRFSDHGHE VHSPFDNHAS HSDSDEAGSP
VDGFSLYRIH WDGYEHHPAD GYDGPQGGGY PAPKGARDIY SYDIKGVAQN IRLNLTDNRS
TGQRLADRFH NAGAMLTQGV GDGFKRATRY SPELDRSGNA AEAFNGTADI VKNIIGAAGE
IVGAGDAVQG ISEGSNIAVM HGLGLLSTEN KMARINDLAD MAQLKDYAAA AIRDWAVQNP
NAAQGIEAVS NIFMAAIPIK GIGAVRGKYG LGGITAHPVK RSQMGAIALP KGKSAVSDNF
ADAAYAKYPS PYHSRNIRSN LEQRYGKENI TSSTVPPSNG KNVKLADQRH PKTGVPFDGK
GFPNFEKHVK YDTKLDIQEL SGGGIPKAKP VFDAKPRWEV DRKLNKLTTR EQVEKNVQET
RRRSQSSQFK AHAQREWENK TGLDFNHFIG GDINKKGTVT GGHSLTRGDV RVIQQTSAPD
KHGVYQATVE IKKPDGSWEV KTKKGGKVMT KHTMFPKDWD EARIRAEVTS AWESRIMLKD
NKWQGTSKSG IKIEGFTEPN RTAYPIYE
SEQ ID NO: 23:
MNNPLVNQAA MVLPVFLLSA CLGGGGSFDL DSVDTEAPRP APKYKDVPSK KPEARKDQGG
YGFAMRFKRR NWYPPSNPKE NEIRLSEGNW EQTDDGEIKT PSKQKNIINA LSGNEGVSLQ
DSSQQGEGIS KVTDHHDFKY VWSGFFYKRI GITTKKDDLS NKIIEARNGP DGYIFYKGTD
PSRKLPVSGS VEYKGTWDFL TDVKANQKFT GLGNTSTKSG DRYSAFSGEL DYIVKKESDK
KDGHVGLGLT TEITVDFGKK TLSGKLIKNN MVINNGDEPT TQYYSLEAQV TGNRFNGKAI
ATDKPKVNET KEHPFVSDSS SLSGGFFGPQ GEELGFRFLS HDNKVAVVGS AKTKDKNANG
NTAAAGTAGA AGMSSEDTKL TTVLDAVELT PDGKKVKNLD NFSDATQLVV DGIMIPLLPT
ESGNGQADKG ENGKTAFIYE ttytpesdkk DTQTGMATNG VQTVSNTAGG TSGKTKTHYK
VQACCSNLNY LKYGLLTREN SNSVMQTVRN SSQAAARTEQ GAQSMFLQGE RTDEKEIPKE
QKVVYLGTWY GHIAANGTSW TGKASDQQSG NRAKFDVNFK DKKITGTLTA ANRQAETFTI
SGMIDGNGFE GTAKTGNGGF ALDANNTAAT HKAHIAEAKV RGGFYGPNAE ELGGWFAYPG
NGQAKNAQAS SGNENSAGSA TWFGAKRQQ LVQ
SEQ ID NO: 24:
MNAPFFRLSL LSLTLAAGFA HAAENNANVA LDTVTVKGDR QGSKIRTNIV TLQQKDESTA
TDMRELLKEE PSIDFGGGNG TSQFLTLRGM GQNSVDIKVD NAYSDSQILY HQGRFIVDPA
LVKVVSVQKG AGSASAGIGA TNGAIIAKTV DAQDLLKGLD KNWGVRLNSG FAGNNGVSYG
ASVFGKEGNF DGLFSYNRND EKDYEAGKGF RNDNGGKTVP YSALDKRSYL AKIGTTFGDG
DHRIVLSHMK DQHRGIRTVR EEFAVSEKNS RITIKRQAPS YRETTQSNTN LAYTGKDLGF
VEKLDANAYV LEKKRYSADD KDNGYAGNVK GPNHTRIATR GMNFNFDSRL AEQTLLKYGI
NYRHQEIKPQ AFLNSEFSIP IKEKKNGQEV DKPMEQQKKD RADEAIVRSY RLTNPTKTDT
GAYIEAIHEI DGFTLTGGLR YDRFKVKTHD GKTVSSSSLN PSFGVIWQPR EHWSFSASHN
YAGRSPRLYD ALQTHGKRGI ISIADGTKAE RARNTEIGFN YNDGTFAANG SYFRQTIKDA
LANPQNRHDS VAVREAVNAG YIKNHGYELG ASYRTGGLTA KVGVSRSKPR FYDTHPKKLL
SANPEFGAQT GRTWTASLAY RFKNPNLEIG WRGRYVQKAT GSILAAGQKD RDGKLENWR
QGFGVNDVFA NWKPLGKDTL NVNLSVNNVF DKFYYPHSQR WTNTLPGVGR DVRLGVNYKF
SEQ ID NO: 25:
MKLKQIASAL MMLGISPLAF ADFTIQDIRV EGLQRTEPST VFNYLPVKVG DTYNDTHGSA
IIKSLYATGF FDDVRVETAD GQLLLTVIER PTIGSLNITG AKMLQNDAIK KNLESFGLAQ
SQYFNQATLN QAVAGLKEEY LGRGKLNIQI TPKVTKLARN RVDIDITIDE GKSAKITDIE
FEGNQVYSDR KLMRQMSLTE GGIWTWLTRS DRFDRQKFAQ DMEKVTDFYQ NNGYFDFRIL
DTDIQTNEDK TRQTIKITVH EGGRFRWGKV SIEGDTNEVP KAELEKLLTM KPGKWYERQQ
MTAVLGEIQN RMGSAGYAYS EISVQPLPNA GTKTVDFVLH IEPGRKIYVN EIHITGNNKT
RDEVVRRELR QMESAPYDTS KLQRSKERVE LLGYFDNVQF DAVPLAGTPD KVDLNMSLTE
RSTGSLDLSA GWVQDTGLVM SAGVSQDNLF GTGKSAALRA SRSKTTLNGS LSFTDPYFTA
DGVSLGYDIY GKAFDPRKAS TSVKQYKTTT AGGGVRMGIP VTEYDRVNFG LAAEHLTVNT
YNKAPKRYAD FIKQYGKTDG ADGSFKGLLY KGTVGWGRNK TDSALWPTRG YLTGVNAEIA
LPGSKLQYYS ATHNQTWFFP LSKTFTLMLG GEVGIAGGYG RTKEIPFFEN FYGGGLGSVR
GYESGTLGPK VYDEYGEKIS YGGNKKANVS AELLFPMPGA KDARTVRLSL FADAGSVWDG
RTYTAAENGN NKSVYSENAH KSTFTNELRY SAGGAVTWLS PLGPMKFSYA YPLKKKPEDE
IQRFQFQLGT TF
SEQ ID NO: 26:
MARLFSLKPL VLALGFCFGT HCAADTVAAE EADGRVAEGG AQGASESAQA SDLTLGSTCL
FCSNESGSPE RTEAAVQGSG EASVPEDYTR IVADRMEGQS QVKVRAEGSV IIERDGAVLN
TDWADYDQSG DTVTVGDRFA LQQDGTLIRG ETLTYNLDQQ TGEAHNVRME TEQGGRRLQS
VSRTAEMLGE GRYKLTETQF NTCSAGDAGW YVKAASVEAD RGKGIGVAKH AAFVFGGVPL
FYTPWADFPL DGNRKSGLLV PSVSAGSDGV SLSVPYYFNL APNFDATFAP GIIGERGATF
DGQIRYLRPD YSGQTDLTWL PHDKKSGRNN RYQAKWQHRH DISDTLQAGV DFNQVSDSGY
YRDFYGGEEI AGNVNLNRRV WLDYGGRAAG GSLNAGLSVQ KYQTLANQSG YKDEPYAIMP
RLSADWHKNA GRAQIGVSAQ FTRFSHDGRQ DGSRLVVYPG IKWDFSNSWG YVRPKLGLHA
TYYSLDSFGG KASRSVGRVL PVVNIDGGTT FERNTRLFGG GVVQTIEPRL FYNYIPAKSQ
NDLPNFDSSE SSFGYGQLFR ENLYYGNDRI NAANSLSTAV QSRILDGATG EERFRAGIGQ
KFYFKDDAVM LDGSVGKNPR SRSDWVAFAS GGIGGRFTLD SSIHYNQNDK RAEHYAVGAG
YRPAPGKVLN ARYKYGRNEK IYLQADGSYF YDKLSQLDLS AQWPLTRNLS AWRYNYGFE
AKKPIEMLAG AEYKSSCGCW GAGVYAQRYV TGENTYKNAV FFSLQLKDLS SVGRNPAGRM
DVAVPGYIPA HSLSAGRNKR P
SEQ ID NO: 27:
MPIPFKPVLA AAAIAQAFPA FAADPAPQSA QTLNEITVTG THKTQKLGEE KIRRKTLDKL
LTNDEHDLVR YDPGISVVEG GRAGSNGFTI RGVDKDRVAI NVDGLAQAES RSSEAFQELF
GAYGNFNANR NTSEPENFSE VTITKGADSL KSGSGALGGA VNYQTKSASD YVSEDKPYHL
GIKGGSVGKN SQKFSSITAA GRLFGLDALL VYTRRFGKET KNRSTEGDVE IKNDGYVFDP
ANPSPSRYLT YKATGVARSQ PDPQEWVNKS TLFKLGYNFN DRNRIGWIFE DSRTDRFTNE
LSNLWTGTTT SAATGDYRHR QDVSYRRRTG VEYKNELEHG PWDSLKLRYD KQRIDMNTWT
WDIPKNYDTN GINGEVYHSF RHIRQNTAQW TADFEKQLDF SKAVWAAQYG LGGGRGDNAN
SDYSYFAKLY DPKILASNQA KITMLIENRS KYKFAYWNNV FHLGGNDRFR LNAGIRYDKN
SSSAKDDPKY TTAIRGQIPH LGSERAHAGF SYGTGFDWRF TKHLHLLAKY STGFRAPTSD
ETWLLFPHPD FYLKTNPELK AEKAKNWELG LAGSGKAGSF KLSGFKTKYR DFIELTYMGV
SSDDKNNPRY APLSDGTALV SSPVWQNQNR TAAWVEGIEF NGTWNLDSIG LPKGLHTGLN
VSYIKGKATQ NNGKETPINA LSPWTAVYSL GYDAPSKRWG VNAYAARTAA KKPSDTVHSN
DDLNKPWPYA KHSKAYTLFD LSAYLNIGKQ VTLRAAAYNI TNKQYYTWES LRSVREFGTV
NRVNNKTHAG IQRFTSPGRS YNFTIEAKF
SEQ ID NO: 28:
MQQQHLFRFN ILCLSLMTAL PAYAENVQAG QAQEKQLDTI QVKAKKQKTR RDNEVTGLGK
LVKTADTLSK EQVLDIRDLT RYDPGIAVVE QGRGASSGYS IRGMDKNRVA LTVDGLAQIQ
SYTAQAALGG TRTAGSSGAI NEIEYENVKA VEISKGSNSV EQGSGALAGS VAFQTKTADD
VIGEGRQWGI QSKTAYSGKN RGLTQSIALA GRIGGAEALL IRTGRHAGEI RAHEAAGRGV
QSFNRLVPVD DASTYAYFIV EEECKNGGYE KCKAKKDVDG KDERQTVSTR DYTGPNRFLA
DPLSYESRSW LFRPGFRFEN KRHYIGGILE RTQQTFDTRD MTVPAFLTKA VFDENKKYGS
IRGYGKYAGD HRYGGLITNS ENGAQVGAEY GTGVFYDETH TKSRYGLEYV YTNADKDTWA
DYARLSYDRQ GIGLDNHFQQ THCSADGSDK YCRPSADKPS SYYKSDRVIY GESHRLLQAA
FKKSFDTAKI RHNLSVNLGY DRFGSDLRHQ DYYYQHANRA YSSKTPPQNN GKKTSPNGSN
TSPYWVSIGG GNVVTGQICL FGNNTYTDCT PRSINGKSYY AAVRDNVRLG RWADVGAGLR
YDYRSTHSDD GSVSTGTHRT LSWNAGIVLK PADWLDLTYR TSTGFRLPSF AEMYGWRSGD
KIKAVKIDPE KSFNKEAGIV FKGDFGNLEA SWFDNAYRDL IVRGYEVQIK DGKEQVKGDP
AYLNAQSARI TGINILGKID WNGVWDKLPE GWYSTFAYNR VRVRDIKKRA DRTDIQSHLF
DAIQPSRYVV GSGYDQPEGK WGVNGMLTYS KAKEITELLG SRALLNGNSR NTKATARRTR
PWYIVDVSGY YTVKKHFTLR AGVYNLLNHR YVTWENVRQT AAGAVNQHKN VGVYNRYAAP
GRNYTFSLEM KF
SEQ ID NO: 29:
MKRMLFNATQ AEELRVAIVD GQNLLDLDIE TLGKEQRKGN IYKGIITRIE PSLEACFVDY
GTDRHGFLPF KEVSRSYFLG YEGGRARIQD VLKEGMEVIV QVEKDERGNK GAALTTFISL
AGRYLVLMPN NPRGGGVSRR IEGEERQELK AAMAQLDIPN GMSIIARTAG IGRSAEELEW
DLNYLKQLWQ AIEEAGKAHH DPYLLFMESS LLIRAIRDYF RPDIGEILVD NQEVYDQVAE
FMSYVMPGNA GRLKLYEDHT PLFSRFQIEH QIESAFSRSV SLPSGGAIVI DHTEALVSID
VNSARATRGA DIEDTAFKTN MEAAEEVARQ MRLRDLGGLV VIDFIDMENP KHQRDVENVL
RDALKKDRAR VQMGKLSRFG LLELSRQRLK PALGESSHAA CPRCAGTGVI RGIESTALHV
LRMVQEEAMK DNTGEVRAQV PVDVATFLLN EKRAELFAME ERLDVNVVLI PNIHLENPHY
EINRIRTDDV EEDGEPSYKR VAEPEEDESA KPFGGEKAKA ARPEPAVKGV RHTSPAPTAA
PEKKTSWWDS FKAWLKRIFG GSETQAVPAA ETSEKRSTAN RSGSRANNRR QNPRRSKREG
SKIEVREAAG KTAGQKARAD KAETRNNGNR RRNERGDRAT ERANEAEIQS RNVQPAAPVA
DAAPPETEGQ TGKRRRNGSR NERGQTAPET AAVAETAVQT AENTPPEPYT AEDKGSKPKS
ERNRRERDSR DAKERRERNN QRDRRQNGKK RNIPSAAKIE QYLNIHDTAD KVRSAAAHVF
GETDANAPIT VSIADPLIAT PVQTASSAVS NGDALIYDAA EKIRRAAADI LPEGAAPKAA
AQEMPSETAT FTAAAEQARE TAQTGGLVLI ETDPAALKAW AAQPEVQAGR GLRRSEQPKP
SEAATVPAEE MIQVETRQG
SEQ ID NO: 30:
MRSSFRLKPI CFYLMGVMLY HHSYAEDAGR AGSEAQIQVL EDVHVKAKRV PKDKKVFTDA
RAVSTRQDIF KSGENLDNIV RSIPGAFTQQ DKSSGIVSLN IRGDSGFGRV NTMVDGITQT
FYSTSTDAGR AGGSSQFGAS VDSNFIAGLD VVKGSFSGSA GINSLAGSAN LRTLGVDDVV
QGNNTYGLLL KGLTGTNSTK GNAMAAIGAR KWLESGASVG VLYGHSRRGV AQNYRVGGGG
QHIGNFGAEY LERRKQRYFV QEGGLKFNSN SGKWERDFQR PYWKTKWYQK YNDPQELQKY
IEGHDKSWRE NLAPQYDITP IDPSGLKQQS AGNLFKLEYD GVFNKYTAQF RDLNTKIGSR
KIINRNYQFN YGLSLNPYTN LNLTAAYNSG RQKYPKGSKF TGWGLLKDFE TYNNAKILDL
NNTATFRLPR ETELQTTLGF NYFHNEYGKN RFPEELGLFF DGPDQDNGLY SYLGRFKGDK
GLLPQKSTIV QPAGSQYFNT FYFDAALKKD IYRLNYSTNA INYRFGGEYT GYYGSENEFK
RAFGENSPAY KEHCDPSCGL YEPVLKKYGK KRANNHSVSI SADFGDYFMP FAGYSRTHRM
PNIQEMYFSQ IGDSGVHTAL KPERANTWQF GFNTYKKGLL KQDDILGLKL VGYRSRIDNY
IHNVYGKWWD LNGDIPSWVG STGLAYTIRH RNFKDKVHKH GFELELNYDY GRFFTNLSYA
YQKSTQPTNF SDASESPNNA SKEDQLKQGY GLSRVSALPR DYGRLEVGTR WLGNKLTLGG
AMRYFGKSIR ATAEERYIDG TNGGNTSNVR QLGKRSIKQT ETLARQPLIF DFYAAYEPKK
NLIFRAEVKN LFDRRYIDPL DAGNDAATQR YYSSFDPKDK DEDVTCNADK TLCNGKYGGT
SKSVLTNFAR GRTFLMTMSY KF
SEQ ID NO: 31:
MSNTTVEQFA AELKRPVEDL LKQLKEAGVS KNSGSDSLTL DDKQLLNAYL TKKNGSNGGT
ISIRRTKTEV STVDGVKVET RKRGRTVNIP SAEELAAQVK AAQTQAAPVQ PEQTAEDAVK
ARAEAAARAE ARAKAEAEAA KLKAAKAGNK AKPAAQKPTE AKAETAPVAA ETKPAEPKEK
AVKPKHERNG KGKDAKKPAK PAAPAVPQPV VSAEEQAQRD EEARRAAALR AHQEALLKEK
QERQARREAM KQQAEQQAKA AQEAKTGRQR PAKPAEKPQA AAPAVENKPV NPAKAKKEDR
RNRDDEGQGR NAKGKGAKGG RDRNNARNGG DERVRGGKKG KKLKLEPNQH AFQAPTEPW
HEVLVPETIT VADLAHKMAV KGVEVVKALM KMGMMVTINQ SIDQDTALIV VEELGHIGKP
AAADDPEAFL GEGAEAVEAE ALPRPPVVTV MGHVDHGKTS LLDYIRRAKV VQGEAGGITQ
HIGAYHVKTP RGVITFLDTP GHEAFTAMRA RGAKATDIVI LVVAADDGVM PQTIEAIAHA
KAAGVPIVVA VNKIDKDTAN PERIRQELTQ HEVIPDDWGG TVQFIDVSAK KGTNIDALLE
AVLLEAEVLE LTAPVDAPAK GIIVEARLDK GRGAVATLLV QNGTLKKGDM LLAGTAFGKI
RAMVDENGKS ITEAGPSIPV EILGLSDVPN AGEDAMVLAD EKKAREIALF RQGKYRDVRL
AKQQAAKLEN MFNNMGETQA QSLSVIIKAD VQGSYEALAG SLKKLSADEV KVNVLHSGVG
GITESDVNLA IASGAFIIGF NVRADASSRK LAENENVEIR YYNIIYDAID DVKAAMSGML
SPEKKEQVTG TVEIRQVISV SKVGNIAGGM VTDGVVKRDS HIRLIRNNVV IHTGELASLK
RYKDDVKEVR MGFECGLMLK GYNEIMEGDQ LECFDIVEVA RTL
SEQ ID NO: 32:
MFWIVLIVIL LLALAGLFFV RAQSEREWMR EVSAWQEKKG EKQAELPEIK DGMPDFPEFS
LMLFHAVKTA VYWLFVGVVR FCRNYLAHES EPDRPVPPAS ANRADVPTAS DGYSDSGNGT
EEAETEAAEA AEEEAADTED IATAVIDNRR IPFDRSIAEG LMQSESKTSP VRPVFKEITL
EEATRALSSA ALRETKKRYI DAFEKNGTAV PKVRVSDTPM EGLQIIGLDD PVLQRTYSRM
FDADKEAFSE SADYGFEPYF EKQHPSAFSA VKAENARNAP FRRHAGQEKG QAEAKSPDVS
QGQSVSDGTA VRDARRRVSV NLKEPNKATV SAEARISRLI PESRTVVGKR DVEMPSETEN
VFTETVSSVG YGGPVYDEAA DIHIEEPAAP DAWVVEPPEV PEVAVPEIDI LPPPPVSEIY
NRTYEPPAGF EQAQRSRIAE TDHLAADVLN GGWQEETAAI ADDGSEGAAE RSSGQYLSET
EAFGHDSQAV CPFEDVPSER PSCRVSDTEA DEGAFQSEET GAVSEHLPTT DLLLPPLFNP
EATQTEEELL ENSITIEEKL AEFKVKVKVV DSYSGPVITR YEIEPDVGVR GNSVLNLEKD
LARSLGVASI RVVETIPGKT CMGLELPNPK RQMIRLSEIF NSPEFAESKS KLTLALGQDI
TGQPVVTDLG KAPHLLVAGT TGSGKSVGVN AMILSMLFKA APEDVRMIMI DPKMLELSIY
EGITHLLAPV VTDMKLAANA LNWCVNEMEK RYRLMSFMGV RNLAGFNQKI AEAAARGEKI
GNPFSLTPDD PEPLEKLPFI VVVVDEFADL MMTAGKKIEE LIARLAQKAR AAGIHLILAT
QRPSVDVITG LIKANIPTRI AFQVSSKIDS RTILDQMGAE NLLGQGDMLF LPPGTAYPQR
VHGAFASDEE VHRVVEYLKQ FGEPDYVDDI LSGGGSEELP GIGRSGDGET DPMYDEAVSV
VLKTRKASIS GVQRALRIGY NRAARLIDQM EAEGIVSAPE HNGNRTILVP LDNA
SEQ ID NO: 33:
MKAKRFKINA ISLSIFLAYA LTPYSEAALV RDDVDYQIFR DFAENKGKFF VGATDLSVKN
KRGQNIGNAL SNVPMIDFSV ADVNKRIATV VDPQYAVSVK HAKAEVHTFY YGQYNGHNDV
ADKENEYRVV EQNNYEPHKA WGASNLGRLE DYNMARFNKF VTEVAPIAPT DAGGGLDTYK
DKNRFSSFVR IGAGRQLVYE KGVYHQEGNE KGYDLRDLSQ AYRYAIAGTP YKDINIDQTM
NTEGLIGFGN HNKQYSAEEL KQALSQDALT NYGVLGDSGS PLFAFDKQKN QWVFLGTYDY
WAGYGKKSWQ EWNIYKKEFA DKIKQHDNAG TVKGNGEHHW KTTGTNSHIG STAVRLANNE
GDANNGQNVT FEDNGTLVLD QNINQGAGGL FFKGDYTVKG ANNDITWLGA GIDVADGKKV
VWQVKNPNGD RLAKIGKGTL EINGTGVNQG QLKVGDGTVI LNQKADADKK VQAFSQVGIV
SGRGTLVLNS SNQINPDNLY FGFRGGRLDA NGNDLTFEHI RNVDEGARIV NHNTDHASTI
TLTGKSLITN PNSLSVHSIQ NDYDEDDYSY YYRPRRPIPQ GKDLYYKNYR YYALKSGGNV
NAPMPENGVA ENNDWVFMGY TQEEAKKNAM NHKNNQRISG FSGFFGEENE KGHNGALNLN
FNGKSAQNRF LLTGGANLNG KISVTQGNVL LSGRPTPHAR DFVNKSSARK DAHFSKNNEV
VFEDDWINRT FKAAEIAVNQ SASFSSGRNV SDITANITAT DNAKVNLGYK NGDEVCVRSD
YTGYVTCNTG NLSDKALNSF DATRINGNVN LSQNAALVLG KAALWGQIQG QGNSRVSLNQ
HSKWHLTGDS QVHNLSLADS HIHLNNASDA QSANKYHTIK INHLSGNGHF HYLTDLAKNL
GDKVLVKESA SGHYQLHVQN KTGEPNQEGL DLFDASSVQD RSRLFVSLAN HYVDLGALRY
TIKTENGITR LYNPYAGNRR PVKPAPSPAA NTASQAQKAT QTDGAQIAKP QDIWAPPSP
QANQAEEAKR QQAKAEQVKR QQAEAGRKSA ELSAKQRAGE EERRQTAQSQ PQRRK
SEQ ID NO: 34 :
MKTTDKRTTE THRKAPKTGR IRFSPAYLAI CLSFGILPQA RAGHTYFGIN YQYYRDFAEN
KGKFAVGAKD IEVYNKKGEL VGKSMTKAPM IDFSVVSRNG VAALAGDQYI VSVAHNGGYN
NVDFGAEGSN PDQHRFSYQI VKRNNYKAGT NGHPYGGDYH MPRLHKFVTD AEPVEMTSYM
DGWKYADLNK YPDRVRIGAG RQYWRSDEDE PNNRESSYHI ASAYSWLVGG NTFAQNGSGG
GTVNLGSEKI KHSPYGFLPT GGSFGDSGSP MFIYDAQKQK WLINGVLQTG NPYIGKSNGF
QLVRKDWEYD EIFAGDTHSV FYEPHQNGKY FFNDNNNGAG KIDAKHKHYS LPYRLKTRTV
QLFNVSLSET AREPVYHAAG GVNSYRPRLN NGENISFIDK GKGELILTSN INQGAGGLYF
EGNFTVSPKN NETWQGAGVH ISDGSTVTWK VNGVANDRLS KIGKGTLLVQ AKGENQGSVS
VGDGKVILDQ QADDQGKKQA FSEIGLVSGR GTVQLNADNQ FNPDKLYFGF RGGRLDLNGH
SLSFHRIQNT DEGAMIVNHN QDKESTVTIT GNKDITTTGN NNNLDSKKEI AYNGWFGEKD
ATKTNGRLNL NYQPEEADRT LLLSGGTNLN GNITQTNGKL FFSGRPTPHA YNHLGSGWSK
MEGIPQGEIV WDNDWIDRTF KAENFHIQGG QAVVSRNVAK VEGDWHLSNH AQAVFGVAPH
QSHTICTRSD WTGLTSCTEK TITDDKVIAS LSKTDIRGNV SLADHAHLNL TGLATLNGNL
SAGGDTHYTV TRNATQNGNL SLVGNAQATF NQATLNGNTS ASDNASFNLS NNAVQNGSLT
LSDNAKANVS HSALNGNVSL ADKAVFHFEN SRFTGKISGG KDTALHLKDS EWTLPSGTEL
GNLNLDNATI TLNSAYRHDA AGAQTGSAAD APRRRSRRSL LSVTPPTSAE SRFNTLTVNG
KLNGQGTFRF MSELFGYRSG KLKLAESSEG TYTLAVNNTG NEPVSLEQLT WEGKDNTPL
SENLNFTLQN EHVDAGAWRY QLIRKDGEFR LHNPVKEQEL SDKLGKAGET EAALTAKQAQ
LAAKQQAEKD NAQSLDALIA AGRNATEKAE SVAEPARQAG GENAGIMQAE EEKKRVQADK
DTALAKQREA ETRPATTAFP RARRARRDLP QPQPQPQPQP QRDLISRYAN SGLSEFSATL
NSVFAVQDEL DRVFAEDRRN AVWTSGIRDT KHYRSQDFRA YRQQTDLRQI GMQKNLGSGR
VGILFSHNRT GNTFDDGIGN SARLAHGAVF GQYGIGRFDI GISAGAGFSS GSLSDGIRGK
IRRRVLHYGI QARYRAGFGG FGIEPHIGAT RYFVQKADYR YENVNIATPG LAFNRYRAGI
KADYSFKPAQ HISITPYLSL SYTDAASGKV RTRVNTAVLA QDFGKTRSAE WGVNAEIKGF
TLSLHAAAAK GPQLEAQHSA GIKLGYRW
SEQ ID NO: 35:
MPDGLARQYA DMAAKYRAKP SEAVGIDPDD GAVSAAAALA ADSGAAVPSA VSDDMEARSV
ADDAPSGRSA DADRGGVPSA YGNVRPGGAP RGAASVAPGG SAAAASGGIA RVAPLPAGQY
FDGLDTRGRK ALAKEAGLDI KGVADFGQIA APVRRKIEQA YHARIEADYQ AASEAKQGYL
PPPVRMADAV PVPKKGFSVP ADALDKESRR RFDALPEWVR RHAQTVADYT ADGIMRREAG
MADMRGHYPE GLAESAGAVR AYRAQHPESA DVLDRLNRAV YGYRRNNGWS VPLLSREGER
LQGVRTALPD DGASEAVVGG GRGLTRALPT EDKGLAQDVR QDVRQGLTQG GRGLTPDAGA
DANAAALQGL PGSAVASGNA PARRQNLQVR ARAEGAAPGL SASENLAGTD GGKRAPVAGK
RPDTVLPVLN PQVAESAGRV SPKKRMADAA ADFTRRLAAD RRRPEKAGVP LGGGEYRFEH
TDRRHIDALA GVPGRPGKGG MPEEFADMAG PSNSDGLVSD GRRYLKGREA ETLRAGGLSE
AVPSEPGRDY RPTQEARAPA KVMARPRDAA ADGKPAGRAQ PARAKDTPVA GKAAAAKNAA
TEKPSSDKVR NIEAGKSRFD GGKGKSAAAQ GAATEKPSEK TGKAKPETFA KTASDNPEEA
RRKARVLQGG PVYTVKERQA PQGFKALREH AESIKKRLAE SIGGLAERVD VAAVSETAPD
KAQMLLSQRV EGWFDGRTGK ITLVAENLTP ERAVWAAWHE LGHRGFAADG FAKYREELER
ADGNGLIRRI ADAVQEGREG TGDAAASVRP AAVEEAVAEL YAAQRTGGWA GIENRYGVKV
GNGLKRGIAG VLARIGALLR RVLQRLAGKA GGAMSDADVE AMLADLHGNV EGARDAPWGG
NHRAVMFARA EDGAAERSKS ESLEKLRRAE TIRISGREVP EGGNLREYKR NALEYGKSLR
GPYVNKDTGR EISLGRSGIT EILRHDYKDA EHLQSIAAIP QIIENAVYID TLPNEDLAKN
GDIQGYEYYV SGLNVGGADY TVRAAVAVSR NGNRYYDHKL TKIEKGNLLS LLDRVSTTGA
SESKSPLSGI DDKRLLQILQ DKDAGKGGIA DFDTEAVRFS RAANIEAAIG RITGKKSDLR
NALKDRWDAS KGIQLQFLGR RQIEDIYGGV LDGLKEYGRL SELFGADANK AVTEADKWR
EWGRLKEEDA KALADLMHDA TLAKVDADPL MRKDAQKRLD GIRTALDIAD GKIEKAEAAV
ASAGARIARA DAAYNKAQRA ADKAAYALEK AQEKHGREIL ADEADMRLRR LFYADSEAKR
ALRRAGADVA AESRAKTDAV RMLEQARADV KRLEKDEVGA QKALEGLALL NRRFAGLPDA
AQRVYRKARD DYRAHFGQVR DALAERLARA GQDAETVRRL KERFDNELGG VYFPLARFGD
YLVVVKDADG NSANVSRAET LSEAEKLRDA LKADFGAGFK VSPVMKSRDY IRSRDAVGSG
FMRELGEAVG MLDLDPAQRA RLNDTLTQLY LNSLPDTSWA KHGIHRKGVP GFSDDARRAY
AQNMGSGANY LAKLRYADRM AEQLDVMQDF VDGRKYEEGF DQRQLQRVAD EMRKRHEAVM
NPNPSKLAQA LTGFGFLWMM GMSPASAVVN LSQTAMVAYP VMAAKWGYAG AARELLRASK
QIGLRFGEKF NTIEDSLNGD EKAAFRKAAD YGVIDLSQAH DLAGVANGDP GLAGSAWQKV
MDKAAWLFHH AEKFNRQVTF VAAYRLAKRA GADSEAAFEQ AKKATYDGHF DYAAQNRPRF
MMGNAAKVVF LFKQYSQNIL YALGRNAYLA FKGDKEARKT LAGLLVSHAM ASGILGLPFV
STLLAVASML GSDDDDPWDA EAALRNMLAD AFGDKAGEVL AKGFSRLTPL DVSGRLGLNQ
LVFPDIQDGL EGKKWAESLV VGSTGAVVGA GIGAADGVRT RSSVPRTANT LSLMKSW

### Example 1

Immunization Study in Mice

Bacterial strains: N. gonorrhoeae strains FA1090, MS11 (Opa-), F62(ΔIgtD), and H041.

Immunization of mice: Six-week-old female BALB/c mice were immunized intramuscularly (IM) with 11 different compositions of recombinant NeGo proteins (15 μg each) and adjuvant (Glucopyranosyl Lipid A-stable emulsion; hereinafter GLA-SE) (5 μg) or GLA-SE (5 μg)+AlOH₃ or with positive control TMCP2 (Gulati et al. 2019) (50 μg) and adjuvant GLA-SE (5 μg). Control mice received GLA-SE (5 μg) adjuvant alone. Mice were immunized by schedule: Primary immunization (day 0) and boosts (day 20 and 39).

The compositions of the 11 compositions and the negative and positive controls are given in the following table:

| | Protein ID | | | | | Construct | | Dose of Adjuvant | No |
|---|---|---|---|---|---|---|---|---|---|
| | (strain | Amount | | Formu- | | Length | | formulation per | of |
| Group | FA1090) | (ug) | Solublity | lation | Contruct ID | (AA) | Adjuvant | animal (μg) | Mice |
| 1 | — | 50 | — | — | TMCP2 | — | GLA-SE | 5 | 15 |
| 2 | NGO1496 | 15 | soluble | NaCl | cNGO1496-1-693 | 693 | GLA-SE | 5 | 15 |
| | NGO0571 | 15 | soluble | NaCl | cNGO0571-21-598 | 578 | | | |
| 3 | NGO1379 | 15 | soluble | NaCl | NGO1379-28-283 | 256 | GLA-SE | 5 | 15 |
| | NGO0725 | 15 | soluble | NaCl | cNGO0725-1-109 | 109 | | | |
| 4 | NGO1158 | 15 | soluble | NaCl | NG01158-27-422 | 396 | GLA-SE | 5 | 15 |
| | NGO0182 | 15 | soluble | NaCl | NGO0182-26-228 | 203 | | | |
| 5 | NGO0721 | 15 | soluble | NaCl | NGO0721-22-337 | 316 | GLA-SE | 5 | 15 |
| | NGO2105 | 15 | soluble | NaCl | NGO2105-44-1468 | 1425 | | | |
| 6 | NGO1094 | 15 | soluble | NaCl | cNGO1094-1-398 | 398 | GLA-SE | 5 | 15 |
| | NGO1043 | 15 | soluble | NaCl | NGO1043-22-114 | 93 | | | |
| | NGO2059 | 15 | soluble | NaCl | NGO2059-22-522 | 501 | | | |
| 7 | NGO1984 | 15 | soluble | NaCl | cNGO1984-59-216 | 158 | GLA-SE | 5 | 15 |
| | NGO1286 | 15 | soluble | NaCl | cNGO1286-1-943 | 943 | | | |
| | NGO1092 | 15 | soluble | NaCl | NGO1092-1-649 | 649 | | | |
| 8 | NGO0275 | 15 | unsoluble | Urea | cNGO0275-28-1075 | 1048 | GLA-SE + AlOH3 | 5 | 15 |
| | NGO0225 | 15 | unsoluble | Urea | NGO0225-25-628 | 604 | | | |
| 9 | NGO1495 | 15 | unsoluble | Urea | cNGO1495-25-912 | 888 | GLA-SE + AlOH3 | 5 | 15 |
| | NGO2093 | 15 | unsoluble | Urea | cNGO2093-23-720 | 698 | | | |
| 10 | NGO1392 | 15 | unsoluble | Urea | cNGO1392-28-439 | 412 | GLA-SE +AlOH3 | 5 | 15 |
| | NGO1585 | 15 | unsoluble | Urea | cNGO1585-28-576 | 549 | | | |
| | NGO2109 | 15 | unsoluble | Urea | cNGO2109-23-809 | 787 | | | |
| 11 | NGO1801 | 15 | unsoluble | Urea | CNGO1801-22-792 | 771 | GLA-SE + AlOH3 | 5 | 15 |
| | NGO0952 | 15 | unsoluble | Urea | CNGO0952-26-922 | 897 | | | |
| | NGO1715 | 15 | unsoluble | Urea | cNGO1715-25-801 | 777 | | | |
| 12 | NGO1549 | 15 | soluble | NaCl | NG01549-35-289 | 255 | GLA-SE | 5 | 15 |
| | NGO0265 | 15 | soluble | NaCl | NGO0265-44-346 | 303 | | | |
| 13 | — | — | — | — | Control (adjuvant) | — | GLA-SE | 5 | 15 |

Bleeding of mice: Mice were bled on days −1, 13, 32, 46, 60 and 71 relative to the first immunization.

Infection of mice: Mice were infected day 57 after the first immunization.

ELISA to measure levels of antibody directed against recombinant NeGo proteins and whole cell lysates: Microtiter wells were coated with recombinant proteins or whole cell lysate from Ng strains FA1090, MS11 (Opa-), F62(ΔD) or H041 in phosphate-buffered saline (PBS) (cf. Gulati et al. 2013). Serial dilutions of immune sera were dispensed into wells, and bound antibody was disclosed with anti-mouse IgG conjugated to alkaline phosphatase. A standard curve for mouse IgG was generated by coating wells with anti-mouse IgG (Sigma) and pure mouse IgG (Sigma) (cf. Gulati et al. 2013) and aliquots with known concentrations of pure mouse IgG dispensed to wells. ELISAs were performed on pooled antisera from groups of the same 5 mice bled at day −1, 13, 32 and 46. Ten mice from each group that were in the diestrus phase of the estrous cycle and thus suitable for challenge with Ng (5 mice with Ng strain MS11 and 5 mice with 5 mice with Ng strain H041) were infected on day 57. ELISA was performed on pooled antisera from 5 uninfected mice bleed at day 60. ELISA was performed on pooled antisera from infected mice bleed at day 71. Not all the 5 mice that were bled at day −1, 13, 32, and 46 ended up being infected. But all the mice bleed after infection (3-5 mice) are the same among the 5 mice that were bled at day −1, 13, 32, and 46.

Mouse protection experiments: Use of animals in this study was performed in strict accordance with the recommendations in the Guide for the Care and Use of Laboratory Animals of the National Institutes of Health 2011. The protocol was approved by the Institutional Animal Care and Use Committee (IACUC) at the University of Massachusetts Medical School. The BALB/c mouse model of vaginal colonization described in Jerse 1999 was used. Two weeks after the last immunization, mice in the diestrus phase of the estrous cycle were started on treatment (that day) with 0.1 mg Premarin (Pfizer) in 200 μl of water, given subcutaneously on each of 3 days: days 55, 57, and 59 (before, the day of, and after gonococcal inoculation) to prolong the estrus phase of the reproductive cycle and promote susceptibility to N. gonorrhoeae infection. Antibiotics (vancomycin and streptomycin) ineffective against N. gonorrhoeae were also used to reduce competitive microflora (Jerse et al. 2011). Immunized mice and placebo control mice were infected on day 57 with either strain MS11 (inoculum dose: 2.6×10⁷ CFU) or H041 (inoculum dose: 3.8×10⁷ CFU). Vaginas were swabbed daily to enumerate CFUs. Efficacy of the vaccine groups were measured using: i) time to clearance of infection, ii) log 10 CFU vs time and iii) Area Under curve analysis.

Statistical analyses: Experiments that compared clearance of N. gonorrhoeae in independent groups of mice estimated and tested three characteristics of the data (cf. Gulati et al. 2013): time to clearance; longitudinal trends in mean log 10 CFU and the cumulative CFU as area under the concentration-time curve (AUC). Statistical analyses were performed using mice that initially yielded bacterial colonies on day 1 and/or 2 (cf. Gulati et al. 2019). Median time to clearance was estimated using Kaplan-Meier survival curves; times to clearance were compared between groups using the Mantel-Cox log-rank test and Gehan-Breslow-Wilcoxon test. The mean AUC (log 10 CFU versus time) was computed for each mouse to estimate the bacterial burden over time (cumulative infection); the means under the curves were compared between groups using the nonparametric two-sample Wilcoxon rank-sum (Mann-Whitney) test because distributions were skewed or kurtotic. The median AUC (log 10 CFU versus time) percent reduction (test group vs placebo control group) were calculated.

ELISA Results

The following tables show the results of the ELISA testing of mice antisera against the various immunogens used in the immunization study. Data are shown as gross reading minus substrate control (OD 405 nm):

Plate 23

| | GROUP 1 | | Immunogen | TMCP2 | | | | | |
|---|---|---|---|---|---|---|---|---|---|
| | Pool | | Wk | 0 | 2 | 5 | 7 | 9 | 11 |
| | Serum | | Days | −1 | 13 | 32 | 46 | 60 | 71 |
| Wells | | | | | | | | | |
| Coated | | Serum | | | | | | | |
| with | | dilution | | 1 | 2 | 3 | 4 | 5 | 6 |
| TMCP2 | | 100 | E | 0.008 | 0.165 | 0.183 | 0.171 | 0.141 | 0.191 |
| | | 500 | F | 0.005 | 0.067 | 0.065 | 0.068 | 0.074 | 0.089 |
| | | 5000 | G | 0.009 | 0.032 | 0.030 | 0.027 | 0.034 | 0.035 |
| | | 25000 | H | 0.006 | 0.013 | 0.006 | 0.007 | 0.013 | 0.020 |

Plate 24

| _ | GROUP 1 | | Immunogen | TMCP2 | | | | | | | | | | | |
|---|---|---|---|---|---|---|---|---|---|---|---|---|---|---|---|
| | Pool | | Wk | 0 | 2 | 5 | 7 | 9 | 11 | 0 | 2 | 5 | 7 | 9 | 11 |
| | Serum | | Days | −1 | 13 | 32 | 46 | 60 | 71 | −1 | 13 | 32 | 46 | 60 | 71 |
| Wells | | | | | | | | | | | | | | | |
| Coated with | | Serum | | | | | | | | | | | | | |
| whole cell lysate | | dilution | | 1 | 2 | 3 | 4 | 5 | 6 | 7 | 8 | 9 | 10 | 11 | 12 |
| FA1090: | | 100 | A | 0.021 | 0.580 | 0.773 | 0.934 | 1.260 | 1.360 | 0.019 | 0.332 | 0.508 | 0.593 | 0.716 | 0.733 |
| [A-C; 1-6] | | | | | | | | | | | | | | | |
| F62(ΔD); | | 500 | B | 0.017 | 0.127 | 0.074 | 0.143 | 0.216 | 0.199 | 0.016 | 0.027 | 0.065 | 0.125 | 0.099 | 0.334 |
| [A-C; 7-12] | | | | | | | | | | | | | | | |
| MS11(Opa−): | | 1000 | C | 0.023 | 0.034 | 0.013 | 0.047 | 0.091 | 0.072 | 0.008 | 0.012 | 0.013 | 0.016 | 0.011 | 0.035 |
| [D-F; 1-6] | | | | | | | | | | | | | | | |
| H041: | | 100 | D | 0.015 | 0.711 | 1.017 | 1.142 | 1.349 | 1.533 | 0.020 | 0.033 | 0 069 | 0.082 | 0.185 | 0.201 |
| [D-F; 7-12] | | | | | | | | | | | | | | | |
| Antibody Control: | | 500 | E | 0.019 | 0.091 | 0.151 | 0.183 | 0.233 | 0.287 | 0.017 | 0.016 | 0.025 | 0.021 | 0.024 | 0.054 |
| [G-H; 1-6] | | | | | | | | | | | | | | | |
| Coat Control: | | 1000 | F | 0.009 | 0.013 | 0.045 | 0.079 | 0.095 | 0.128 | 0.015 | 0.012 | 0.015 | 0.013 | 0.016 | 0.009 |
| [G-H; 7-10] | | | | | | | | | | | | | | | |
| Conjugate Control: | | 100 | G | 0.013 | 0.007 | 0.009 | 0.009 | 0.007 | 0.006 | 0.011 | 0.020 | 0.014 | 0.009 | 0.009 | 0.001 |
| [G-H; 11] | | | | | | | | | | | | | | | |
| Substrate Control: | | 100 | H | 0.020 | 0.012 | 0.008 | 0.012 | 0.011 | 0.009 | 0.012 | 0.018 | 0.013 | 0.018 | 0.016 | −0.001 |
| [G-H; 12] | | | | | | | | | | | | | | | |

Plate 1

| | | GROUP 2 | | | | | | |
|---|---|---|---|---|---|---|---|---|
| | | Immunogen | | NGO1496 + NGO0571 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO 1496 | 100 | A | 0.043 | 4.193 | 5.669 | 5.735 | 5.609 | 5.433 |
| | 500 | B | 0.034 | 1.622 | 5.435 | 5.561 | 5.512 | 5.256 |
| | 5000 | C | 0.036 | 0.197 | 4.815 | 4.814 | 4.944 | 4.308 |
| | 25000 | D | 0.020 | 0.059 | 4.235 | 4.036 | 4.813 | 3.831 |
| NGO 0571 | 100 | E | 0.033 | 0.941 | 4.052 | 4.610 | 4.398 | 4.308 |
| | 500 | F | 0.036 | 0.429 | 2.233 | 4.309 | 4.081 | 4.190 |
| | 5000 | G | 0.024 | 0.073 | 0.707 | 2.471 | 2.632 | 2.104 |
| | 25000 | H | 0.035 | 0.057 | 0.206 | 0.949 | 0.923 | 0.872 |

Plate 2

| | | GROUP 2 | | | | | | | | | | | | |
|---|---|---|---|---|---|---|---|---|---|---|---|---|---|---|
| | | Immunogen | | NGO1496 + NGO0571 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.043 | 0.588 | 2.577 | 4.613 | 3.547 | 3.149 | 0.027 | 0.667 | 3.082 | 5.943 | 4.335 | 4.149 |
| [A-C; 1-6] | | | | | | | | | | | | | | |
| F62 (ΔD): | 500 | B | 0.050 | 0.192 | 1.663 | 2.650 | 2.086 | 2.131 | 0.015 | 0.201 | 1.889 | 2.956 | 2.204 | 2.788 |
| [A-C; 7-12] | | | | | | | | | | | | | | |
| MS11 (Opa-): | 1000 | C | 0.053 | 0.138 | 0.978 | 1.879 | 1.403 | 1.430 | 0.019 | 0.111 | 1.142 | 2.029 | 1.746 | 1.560 |
| [D-F; 1-6] | | | | | | | | | | | | | | |
| H041: | 100 | D | 0.041 | 0.438 | 2.900 | 4.261 | 3.834 | 3.218 | 0.024 | 0.539 | 1.677 | 3.726 | 3.062 | 2.481 |
| [D-F; 7-12] | | | | | | | | | | | | | | |
| Antibody Control: | 500 | E | 0.032 | 0.135 | 1.539 | 2.515 | 1.831 | 2.267 | 0.026 | 0.180 | 1.182 | 2.198 | 1.703 | 1.414 |
| [G-H; 1-6] | | | | | | | | | | | | | | |
| Coat Control: | 1000 | F | 0.021 | 0.090 | 1.068 | 1.717 | 1.390 | 1.505 | 0.021 | 0.234 | 0.918 | 1.532 | 1.007 | 1.174 |
| [G-H; 7-10] | | | | | | | | | | | | | | |
| Conjugate Control: | 100 | G | 0.003 | 0.009 | −0.001 | 0.008 | 0.006 | −0.007 | −0.001 | −0.011 | −0.003 | −0.015 | −0.015 | −0.003 |
| [G-H; 11] | | | | | | | | | | | | | | |
| Substrate Control: | 100 | H | 0.032 | 0.010 | 0.004 | 0.005 | −0.011 | 0.004 | −0.013 | −0.011 | −0.015 | −0.013 | −0.008 | 0.003 |
| [G-H; 12] | | | | | | | | | | | | | | |

Plate 3

| | | GROUP 3 | | | | | | |
|---|---|---|---|---|---|---|---|---|
| | | Immunogen | | NGO1379 + NGO0725 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO1379 | 100 | A | 0.054 | 0.670 | 3.138 | 5.954 | 5.954 | 5.954 |
| | 500 | B | 0.053 | 0.508 | 1.946 | 2.988 | 2.993 | 3.192 |
| | 5000 | C | 0.037 | 0.140 | 1.467 | 1.459 | 1.561 | 1.791 |
| | 25000 | D | 0.035 | 0.069 | 0.768 | 0.947 | 1.010 | 0.915 |
| NGO0725 | 100 | E | 0.052 | 0.629 | 5.954 | 5.954 | 5.504 | 4.462 |
| | 500 | F | 0.050 | 0.159 | 5.954 | 5.954 | 4.902 | 4.902 |
| | 5000 | G | 0.038 | 0.052 | 2.433 | 4.072 | 2.845 | 3.462 |
| | 25000 | H | 0.042 | 0.041 | 1.005 | 1.691 | 1.235 | 1.339 |

Plate 4

| | | GROUP 3 | | | | | | | | | | | | |
|---|---|---|---|---|---|---|---|---|---|---|---|---|---|---|
| | | Immunogen | | NGO1379 + NGO0725 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.044 | 0.670 | 3.138 | 5.954 | 4.390 | 5.954 | 0.005 | 0.266 | 0.618 | 0.819 | 1.328 | 2.154 |
| [A-C; 1-6] | | | | | | | | | | | | | | |
| F62 (ΔD): | 500 | B | 0.041 | 0.508 | 1.946 | 2.988 | 2.993 | 3.192 | 0.004 | 0.211 | 0.315 | 0.420 | 0.954 | 1.820 |
| [A-C; 7-12] | | | | | | | | | | | | | | |
| MS11 (Opa-): | 1000 | C | 0.037 | 0.140 | 1.467 | 1.459 | 1.561 | 1.791 | 0.007 | 0.108 | 0.225 | 0.222 | 0.715 | 1.200 |
| [D-F; 1-6] | | | | | | | | | | | | | | |
| H041: | 100 | D | 0.035 | 2.069 | 5.954 | 5.954 | 5.954 | 5.954 | 0.025 | 0.199 | 0.604 | 0.717 | 1.314 | 2.206 |
| [D-F; 7-12] | | | | | | | | | | | | | | |
| Antibody Control: | 500 | E | 0.032 | 0.629 | 5.954 | 5.954 | 5.504 | 4.462 | 0.013 | 0.120 | 0.275 | 0.399 | 0.705 | 1.506 |
| [G-H; 1-6] | | | | | | | | | | | | | | |
| Coat Control: | 1000 | F | 0.020 | 0.159 | 5.954 | 5.954 | 4.902 | 4.902 | −0.001 | 0.054 | 0.164 | 0.210 | 0.297 | 0.799 |
| [G-H; 7-10] | | | | | | | | | | | | | | |
| Conjugate Control: | 100 | G | 0.028 | 0.052 | 0.053 | 0.044 | 0.045 | 0.032 | 0.004 | 0.000 | 0.018 | 0.010 | −0.002 | 0.000 |
| [G-H; 11] | | | | | | | | | | | | | | |
| Substrate Control: | 100 | H | 0.042 | 0.041 | 0.035 | 0.044 | 0.035 | 0.039 | 0.021 | 0.019 | 0.015 | 0.002 | 0.001 | 0.000 |
| [G-H; 12] | | | | | | | | | | | | | | |

Plate 5

| | | GROUP 4 | | | | | | |
|---|---|---|---|---|---|---|---|---|
| | | Immunogen | | NGO1158 + NGO0182 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO1158 | 100 | A | 0.040 | 2.815 | 5.949 | 5.949 | 5.949 | 5.949 |
| | 500 | B | 0.021 | 0.906 | 5.949 | 5.949 | 5.949 | 5.949 |
| | 5000 | C | 0.047 | 0.135 | 5.949 | 4.718 | 4.292 | 4.350 |
| | 25000 | D | 0.033 | 0.057 | 2.736 | 2.767 | 2.326 | 2.125 |
| NGO0182 | 100 | E | 0.027 | 1.312 | 4.382 | 5.949 | 4.455 | 4.350 |
| | 500 | F | 0.038 | 0.341 | 5.949 | 4.797 | 4.797 | 4.455 |
| | 5000 | G | 0.033 | 0.056 | 3.863 | 4.350 | 5.949 | 4.496 |
| | 25000 | H | 0.047 | 0.036 | 1.850 | 3.080 | 2.880 | 1.869 |

Plate 6

| | | GROUP 4 | | | | | | | | | | | | |
|---|---|---|---|---|---|---|---|---|---|---|---|---|---|---|
| | | Immunogen | | NGO1158 + NGO0182 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.024 | 0.544 | 0.985 | 1.744 | 2.075 | 2.539 | −0.001 | −0.002 | −0.001 | 0.001 | 0.003 | −0.002 |
| [A-C; 1-6] | | | | | | | | | | | | | | |
| F62 (ΔD): | 500 | B | 0.025 | 0.169 | 0.297 | 0.624 | 0.751 | 0.744 | −0.002 | 0.006 | −0.002 | 0.003 | 0.001 | −0.002 |
| [A-C; 7-12] | | | | | | | | | | | | | | |
| MS11 (Opa-): | 1000 | C | 0.020 | 0.117 | 0.185 | 0.400 | 0.432 | 0.448 | 0.003 | 0.019 | 0.002 | 0.001 | −0.003 | −0.002 |
| [D-F; 1-6] | | | | | | | | | | | | | | |
| H041: | 100 | D | 0.024 | 0.403 | 1.048 | 1.549 | 1.858 | 2.644 | −0.002 | −0.002 | −0.002 | −0.003 | −0.003 | −0.006 |
| [D-F; 7-12] | | | | | | | | | | | | | | |
| Antibody Control: | 500 | E | 0.016 | 0.129 | 0.305 | 0.652 | 0.757 | 0.855 | 0.043 | 0.036 | −0.002 | −0.002 | 0.001 | −0.003 |
| [G-H; 1-6] | | | | | | | | | | | | | | |
| Coat Control: | 1000 | F | 0.019 | 0.091 | 0.192 | 0.423 | 0.362 | 0.512 | −0.002 | 0.003 | −0.003 | −0.005 | −0.004 | 0.003 |
| [G-H; 7-10] | | | | | | | | | | | | | | |
| Conjugate Control: | 100 | G | 0.032 | 0.032 | 0.031 | 0.033 | 0.034 | 0.039 | 0.004 | 0.005 | 0.014 | 0.015 | 0.016 | 0.002 |
| [G-H; 11] | | | | | | | | | | | | | | |
| Substrate Control: | 100 | H | 0.029 | 0.032 | 0.028 | 0.029 | 0.029 | 0.032 | 0.009 | 0.011 | 0.014 | 0.008 | 0.020 | −0.002 |
| [G-H; 12] | | | | | | | | | | | | | | |

Plate 7

| | | GROUP 5 | | | | | | |
|---|---|---|---|---|---|---|---|---|
| | | Immunogen | | NGO0721 + NGO2105 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO0721 | 100 | A | 0.030 | 2.315 | 4.064 | 4.909 | 5.033 | 4.954 |
| | 500 | B | 0.023 | 1.779 | 3.673 | 4.232 | 4.130 | 4.665 |
| | 5000 | C | 0.039 | 0.506 | 2.954 | 4.256 | 3.786 | 3.857 |
| | 25000 | D | 0.039 | 0.179 | 2.529 | 4.049 | 2.399 | 2.425 |
| NGO2105 | 100 | E | 0.045 | 4.151 | 5.954 | 5.954 | 5.954 | 5.954 |
| | 500 | F | 0.043 | 1.097 | 5.511 | 5.954 | 5.954 | 5.954 |
| | 5000 | G | 0.046 | 0.151 | 4.608 | 5.954 | 5.033 | 5.954 |
| | 25000 | H | 0.044 | 0.070 | 3.096 | 5.954 | 4.556 | 5.209 |

Plate 8

| | | GROUP 5 | | | | | | | | | | | | |
|---|---|---|---|---|---|---|---|---|---|---|---|---|---|---|
| | | Immunogen | | NGO0721 + NGO2105 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.022 | 0.529 | 1.054 | 2.013 | 2.214 | 4.560 | 0.027 | 0.410 | 1.091 | 1.847 | 2.015 | 5.951 |
| [A-C; 1-6] | | | | | | | | | | | | | | |
| F62 (ΔD): | 500 | B | 0.021 | 0.183 | 0.322 | 0.891 | 0.659 | 1.879 | 0.013 | 0.172 | 0.331 | 0.817 | 0.719 | 1.808 |
| [A-C; 7-12] | | | | | | | | | | | | | | |
| MS11 (Opa-): | 1000 | C | 0.021 | 0.132 | 0.186 | 0.494 | 0.589 | 1.198 | 0.021 | 0.121 | 0.240 | 0.618 | 0.488 | 1.029 |
| [D-F; 1-6] | | | | | | | | | | | | | | |
| H041: | 100 | D | 0.016 | 0.330 | 1.103 | 1.484 | 1.727 | 4.669 | 0.027 | 0.407 | 0.717 | 1.241 | 1.467 | 5.210 |
| [D-F; 7-12] | | | | | | | | | | | | | | |
| Antibody Control: | 500 | E | 0.022 | 0.179 | 0.321 | 0.591 | 0.505 | 1.541 | 0.028 | 0.153 | 0.287 | 1.409 | 0.495 | 1.499 |
| [G-H; 1-6] | | | | | | | | | | | | | | |
| Coat Control: | 1000 | F | 0.023 | 0.139 | 0.212 | 0.438 | 0.572 | 0.993 | 0.027 | 0.104 | 0.159 | 0.251 | 0.288 | 0.820 |
| [G-H; 7-10] | | | | | | | | | | | | | | |
| Conjugate Control: | 100 | G | −0.003 | −0.003 | −0.002 | −0.003 | −0.003 | 0.002 | 0.000 | 0.063 | −0.004 | 0.021 | 0.010 | 0.003 |
| [G-H; 11] | | | | | | | | | | | | | | |
| Substrate Control: | 100 | H | −0.004 | 0.002 | −0.002 | −0.004 | −0.003 | 0.001 | 0.048 | −0.003 | 0.013 | −0.005 | 0.035 | −0.003 |
| [G-H; 12] | | | | | | | | | | | | | | |

Plate 9

| | | GROUP 6 | | | | | | | | | | | | | |
|---|---|---|---|---|---|---|---|---|---|---|---|---|---|---|---|
| | | Immunogen | | NGO1094 + NGO1043 + NGO2059 | | | | | | | | | | | |
| | | Pool Serum | | | | | | | | | | | | | |
| | | Wk | | | | | | | | | | | | | |
| | | | 0 | 2 | 5 | 7 | 9 | 11 | 0 | 2 | 5 | 7 | 9 | 11 | |
| | | Days | | | | | | | | | | | | | |
| Wells | | | −1 | 13 | 32 | 46 | 60 | 71 | −1 | 13 | 32 | 46 | 60 | 71 | |
| Coated | Serum | | | | | | | | | | | | | | |
| with | dilution | | 1 | 2 | 3 | 4 | 5 | 6 | 7 | 8 | 9 | 10 | 11 | 12 | |
| NGO1094 | 100 | A | 0.044 | 0.109 | 3.169 | 4.831 | 3.610 | 4.625 | 0.040 | 0.328 | 2.802 | 4.826 | 5.048 | 5.954 | NGO2059 |
| | 500 | B | 0.045 | 0.128 | 1.929 | 4.928 | 2.762 | 4.487 | 0.039 | 0.090 | 0.888 | 4.349 | 5.954 | 5.954 | |
| | 5000 | C | 0.047 | 0.086 | 0.403 | 1.828 | 1.767 | 2.032 | 0.036 | 0.088 | 0.219 | 2.159 | 1.765 | 2.205 | |
| | 25000 | D | 0.045 | 0.092 | 0.281 | 1.052 | 0.679 | 1.053 | 0.040 | 0.078 | 0.108 | 1.173 | 0.727 | 0.669 | |
| NGO1043 | 100 | E | 0.041 | 0.172 | 0.334 | 0.652 | 0.502 | 0.433 | | | | | | | |
| | 500 | F | 0.035 | 0.085 | 0.275 | 0.489 | 0.457 | 0.292 | | | | | | | |
| | 5000 | G | 0.035 | 0.067 | 0.254 | 0.244 | 0.289 | 0.195 | | | | | | | |
| | 25000 | H | 0.042 | 0.054 | 0.177 | 0.086 | 0.157 | 0.160 | | | | | | | |

Plate 10

| | | GROUP 6 | | | | | | | | | | | | |
|---|---|---|---|---|---|---|---|---|---|---|---|---|---|---|
| | | Immunogen | | NGO1094 + NGO1043 + NGO2059 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.045 | 0.931 | 1.540 | 2.037 | 1.643 | 1.668 | 0.030 | 0.845 | 1.456 | 1.828 | 1.453 | 1.937 |
| [A-C; 1-6] | | | | | | | | | | | | | | |
| F62 (ΔD): | 500 | B | 0.044 | 0.480 | 0.924 | 1.019 | 0.890 | 1.080 | 0.028 | 0.395 | 0.760 | 1.161 | 0.905 | 0.956 |
| [A-C; 7-12] | | | | | | | | | | | | | | |
| MS11 (Opa-): | 1000 | C | 0.040 | 0.395 | 0.545 | 0.964 | 0.527 | 0.690 | 0.030 | 0.273 | 0.587 | 1.041 | 0.566 | 0.890 |
| [D-F; 1-6] | | | | | | | | | | | | | | |
| H041: | 100 | D | 0.039 | 0.595 | 0.965 | 1.640 | 1.472 | 1.721 | 0.046 | 0.790 | 1.073 | 1.553 | 1.220 | 1.515 |
| [D-F; 7-12] | | | | | | | | | | | | | | |
| Antibody Control: | 500 | E | 0.046 | 0.322 | 0.618 | 0.808 | 0.665 | 1.047 | 0.044 | 0.296 | 0.593 | 0.835 | 0.625 | 0.885 |
| [G-H; 1-6] | | | | | | | | | | | | | | |
| Coat Control: | 1000 | F | 0.038 | 0.256 | 0.350 | 0.588 | 0.449 | 0.767 | 0.038 | 0.238 | 0.447 | 0.731 | 0.480 | 0.598 |
| [G-H; 7-10] | | | | | | | | | | | | | | |
| Conjugate Control: | 100 | G | 0.002 | 0.005 | 0.000 | 0.003 | 0.020 | 0.028 | 0.001 | 0.002 | 0.019 | 0.001 | 0.001 | 0.002 |
| [G-H; 11] | | | | | | | | | | | | | | |
| Substrate Control: | 100 | H | 0.001 | 0.001 | 0.000 | 0.001 | 0.001 | 0.020 | 0.026 | 0.003 | 0.016 | 0.002 | 0.005 | −0.002 |
| [G-H; 12] | | | | | | | | | | | | | | |

Plate 11

| | | GROUP 7 | | | | | | |
|---|---|---|---|---|---|---|---|---|
| | | Immunogen | | NGO1984 + NGO1286 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO1984 | 100 | A | 0.042 | 0.061 | 0.308 | 1.037 | 1.023 | 0.837 |
| | 500 | B | 0.036 | 0.045 | 0.147 | 0.626 | 0.538 | 0.614 |
| | 5000 | C | 0.040 | 0.054 | 0.099 | 0.232 | 0.200 | 0.169 |
| | 25000 | D | 0.035 | 0.041 | 0.082 | 0.141 | 0.120 | 0.116 |
| NGO1286 | 100 | E | 0.039 | 4.602 | 5.956 | 5.956 | 5.956 | 5.956 |
| | 500 | F | 0.043 | 3.501 | 5.204 | 5.956 | 5.956 | 5.956 |
| | 5000 | G | 0.037 | 0.462 | 4.226 | 4.329 | 5.204 | 4.551 |
| | 25000 | H | 0.043 | 0.175 | 2.962 | 3.974 | 3.275 | 3.099 |

Plate 12

| | | GROUP 7 | | | | | | | | | | | | |
|---|---|---|---|---|---|---|---|---|---|---|---|---|---|---|
| | | Immunogen | | NGO1984 + NGO1286 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.042 | 1.359 | 4.597 | 5.954 | 5.954 | 4.801 | 0.021 | 1.856 | 5.954 | 5.198 | 5.023 | 5.597 |
| [A-C; 1-6] | | | | | | | | | | | | | | |
| F62 (ΔD): | 500 | B | 0.041 | 0.649 | 4.898 | 5.954 | 4.898 | 4.597 | 0.035 | 0.845 | 4.545 | 4.800 | 4.898 | 5.500 |
| [A-C; 7-12] | | | | | | | | | | | | | | |
| MS11 (Opa-): | 1000 | C | 0.032 | 0.410 | 3.944 | 4.597 | 4.296 | 4.354 | 0.027 | 0.528 | 4.385 | 5.022 | 4.138 | 5.954 |
| [D-F; 1-6] | | | | | | | | | | | | | | |
| H041: | 100 | D | 0.026 | 1.279 | 4.722 | 5.954 | 4.655 | 5.500 | 0.037 | 1.665 | 4.295 | 4.545 | 4.655 | 5.500 |
| [D-F; 7-12] | | | | | | | | | | | | | | |
| Antibody Control: | 500 | E | 0.025 | 0.466 | 3.643 | 4.546 | 4.898 | 4.722 | 0.044 | 0.776 | 3.908 | 4.721 | 4.500 | 5.954 |
| [G-H; 1-6] | | | | | | | | | | | | | | |
| Coat Control: | 1000 | F | 0.030 | 0.442 | 4.178 | 4.722 | 4.454 | 4.065 | 0.041 | 0.516 | 3.783 | 4.545 | 4.296 | 5.500 |
| [G-H; 7-10] | | | | | | | | | | | | | | |
| Conjugate Control: | 100 | G | −0.002 | −0.004 | −0.004 | −0.001 | 0.015 | 0.025 | −0.003 | 0.017 | 0.016 | 0.012 | −0.004 | 0.004 |
| [G-H; 11] | | | | | | | | | | | | | | |
| Substrate Control: | 100 | H | −0.004 | −0.003 | −0.003 | −0.003 | −0.003 | −0.002 | 0.009 | 0.019 | 0.013 | 0.007 | 0.024 | −0.004 |
| [G-H; 12] | | | | | | | | | | | | | | |

Plate 13

| | | GROUP 8 | | | | | | |
|---|---|---|---|---|---|---|---|---|
| | | Immunogen | | NGO0275 + NGO0225 | | | | |
| | | Pool Serum | | | | | | |
| | | Wk | | | | | | |
| | | | 0 | 2 | 5 | 7 | 9 | 11 |
| | | Days | | | | | | |
| Wells | | | 4 | 13 | 32 | 46 | 60 | 71 |
| Coated | Serum | | | | | | | |
| with | dilution | | 1 | 2 | 3 | 4 | 5 | 6 |
| NGO0275 | 100 | A | 0.031 | 4.236 | 5.947 | 5.947 | 4.558 | 4.909 |
| | 500 | B | 0.028 | 1.428 | 5.947 | 5.947 | 4.558 | 4.256 |
| | 5000 | C | 0.029 | 0.257 | 4.337 | 4.667 | 3.947 | 3.034 |
| | 25000 | D | 0.030 | 0.115 | 3.080 | 3.583 | 3.516 | 1.793 |
| NGO0225 | 100 | E | 0.031 | 4.815 | 5.947 | 5.947 | 5.947 | 5.947 |
| | 500 | F | 0.031 | 4.514 | 5.947 | 5.947 | 5.947 | 5.947 |
| | 5000 | G | 0.027 | 3.194 | 5.947 | 5.947 | 5.947 | 5.947 |
| | 25000 | H | 0.036 | 2.327 | 4.559 | 5.947 | 5.947 | 5.947 |

Plate 14

| | | GROUP 8 | | | | | | | | | | | | |
|---|---|---|---|---|---|---|---|---|---|---|---|---|---|---|
| | | Immunogen | | NGO0275 + NGO0225 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.043 | 0.851 | 1.273 | 1.467 | 1.300 | 0.833 | 0.044 | 0.749 | 1.239 | 1.488 | 1.604 | 1.318 |
| [A-C; 1-6] | | | | | | | | | | | | | | |
| F62 (ΔD): | 500 | B | 0.045 | 0.343 | 0.817 | 0.504 | 0.583 | 0.463 | 0.035 | 0.404 | 0.856 | 0.586 | 0.965 | 0.856 |
| [A-C; 7-12] | | | | | | | | | | | | | | |
| MS11 (Opa-): | 1000 | C | 0.036 | 0.234 | 0.743 | 0.356 | 0.362 | 1.851 | 0.039 | 0.178 | 0.530 | 0.421 | 0.750 | 0.684 |
| [D-F; 1-6] | | | | | | | | | | | | | | |
| H041: | 100 | D | 0.031 | 0.317 | 0.650 | 0.935 | 1.046 | 1.120 | 0.036 | 0.577 | 0.759 | 0.987 | 1.153 | 0.518 |
| [D-F; 7-12] | | | | | | | | | | | | | | |
| Antibody Control: | 500 | E | 0.041 | 0.144 | 0.410 | 0.337 | 0.630 | 0.582 | 0.042 | 0.205 | 0.510 | 0.311 | 0.584 | 0.583 |
| [G-H; 1-6] | | | | | | | | | | | | | | |
| Coat Control: | 1000 | F | 0.040 | 0.129 | 0.378 | 0.216 | 0.455 | 0.553 | 0.036 | 0.153 | 0.364 | 0.250 | 0.475 | 0.419 |
| [G-H; 7-10] | | | | | | | | | | | | | | |
| Conjugate Control: | 100 | G | −0.002 | 0.028 | −0.004 | 0.030 | 0.020 | 0.032 | 0.016 | 0.031 | 0.016 | −0.001 | 0.013 | 0.001 |
| [G-H; 11] | | | | | | | | | | | | | | |
| Substrate Control: | 100 | H | 0.001 | −0.001 | −0.002 | 0.014 | −0.002 | 0.014 | 0.013 | 0.043 | 0.013 | 0.031 | 0.003 | −0.001 |
| [G-H; 12] | | | | | | | | | | | | | | |

Plate 15

| | | GROUP 9 | | | | | | |
|---|---|---|---|---|---|---|---|---|
| | | Immunogen | | NGO1495 + NGO2093 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO1495 | 100 | A | 0.032 | 0.415 | 5.948 | 5.948 | 5.211 | 5.948 |
| | 500 | B | 0.029 | 0.174 | 2.606 | 5.948 | 4.813 | 5.948 |
| | 5000 | C | 0.038 | 0.053 | 0.664 | 1.705 | 1.157 | 1.038 |
| | 25000 | D | 0.033 | 0.047 | 0.162 | 0.489 | 0.378 | 0.397 |
| NGO2093 | 100 | E | 0.030 | 4.815 | 5.948 | 5.948 | 5.948 | 5.948 |
| | 500 | F | 0.030 | 2.622 | 5.948 | 5.948 | 5.948 | 5.948 |
| | 5000 | G | 0.033 | 0.322 | 5.948 | 5.948 | 4.609 | 5.948 |
| | 25000 | H | 0.032 | 0.142 | 4.559 | 5.513 | 4.366 | 4.281 |

Plate 16

| | | GROUP 9 | | | | | | | | | | | | |
|---|---|---|---|---|---|---|---|---|---|---|---|---|---|---|
| | | Immunogen | | NGO1495 + NGO2093 | | | | | | | | | | |
| | | Wk | | | | | | | | | | | | |
| | | | 0 | 2 | 5 | 7 | 9 | 11 | 0 | 2 | 5 | 7 | 9 | 11 |
| | | Pool Serum | | | | | | | | | | | | |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.038 | 0.834 | 2.021 | 2.612 | 3.687 | 2.840 | 0.042 | 1.069 | 2.427 | 3.290 | 4.068 | 3.351 |
| [A-C; 1-6] | | | | | | | | | | | | | | |
| F62 (ΔD): | 500 | B | 0.035 | 0.350 | 1.001 | 1.370 | 1.904 | 1.743 | 0.035 | 0.339 | 0.961 | 1.724 | 1.683 | 1.711 |
| [A-C; 7-12] | | | | | | | | | | | | | | |
| MS11 (Opa-): | 1000 | C | 0.032 | 0.315 | 0.733 | 1.025 | 1.315 | 1.446 | 0.034 | 0.193 | 0.614 | 1.260 | 1.408 | 1.668 |
| [D-F; 1-6] | | | | | | | | | | | | | | |
| H041: | 100 | D | 0.037 | 0.438 | 1.490 | 2.209 | 3.661 | 2.167 | 0.035 | 0.629 | 2.012 | 2.702 | 3.847 | 3.240 |
| [D-F; 7-12] | | | | | | | | | | | | | | |
| Antibody Control: | 500 | E | 0.032 | 0.196 | 0.706 | 1.120 | 1.413 | 1.455 | 0.039 | 0.263 | 0.905 | 1.817 | 1.950 | 2.079 |
| [G-H; 1-6] | | | | | | | | | | | | | | |
| Coat Control: | 1000 | F | 0.038 | 0.118 | 0.513 | 0.956 | 1.110 | 1.291 | 0.032 | 0.171 | 0.543 | 1.590 | 1.214 | 1.886 |
| [G-H; 7-10] | | | | | | | | | | | | | | |
| Conjugate Control: | 100 | G | −0.001 | −0.005 | 0.019 | 0.018 | −0.002 | 0.018 | −0.002 | −0.002 | 0.015 | 0.010 | 0.012 | −0.004 |
| [G-H; 11] | | | | | | | | | | | | | | |
| Substrate Control: | 100 | H | 0.002 | −0.004 | 0.001 | −0.005 | −0.004 | 0.006 | 0.011 | −0.001 | −0.006 | −0.005 | 0.006 | 0.004 |
| [G-H; 12] | | | | | | | | | | | | | | |

Plate 17

| | | GROUP 10 | | | | | | | | | | | | | |
|---|---|---|---|---|---|---|---|---|---|---|---|---|---|---|---|
| | | Immunogen | | NGO1392 + NGO1585 + NGO2109 | | | | | | | | | | | |
| | | Pool Serum | | | | | | | | | | | | | |
| | | Wk | | | | | | | | | | | | | |
| | | | 0 | 2 | 5 | 7 | 9 | 11 | 0 | 2 | 5 | 7 | 9 | 11 | |
| | | Days | | | | | | | | | | | | | |
| Wells | | | −1 | 13 | 32 | 46 | 60 | 71 | −1 | 13 | 32 | 46 | 60 | 71 | |
| Coated | Serum | | | | | | | | | | | | | | |
| with | dilution | | 1 | 2 | 3 | 4 | 5 | 6 | 7 | 8 | 9 | 10 | 11 | 12 | |
| NGO1392 | 100 | A | 0.019 | 4.702 | 5.938 | 5.938 | 5.938 | 5.938 | 0.018 | 0.200 | 0.502 | 3.010 | 4.781 | 1.586 | NGO2109 |
| | 500 | B | 0.016 | 3.044 | 5.003 | 5.938 | 4.635 | 5.480 | 0.026 | 0.136 | 0.150 | 0.543 | 1.575 | 0.773 | |
| | 5000 | C | 0.036 | 0.775 | 4.439 | 5.938 | 4.439 | 4.878 | 0.027 | 0.129 | 0.129 | 0.139 | 0.257 | 0.115 | |
| | 25000 | D | 0.020 | 0.376 | 4.249 | 4.781 | 4.334 | 4.158 | 0.019 | 0.066 | 0.067 | 0.138 | 0.100 | 0.097 | |
| NGO1585 | 100 | E | 0.025 | 5.938 | 5.938 | 5.938 | 5.938 | 5.938 | | | | | | | |
| | 500 | F | 0.017 | 5.938 | 5.938 | 5.938 | 5.938 | 5.938 | | | | | | | |
| | 5000 | G | 0.023 | 1.303 | 5.938 | 5.938 | 5.938 | 5.938 | | | | | | | |
| | 25000 | H | 0.022 | 0.386 | 4.304 | 4.577 | 4.401 | 4.276 | | | | | | | |

Plate 18

| | | GROUP 10 | | | | | | | | | | | | |
|---|---|---|---|---|---|---|---|---|---|---|---|---|---|---|
| | | Immunogen | | NGO1392 + NG01585 + NGO2109 | | | | | | | | | | |
| | | Pool Serum | | | | | | | | | | | | |
| | | Wk | | | | | | | | | | | | |
| | | | 0 | 2 | 5 | 7 | 9 | 11 | 0 | 2 | 5 | 7 | 9 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.023 | 0.425 | 0.524 | 0.848 | 1.288 | 0.928 | 0.015 | 0.321 | 0.687 | 0.956 | 0.883 | 0.783 |
| [A-C; 1-6] | | | | | | | | | | | | | | |
| F62 (ΔD): | 500 | B | 0.017 | 0.100 | 0.177 | 0.141 | 0.380 | 0.227 | 0.013 | 0.084 | 0.179 | 0.233 | 0.399 | 0.258 |
| [A-C; 7-12] | | | | | | | | | | | | | | |
| MS11 (Opa-): | 1000 | C | 0.016 | 0.076 | 0.111 | 0.095 | 0.186 | 0.232 | 0.022 | 0.074 | 0.132 | 0.159 | 0.205 | 0.177 |
| [D-F; 1-6] | | | | | | | | | | | | | | |
| H041: | 100 | D | 0.013 | 0.324 | 0.475 | 0.663 | 1.631 | 0.433 | 0.007 | 1.021 | 0.982 | 1.352 | 2.388 | 1.212 |
| [D-F; 7-12] | | | | | | | | | | | | | | |
| Antibody Control: | 500 | E | 0.015 | 0.077 | 0.153 | 0.150 | 0.229 | 0.230 | 0.015 | 0.203 | 0.354 | 0.250 | 0.408 | 0.316 |
| [G-H; 1-6] | | | | | | | | | | | | | | |
| Coat Control: | 1000 | F | 0.012 | 0.060 | 0.109 | 0.093 | 0.146 | 0.115 | 0.009 | 0.137 | 0.227 | 0.166 | 0.272 | 0.259 |
| [G-H; 7-10] | | | | | | | | | | | | | | |
| Conjugate Control: | 100 | G | 0.018 | −0.003 | 0.008 | 0.011 | 0.008 | 0.009 | 0.017 | 0.012 | 0.009 | 0.016 | 0.006 | 0.001 |
| [G-H; 11] | | | | | | | | | | | | | | |
| Substrate Control: | 100 | H | 0.008 | 0.021 | 0.008 | 0.009 | 0.006 | 0.008 | 0.007 | 0.018 | 0.025 | 0.024 | 0.005 | −0.001 |
| [G-H; 12] | | | | | | | | | | | | | | |

Plate 19

| | | GROUP 11 | | | | | | | | | | | | | |
|---|---|---|---|---|---|---|---|---|---|---|---|---|---|---|---|
| | | Immunogen | | NGO1801 + NGO0952 + NGO1715 | | | | | | | | | | | |
| | | Pool Serum | | | | | | | | | | | | | |
| | | Wk | | | | | | | | | | | | | |
| | | | 0 | 2 | 5 | 7 | 9 | 11 | 0 | 2 | 5 | 7 | 9 | 11 | |
| | | Days | | | | | | | | | | | | | |
| Wells | | | −1 | 13 | 32 | 46 | 60 | 71 | −1 | 13 | 32 | 46 | 60 | 71 | |
| Coated | Serum | | | | | | | | | | | | | | |
| with | dilution | | 1 | 2 | 3 | 4 | 5 | 6 | 7 | 8 | 9 | 10 | 11 | 12 | |
| NGO1801 | 100 | A | 0.007 | 5.936 | 5.936 | 5.936 | 4.482 | 5.936 | 0.016 | 5.936 | 5.936 | 5.936 | 5.936 | 5.181 | NG01715 |
| | 500 | B | 0.014 | 1.833 | 5.482 | 5.936 | 4.368 | 5.936 | 0.017 | 2.235 | 5.936 | 5.936 | 5.936 | 4.482 | |
| | 5000 | C | 0.012 | 0.407 | 5.005 | 5.936 | 4.203 | 5.005 | 0.018 | 0.466 | 5.936 | 5.936 | 5.936 | 4.528 | |
| | 25000 | D | 0.013 | 0.207 | 4.067 | 3.625 | 3.977 | 3.683 | 0.019 | 0.255 | 4.084 | 3.711 | 4.368 | 3.656 | |
| NGO0952 | 100 | E | 0.017 | 2.110 | 5.936 | 5.936 | 5.936 | 5.936 | | | | | | | |
| | 500 | F | 0.024 | 1.718 | 5.936 | 5.936 | 5.936 | 5.936 | | | | | | | |
| | 5000 | G | 0.019 | 1.818 | 5.005 | 4.783 | 4.482 | 4.278 | | | | | | | |
| | 25000 | H | 0.025 | 0.839 | 3.656 | 3.445 | 2.826 | 3.352 | | | | | | | |

Plate 20

| | | GROUP 11 | | | | | | | | | | | | |
|---|---|---|---|---|---|---|---|---|---|---|---|---|---|---|
| | | Immunogen | | NGO1801 + NGO0952 + NG01715 | | | | | | | | | | |
| | | Pool Serum | | | | | | | | | | | | |
| | | Wk | | | | | | | | | | | | |
| | | | 0 | 2 | 5 | 7 | 9 | 11 | 2 | 5 | 7 | 9 | 2 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.017 | 0.369 | 0.831 | 1.300 | 1.906 | 1.782 | 0.026 | 0.334 | 1.256 | 1.481 | 1.206 | 0.935 |
| [A-C; 1-6] | | | | | | | | | | | | | | |
| F62 (ΔD): | 500 | B | 0.021 | 0.132 | 0.319 | 0.409 | 0.723 | 0.555 | 0.025 | 0.121 | 0.392 | 0.605 | 0.615 | 0.288 |
| [A-C; 7-12] | | | | | | | | | | | | | | |
| MS11 (Opa-): | 1000 | C | 0.023 | 0.106 | 0.244 | 0.263 | 0.546 | 0.377 | 0.027 | 0.077 | 0.262 | 0.487 | 0.595 | 0.285 |
| [D-F; 1-6] | | | | | | | | | | | | | | |
| H041: | 100 | D | 0.032 | 0.306 | 0.870 | 0.854 | 1.158 | 1.001 | 0.030 | 0.358 | 0.721 | 1.059 | 1.146 | 1.261 |
| [D-F; 7-12] | | | | | | | | | | | | | | |
| Antibody Control: | 500 | E | 0.027 | 0.135 | 0.293 | 0.338 | 0.444 | 0.418 | 0.029 | 0.094 | 0.264 | 0.339 | 0.610 | 0.598 |
| [G-H; 1-6] | | | | | | | | | | | | | | |
| Coat Control: | 1000 | F | 0.029 | 0.088 | 0.195 | 0.200 | 0.328 | 0.279 | 0.028 | 0.071 | 0.160 | 0.204 | 0.443 | 0.308 |
| [G-H; 7-10] | | | | | | | | | | | | | | |
| Conjugate Control: | 100 | G | 0.005 | 0.006 | 0.005 | 0.006 | 0.006 | 0.009 | 0.007 | 0.008 | 0.012 | 0.006 | 0.005 | 0.001 |
| [G-H; 11] | | | | | | | | | | | | | | |
| Substrate Control: | 100 | H | 0.009 | 0.008 | 0.006 | 0.007 | 0.005 | 0.006 | 0.005 | 0.005 | 0.001 | 0.016 | 0.004 | −0.001 |
| [G-H; 12] | | | | | | | | | | | | | | |

Plate 21

| | | GROUP 12 | | | | | | |
|---|---|---|---|---|---|---|---|---|
| | | Immunogen | | NGO1549 + NGO0265 | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| NGO1549 | 100 | A | 0.019 | 0.090 | 0.155 | 4.498 | 4.418 | 5.195 |
| | 500 | B | 0.028 | 0.023 | 0.047 | 3.280 | 2.559 | 3.236 |
| | 5000 | C | 0.017 | 0.023 | 0.048 | 0.932 | 0.850 | 0.696 |
| | 25000 | D | 0.014 | 0.066 | 0.047 | 0.318 | 0.245 | 0.259 |
| NGO0265 | 100 | E | 0.020 | 0.059 | 0.127 | 0.672 | 0.886 | 1.157 |
| | 500 | F | 0.019 | 0.022 | 0.022 | 0.383 | 0.270 | 0.422 |
| | 5000 | G | 0.018 | 0.012 | 0.034 | 0.175 | 0.106 | 0.184 |
| | 25000 | H | 0.021 | 0.009 | 0.011 | 0.092 | 0.051 | 0.129 |

Plate 22

| | | GROUP 12 | | | | | | | | | | | | |
|---|---|---|---|---|---|---|---|---|---|---|---|---|---|---|
| | | Immunogen | | NGO1549 + NGO0265 | | | | | | | | | | |
| | | Pool Serum | | | | | | | | | | | | |
| | | Wk | | | | | | | | | | | | |
| | | | 0 | 2 | 5 | 7 | 9 | 11 | 2 | 5 | 7 | 9 | 2 | 11 |
| | | Days | | | | | | | | | | | | |
| Wells Coated | | | −1 | 13 | 32 | 46 | 60 | 71 | −1 | 13 | 32 | 46 | 60 | 71 |
| with whole | Serum | | | | | | | | | | | | | |
| cell lysate: | dilution | | 1 | 2 | 3 | 4 | 5 | 6 | 7 | 8 | 9 | 10 | 11 | 12 |
| FA1090: | 100 | A | 0.011 | 0.609 | 0.510 | 1.068 | 0.986 | 1.142 | 0.009 | 0.426 | 0.410 | 0.893 | 0.753 | 0.680 |
| [A-C; 1-6] | | | | | | | | | | | | | | |
| F62 (ΔD): | 500 | B | 0.008 | 0.185 | 0.111 | 0.274 | 0.268 | 0.371 | 0.011 | 0.153 | 0.098 | 0.303 | 0.280 | 0.331 |
| [A-C; 7-12] | | | | | | | | | | | | | | |
| MS11 (Opa-): | 1000 | C | 0.006 | 0.125 | 0.081 | 0.199 | 0.171 | 0.238 | 0.005 | 0.152 | 0.065 | 0.184 | 0.155 | 0.182 |
| [D-F; 1-6] | | | | | | | | | | | | | | |
| H041: | 100 | D | 0.011 | 0.356 | 0.359 | 0.780 | 1.013 | 0.544 | 0.011 | 0.370 | 0.403 | 1.322 | 0.768 | 1.631 |
| [D-F; 7-12] | | | | | | | | | | | | | | |
| Antibody Control: | 500 | E | 0.014 | 0.129 | 0.089 | 0.277 | 0.278 | 0.276 | 0.004 | 0.131 | 0.096 | 0.650 | 0.230 | 0.713 |
| [G-H; 1-6] | | | | | | | | | | | | | | |
| Coat Control: | 1000 | F | 0.009 | 0.095 | 0.065 | 0.187 | 0.219 | 0.190 | 0.010 | 0.099 | 0.068 | 0.424 | 0.173 | 0.442 |
| [G-H; 7-10] | | | | | | | | | | | | | | |
| Conjugate Control: | 100 | G | 0.006 | 0.007 | 0.004 | 0.005 | 0.011 | 0.011 | 0.005 | 0.006 | 0.005 | 0.006 | 0.004 | 0.001 |
| [G-H; 11] | | | | | | | | | | | | | | |
| Substrate Control: | 100 | H | 0.006 | 0.011 | 0.004 | 0.007 | 0.005 | 0.008 | 0.012 | 0.007 | 0.005 | 0.012 | 0.003 | −0.001 |
| [G-H; 12] | | | | | | | | | | | | | | |

Plate 23

| | | GROUP 13 | | | | | | |
|---|---|---|---|---|---|---|---|---|
| | | Immunogen | | GLA-SE alone | | | | |
| | | Pool Serum | | | | | | |
| | | Wk | | | | | | |
| | | | 0 | 2 | 5 | 7 | 9 | 11 |
| | | Days | | | | | | |
| Wells | | | −1 | 13 | 32 | 46 | 60 | 71 |
| Coated | Serum | | | | | | | |
| with | dilution | | 1 | 2 | 3 | 4 | 5 | 6 |
| Protein mix | 100 | A | 0.014 | 0.139 | 0.135 | 0.158 | 0.166 | 0.155 |
| | 500 | B | 0.011 | 0.071 | 0.105 | 0.097 | 0.101 | 0.088 |
| | 5000 | C | 0.011 | 0.045 | 0.052 | 0.066 | 0.047 | 0.035 |
| | 25000 | D | 0.007 | 0.006 | 0.022 | 0.012 | 0.008 | 0.009 |

Plate 25

| | | | GROUP 13 | | | | |
|---|---|---|---|---|---|---|---|
| | | | Immunogen | | GLA-SE alone | | |
| Wells Coated | | | Pool Serum | | | | |
| with whole | | Serum | | | | | |
| cell lysate: | | dilution | | 1 | 2 | 3 | 4 |
| FA1090: | Day 0 | 100 | A | 0.024 | 0.023 | 0.022 | 0.018 |
| [A-H; 1] | | | | | | | |
| MS11 (Opa-): | | 500 | B | 0.014 | 0.012 | 0.006 | 0.011 |
| [A-H; 2] | | | | | | | |
| F62 (ΔD): | | 5000 | C | 0.016 | 0.020 | 0.021 | 0.020 |
| [A-H; 3] | | | | | | | |
| H041: | | 25000 | D | 0.028 | 0.030 | 0.021 | 0.026 |
| [A-H; 4] | | | | | | | |
| | Day 60 | 100 | E | 0.150 | 0.152 | 0.143 | 0.235 |
| | | 500 | F | 0.025 | 0.035 | 0.030 | 0.040 |
| | | 5000 | G | 0.016 | 0.019 | 0.017 | 0.026 |
| | | 25000 | H | 0.013 | 0.017 | 0.007 | 0.017 |

The results provided above are summarized in the following table, where reference is made to the individual tables above. As appears strong antigen-specific antibody responses (“+++”) could be detected for a majority of the constructs, and all provided for antibody responses that recognized the 4 different whole cells tested:

| | | | | Antisera from Day 46 + 60 | | | | |
|---|---|---|---|---|---|---|---|---|
| | | | | Antibody Level range:[−, +, ++, +++] | | | | |
| | | | | ELISA | whole cell ELISA | | | |
| | Protein ID | | | | | | F62 | |
| | (strain | | | Recombinant | | MS11 | (delta | |
| Group | FA1090) | Contruct ID | Tables | Protein | FA1090 | (Opa-) | lgtD) | H041 |
| 1 | — | TMCP2 | tables (group 1, ELISA) | −* | + | + | + | − |
| 2 | NGO1496 | cNGO1496-1-693 | tables (group 2, ELISA) | +++ | ++ | ++ | +++ | ++ |
| | NGO0571 | cNGO0571-21-598 | | ++ | | | | |
| 3 | NGO1379 | NGO1379-28-283 | tables (group 3, ELISA) | +++ | +++ | +++ | + | + |
| | NGO0725 | cNGO0725-1-109 | | +++ | | | | |
| 4 | NGO1158 | NGO1158-27-422 | tables (group 4, ELISA) | +++ | ++ | ++ | − | − |
| | NGO0182 | NGO0182-26-228 | | +++ | | | | |
| 5 | NGO0721 | NGO0721-22-337 | tables (group 5, ELISA) | +++ | ++ | ++ | ++ | + |
| | NGO2105 | NGO2105-44-1468 | | +++ | | | | |
| 6 | NGO1094 | cNGO1094-1-398 | tables (group 6, ELISA) | ++ | + | + | + | + |
| | NGO1043 | NGO1043-22-114 | | − | | | | |
| | NGO2059 | NGO2059-22-522 | | ++ | | | | |
| 7 | NGO1984 | cNGO1984-59-216 | tables (group 7, ELISA) | + | +++ | +++ | +++ | +++ |
| | NGO1286 | cNGO1286-1-943 | | ++ | | | | |
| | NGO1092 | NGO1092-1-649 | | n/a** | | | | |
| 8 | NGO0275 | cNGO0275-28-1075 | tables (group 8, ELISA) | +++ | + | + | + | + |
| | NGO0225 | NGO0225-25-628 | | +++ | | | | |
| 9 | NGO1495 | cNGO1495-25-912 | tables (group 9, ELISA) | +++ | ++ | ++ | ++ | ++ |
| | NGO2093 | cNGO2093-23-720 | | +++ | | | | |
| 10 | NGO1392 | cNGO1392-28-439 | tables (group 10, ELISA) | +++ | + | + | + | + |
| | NGO1585 | cNGO1585-28-576 | | +++ | | | | |
| | NGO2109 | cNGO2109-23-809 | | + | | | | |
| 11 | NGO1801 | cNGO1801-22-792 | tables (group 11, ELISA) | +++ | + | + | + | + |
| | NGO0952 | cNGO0952-26-922 | | +++ | | | | |
| | NGO1715 | cNGO1715-25-801 | | +++ | | | | |
| 12 | NGO1549 | NGO1549-35-289 | tables (group 12, ELISA) | ++ | + | + | + | + |
| | NGO0265 | NGO0265-44-346 | | + | | | | |
| 13 | — | Control (GLA-SE) | tables (group 13, ELISA) | − | − | − | − | − |

*)TMCP2 peptide construct collapsed on ELISA plate, hence no result
**)No more protein for ELISA

Protection Against Challenge—Results

Results from the challenge experiments are summarized below:

| | | | PROTECTION (in vivo) | | | | | | |
|---|---|---|---|---|---|---|---|---|---|
| | | | Strain MS11 | | | | | | |
| | | | | | | | AUC (log10CFU) | | |
| | | | | | | | | | % Median |
| | | | % Infected | | | | | | Reduction |
| | | | Log-rank test | | Gehan-Breslow- | | Mann | | (Test |
| | | | (Mantel-Cox) | | Wilcoxon test | | Whitney test | | group |
| | | No of | | Significant | | Significant | | Significant | vs Cntrl |
| Group | Protein ID | Mice | P-value | (P < 0.05) | P-value | (P < 0.05) | P-value | (P < 0.05) | group) |
| 1 | TMCP2 | 5 | 0.002 | yes | 0.0039 | yes | 0.0079 | yes | 73% |
| 2 | NGO1496 | 5 | 0.0041 | yes | 0.006 | yes | 0.0079 | yes | 37% |
| | NGO0571 | | | | | | | | |
| 3 | NGO1379 | 5 | 0.7645 | no | 0.4446 | no | 0.0079 | yes | 26% |
| | NGO0725 | | | | | | | | |
| 4 | NGO1158 | 5 | 0.5485 | no | 0.6069 | no | 0.0159 | yes | 28% |
| | NGO0182 | | | | | | | | |
| 5 | NGO0721 | 5 | 0.1256 | no | 0.1133 | no | 0.0159 | yes | 27% |
| | NGO2105 | | | | | | | | |
| 6 | NGO1094 | 5 | 0.6135 | no | 0.6521 | no | 0.0317 | yes | 17% |
| | NGO1043 | | | | | | | | |
| | NGO2059 | | | | | | | | |
| 7 | NGO1984 | 5 | 0.7198 | no | 0.6513 | no | 0.2222 | yes | 19% |
| | NGO1286 | | | | | | | | |
| | NGO1092 | | | | | | | | |
| 8 | NGO0275 | 5 | 0.0579 | no | 0.0558 | no | 0.317 | yes | 24% |
| | NGO0225 | | | | | | | | |
| 9 | NGO1495 | 5 | 0.0035 | yes | 0.0039 | yes | 0.0079 | yes | 34% |
| | NGO2093 | | | | | | | | |
| 10 | NGO1392 | 5 | 0.2862 | no | 0.1486 | no | 0.0556 | no | 13% |
| | NGO1585 | | | | | | | | |
| | NGO2109 | | | | | | | | |
| 11 | NGO1801 | 5 | 0.2802 | no | 0.2888 | no | 0.0556 | no | 21% |
| | NGO0952 | | | | | | | | |
| | NGO1715 | | | | | | | | |
| 12 | NGO1549 | 5 | 0.6992 | no | 0.2722 | no | 0.0159 | yes | 37% |
| | NGO0265 | | | | | | | | |
| | | | PROTECTION (in vivo) | | | | | | |
| | | | Strain H041 | | | | | | |
| | | | | | | | AUC (log10CFU) | | |
| | | | | | | | | | % Median |
| | | | % Infected | | | | | | Reduction |
| | | | Log-rank test | | Gehan-Breslow- | | Mann | | (Test |
| | | | (Mantel-Cox) | | Wilcoxon test | | Whitney test | | group |
| | | No of | | Significant | | Significant | | Significant | vs Cntrl |
| Group | Protein ID | Mice | P-value | (P < 0.05) | P-value | (P < 0.05) | P-value | (P < 0.05) | group) |
| 1 | TMCP2 | 5 | 0.0031 | yes | 0.0043 | yes | 0.2222 | no | 18% |
| 2 | NGO1496 | 5 | 0.2966 | no | 0.2074 | no | 0.2222 | no | 16% |
| | NGO0571 | | | | | | | | |
| 3 | NGO1379 | 5 | 0.0771 | no | 0.0372 | yes | 0.5476 | no | 14% |
| | NGO0725 | | | | | | | | |
| 4 | NGO1158 | 5 | 0.0637 | no | 0.0417 | yes | 0.4206 | no | 3% |
| | NGO0182 | | | | | | | | |
| 5 | NGO0721 | 5 | 0.8527 | no | 0.8111 | no | 0.5476 | no | −8% |
| | NGO2105 | | | | | | | | |
| 6 | NGO1094 | 5 | 0.1297 | no | 0.1146 | no | 0.8413 | no | 2% |
| | NGO1043 | | | | | | | | |
| | NGO2059 | | | | | | | | |
| 7 | NGO1984 | 5 | 0.0771 | no | 0.0372 | yes | 0.2222 | no | 29% |
| | NGO1286 | | | | | | | | |
| | NGO1092 | | | | | | | | |
| 8 | NGO0275 | 5 | 0.0291 | yes | 0.0305 | yes | 0.6905 | no | 7% |
| | NGO0225 | | | | | | | | |
| 9 | NGO1495 | 5 | 0.3038 | no | 0.209 | no | 0.2222 | no | 13% |
| | NGO2093 | | | | | | | | |
| 10 | NGO1392 | 5 | 0.0291 | yes | 0.0305 | yes | 0.2222 | no | 16% |
| | NGO1585 | | | | | | | | |
| | NGO2109 | | | | | | | | |
| 11 | NGO1801 | 5 | 0.438 | no | 0.4189 | no | 0.5476 | no | 24% |
| | NGO0952 | | | | | | | | |
| | NGO1715 | | | | | | | | |
| 12 | NGO1549 | 5 | 0.0025 | yes | 0.0042 | yes | 0.0079 | yes | 53% |
| | NGO0265 | | | | | | | | |

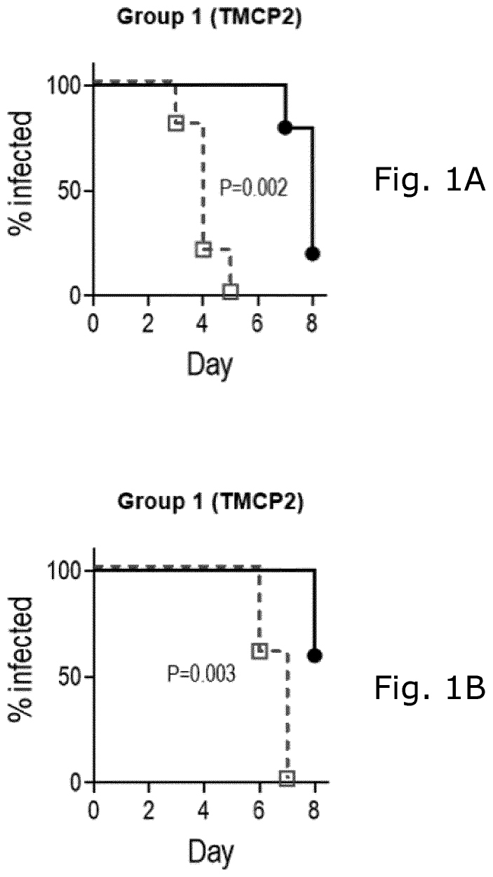

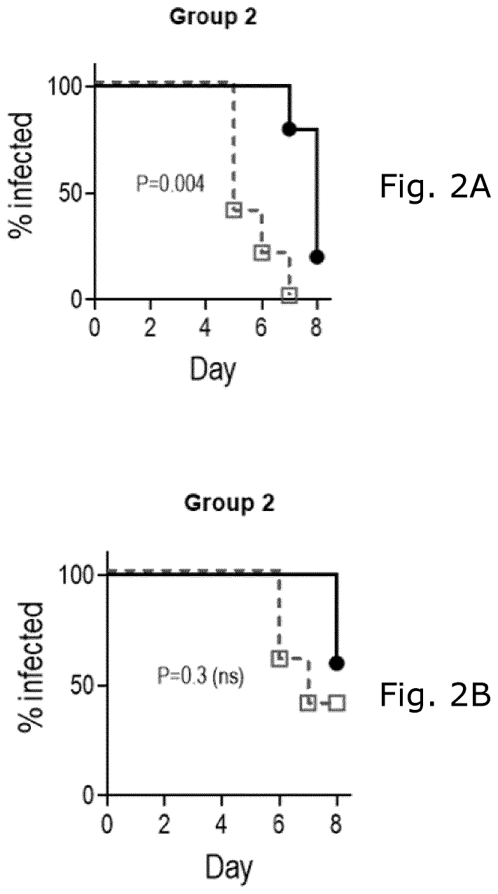

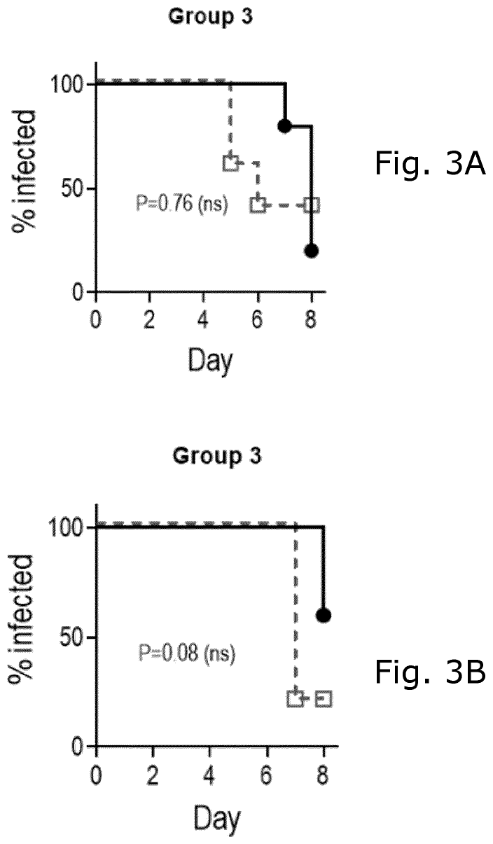

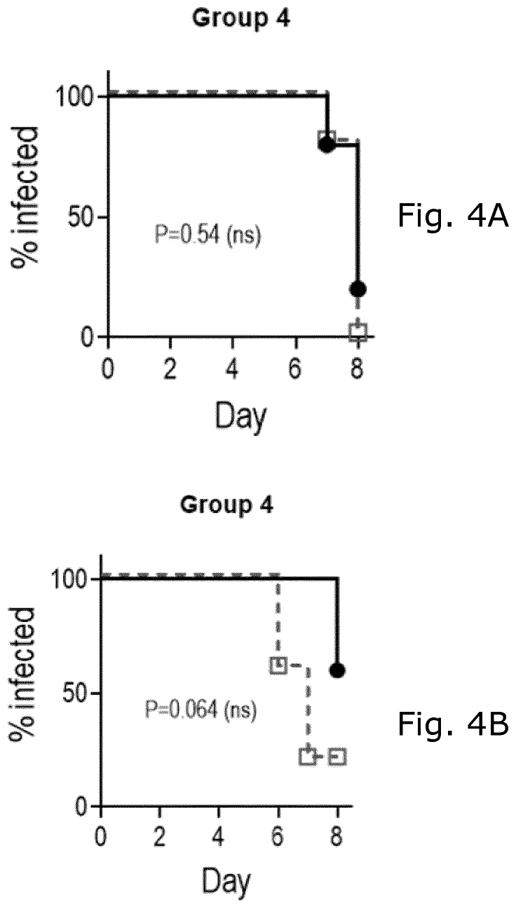

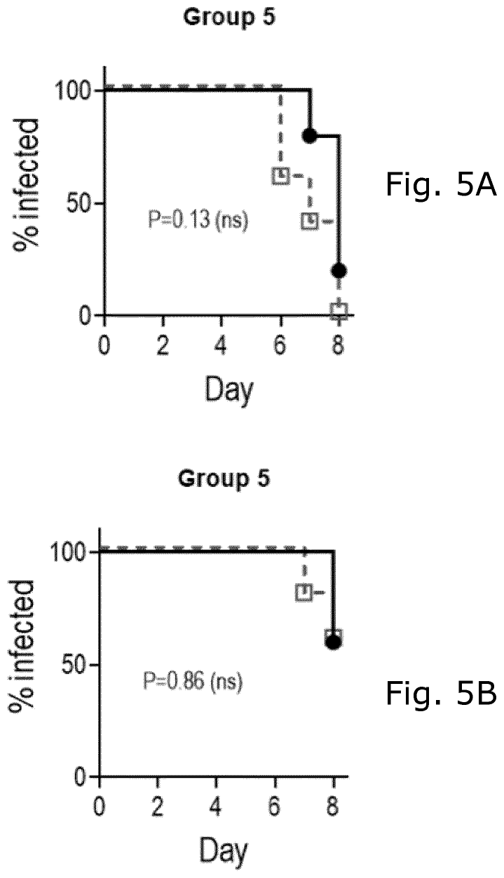

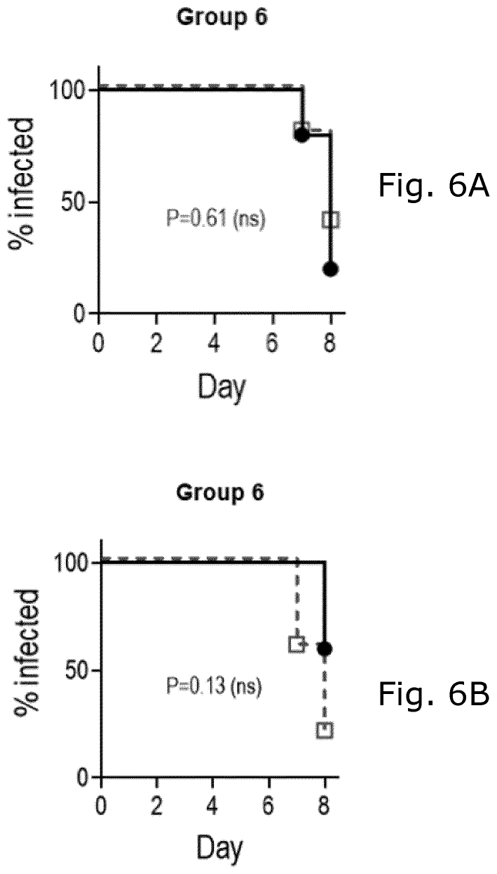

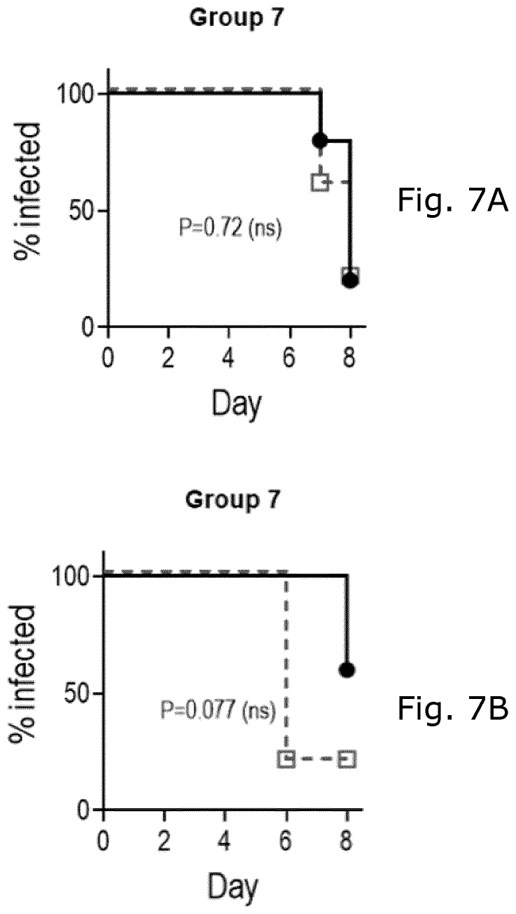

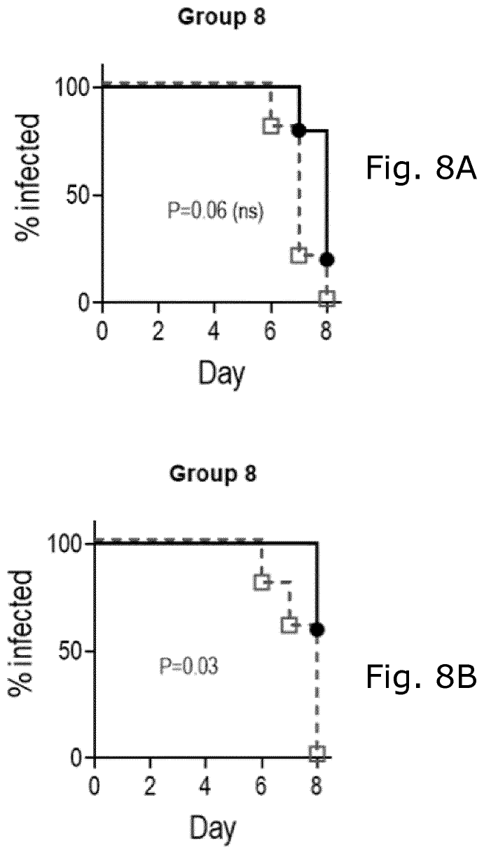

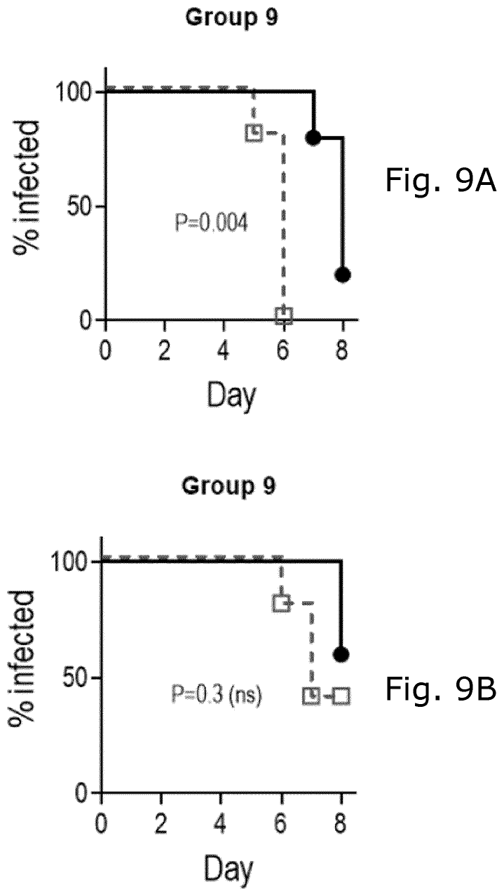

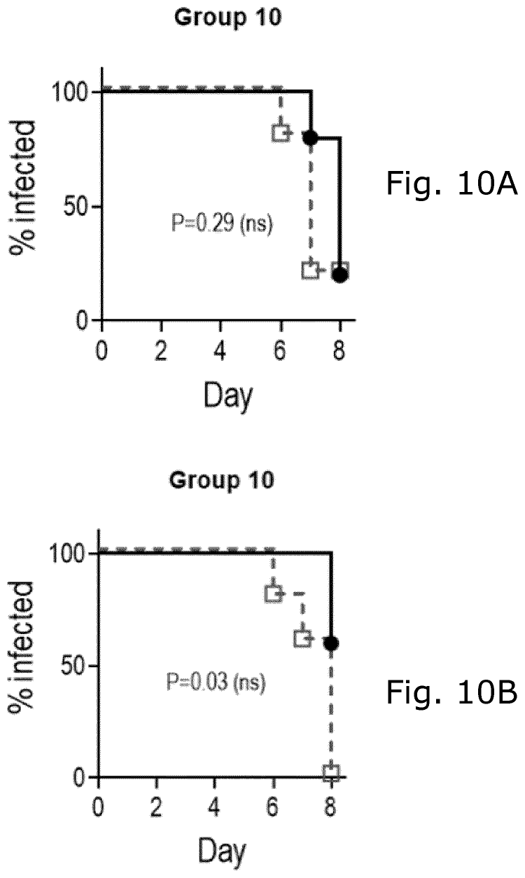

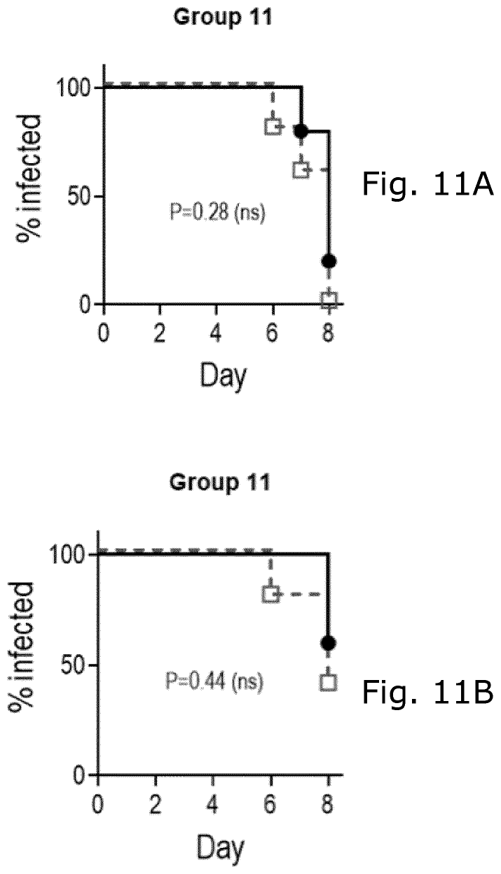

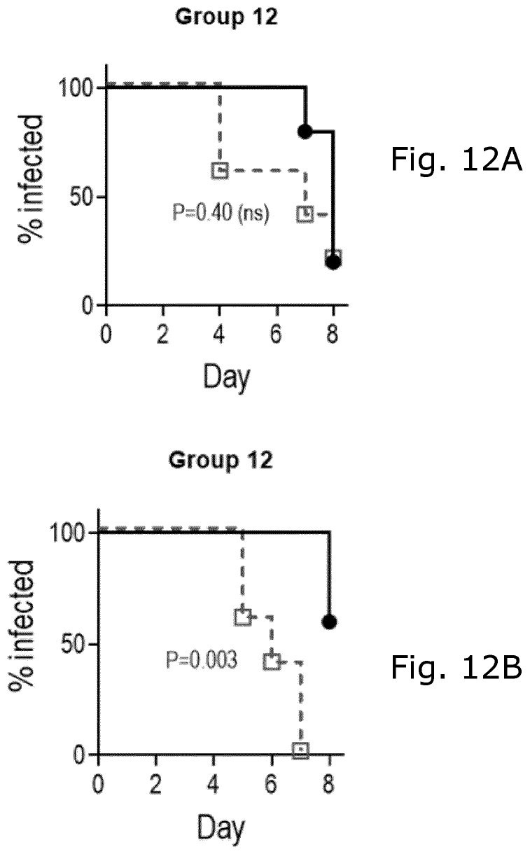

Data are also presented in FIGS. 1-12, which show Kaplan-Meyer plots of bacterial clearance in vaccinated mice from the 12 groups compared to clearance rates in mice receiving adjuvant only.

### Example 2

Challenge Experiment, FtSN Proteins (Single and in Combination)

Bacterial strains: N. gonorrhoeae strains FA1090 and MS11.

Immunization of mice: Six-week-old female BALB/c mice were immunized intramuscularly (IM) with a recombinant NeGo protein (15 μg) and adjuvant GLA-SE (5 μg), with a combo of two recombinant NeGo proteins (15 μg each) and GLA-SE (5 μg) or with positive control TMCP2 [0] (50 μg) and adjuvant GLA-SE (5 μg). Control mice received GLA-SE (5 μg) adjuvant alone. Mice were immunized by schedule: Primary immunization (day 0) and boosts (day 14 and 28).

Compositions of the test vaccines and the controls are provided in the following table:

| | | | | | | | | Dose of | |
|---|---|---|---|---|---|---|---|---|---|
| | | | | | | | | Adjuvant | |
| | Protein ID | | | | | Construct | | formulation | |
| | (strain | Amount | | | | Length | | per animal | No of |
| Group | FA1090) | (ug) | Solublity | Formulation | Contruct ID | (AA) | Adjuvant | (μg) | Mice |
| 1 | — | 50 | — | — | TMCP2 | — | GLA-SE | 5 | 10 |
| 2 | NGO1549 | 15 | soluble | NaCl | NGO1549-35-289 | 255 | GLA-SE | 5 | 10 |
| 3 | NGO0265 | 15 | soluble | NaCl | NGO0265-44-346 | 303 | GLA-SE | 5 | 10 |
| 4 | NGO1549 | 15 | soluble | NaCl | NGO1549-35-289 | 255 | GLA-SE | 5 | 10 |
| | NGO0265 | 15 | soluble | NaCl | NGO0265-44-346 | 303 | | | |
| 5 | — | — | — | — | Control (adjuvant) | — | GLA-SE | 5 | 10 |

Infection of mice: Mice were infected on day 42 post first immunization.

Mouse protection experiments: Use of animals in this study was performed in strict accordance with the recommendations in the Guide for the Care and Use of Laboratory Animals of the National Institutes of Health 2011. The protocol was approved by the Institutional Animal Care and Use Committee (IACUC) at the University of Massachusetts Medical School. The BALB/c mouse model of vaginal colonization described by Jerse 1999 was used. Two weeks after the last immunization, mice in the diestrus phase of the estrous cycle were started on treatment (that day) with 0.1 mg Premarin (Pfizer) in 200 μl of water, given subcutaneously on each of 3 days: days 55, 57, and 59 (before, the day of, and after gonococcal inoculation) to prolong the estrus phase of the reproductive cycle and promote susceptibility to N. gonorrhoeae infection. Antibiotics (vancomycin and streptomycin) ineffective against N. gonorrhoeae were also used to reduce competitive microflora (cf. Jerse et al. 2011). Immunized mice and placebo control mice were infected on day 42 with either strain MS11 (inoculum dose: 2.8×10⁷ CFU) or FA1090 (inoculum dose: 3.6×10⁷ CFU). Vaginas were swabbed daily to enumerate CFUs. Efficacy of the vaccine groups were measured using: i) time to clearance of infection, ii) log 10 CFU vs time and iii) Area Under curve analysis.

Statistical analyses: Experiments that compared clearance of N. gonorrhoeae in independent groups of mice estimated and tested three characteristics of the data (Gulati et al. 2013): time to clearance; longitudinal trends in mean log 10 CFU and the cumulative CFU as area under the concentration-time curve (AUC). Statistical analyses were performed using mice that initially yielded bacterial colonies on day 1 and/or 2 (Gulati et al. 2019). Median time to clearance was estimated using Kaplan-Meier survival curves; times to clearance were compared between groups using the Mantel-Cox log-rank test and Gehan-Breslow-Wilcoxon test. The mean AUC (log 10 CFU versus time) was computed for each mouse to estimate the bacterial burden over time (cumulative infection); the means under the curves were compared between groups using the nonparametric two-sample Wilcoxon rank-sum (Mann-Whitney) test because distributions were skewed or kurtotic. The median AUC (log 10 CFU versus time) percent reduction (test group vs placebo control group) were calculated

Results

Results from the challenge experiments are summarized in the table below. As is evident vaccination with NGO1549 and NGO0265 (alone as well as when combined) provided for significant protection against NeGo challenge infection with the 2 strains MS11 and FA1090; most strikingly when evaluating AUC (log 10 CFU).

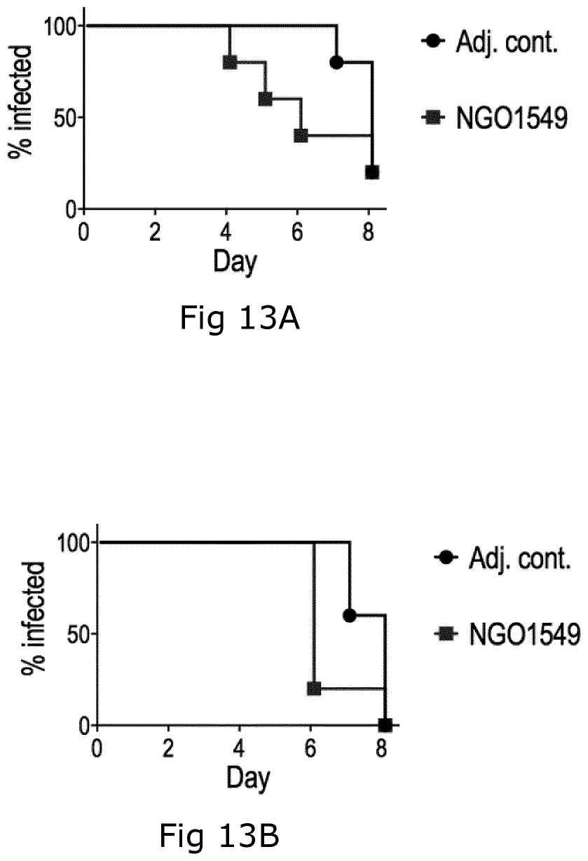

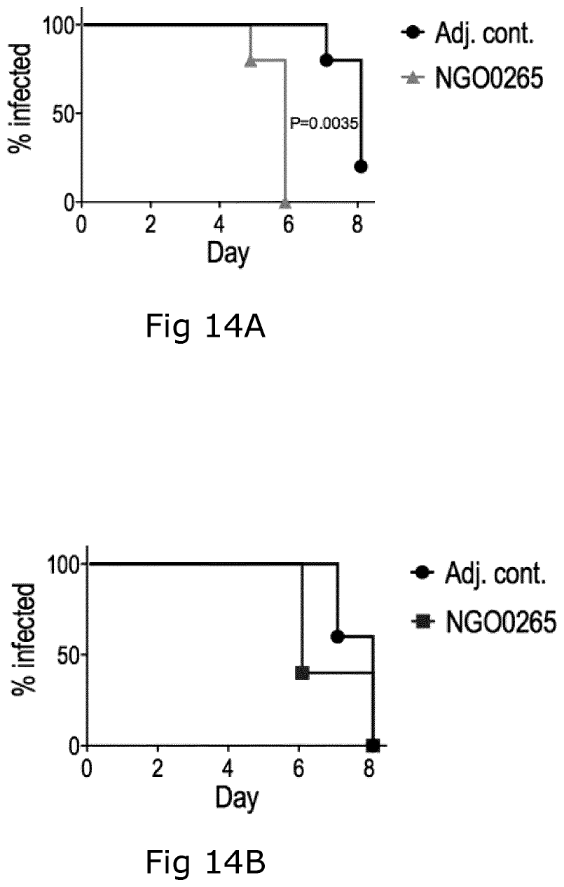

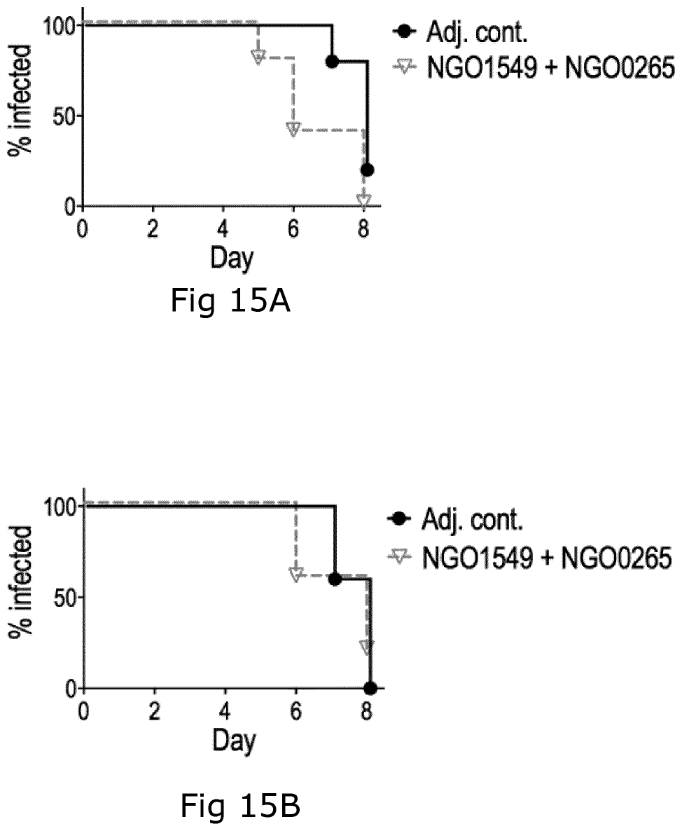

The time to clearance data for the mice in Groups 2-4 are shown as Kaplan-Meyer plots in FIGS. 13-15. Due to the small number of experimental animals in each vaccinated group, not all experiments with the proteins disclosed herein provided for significant protection in terms of faster clearance rate, but as is evident from the Kaplan-Meyer plots, all the vaccinated animals cleared the bacteria faster than animals in group 5 (negative control).

| | | | PROTECTION (in vivo) | | | | | | |
|---|---|---|---|---|---|---|---|---|---|
| | | | Strain MS11 | | | | | | |
| | | | | | | | AUC (log10CFU) | | |
| | | | | | | | | | % Median |
| | | | % Infected | | | | | | Reduction |
| | | | Log-rank test | | Gehan-Breslow- | | Mann | | (Test |
| | | | (Mantel-Cox) | | Wilcoxon test | | Whitney Test | | group |
| | | No of | | Signficant | | Signficant | | Signficant | vs Ctrl |
| Group | Protein ID | Mice | P-value | (P < 0.05) | P-value | (P < 0.05) | P-value | (P < 0.05) | group) |
| 1 | TMCP2 | 5 | 0.0052 | yes | 0.0066 | yes | 0.0079 | yes | 71% |
| 2 | NGO1549 | 5 | 0.3502 | no | 0.2267 | no | 0.0159 | yes | 53% |
| 3 | NGO0265 | 5 | 0.0035 | yes | 0.0039 | yes | 0.0079 | yes | 49% |
| 4 | NGO1549 | 5 | 0.1151 | no | 0.0894 | no | 0.0079 | yes | 49% |
| | NGO0265 | | | | | | | | |
| | | | PROTECTION (in vivo) | | | | | | |
| | | | Strain FA1090 | | | | | | |
| | | | | | | | AUC (log10CFU) | | |
| | | | | | | | | | % Median |
| | | | % Infected | | | | | | Reduction |
| | | | Log-rank test | | Gehan-Breslow- | | Mann | | (Test |
| | | | (Mantel-Cox) | | Wilcoxon test | | Whitney Test | | group |
| | | No of | | Signficant | | Signficant | | Signficant | vs Ctrl |
| Group | Protein ID | Mice | P-value | (P < 0.05) | P-value | (P < 0.05) | P-value | (P < 0.05) | group) |
| 1 | TMCP2 | 5 | 0.0023 | yes | 0.0039 | yes | 0.0079 | yes | 71% |
| 2 | NGO1549 | 5 | 0.0771 | no | 0.0372 | yes | 0.0079 | yes | 29% |
| 3 | NGO0265 | 5 | 0.3339 | no | 0.204 | no | 0.0317 | yes | 27% |
| 4 | NGO1549 | 5 | 0.8113 | no | 0.9105 | no | 0.0079 | yes | 29% |
| | NGO0265 | | | | | | | | |

### Example 3

Bactericidal Test

Bacterial strains: N. gonorrhoeae strains FA1090, MS11 (Opa-), F62(ΔIgtD) and H041.

Immunization and bleeding of mice: Six-week-old female BALB/c mice were immunized intramuscularly (IM) with a recombinant NeGo protein (15 μg) and adjuvant GLA-SE (5 μg). Positive control protein NGO1363 (MtrE) and TMCP2 (50 μg) were used based on published bactericidal effect (cf. Rice et al. 2017 and Gulati et al. 2019). Control mice received GLA-SE (5 μg) adjuvant alone. Mice were immunized by schedule: Primary immunization (week 0) and boosts (week 2 and 4). Bled of mice in week 6.

The mice groups were immunized with the following antigens and controls:

| | | | | | | | Dose of | |
|---|---|---|---|---|---|---|---|---|
| | | | | | | | Adjuvant | |
| | | | | Construct | | | formulation | |
| | | | | Length | | Amount | per animal | No of |
| Groups | Contruct ID | Solublity | Formulation | (AA) | Adjuvant | (ug) | (μg) | Mice |
| NGO0571 | cNGO0571-21-598 | soluble | NaCl | 578 | GLA-SE | 15 | 5 | 5 |
| NGO1549 | NGO1549-35-289 | soluble | NaCl | 255 | GLA-SE | 15 | 5 | 5 |
| NGO1379 | NGO1379-28-283 | soluble | NaCl | 256 | GLA-SE | 15 | 5 | 5 |
| NGO0265 | NGO0265-44-346 | soluble | NaCl | 303 | GLA-SE | 15 | 5 | 5 |
| NGO1958 | cNGO1958-20-426 | soluble | NaCl | 407 | GLA-SE | 15 | 5 | 5 |
| NGO1495 | cNGO1495-25-912 | unsoluble | urea | 888 | GLA-SE | 15 | 5 | 5 |
| NGO1363 | cNGO1363-23-467 | soluble | NaCl | 445 | GLA-SE | 15 | 5 | 5 |
| (positive control) | | | | | | | | |
| Control (adjuvant) | Control (adjuvant) | — | — | — | GLA-SE | — | 5 | 5 |
| TMCP2 | TMCP2 | — | — | — | GLA-SE | 50 | 5 | 11 |

Serum bactericidal assays: Serum bactericidal assays were performed as described previously (cf. Gulati et al. 2012). Bacteria that had been harvested from an overnight culture on chocolate agar plates were passaged again onto fresh chocolate agar and allowed to grow for 6 h at 37° C. in an atmosphere containing 5% CO2. Bacteria were then suspended in Hanks' balanced salt solution (HBSS) containing 1 mM MgCl₂ and 0.15 mM CaCl₂ (HBSS++) for use in serum bactericidal assays. About 2,000 CFU was incubated with serial dilutions of immune mouse sera (heat-inactivated and IgM depleted) in the presence or absence of 20% normal human serum (NHS) as a source of human complement. Serum bactericidal assays with strain MS11 were performed with IgG- and IgM-depleted NHS (human complement; Pel-Freez) because MS11 is susceptible to killing by NHS. The final concentration of mouse serum used in the assay was 67% (50 μl of immune serum in a final reaction volume of 80 μl). Complement source: Normal human serum depleted of IgG and IgM (Pel-Freez); 25% complement used with FA1090; 12.5% complement used for MS11, F62 and H041. Aliquots of 25 μl reaction mixtures were plated onto chocolate agar in duplicate at the beginning of the assay (time zero [t0]) and after incubation at 37° C. for 30 min (t30). Survival was calculated as the number of viable colonies at t30 relative to to.

Results

Survival rates in bacterial colonies in the above described assay were as follows:

| | | | Pooled Antisera | | | |
|---|---|---|---|---|---|---|
| | | | from week 6 | | | |
| | | No of | % Survival | | | |
| Group | Contuct ID | Mice | FA1090 | MS11 | F62 | H041 |
| NGO0571 | cNGO0571-21-598 | 5 | 112 | 4 | 114 | 113 |
| NGO1549 | NGO1549-35-289 | 5 | 49 | 0 | 73 | 18 |
| NGO1379 | NGO1379-28-283 | 5 | 117 | 0 | 119 | 114 |
| NGO0265 | NGO0265-44-346 | 5 | 63 | 0 | 84 | 1 |
| NGO1958 | cNGO1958-20-426 | 5 | 117 | 0 | 114 | 19 |
| NGO1495 | cNGO1495-25-912 | 5 | 112 | 0 | 110 | 109 |
| NGO1363 | cNGO1363-23-467 | 5 | 120 | 0 | 114 | 37 |
| (positive control) | | | | | | |
| Control (adjuvant) | — | 5 | 125 | 122 | 114 | 121 |
| Control (no serum) | — | 5 | 121 | 109 | 120 | 117 |

As apparent, pooled sera from mice immunized with several of the constructs (notably NGO1549-35-289 and NGO0265-44-346) were capable of reducing bacterial survival to a significant degree. In comparison, sera from mice immunized with TMCP2, provided for a mean survival rate of FA1090 in the same assay of 46.8%.

To compare, the antibody sera induced in the 9 different groups exhibited the following titres against the immunogen and FA1090:

| | | \|Antisera from week 6 | |
|---|---|---|---|
| | | Antibody Level | |
| | | range: [−, +, ++, +++] | |
| | | ELISA | whole cell |
| | | Recombinant | ELISA |
| Group | Contract ID | Protein | FA1090 |
| NGO0571 | cNGO0571-21-598 | +++ | + |
| NGO1549 | NGO1549-35-289 | +++ | ++ |
| NGO1379 | NGO1379-28-283 | ++ | − |
| NGO0265 | NGO0265-44-346 | + | ++ |
| NGO1958 | cNGO1958-20-426 | ++ | ++ |
| NGO1495 | cNGO1495-25-912 | +++ | +++ |
| NGO1363 (positive | cNGO1363-23-467 | +++ | − |
| control) | | | |
| Control (adjuvant) | Control (adjuvant) | − | − |
| TMCP2 (positive control) | TMCP2 | ++* | n/a |
| Control (adjuvant) | Control (adjuvant) | − | n/a |

*plate coated with lipooligosaccharide 15253 LOS

### LIST OF REFERENCES RELEVANT FOR EXAMPLES

1. Gulati S at al. 2019, Preclinical efficacy of a lipooligosaccharide peptide mimic candidate gonococcal vaccine. mBio.02552-19.
2. Gulati S et al. 2013, Immunization against a saccharide epitope accelerates clearance of experimental gonococcal infection. PLoS Pathog 9:e1003559.
3. National Research Council 2011, Guide for the care and use of laboratory animals, 8th ed. National Academies Press, Washington, D.C.
4. Jerse A E 1999, Experimental gonococcal genital tract infection and opacity protein expression in estradiol-treated mice. Infect Immun 67: 5699-5708.
5. Jerse A E et al. 2011, Estradiol-treated female mice as surrogate hosts for Neisseria gonorrhoeae genital tract infections. Front Microbiol 2: 107.
6. Gulati S et al., 2012. Properdin is critical for antibody-dependent bactericidal activity against Neisseria gonorrhoeae that recruit C4b-binding protein. J Immunol 188: 3416-3425.
7. Rice P A, et al., 2017. Annu Rev Microbiol.; 71:665-686. doi: 10.1146/annurev-micro-090816-093530.

1. A pharmaceutical composition comprising a polypeptide comprising
a) an amino acid sequence selected from the group consisting of any one of SEQ ID NOs: 8, 1-7, and 9-35, or
b) an amino acid sequence consisting of at least or exactly 35 contiguous amino acid residues from any one of SEQ ID NOs: 8, 1-7, and 9-35, or
c) an amino acid sequence having a sequence identity of at least 60% with the amino acid sequence of a) or b),
said polypeptide being antigenic in a mammal; and
an immunological adjuvant and a pharmaceutically acceptable carrier, vehicle or diluent.

2. The pharmaceutical composition according to claim 1, wherein the at least or exactly 35 contiguous amino acids are at least or exactly or at most 36, at least or exactly or at most 37, at least or exactly or at most 38, at least or exactly or at most 39, at least or exactly or at most 30, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, or at least or exactly or at most 52 contiguous amino acid residues.

3. The polypeptide according to claim 1, wherein the sequence identity with the amino acid sequence of a) or b), which is defined in c), is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

4. (canceled)

5. The polypeptide according to claim 1, wherein the at least 5 contiguous amino acid residues has an N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49 in any one of SEQ ID NOs: 1-35, or
any one of amino acid residues 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 97 and 98 in any one of SEQ ID NOs: 2-35, or
any one of amino acid residues 99, 100, 101, 102, 103, 104, and 105 in any one of SEQ ID NOs: 3-35, or
any one of amino acid residues 106, 107, 108, 109, and 110 in any one of SEQ ID NOs: 4-35, or
any one of amino acid residues 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, and 212 in any one of SEQ ID NOs: 5-53, or
an one of amino acid residues 213, 214, 215, 216, 217, 218, 219, 220, 221, 222, 223, and 224 in an one of SEQ ID NOs: 6-35, or
any one of amino acid residues 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, and 279 in an one of SEQ ID NOs: 7-35, or
any one of amino acid residues, 280, 281, 282, 283, 284, and 285 in any one of SEQ ID NOs: 8-35, or
any one of amino acid residues 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, and 333 in any one of SEQ ID NOs: 9-35, or
any one of amino acid residues 334, 335, 336, 337, 338, 339, 340, 341 and 342 in any one of SEQ ID NOs: 10-35, or
any one of amino acid residues 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, and 373 in any one of SEQ ID NOs: 11-35, or
any one of amino acid residues 374, 375, 376, 377, 378, 379, 380, 381, 382, 383, 384, 385, 386, 387, 388, 389, 390, 391, 392, 393, and 394 in any one of SEQ ID NOs: 12-35, or
any one of amino acid residues 395, 396, 397, 398, 399, 400, 401, 402, 403, 404, 405, 406, 407, 408, 409, 410, 411, 412, 413, 414, 415, 416, 417, and 418 in any one of SEQ ID NOs: 13-35, or
any one of amino acid residues 419, 420, 421, and 422 in any one of SEQ ID NOs: 14-35, or
any one of amino acid residues 423, 424, 425, 426, 427, 428, 429, 430, 431, 432, 433, 434, and 435 in any one of SEQ ID NOs: 15-35, or
any one of amino acid residues 436, 437, 438, 439, 440, 441, 442, 443, 444, 445, 446, 447, 448, 449, 450, 452, 453, 454, 455, 456, 457, 458, 459, 460, 461, 462, 463, and 464 in any one of SEQ ID NOs; 16-35, or
any one of amino acid residues 465, 466, 467, 468, 469, 470, 471, 472, 473, 474, 475, 476, 477, 478, 479, 480, 481, 482, 483, 484, 485, 486, 487, 488, 489, 490, 491, 492, 493, and 494 in any one of SEQ ID NOs: 17-35, or
any one of amino acid residues 495, 496, 497, 498, 499, 500, 501, 502, 503, 504, 505, 506, 507, 508, 509, 510, 511, 512, 513, 514, 515, 516, 517, and 518 in any one of SEQ ID NOs: 18-35, or
any one of amino acid residues 519, 520, 521, 522, and 523 in any one of SEQ ID NOs: 19-35, or any one of amino acid residues 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 571, and 572 in any one of SEQ ID NOs: 20-35, or
any one of amino acid residues 573, 574, 575, 576, 577, 578, 579, 580, 581, 582, 583, 584, 585, 586, 587, 588, 589, 590, 591, 592, 593, and 594 in any one of SEQ ID NOs: 21-35, or
any one of amino acid residues 595, 596, 597, 598, 599, 600, 601, 602, 603, 604, 605, 606, 607, 608, 609, 610, 611, 612, 613, 614, 615, 616, 617, 618, 619, 620, 621, 622, 623, and 624 in an one of SEQ ID NOs: 22-35, or
any one of amino acid residues 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, and 689 in any one of SEQ ID NOs 23-35, or
any one of amino acid residues 690, 691, 692, 693, 694, 695, 696, 697, 698, 699, 700, 701, 702, 703, 704, 705, 706, 707, 708, 709, 710, 711, 712, 713, 714, 715, and 716 in any one of SEQ ID NOs: 24-35, or
any one of amino acid residues 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, and 788 in any one of SEQ ID NOs: 25-35, or
any one of amino acid residues 789, 790, 791, 792, 793, 794, 795, 796, and 797 in any one of SEO ID NOs: 26-35, or
any one of amino acid residues 798, 799, 800, 801, 802, 803, 804, and 805 in any one of SEQ ID NOs: 27-35, or
any one of amino acid residues 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, and 908 in any one of SEQ ID NOs: 28-35, or
any one of amino acid residues 909, 910, 911, 912, 913, 914, and 915 in any one of SEQ ID NOs: 29-35, or
any one of amino acid residues 916, 917, and 918 in any one of SEQ ID NOs: 30-35, or
any one of amino acid residues 919, 920, 921, 922, 923, 924, 925, 926, 927, 928, 929, 930, 931, 932, 933, 934, 935, 936, 937, 938, and 939 in any one of SEQ ID NOs: 31-35, or
any one of amino acid residues 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, and 1010 in any one of SEQ ID NOs: 32-35, or
an one of amino acid residues 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, and 1071 in any one of SEQ ID NOs: 33-35, or
any one of amino acid residues 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, and 1464 in SEQ ID NO: 34 or 35, or
any one of amino acid residues 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 41523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 17724, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1801, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, and 1973 in SEQ ID NO: 35,
with the proviso that the selected amino acid residue satisfies the formula N≤L−n+1, where N is the number of the selected residue, L is the number of amino acid residues in the sequence from which the residue is selected, and n is the number of consecutive amino acid residues.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The pharmaceutical composition according to claim 1, wherein the polypeptide
is fused or conjugated to an immunogenic carrier molecule, and/or
is fused or conjugated to a different polypeptide according to any one of the preceding claims, preferably derived from a different SEQ ID NO.

41. The pharmaceutical composition according to claim 1, wherein the immunogenic carrier molecule is a polypeptide that induces T-helper lymphocyte responses in a majority of humans, such as immunogenic carrier proteins selected from the group consisting of keyhole limpet hemocyanin or a fragment thereof, tetanus toxoid or a fragment thereof, dipththeria toxoid or a fragment thereof.

42. The pharmaceutical composition according to claim 1, wherein the polypeptide is capable of inducing an adaptive immune response against the polypeptide in a mammal, in particular in a human being.

43. The pharmaceutical composition according to claim 42, wherein the polypeptide is capable of inducing, in the mammal, a protective adaptive immune response against infection with NeGo.

44. The pharmaceutical composition according to claim 42, which induces a humoral and/or a cellular immune response.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. A method for inducing immunity in an animal by administering at least once an immunogenically effective amount of a pharmaceutical composition according to claim 1 so as to induce adaptive immunity against NeGo in the animal.

71. The method according to claim 70, wherein, the animal receives between 0.5 and 5,000 μg of the polypeptide defined in claim 1.

72. The method according to claim 70, wherein the animal receives a priming administration and one or more booster administrations.

73. The method according to claim 70, wherein the animal is a human being.

74. The method according to claim 70, wherein the administration is for the purpose of inducing protective immunity against NeGo.

75. The method according to claim 74, wherein the protective immunity is effective in reducing the risk of contracting infection with NeGo or is effective in treating or ameliorating infection with NeGo.

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)
